(19) United States
(12) Patent Application Publication
(10) Pub. No.: US 2026/0201398 A1
(43) Pub. Date: Jul. 16, 2026

(54) MODIFIED PLANTS AND PLANT CELLS

(71) Applicant: NO REGRETS 2050 LTD., London (GB)

(72) Inventors: Christopher Peacock, London (GB); Simon Kirkham, London (GB)

(73) Assignee: NO REGRETS 2050 LTD., London (GB)

(21) Appl. No.: 19/132,561

(22) PCT Filed: Nov. 27, 2023
(86) PCT No.: PCT/EP2023/083234

§ 371 Date: May 23, 2025

(30) Foreign Application Priority Data
Nov. 25, 2022 (GB) 2217740.6

(51) Int. Cl. C12N 15/82; A23K 10/30; A23K 20/189; C12N 9/08

(52) U.S. Cl. CPC C12N 15/8257; A23K 10/30; A23K 20/189; C12N 9/0065; C07K 2319/08; C12Y 111/01018

(57) ABSTRACT

A nucleic acid construct for inducing expression of a polypeptide in a plant cell, the nucleic acid construct comprising a coding sequence encoding a polypeptide comprising a vanadate-dependent haloperoxidase (VHPO), or a functional fragment or homologue thereof; wherein the coding sequence is operably linked to one or more regulatory elements, suitable to drive expression of the polypeptide in a plant cell.

### CROSS-REFERENCE TO RELATED APPLICATIONS

This application is for entry into the U.S. National Phase from which priority is claimed under all applicable sections of Title 35 of the United States Code including, but not limited to, Sections 120, 363, and 365(c) to International Application Serial No. PCT/EP2023/083234, filed on Nov. 27, 2023 and entitled “MODIFIED PLANTS AND PLANT CELLS,” which in turn claims priority to UK Patent Application Serial No. GB2217740.6, filed on Nov. 25, 2022 and entitled “MODIFIED PLANTS AND PLANT CELLS.” Each of International Application Serial No. PCT/EP2023/083234 and UK Patent Application Serial No. GB2217740.6 is incorporated by reference herein in its entirety.

### REFERENCE TO SEQUENCE LISTING

This specification includes a sequence listing submitted herewith, which includes the file entitled 1644-001USU1_Sequence_Listing.xml having the following size: 200,509 bytes which was created on Nov. 24, 2023, the contents of which are incorporated by reference herein.

### FIELD OF THE INVENTION

The present invention relates to modified plants, methods of preparing same, and methods of reducing methane emissions in animals, in particular in ruminant livestock.

### BACKGROUND

Ruminants are animals with multiple stomach compartments and include cows, sheep, goats, buffalo, deer, elk, giraffes and camels. While ruminants are important for ecological as well as human economic and cultural reasons, they are also associated with a number of environmental and economic difficulties.

Firstly, ruminants emit methane, a potent greenhouse gas, primarily via belches when digesting food during enteric fermentation, and also via excretion. This contributes towards climate change, with 6% of global greenhouse emissions estimated to result from ruminant methane emissions. It is also thought that methane contributes to the formation of ground level ozone, which is harmful both to humans, causing lung illnesses and deaths, and to ecology, inhibiting and destroying plants and crops.

Secondly, ruminants have a relatively low feed efficiency rate, due in part to the energy ruminants expend on belching. This results in ruminants requiring a lot of land space for crops grown to feed them and for grazing. Specifically, ruminants require approximately 30% of the earth's habitable land, which in the context of a growing global population places a growing pressure on land use. This can affect the sustenance of farmers, particularly in developing economies

Existing approaches to reducing ruminant methane emissions and land use requirements are limited in terms of worldwide scalability, effectiveness and financial viability for farmers. Such approaches include changing farming systems to improve feed efficiency and manure management, which is labour- and resource-intensive, and requires training, adoption by farmers and consistency in implementing; direct modification of the ruminants themselves through breeding and/or genetic modification; and vaccine-type treatments to generate antibodies in the saliva that suppress the growth of methanogens in the rumen.

In Winichayakul et al “In vitro gas production and rumen fermentation profile of fresh and ensiled genetically modified high-metabolizable energy ryegrass”, Journal of Dairy Science, 2020, perennial ryegrass plants were genetically modified to increase leaf lipid content, leading to a decrease in methane proportion of the total gas production. However, the effects on plant viability and growth are not known.

Other approaches involve changing of ruminant diets through feed supplements, which can include methane-reducing inhibitor chemical compounds. Some of these approaches involve the chemical properties of certain seaweed species.

Studies have shown that red macroalgae Asparagopsis seaweed can reduce methane emissions and increase feed efficiency substantially when a small amount is added as a supplement (Abbott et al “Seaweed and Seaweed Bioactives for Mitigation of Enteric Methane: Challenges and Opportunities” Animals 2020). WO2021022341A1, WO2018018062A1, WO2020113279A1 and WO2020124167A1 describe the use of Asparagopsis seaweed for use as an animal feed supplement to reduce methane emissions and boost immunity. However, it has also been identified that application of these inventions requires daily feeding of the supplements to ruminants, which is resource intensive and not practical for many farmers, especially if ruminants are grazing. Furthermore, growing Asparagopsis seaweed at scale can be difficult and potentially very damaging to surrounding ecosystems, while the processing and transportation of such seaweed and downstream products can also have environmental impacts.

WO2020243792A1 discusses a genetically modified yeast for use as a component of animal feed. The yeast are used to produce bromoform, which is thought to reduce methane production when consumed by ruminants. The application of this invention requires effort from farmers and feed crop suppliers in the addition of the yeast to the feed, separate production and quality control of the produced yeast, and monitoring of appropriate levels of the additive.

WO2020210074A1 discuss treating pasture with beneficial microbes and/or their growth by-products, to reduce greenhouse gas emissions in one or more ways. WO2019021250A1 describes treating plants or seeds with an algal granular composition. Applying these concepts poses similar problems as discussed above in terms of growing sufficient quantities of the microalgae, the pasture treatment itself, and the effectiveness of the approaches is also yet to be demonstrated.

Existing approaches to reducing ruminant land requirements include increasing efficiency through managing storage losses, inaccuracy in delivery of feed, animal feed waste, variation in feed nutrients, mould, pest damage, and bunk management. Bunk management involves improving consistency of feed intake through matching the amount of feed delivered to the amount of feed a ruminant can handle. Forage processing is also used, which can involve chopping and processing grains to reduce particle size and increase digestibility, which in turn can improve feed efficiency. Programme feeding involves feeding routines to achieve a specific rate of gain at a restricted feed intake, which can increase feed efficiency and reduce manure production, especially in growing rather than finishing diets. All of these approaches require effort on the part of farmers, and achieving lower land requirements at scale using these methods is not practical or economically viable for many farmers.

There is therefore a need to provide improved and more efficient ways to reduce methane production and other environmental damage by ruminants, and to improve feed efficiency, without the drawbacks associated with the existing methods.

### SUMMARY OF THE INVENTION

In a first aspect, there is provided a nucleic acid construct for inducing expression of a polypeptide in a plant cell, the nucleic acid construct comprising a coding sequence encoding a polypeptide comprising a vanadate-dependent haloperoxidase (VHPO), or a functional fragment or homologue thereof. The coding sequence is operably linked to one or more regulatory elements, suitable to drive expression of the polypeptide in a plant cell.

Also provided herein is a modified plant cell comprising a nucleic acid coding sequence encoding a polypeptide comprising a vanadate-dependent haloperoxidase (VHPO), or a functional fragment or homologue thereof. In some embodiments, the modified plant cell comprises the construct described above.

Also provided herein is a plant comprising the modified plant cell as described. In some embodiments, the plant expresses VHPO in parts which are edible by livestock, suitably in its leaves and/or grains.

The plant or plant cells described may produce a brominated hydrocarbon, suitably bromoform. The VHPO may be monomeric. In some embodiments, the VHPO is a vanadate-dependent bromoperoxidase (VBPO).

The nucleic acid of the coding sequence may be DNA, which may be in the form of a plasmid.

The VHPO polypeptide may be derived from a species selected from Asparagopsis spp. (e.g. A. taxiformis, A. armata), Acaryochloris marina, Alaria esculenta, Ascophyllum nodosum, Alteromonas naphthalenivorans; Caulerpa spp. (e.g. Caulerpa taxifolia); Chaetomorpha spp. (e.g. Chaetomorpha linum), Chondrus crispus, Colpomenia sinuosa, Corallina officinalis, Corallina pilulifera, Cystoseira trinodis, Furcellaria spp. Gracilaria spp. (e.g. Gracilaria changii, Gracilaria vermiculophylla); Hormophysa triquetra, Hypnea pannosa, Laminaria spp. (e.g. L. digitata, L. saccharina), Laurencia filiformis, Macrocystis pyrifera, Sargassum flavicans, Zonaria farlowii, Cladophora patentiramea, Dictyota bartayresii, Gigartina spp. (e.g. Gigartina stellata), Oedogonium spp., Padina spp. (e.g. Padina australis), Pterocladia capillacea and Ulva spp. (e.g. U. intestinalis, U. linza, U. lactuca).

In some embodiments, the VHPO polypeptide is derived from a microalgal or macroalgal species. The microalgal species may be a member of the genus Emiliana, Calcidiscus or Chaetocerus. The macroalgal species may be a member of the genus Asparagopsis or Chondrus, and in some embodiments the VHPO polypeptide is derived from Asparagopsis armata or Asparagopsis taxiformis, and optionally from Asparagopsis taxiformis Azores, Asparagopsis armata Azores, or Asparagopsis armata Ireland. In some embodiments, the VBPO is selected from Mbb1, Mbb3 and Mbb4 of A. taxiformis.

In some embodiments, the coding sequence comprises a sequence at least 80%, at least 85%, at least 90%, at least 95%, at least 97%, at least 99%, or 100% identical to any one of SEQ ID NOs: 47, 49 to 54, 57 to 60, 64 to 67, 71 to 74, 77 to 80, and 82 to 87. In some embodiments, the nucleic acid construct, plant cell or plant of any preceding claim, wherein the coding sequence comprises a sequence at least 80%, at least 85%, at least 90%, at least 95%, at least 97%, at least 99%, or 100% identical to any one of SEQ ID NOs: 47, 49 and 50 (Mbb1, Mbb3 or Mbb4 of A. taxiformis). In some embodiments, the coding sequence comprises a sequence at least 80%, at least 85%, at least 90%, at least 95%, at least 97%, at least 99%, or 100% identical to any one of SEQ ID NOs: 54, 57 to 60, 64 to 67, 71 to 74, and 77 to 79 (VHPO of A. armata).

In some embodiments, the construct comprises a sequence at least 80%, at least 85%, at least 90%, at least 95%, at least 97%, at least 99%, or 100% identical to any one of SEQ ID NOs: 52, 58, or 80.

In some embodiments, the polypeptide comprising the VHPO comprises an amino acid sequence at least 80%, at least 85%, at least 90%, at least 95%, at least 97%, at least 99%, or 100% identical to SEQ ID NOs: 1, 3 to 15, 18 to 21, 25 to 28, 32 to 35, 38 to 41, and 43 to 46. In some embodiments, the polypeptide comprising the VHPO comprises an amino acid sequence at least 80%, at least 85%, at least 90%, at least 95%, at least 97%, at least 99%, or 100% identical to any one of Mbb1, Mbb3 and Mbb4 of A. taxiformis (SEQ ID NOs: 1, 3 and 4).

In some embodiments, the polypeptide comprising the VHPO comprises an amino acid sequence at least 80%, at least 85%, at least 90%, at least 95%, at least 97%, at least 99%, or 100% identical to any one of SEQ ID NOs: 15, 18 to 21, 25 to 28, 32 to 35, and 38 to 40 (VHPO of A. armata). In some embodiments, the polypeptide comprising the VHPO comprises an amino acid sequence at least 80%, at least 85%, at least 90%, at least 95%, at least 97%, at least 99%, or 100% identical to any one of Chondrus crispus vBPO (SEQ ID NOs: 5 to 7). In some embodiments, the polypeptide comprising the VHPO comprises a sequence at least 80%, at least 85%, at least 90%, at least 95%, at least 97%, at least 99%, or 100% identical to any one of Acaryochloris marina vBPO (SEQ ID NO: 8); Alteromonas naphthalenivorans vBPO (SEQ ID NO: 9), Ascophyllum nodosum vBPO (SEQ ID NO: 10), Corallina officinalis vBPO (SEQ ID NO: 11), Corallina pilulifera (SEQ ID NO: 12), Gracilaria changii vBPO (SEQ ID NO: 13), and Laminaria digitata vBPO (SEQ ID NO: 14).

The plant cell or plant may be a terrestrial plant. The plant or plant cell may be a monocotyledon, typically selected from corn, millet, sorghum or pasture grasses. The plant or plant cell may be a dicotyledon, suitably selected from alfalfa or clover.

In some embodiments, the coding sequence is operably linked to a promoter for driving expression of the polypeptide in a plant cell. The promoter may be a constitutive or inducible promoter. The promoter may be selected from AlcR/AlcA (ethanol inducible); GR fusions, GVG, and pOp/LhGR (dexamethasone inducible); XVE/OlexA (beta-estradiol inducible); and heat shock induction.

In some embodiments, the coding sequence is operably linked to a nucleic acid sequence encoding a targeting signal, and/or the polypeptide may include a targeting signal. The targeting signal may be a peroxisomal targeting signal, typically wherein the peroxisomal targeting signal is SEQ ID NO: 90. The targeting signal may be a mitochondrial targeting signal, suitably wherein the mitochondrial targeting signal is SEQ ID NO: 89. The targeting signal may be a chloroplast targeting signal, typically wherein the chloroplast targeting signal is SEQ ID NO: 88.

In a second aspect, there is provided an animal feed or animal supplement comprising the modified plant cell or plants of any described aspect or embodiment.

In a third aspect, there is provided a method for reducing methane production in an animal, comprising growing or culturing the modified plants or plant cells of any described aspect or embodiment under conditions suitable for the production of halogenated hydrocarbons, and feeding an effective amount of the modified plants or plant cells to the animal. A further method for reducing methane production in an animal comprises administering to the animal an effective amount of modified plant cells, plants, or animal feed of any described aspect or embodiment.

In some embodiments, the growing or culturing of the modified plant or plant cells does not include providing substrates or cofactors relevant to the production of a halogenated hydrocarbon. In some embodiments, where the halogenated hydrocarbon is bromoform, the substrates or cofactors relevant to the production of a halogenated hydrocarbon include at least one compound selected from the group consisting of KBr, sodium orthovanadate, pentane-2,4-dione, and H₂O₂.

The animal as described may be a ruminant, typically the animal is bovine. The animal may be a cow, sheep or goat.

In embodiments of the described methods, methane production may be reduced. In some embodiments, feed efficiency is increased.

In a fourth aspect, there is provided a method of preparing a modified plant cell, comprising transforming a plant cell with the nucleic acid construct of any described embodiment. The plant cell may be transformed by Agrobacterium-mediated infiltration. The plant cell may be transformed by particle bombardment.

In a fifth aspect, there is provided a method of producing an animal feed for reduction of methane production in an animal, comprising growing the modified plant cell or plant of any described aspect or embodiment, and processing the modified plant cell or plant into an animal feed.

### BRIEF DESCRIPTION OF THE DRAWINGS

FIGS. 1A to 1D show example constructs for driving expression of various genes in corn protoplasts, with associated reporters and targeting sequences where appropriate.

FIGS. 2A to 2D show example constructs for driving expression of Asparagopsis taxiformis Mbb1 in Arabidopsis, with associated reporters and targeting sequences where appropriate.

FIGS. 3A to 3D show fluorescence microscopy images of protoplasts transfected with AtMbb1+GFP fusion constructs and an mCherry construct, as detailed in Example 1. Images were taken in GFP and mCherry channels at 20× magnification, under the same acquisition settings, and then merged. FIG. 3A shows cells transfected with cytosol-targeting constructs; FIG. 3B shows cells transfected with chloroplast-targeting constructs; FIG. 3C shows cells transfected with mitochondria-targeting constructs; FIG. 3D shows cells transfected with peroxisome-targeting constructs.

FIGS. 4A to 4D show fluorescence microscopy images of protoplasts transfected with CcMbb3+GFP fusion constructs and an mCherry construct, as detailed in Example 1. Images were taken in GFP and mCherry channels at 20× magnification, under the same acquisition settings, and then merged. 4A shows cells transfected with cytosol-targeting constructs; 4B shows cells transfected with chloroplast-targeting constructs; 4C shows cells transfected with mitochondria-targeting constructs; 4D shows cells transfected with peroxisome-targeting constructs.

FIG. 5 shows the morphology of transgenic plant lines 2007, 2011, and 2012 (labelled boxes) and six wild type Arabidopsis plants (not labelled), as described in Example 2.

FIGS. 6A to 6C show results of transcription analysis of transgenic plant lines according to Example 2. At-Mbb1 transcript levels were measured by qRT-PCR and then normalised to expression of a known wild-type gene. FIG. 6A shows the results of Plate 1 also detailed in Table 3. FIG. 6B shows the results of Plate 2 also detailed in Table 4. FIG. 6C shows the results of Plate 1 also detailed in Table 5. Wild-type expression is on the far right of each of FIGS. 6A-C and has a normalized expression of zero.

FIGS. 7A to 7D show extracted ion chromatograms for transgenic plant material according to Example 5. FIG. 7E is a chromatogram of the positive control: 0.25 μg/mL bromoform standard. FIG. 7A is the chromatogram for the 2007 sample. FIG. 7B is the chromatogram for the 2011 sample. FIG. 7C is the chromatogram for the 2012 sample. FIG. 7D is the gas chromatogram for the 2023 sample.

FIG. 8 shows mass spectrometry results for material taken from the 2007 transgenic plant line, along with the NIST reference spectrum for bromoform, as detailed in Example 5. The 2007 sample mass spectrum is shown above, the NIST reference spectrum below.

### DETAILED DESCRIPTION

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, Pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). Each of these general texts is herein incorporated by reference.

Prior to setting forth the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

A ‘polynucleotide’ is a single or double stranded covalently linked sequence of nucleotides in which the 3′ and 5′ ends on each nucleotide are joined by phosphodiester bonds. The polynucleotide may be made up of deoxyribonucleotide bases or ribonucleotide bases. Polynucleotides include DNA and RNA, and may be manufactured synthetically in vitro or isolated from natural sources. Sizes of polynucleotides are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called ‘oligonucleotides’. The term ‘nucleic acid sequence’ as used herein, is a single or double stranded covalently linked sequence of nucleotides in which the 3′ and 5′ ends on each nucleotide are joined by phosphodiester bonds. The polynucleotide may be made up of deoxyribonucleotide bases or ribonucleotide bases. Nucleic acid sequences may include DNA and RNA, and may be manufactured synthetically in vitro or isolated from natural sources.

Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

According to the present invention, similarity to the nucleic acid sequences described herein is not limited simply to 100% sequence identity. In this regard, the term “substantially similar”, relating to two sequences, means that the sequences have at least 70%, 80%, 90%, 95%, 98%, 99% or 100% similarity. Likewise, the term “substantially complementary”, relating to two sequences, means that the sequences are completely complementary, or that at least 70%, 80%, 90%, 95% or 99% of the bases are complementary. That is, mismatches can occur between the bases of the sequences which are intended to hybridise, which can occur between at least 1%, 5%, 10%, 20% or up to 30% of the bases.

The term ‘operably linked’, when applied to nucleic acid sequences, for example in an expression construct, indicates that the sequences are arranged so that they function cooperatively in order to achieve their intended purposes. By way of example, in a DNA vector a promoter sequence allows for initiation of transcription that proceeds through a linked coding sequence as far as a termination sequence. In the case of RNA sequences, one or more untranslated regions (UTRs) may be arranged in relation to a linked polypeptide coding sequence referred to as an open reading frame (ORF). A given mRNA as disclosed herein may comprise more than one ORFs, a so-called polycistronic RNA. An mRNA may encode more than one polypeptide, and may as a result include cleavage sites or other sequences necessary to result in the production of multiple functional products, as known in the art. A UTR may be located 5′ or 3′ in relation to an operatively linked coding sequence ORF. UTRs may comprise sequences typically found in mRNA sequences found in nature, such as any one or more of: Kozak consensus sequences, initiation codons, cis-acting translational regulatory elements, cap-independent translation initiator sequences, poly-A tails, internal ribosome entry sites (IRES), structures regulating mRNA stability and/or longevity, sequences directing the localisation of the mRNA, and so on.

The term ‘expressing a polypeptide’ in the context of the present invention refers to production of a polypeptide for which the polynucleotide sequences described herein code. Typically, this involves transcription of a DNA sequence comprised within a modified plant cell, followed by translation of the resultant mRNA sequence by the ribosomal machinery of the plant cell in which the sequence is comprised.

The term ‘polypeptide’ as used herein is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains. Some proteins are multimeric or involve complexes of polypeptide chains and other molecules such as cofactors. In such cases, polynucleotides coding for such proteins as described herein are intended to encompass the provision of multiple monomers such that a complete or functional resultant protein can be produced. Different monomers can be encoded on the same polynucleotide construct, or separately. Transcription/translation of the coding polynucleotide sequence within a modified cell allows for localised post-translational modification appropriate to the cell type to be applied. Such modifications may regulate folding, localization, interactions, degradation, and activity of the gene product. Typical post translational modifications may include cleavage, refolding and/or chemical modification such as methylation, acetylation or glycosylation.

A ‘promoter’ is defined as an array of nucleic acid control sequences that direct transcription of an operably linked nucleic acid. Promoters include necessary nucleic acid sequences near the start site of transcription, such as, in the case of a polymerase II-type promoter, a TATA element. A promoter also optionally includes distal enhancer or repressor elements, which can be located as much as several thousand base pairs from the start site of transcription.

The present invention seeks to alleviate the disadvantages of existing approaches of reducing ruminant methane emissions and land requirements by offering a solution that is globally scalable, economically advantageous for farmers and has potential to consistently reduce methane emissions and increase feed efficiency rates (and reduce total land requirements of ruminants).

Methane is a greenhouse gas, which can be produced by methanogenic microbes, particularly methanogenic archaea. Without wishing to be bound by theory, it is thought that these microbes are not only present in the environment but also inhabit the gastrointestinal tract of ruminants, contributing to their production of methane. It is also thought that the presence of halogenated hydrocarbons, such as brominated hydrocarbons, can reduce methane production in this context. Brominated hydrocarbons are thought to prevent methane emission from ruminants by interfering with the vitamin B12 cofactor which is used by the methyltransferase enzymes of ruminant methanogenic archaea in the production of methane.

As mentioned, various macro algae have been shown to reduce methane emissions when fed to ruminants, with red macro alga Asparagopsis sp. being shown to be highly effective. These species produce halogenated compounds, including bromoform (CHBr₃) and polybromomethanes such as dibromomethane and chlorodibromomethane, which are thought to be synthesised by haloperoxidases catalysing the conversion of halide anions to hypohalous acid with hydrogen peroxide (H₂O₂) acting as the oxidant. The requirement for H₂O₂ links synthesis to the enzymatic production of reactive oxygen species (ROS).

Gene clusters in Asparagopsis taxiformis that contain reactive oxygen species (ROS)-producing enzymes together with bromoform-producing vanadium-dependent haloperoxidases (VHPOs) were identified in Thapa et al. “Genetic and biochemical reconstitution of bromoform biosynthesis in Asparagopsis lends insights into seaweed ROS enzymology” (ACS Chem Biol, 2020). They identified that Mbb1, Mbb3 and Mbb4 VHPOs can synthesise bromoform in an in vitro assay when the E. coli expressed recombinant proteins are provided sodium orthovanadate, potassium bromide, H₂O₂ and the artificial substrate monochlorodimedone (MCD), proposing the below equations. After testing a range of hydrocarbon substrates, they also proposed that acetoacetyl-S-ACP (an acyl-acyl carrier protein intermediate in plastidial fatty acid synthesis) is the likely physiological substrate for bromoform synthesis in A. taxiformis. The Thapa et al authors were unable to heterologously express the putative ROS-producing enzyme Mbb2 but did identify a Chondrus crispus homologue and demonstrate its NAD(P)H-oxidase (NOX) activity. They also demonstrated activity of homologous C. crispus VHPO proteins.

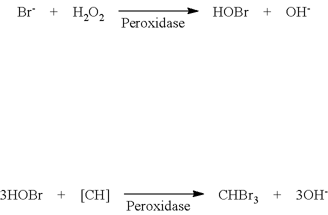

A more general alternative formula for the production of brominated organic compounds is

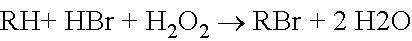

where RH is an organic substrate.

### VHPO Enzyme Sources and Uses

In embodiments of the present invention, plants or plant cells are modified to express substances which reduce methane production in animals, typically ruminants, which ingest the plants, plant cells, or feed prepared from these. In this context, plants are members of the Kingdom Plantae, typically terrestrial members thereof. Sources for genetic modification of such plants are frequently found in aquatic photosynthetic species, such as algal species and phytoplankton, several of which produce substances which reduce methane production in animals. It is contemplated to introduce the genetic basis for the production of these substances into plants, especially ruminant feed plants (forage) to enable efficient ingestion of appropriate amounts of these substances. Accordingly, the plants or plant cells of the present invention may be modified with a construct comprising a coding sequence (for example, a coding sequence encoding a VHPO) which is derived from an aquatic photosynthetic species such as a micro- or macroalgal species. In this context, ‘derived from’ means that the construct encodes a polypeptide similar in function and/or sequence to those produced by wild-type members of these species. Such constructs may differ from the original by the inclusion of codon optimisation, the presence of reporter sequences, targeting sequences, modified nucleobases, and so forth. It is contemplated that VHPO enzymes and other proteins as described herein may be engineered or modified to increase their activity, stability, or other properties, by any of a number of methods known in the art.

In some embodiments, the present invention provides plants and plant cells modified to express VHPO and/or otherwise to produce halogenated hydrocarbons, such as bromoform, in order to reduce methane production in animals. Again, suitable VHPO enzymes are present in a number of aquatic photosynthetic species. VHPO enzymes specialised to produce brominated hydrocarbons can be referred to as vanadium-dependent bromoperoxidases (VBPO), and references herein to VHPO are intended to encompass VBPO. It is expected that the presence of VHPO alone is sufficient for the production of halogenated hydrocarbons by modified plants and plant cells of the invention.

Species which have been determined to express VHPO and/or have methane reduction effects include Asparagopsis spp. (e.g. A. taxiformis, A. armata), Acaryochloris marina, Alaria esculenta, Ascophyllum nodosum, Alteromonas naphthalenivorans; Caulerpa spp. (e.g. Caulerpa taxifolia); Chaetomorpha spp. (e.g. Chaetomorpha linum), Chondrus crispus, Colpomenia sinuosa, Corallina officinalis, Corallina pilulifera, Cystoseira trinodis, Furcellaria spp. Gracilaria spp. (e.g. Gracilaria changii, Gracilaria vermiculophylla); Hormophysa triquetra, Hypnea pannosa, Laminaria spp. (e.g. L. digitata, L. saccharina), Laurencia filiformis, Macrocystis pyrifera, Sargassum flavicans, Zonaria farlowii, Cladophora patentiramea, Dictyota bartayresii, Gigartina spp. (e.g. Gigartina stellata), Oedogonium spp., Padina spp. (e.g. Padina australis), Pterocladia capillacea and Ulva spp. (e.g. U. intestinalis, U. linza, U. lactuca) (see, for example, Thapa et al 2020, WO 2020/243792, Abbott et al 2020, Thapa, H. R., & Agarwal, V. (2021). Obligate Brominating Enzymes Underlie Bromoform Production by Marine Cyanobacteria. Journal of Phycology). Microalgal species determined to express VHPO include members of the genera Emiliana, Calcidiscus and Chaetocerus. Accordingly, nucleic acid sequences encoding polypeptides such as VHPO derived from any of these species may be used in the present context. The VHPOs may or may not be monomeric.

A. taxiformis preferentially grows in tropical to warm temperate waters, compared with A. armata which is adapted to cooler temperate waters, although the species overlap in some environments, such as the Mediterranean (Andreakis et al (2004) “Asparagopsis taxiformis and Asparagopsis armata (Bonnemaisoniales, Rhodophyta): genetic and morphological identification of Mediterranean populations” European Journal of Phycology). Indeed, subspecies or strains of each exist present in different locations, for example, A. taxiformis has an Atlantic and a Mediterranean strain, while A. armata has been isolated near Ireland and the Azores.

To identify further potential VHPO for use in the present context, genomic comparisons were made between the identified Mbb1, Mbb2, Mbb3 and Mbb4 polypeptides identified in A. taxiformis (SEQ ID NOs: 1 to 4) and Chondrus crispus (SEQ ID NOs: 5 to 7), and the related species Asparagopsis armata, isolated from strains in Ireland (Asparagopsis armata Ireland) and the Azores islands (Asparagopsis armata Azores), as well as a strain of A. taxiformis isolated from the Azores islands (Asparagopsis taxiformis Azores).

Multiple collections of macroalgae were made and DNA and RNA preparations generated for sequence analysis using Illumina sequencing technology. Library preparation for genomic DNA sequencing included DNA fragmentation, adapter ligation, size selection and amplification. It included unique dual indexing. The run type was paired end, with read lengths of 2×150 bp. More than 5 million read pairs (10 million reads) were obtained per sample (+/−3%); Additionally, transcriptome sequencing was completed in triplicate using Illumina sequencing technology for each of the samples. The library preparation for RNA sequence analysis included strand-specific cDNA library preparation, purification of poly-A containing mRNA molecules, mRNA fragmentation, random primed cDNA synthesis (strand specific), adapter ligation and adapter specific PCR amplification. The run type was paired-end, with read lengths of 2×150 bp. 30 million read pairs were identified (+/−3%). The approach included long and accurate read lengths to quantitatively evaluate common and rare transcripts. Genomic DNA and transcriptome sequencing data were mapped against taxonomically-distinct reference genome sequences and analysed in other ways for the identification and annotation of gene coding sequences, identification and quantification of transcripts, pairwise comparison of expression levels and determination of significant fold differences, alternative splicing analysis based on known/provided gene models for eukaryotes, and detection and annotation of SNPs and InDels. The transcriptome sequencing data also provided comprehensive information on gene expression, levels, sequence, structure and strand orientation of RNA types. The coding sequences identified in this analysis were searched using BLASTN to identify sequences related to Mbb1, 2, 3 and 4. This analysis resulted in the identification of Mbb1, 2, 3 and 4 coding sequences in Asparagopsis taxiformis Azores, and in Asparagopsis armata Ireland and Azores. Additionally, both Asparagopsis armata strains were found to have a 2^{nd} Mbb4 gene not present in Asparagopsis taxiformis. The identified polypeptide and nucleotide coding sequences are shown in Table 6.

Accordingly, the plant or plant cells described herein can be modified to express one or more proteins identified in Asparagopsis taxiformis and/or Asparagopsis armata, suitably VHPOs identified in one or both of these species. In some cases, the plant or plant cells are modified to express A. armata proteins selected from those identified as Mbb1 like, Mbb3 like, Mbb4 like, and Mbb4-2 like, and in particular, the Mbb4-2 like protein.

### Inducible Expression or Activity of VHPO

In some instances, it can be advantageous to control the degree to which VHPO are expressed by the modified plant cells or plants, and/or the activity of the VHPO polypeptides synthesised by the plants or plant cells. Halogenated hydrocarbons can have toxic effects on the organisms which express them, and/or on the environment. For example, halogenated hydrocarbons are thought to play a role in ozone depletion.

Additionally, it can be beneficial to control expression of the encoded VHPO depending on the cell type and/or location of the cell within the plant. For example, it may be advantageous for efficiency to express the VHPO only in the parts of the plant which are eaten by a particular animal, such as the leaves or grains. This can also limit the impact of any toxicity of producing the VHPO or the subsequently produced halogenated hydrocarbon.

It is contemplated for the desired compounds such as bromoform to be produced in any plant tissue that is commonly fed to ruminant livestock. This can include, but is not limited to leaves and stems (for example from grasses/cereals, legumes, and brassicas), seeds (for example from cereal, pulse and oilseed crops) and roots/tubers (for example from turnip, swede, beet).

Plant gene promoters, including constitutive promoters; those for genes with tissue- or cell-specific or -enhanced expression; and those that are regulated in response to environmental, hormonal, chemical, and/or developmental signals, are discussed, for example, in U.S. Pat. No. 9,556,102 B2, which is incorporated herein by reference.

Accordingly, it is contemplated for the nucleic acid coding sequences described herein to be operably linked to one or more promoters or other control sequences which permit control of the expression of the VHPO, for example, its transcription, translation, or otherwise. Such promoters are also known as ‘inducible promoters’, in contrast to ‘constitutive promoters’ which are active in all circumstances. In some instances, the inducible promoters permit expression only in the presence, or absence, of a particular condition, such as temperature, humidity, or the presence of one or more chemical signals. For example, inducible transgene expression which can be used in modified plant cells are discussed in Borghi “Inducible gene expression systems for plants”. Methods Mol Biol. 2010, and Misra and Ganesan “The impact of inducible promoters in transgenic plant production and crop improvement” Plant Gene, 2021, and include AlcR/AlcA (ethanol inducible); GR fusions, GVG, and pOp/LhGR (dexamethasone inducible); XVE/OlexA (beta-estradiol inducible); and heat shock induction.

Other methods of controlling the expression of the provided polynucleotide coding sequence include the use of post-translational transcription control mechanisms. For example, the provided coding sequence may be operably linked to a microRNA sequence allowing for downregulation of expression in particular cell and/or tissue types and consequent expression in other cell and/or tissue types.

Further methods of controlling the activity of the expressed VHPO include expressing the polypeptide in an inactive propeptide form, which is then activated under specific circumstances. Alternatively, substrates (such as carbon feedstocks), cofactors, and/or other compounds or conditions necessary for the VHPO to function can be withheld until halogenated hydrocarbon production is desired. In some embodiments, the modified plant cells or plants can be grown in the presence of vanadium or vanadium-containing compounds such as sodium orthovanadate, only when halogenated hydrocarbon production is desired. Similarly, the modified plant cells or plants can be grown in the presence of halogen or halides (suitably bromine or bromides such as sodium bromide or potassium bromide), only when halogenated hydrocarbon production is desired. In some embodiments, the modified plants or plant cells can produce or provide, or be modified to further produce or provide, substrates (such as hydrocarbon feedstocks), cofactors, and/or other compounds or conditions necessary for the VHPO to function. In such cases, the production of those secondary factors can alternatively or additionally be controlled by any suitable means, such as those described above.

In such cases, some embodiments of the methods described herein can involve providing necessary inducers, substrates or cofactors as indicated above only when VHPO expression and/or halogenated hydrocarbon production is desired. Similarly, inducers, substrates or cofactors can be depleted or removed when VHPO expression and/or halogenated hydrocarbon production is not desired. For example, vanadium compounds can be chelated to remove their availability.

It is contemplated that bromoform production may be induced in modified plants or plant cells as described herein by subjecting the plant or plant cell to one or more stressors (for example, heat or drought stress). Without being bound by theory, it is thought that levels of reactive oxygen species (ROS) may be increased in stressed plants and thereby more available to the VHPOs described herein.

As mentioned above, the modified plants or plant cells can be modified to further produce or provide, substrates (such as hydrocarbon feedstocks), cofactors, and/or other compounds or conditions necessary for the VHPO to function. For example, the plants or plant cells can be modified to produce further ROS, such as by modification to produce a protein that produces or increases production of ROS. Such a protein may be a NADPH_oxidase, for example, A. taxiformis NADPH_oxidase (Mbb2) or equivalents or homologues thereof. Conversely, when modification only to produce VHPO is desired, the plants or plant cells may not be modified to produce NADPH oxidase (such as Mbb2 and identified equivalents). Such modifications may be performed and controlled by any suitable means, such as those described herein.

### Organelle-Targeted Expression

Since the VHPO proteins are peroxidases, they need H₂O₂, and potentially carbon feedstocks, in order to function. The availability of these substrates varies within the cell, and in particular, may be more abundant in the peroxisome, chloroplast and/or mitochondrion, since these organelles are particularly involved in the production and/or removal of reactive oxygen species. Thus, by targeting proteins such as VHPO to subcellular locations, the invention can reduce or eliminate the need for additional substrates and/or cofactors associated with halogenated hydrocarbon (e.g. bromoform) production to be provided to the plant or plant cell.

Additionally, targeting proteins such as VHPO to subcellular locations can act to protect the cell from any deleterious or toxic effects of any products, such as bromoform.

Accordingly, the polypeptides and/or encoding nucleic acid sequences of the invention may comprise or encode targeting sequences suitable to be targeted to particular cell locations, suitably to the peroxisome, chloroplast and/or mitochondrion. The targeting sequences may be selected to match those used by the species being modified, or may be chosen from another species, where appropriate. Targeting sequences may be located at the C-terminus, N-terminus, or elsewhere on the polypeptides.

### Modification of Plant Cells and Plants for Use

The present invention is intended to encompass the modification of any suitable plant or plant cell, typically terrestrial plants or cells thereof which are suitable for feeding to ruminant animals. However, in some embodiments aquatic plants may be used. Depending on the animal, environment, climate and/or amounts needed to achieve methane reduction, the skilled person will be able to select a suitable candidate for modification.

In the context of this invention, references to plant cells herein are intended to encompass plant protoplasts, that is, plant cells lacking a cell wall, or which have been treated to remove the cell wall. Plant cells are often modified while in a protoplast state. It is envisioned that modified plant cells according to the invention may be cultured in vitro, as protoplasts or otherwise, and fed to the animals in processed or unprocessed form—that is, a process in which whole plants are never regenerated. Similar approaches are contemplated using callus cells or other plant cells grown in suspension.

The plant or plant cell may be a monocotyledon or a dicotyledon. Suitable monocotyledons include corn, millet, sorghum and pasture grasses. Suitable dicotyledons include alfalfa and clover.

Grasses which may be useful in the present context include Agrostis spp. (bentgrasses), Agrostis capillaris (common bentgrass), Agrostis stolonifera (creeping bentgrass), Andropogon hallii (sand bluestem), Arrhenatherum elatius (false oat-grass), Bothriochloa bladhii (Australian bluestem), Bothriochloa pertusa (hurricane grass), Brachiaria decumbens (Surinam grass), Brachiaria humidicola (koronivia grass), Bromus spp. (bromegrasses), Cenchrus ciliaris (buffelgrass), Chloris gayana (Rhodes grass), Cynodon dactylon (bermudagrass), Dactylis glomerata (orchard grass), Echinochloa pyramidalis (antelope grass), Entolasia imbricata (bungoma grass), Festuca spp. (fescues), Festuca arundinacea (tall fescue), Festuca pratensis (meadow fescue), Festuca rubra (red fescue), Festulolium (Festuca-Lolium hybrids), Heteropogon contortus (black spear grass), Hymenachne amplexicaulis (West Indian marsh grass), Hyparrhenia rufa (jaragua), Leersia hexandra (southern cutgrass), Lolium spp. (ryegrasses), Lolium multiflorum (Italian ryegrass), Lolium perenne (perennial ryegrass), Megathyrsus maximus (Guinea grass), Melinis minutiflora (molasses grass), Paspalum conjugatum (carabao grass), Paspalum dilatatum (dallisgrass), Phalaris arundinacea (reed canarygrass), Phleum pratense (timothy), Poa spp. (bluegrasses and meadow-grasses), Poa arachnifera (Texas bluegrass), Poa pratensis (Kentucky bluegrass), Poa trivialis (rough bluegrass), Setaria sphacelata (African bristlegrass), Themeda triandra (kangaroo grass), Thinopyrum intermedium (intermediate wheatgrass).

Legumes which may be useful in the present context include Arachis pintoi (pinto peanut), Astragalus cicer (cicer milkvetch), Chamaecrista rotundifolia (roundleaf sensitive pea), Clitoria ternatea (butterfly-pea), Kummerowia (annual lespedezas), Kummerowia stipulacea (Korean clover, Korean lespedeza), Kummerowia striata (Japanese clover and common lespedeza), Lotus corniculatus (bird's-foot trefoil), Macroptilium atropurpureum (purple bush-bean), Macroptilium bracteatum (burgundy bean), Medicago spp. (medics), Medicago sativa (alfalfa, lucerne), Medicago truncatula (barrel medic), Melilotus spp. (sweetclovers), Neonotonia wightii (perennial soybean), Onobrychis viciifolia (common sainfoin), Stylosanthes spp. (stylo), Stylosanthes humilis (Townsville stylo), Stylosanthes scabra (shrubby stylo), Trifolium spp. (clovers), Trifolium hybridum (alsike clover), Trifolium incarnatum (crimson clover), Trifolium pratense (red clover), Trifolium repens (white clover), Vicia spp. (vetches), Vicia articulata (oneflower vetch), Vicia ervilia (bitter vetch), Vicia narbonensis (narbon vetch), Vicia sativa (common vetch, tare), Vicia villosa (hairy vetch), Vigna parkeri (creeping vigna).

Cereals which may be useful in the present context include Zea mays (maize, corn) Triticum spp., Triticum aestivum (bread wheat), Oryza sativa (Asian rice), Oryza glaberrima (African rice), Hordeum vulgare (barley), Avena sativa (oats), Secale cereale (rye), Sorghum spp., Sorghum bicolor, Millets (e.g. Panicum miliaceum, Panicum sumatrense, Panicum sonorum, Pennisetum glaucum).

Root crops which may be useful in the present context include Raphanus sativus (Daikon, radish), Brassica rapa (turnip), Brassica napus (Rutabaga, Swede, forage rape), Beta vulgaris (sugar beet)

Pulses and oilseeds which may be useful in the present context include Glycine max (Soybean), Phaseolus spp., Phaseolus vulgaris (Kidney bean, navy bean, pinto bean, black turtle bean, haricot bean), Vicia faba (broad bean, field bean), Pisum sativum (garden pea), Lupinus albus (lupin), Brassica napus (oilseed rape, canola), Helianthus annuus (sunflower), Arachis hypogaea (peanut, groundnut).

In addition to forage plants for direct feeding to ruminant animals, it is also contemplated that plants and plant cells of other species may be transformed as discussed herein. For example, model plant species such as Nicotiana benthamiana, Arabidopsis thaliana (both eudicotyledons); and Lemnoideae (including Lemna spp.) or Oryza sativa (monocotyledons) may be used.

Any suitable method can be used to transiently and/or stably transform plant cells, as will be evident to the skilled person in context. For example, Agrobacterium-meditated transformation, chemical procedures, electroporation, and/or the use of high-velocity particles (particle bombardment) may be used. Genomic editing techniques as known in the art may also be used. For example, in U.S. Pat. No. 9,556,102 B2, which is incorporated herein by reference, discusses methods for transformation of plants, plastid transformation; and the regeneration, development, and cultivation of plants from single plant protoplast transformants.

Similarly, the nucleic acid constructs of the invention or those comprised by the plant or plant cells of the invention can be of any suitable form. DNA or RNA sequences may be used. mRNA sequences may be used, suitably where short-term or transient production is preferred, as provided mRNA represents an exhaustible supply of genetic code for the production of a particular polypeptide. Circular RNA may also be used. DNA sequences may likewise be in any suitable form, such as a plasmid. The nucleic acid constructs and/or the method of transformation may be selected for stable transformation, such as by genomic integration.

The nucleic acid constructs may be modified to include one or more reporters by any suitable method, for example, by creating GFP fusions, which may aid in locating or identifying successful transformations.

The modified plants or plant cells are capable of producing halogenated hydrocarbons, such as bromoform.

### Preparation and Use of Animal Feed

The modified plants or plant cells produced can be expanded and propagated in any suitable way, such as by runners, cuttings, and the preparation of seed. The modified plants, whether or not subjected to further processing, can be used as an animal feed or feed supplement, and/or grown in a location where it can be eaten by said animal during normal feeding. As mentioned, the expression of the provided polypeptide can be induced by a suitable method. Where and when produced, the bromoform or other methane-reducing substance can inhibit the production of methane by the recipient animal, for example by inhibition of microbial production of methane.

Typically, the animal is a ruminant, and may be bovine, ovine, equine, camelid, or cervine. The animal may be a cow, sheep or goat. The feed efficiency may be increased, given that that energy-dense methane is not produced. The animal may be fed with the modified plant or derived feed intermittently or continuously, as appropriate.

The modified plant may be grown as an annual or perennial crop, depending on the species and cultivar, and either as a mixture together with other appropriate species or as a monoculture. Its tissues may be used as an agricultural foodstuff that livestock forage for themselves. Alternatively, the modified plants tissues may be harvested and processed in any way that is appropriate to provide fodder (or animal feed), which includes, but is not limited to, hay, straw, silage, cereal grains, pulses, oilseeds, tubers, mixed rations, compressed and pelleted rations. Extracts from the modified plants can also be fed to livestock in their drinking water or in licking blocks, or the like. Any means may be used that enables the modified plant material or an extract from that material to enter the rumen of the livestock. The content of bromoform, related halogenated compounds, or other components may be measured before feeding the modified plant material or extracts from that material to the animal, to determine an appropriate amount to be fed.

### EXAMPLES

The invention is further illustrated by the following, non-limiting, examples. The below experiments demonstrate some embodiments of the invention in practice.

### Example 1—Transient Expression of AtMbb1, CcMbb3 and AaMbb4-2 in Corn Protoplasts

Asparagopsis taxiformis Mbb1 protein (AtMbb1, SEQ ID NO: 41), Chondrus crispus Mbb3 protein (CcMbb3 SEQ ID NO: 6) and A. armata Mbb4-2 protein(AaMbb4-2 SEQ ID NO: 19) were expressed in a corn protoplast system. Four green fluorescent protein (GFP) fusion constructs were designed for each gene, and signal peptides were added to direct the expressed fusion protein to the cytosol, chloroplast, mitochondria and peroxisome respectively. The gene sequences for AtMbb1 (SEQ ID NO: 80), AaMbb4-2 (SEQ ID NO: 58), and CcMbb3 (SEQ ID NO: 52) were codon optimized for expression in a corn system. Thus, a total of 12 vectors were designed and made as indicated below for transient expression in corn protoplasts.

Construct design: Chloroplast transit peptide (SEQ ID NO: 88), mitochondria targeting peptide (SEQ ID NO: 89), and peroxisome targeting peptide (SEQ ID NO: 90) were found from the typical maize chloroplast-localized protein (ribulose bisphosphate carboxylase small subunit 2, GenBank accession: NP_001338725), mitochondria-localized protein (superoxide dismutase 3, GenBank accession: NP_001105742) and peroxisome-localized protein (silkless ears1, GenBank accession: NP_001131410). These signal peptides were fused to the N-terminus (chloroplast transit peptide, mitochondria targeting peptide) or C-terminus (peroxisome targeting peptide) of AtMbb1, AaMbb4-2, and CcMbb3, while GFP (eGFP, SEQ ID NO: 97) was fused to the C-terminus of these genes.

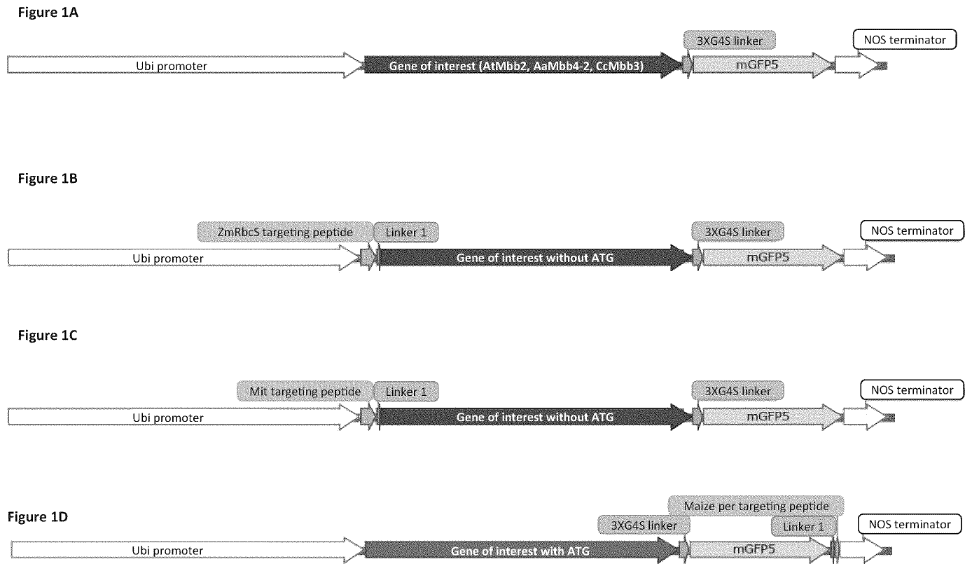

Two linkers (Linker 1 (SEQ ID NO: 93) and 3XGGGGS (SEQ ID NO: 94)) were used between the domains of the fusion proteins. No signal peptide was found in AtMbb1, AaMbb4-2 and CcMbb3, indicating that they are cytosol-localized proteins, so AtMbb1-GFP, AaMbb4-2-GFP, and CcMbb3-GFP without additional signal peptide were considered as cytosol-localized. The chimeric genes were driven by a maize Ubi-1 promoter (SEQ ID NO: 91). The gene cassettes were cloned into a pUC57 vector Example indications of how such constructs were structured can be seen in FIGS. 1A to 1D. FIG. 1A shows a cytosol localised protein, the gene sequence fused with 3XG4S linker and GFP, and driven by a maize ubi-1 promoter and followed by a NOS terminator (SEQ ID NO: 96). The fragment was synthesized and cloned into pUC57. FIG. 1B shows a chloroplast-localised protein, with a signal peptide from a maize RbcS protein fused to the N-terminus of the gene of interest, followed by GFP. The start codon (ATG) of the gene of interest was removed and two linkers added to improve protein expression. The chimeric gene was inserted between the maize ubi-1 promoter and the NOS terminator. FIG. 1C shows a mitochondrion localised protein, with a mitochondrial targeting peptide from maize SOD3 fused to the N-terminus of the gene of interest, followed by GFP. The start codon (ATG) of the gene of interest was removed and two linkers added to improve protein expression. The chimeric gene was inserted between the maize ubi-1 promoter and the NOS terminator. FIG. 1D shows a peroxisome localised protein, with the gene of interest fused with GFP, followed by a peroxisome targeting peptide. Two linkers were added to improve protein expression. The chimeric gene was inserted between the maize ubi-1 promoter and the NOS terminator. In the context of the invention, alternative reporters, genes (such as those described herein), promoters, terminators and linkers can of course be used where appropriate, and some components such as reporters can be omitted entirely if desired.

Protoplast isolation and transfection: Protoplasts were isolated from leaf tissues of 7-day-old etiolated corn seedlings (Coy et al., 2022. “Protoplast isolation and transfection in maize.” Methods Mol Biol.). Briefly, the first true leaf was collected and the tip and the base removed. Leaf tissue was sliced into 0.5-1 mm slices using a new scalpel and distributed in Petri dishes containing digestion solution. The plates were placed in a vacuum chamber under house vacuum for 30 min, and then moved into an orbital shaking incubator at 28° C. Leaf tissue was incubated for 4 hr until protoplasts were released. The protoplast solution was collected using a 0.45 μm Steriflip filter device (Millipore), and was washed three times by adding 10 mL of the MMg solution. The protoplasts were counted by using a hemocytometer.

Plasmid DNA was received and transformed into E. coli, DNA was extracted and confirmed via PCR. To obtain the large amount of DNA required for protoplast transfection DNA maxi-preparation was carried out. For transfection, 40% polyethylene glycol (PEG) was added into the mixture of MMg solution, plasmid DNA, and protoplasts. Each plasmid was co-transfected with a reference plasmid expressing the fluorescent protein mCherry under the control of a constitutive promoter as a transfection efficiency control. mCherry was employed as a positive control for subcellular localisation. mCherry is a derivative of RFP, and used to co-localise GFP-fused proteins. For each target, there was an mCherry control associated with it.

Finally, the protoplasts were incubated at 28° C. overnight in dark. The cells were used for localization studies by a fluorescent microscope. Expression of the mCherry gene was used to determine transfection efficiency. Transfection efficiency may be calculated as the percentage of fluorescent protoplasts to the total protoplasts, monitored under a fluorescent microscope. Detection of GFP by fluorescence microscopy confirmed successful transfection and expression of the fusion construct.

Fluorescence microscopy: In order to determine the expression of the AtMbb1 and CcMbb3 constructs, in the transfected corn protoplast cells, transient expression analysis by fluorescence microscopy analysis was carried out. The corn protoplasts were transfected as described above and then analysed to determine the subcellular localization of the expressed protein.

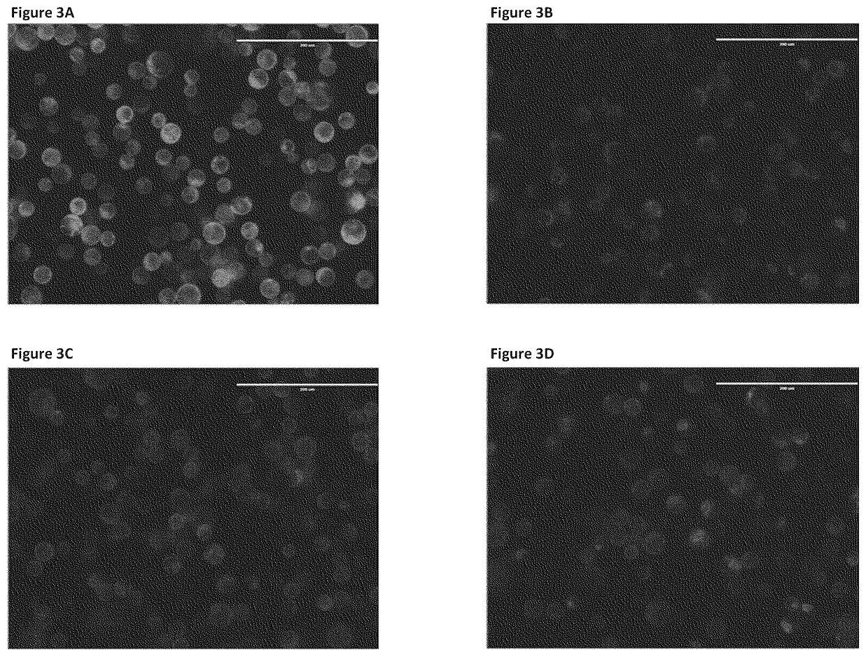

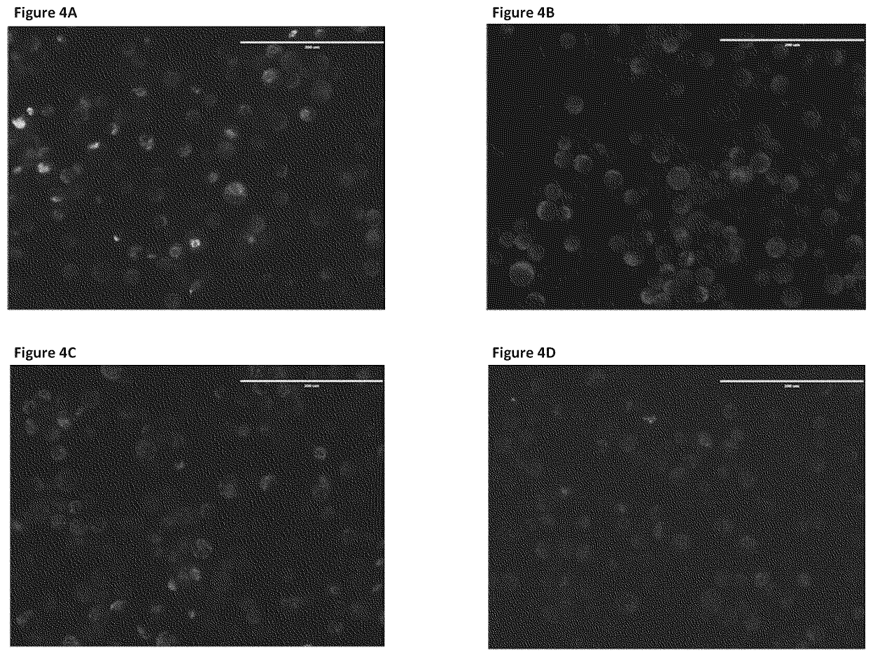

Imaging was performed 48 hours post-transfection and collected via stereoscope to determine proper subcellular localization. Images were taken in GFP and mCherry channels at 20× magnification, under the same acquisition settings, and then merged to produce FIGS. 3 and 4. Expression and localisation results of the fluorescence microscopy are summarised in Table 1.

FIG. 3 depicts the resulting fluorescence microscopy images for AtMbb1 constructs. FIG. 3A shows cells transfected with cytosol-targeting constructs; FIG. 3B shows cells transfected with chloroplast-targeting constructs; FIG. 3C shows cells transfected with mitochondria-targeting constructs; FIG. 3D shows cells transfected with peroxisome-targeting constructs.

FIG. 4 depicts the resulting fluorescence microscopy images for CcMbb3 constructs. 4A shows cells transfected with cytosol-targeting constructs; 4B shows cells transfected with chloroplast-targeting constructs; 4C shows cells transfected with mitochondria-targeting constructs; 4D shows cells transfected with peroxisome-targeting constructs.

TABLE 1
Fluorescence microscopy results

| Gene of | | | | |
|---|---|---|---|---|
| Interest | Cytosol | Chloroplast | Mitochondria | Peroxisome |
| AtMbb1 | Strong | Moderate | Moderate | Weak |
| | expression, | expression, | expression, | expression, |
| | proper | proper | slight cytosolic | proper |
| | localization | localization | localization | localization |
| CcMbb3 | Strong | Weak, | Moderate, slight | Weak |
| | expression, | proper | cytosolic | expression, |
| | chloroplast | localization | localization | proper |
| | localization | | | localization |

Protein expression is confirmed in all AtMbb1 and CcMbb3 transfections. We have confirmed plasmid sequence via PCR and digestion for all 12 constructs. Targeting sequences are functioning for most constructs.

### Example 2—Expression of Asparagopsis taxiformis Mbb1 (AtMbb1) in Arabidopsis

Four vectors suitable for the stable transformation of A. taxiformis Mbb1 (AtMbb1, SEQ ID NO: 80) were designed and introduced into Arabidopsis. The gene sequence underwent codon optimisation for expression in Arabidopsis. A 3XHA epitope tag (SEQ ID NO: 95) was added to the gene to facilitate detection and quantification of the expressed protein. Signal peptides were added to direct Mbb1 expression to the chloroplast, mitochondria and peroxisome, respectively. Mbb1 expression was driven by a CaMV 35S promoter (SEQ ID NO: 92). Transgenic plants were further analysed by molecular analysis to determine whether the plants had been successfully modified, whether the constructs were successfully transcribed into mRNA, and whether the constructs were successfully translated into mature protein, as described herein.

Construct design: For consistency with the transient expression experiment for the three proteins described in Example 1, the same signal peptides used in transient expression were utilized for making transgenic Arabidopsis plants expressing fusion proteins that are targeted into different organelles. These signal peptides were fused to the N- or C-terminus of AtMbb1, while 3XHA tag was fused to the C-terminus of this gene. Two linkers (Linker 1 (SEQ ID NO: 93) and 3XGGGGS (SEQ ID NO: 94)) were used for linking the different components (a signal peptide, AtMbb1 protein and the 3XHA tag) to make fusion proteins. No signal peptide was found in AtMbb1, suggesting that it is a cytosol-localized protein, so AtMbb1-HA without additional signal peptide was considered as cytosol-localized. The coding sequences were codon optimized for expression in Arabidopsis, the gene cassettes synthesized and cloned into pCAMBIA1300-35S vector where they are driven by an enhanced cauliflower mosaic virus 35S promoter.

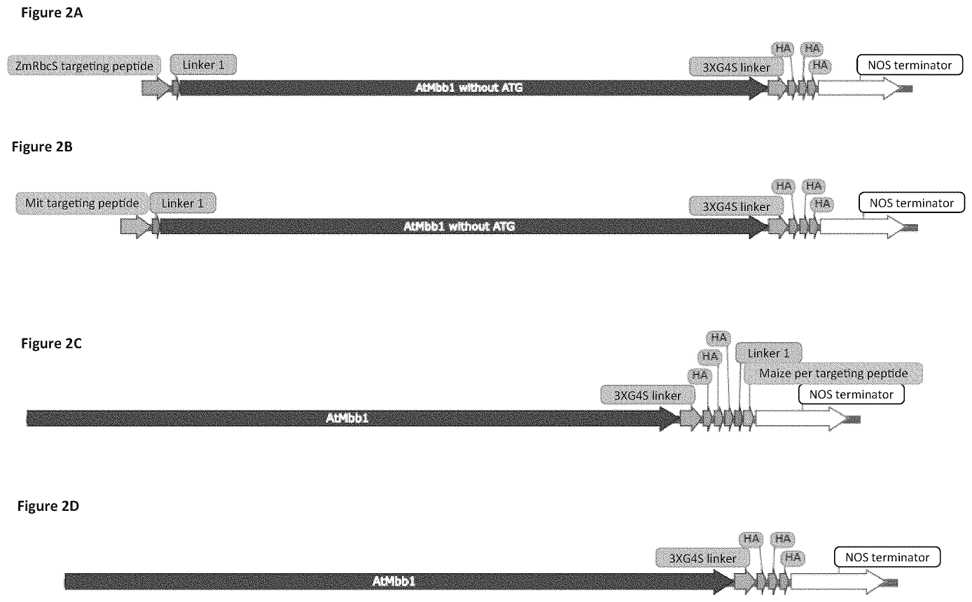

Example indication of how such constructs may be structured can be seen in FIGS. 2A to 2D. In the context of the invention, alternative epitope tags, genes (such as those described herein), promoters, terminators and linkers can of course be used where appropriate, and some components such as epitope tags can be omitted entirely if desired. FIG. 2A shows Chloroplast-localized AtMbb1. A signal peptide from maize RbcS protein was put in the N-terminus of AtMbb1, followed by 3XHA. ATG of AtMbb1 was removed and two linkers added to optimize protein expression. The cassette was synthesized and cloned into pCAMBIA1300-35S, where it is driven by an enhanced 35S promoter.

FIG. 2B shows Mitochondria-localized AtMbb1. A mitochondrial targeting peptide from maize SOD3 was fused into the N-terminus of AtMbb1, followed by 3XHA. ATG of AtMbb1 was removed and two linkers added to improve protein expression. The cassette was synthesized and cloned into pCAMBIA1300-35S, where it is driven by an enhanced 35S promoter.

FIG. 2C shows Peroxisome-localized AtMbb1. AtMbb1 was fused with 3XHA, followed by a peroxisome targeting peptide. Two linkers were added to improve protein expression. The cassette was synthesized and cloned into pCAMBIA1300-35S, where it is driven by an enhanced 35S promoter.

FIG. 2D shows Cytosol-localized AtMbb1. No signal peptide was found in AtMbb1, indicating AtMbb1 is a cytosol localized protein. So AtMbb1 was fused with 3XG4S linker and 3XHA. The cassette was synthesized and cloned into pCAMBIA1300-35S, where it is driven by an enhanced 35S promoter.

Introduction of constructs into Agrobacterium: Plasmid DNA was transformed into E. coli, DNA was extracted and confirmed via PCR, and subsequently transformed into Agrobacterium. The finished constructs were introduced into Agrobacterium strain GV3101 by electroporation. The vector identity was confirmed by PCR after transformation.

Agrobacterium-mediated transformation in Arabidopsis: Arabidopsis ecotype Columbia (Col-0) was used in Agrobacterium-mediated transformation by using the floral dip approach (Clough and Bent, 1998. Floral dip: a simplified method for Agrobacterium-mediated transformation of Arabidopsis thaliana. Plant J.). Seeds were collected from mature plants after transformation. Seeds were harvested from the plants, sterilized, and plated on hygromycin selective growth media. Plants exhibiting an elongated hypocotyl after a prolonged dark treatment were transferred to soil for genotype analysis. Tissue was harvested from rosette leaves of young plants and either DNA was isolated or the tissue was flash frozen for RNA and protein extraction for downstream assays.

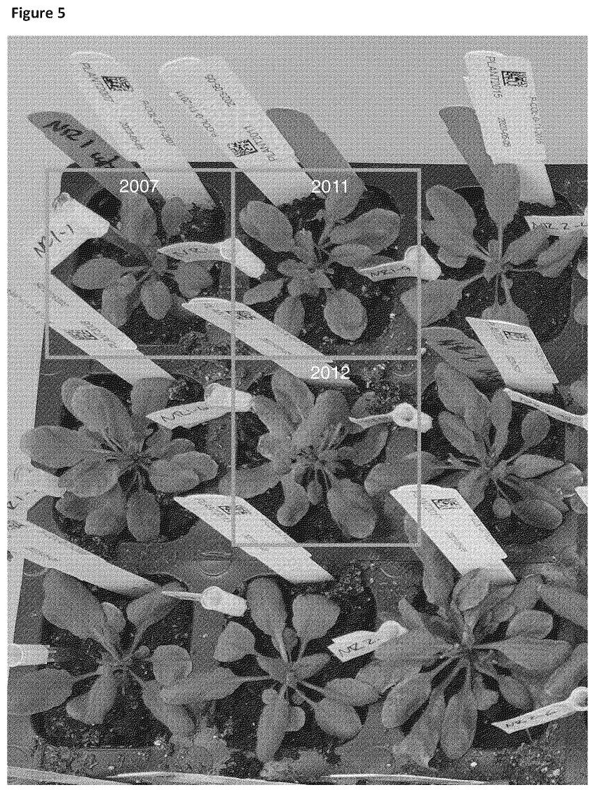

Plant growth phenotype: Plants were grown and assessed for morphology and growth phenotype. FIG. 5 shows transgenic plant lines 2007, 2011, and 2012 (labelled boxes) and six wild type Arabidopsis plants (not labelled). There is no discernible difference between the phenotypes of the plants, confirming that transgenic plants exhibit a normal phenotype and grow normally.

Selection of transgenic Arabidopsis: Screening of transgenic Arabidopsis was conducted by placing seeds on the selection medium (half-strength MS) with Hygromycin (50 mg/L). Two weeks later, the resistant seedlings were transferred into soil.

Genotyping: Genotyping of transgenic plants was conducted by PCR using primers specific to the selectable marker (hyg—hygromycin resistance gene) used for selection of transgenic events. A total of 74 plants tested positive for the presence of the hyg marker by PCR, with results visualised on agarose gel. 32 hyg-positive plant lines were carried forward for further analysis. The number of copies of the T-DNA present in the Arabidopsis genome in each event was determined by digital droplet PCR analysis, the results of which are set out in Table 2, below. DNA was isolated from the previous genotyping assay for copy number analysis. Probes targeting the transgene were compared to a gene with known copy number to arrive at a copy number variation (CNV) value which is relative to the copy number of the known gene. Plants are grouped by the target signal of the transfection construct. The results confirm that plants were successfully modified with the constructs.

TABLE 2
Plant copy number results

| Plant | | | | Plant | | | |
|---|---|---|---|---|---|---|---|
| ID | Copy | Plant ID | Copy | ID | Copy | Plant ID | Copy |
| (cyto) | Number | (chloro) | Number | (mito) | Number | (peroxi) | Number |
| 2007 | 0.98 | 2016 | 2.95 | 2027 | 4.23 | 2030 | 2.05 |
| 2008 | 12.5 | 2018 | 0.92 | 2344 | 3.98 | 2031 | 1.48 |
| 2010 | 11.2 | 2019 | 2.84 | 2345 | 30.7 | 2032 | 1.03 |
| 2011 | 3.51 | 2022 | 4.55 | 2346 | 31.1 | 2033 | 12.01 |
| 2012 | 4.49 | 2023 | 2.29 | 2347 | 7.8 | 2034 | 0.8 |
| 2013 | 35.4 | 2024 | 2.51 | 2348 | 5.2 | 2035 | 20.1 |
| 2014 | 2.2 | 2025 | 6.63 | 2349 | 0 | 2036 | 18.68 |
| 2015 | 7.6 | 2026 | 0.73 | 2350 | 20 | 2037 | 1.7 |

Genotyping was further carried out on T2 (second generation) plants descended from the above lines. PCR was carried out on T2 plant material using the same methods as described above. These results confirmed that the transgenes present in T1 (first generation) plants were inherited by T2 plants.

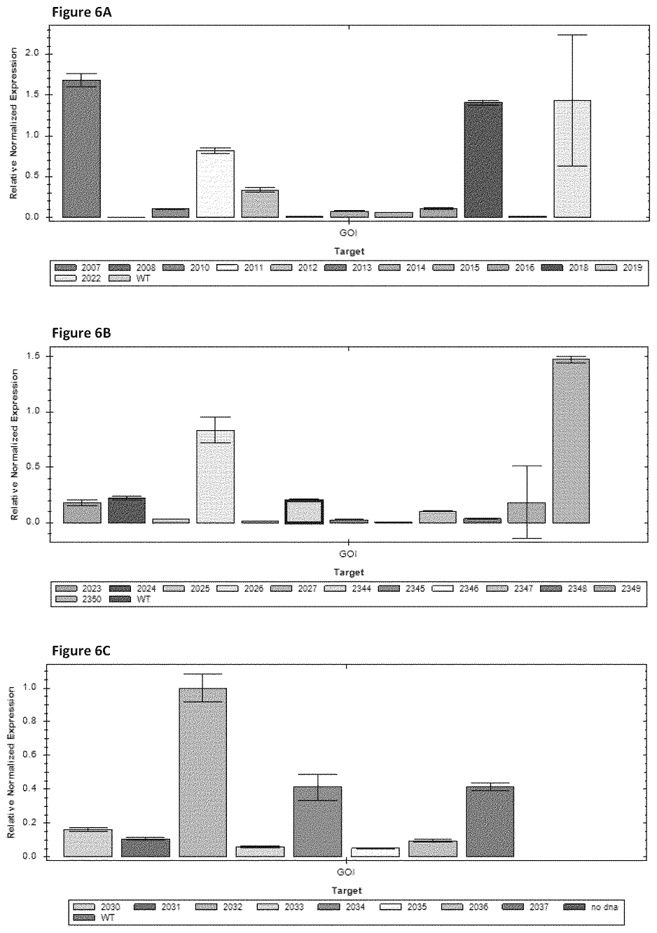

Transcription analysis: The levels of Mbb1 transcripts were then measured by qRT-PCR in each event. RNA was extracted from rosette leaves, and cDNA concentration was normalised across all samples. Gene of interest (AtMbb1) expression was normalized to WT-Actin using ΔΔCq analysis. The results are shown in FIGS. 6A-C. Table 3 recites the results of Plate 1 depicted in FIG. 6A; Table 4 recites the results of Plate 2 depicted in FIG. 6B; Table 5 recites the results of Plate 3 depicted in FIG. 6C. Samples were split across three different plates and cannot be directly compared. WT expression is on the far right of each of FIGS. 6A-C and has a normalized expression of zero. The results show that constructs are successfully transcribed by the plants.

TABLE 3
Plate 1 transcription results

| Plant ID | Expression |
|---|---|
| 2007 | 1.6813888 |
| 2008 | 0.0093421 |
| 2010 | 0.1086749 |
| 2011 | 0.8176041 |
| 2012 | 0.3359215 |
| 2013 | 0.020181 |
| 2014 | 0.0806747 |
| 2015 | 0.0665885 |
| 2016 | 0.1098342 |
| 2018 | 1.4071898 |
| 2019 | 0.0210561 |
| 2022 | 1.4357404 |

TABLE 4
Plate 2 transcription results

| Plant ID | Expression |
|---|---|
| 2023 | 0.17853 |
| 2024 | 0.22615 |
| 2025 | 0.03234 |
| 2026 | 0.83602 |
| 2027 | 0.01342 |
| 2344 | 0.19992 |
| 2345 | 0.02670 |
| 2346 | 0.00739 |
| 2347 | 0.10461 |
| 2348 | 0.03683 |
| 2349 | 0.18252 |
| 2350 | 1.47380 |

TABLE 5
Plate 3 transcription results

| Plant ID | Expression |
|---|---|
| 2030 | 0.16285 |
| 2031 | 0.10667 |
| 2032 | 1.00000 |
| 2033 | 0.06003 |
| 2034 | 0.41210 |
| 2035 | 0.05307 |
| 2036 | 0.09461 |
| 2037 | 0.41223 |

Translation analysis (T1): The production of the Mbb1 protein was analyzed by Western blotting using antibodies against the HA tag. Transgenic Arabidopsis previously identified as being selection marker-positive were grown in a growth chamber under long day conditions to increase leaf material for sampling, with seeds harvested and preserved. Leaf tissue was flash frozen in liquid nitrogen and stored at −80 C until needed for total protein extraction. Frozen tissue was homogenized using a bead beater and protein extracted in an extraction buffer along with centrifugation to remove excess plant material. Protein concentrations were measured via Bradford assay against known BSA standards. Samples for Western blot analysis were normalized to 25 ug total protein, run on Tris-Glycine gels and transferred to PVDF. Equal loading was determined by Ponceau staining. Antibodies raised against the HA-tag were used to detect protein, with a positive control confirming correct detection.

Presence of the transgene-encoded protein was confirmed by Western blot performed using the above procedure, confirming successful translation. Lines which tested positive for protein were: (Cytosol-targeting) 2007, 2011, 2012, 2331, 2332, 2333, 2335; (Chloroplast-targeting) 2018, 2023, 2026; (Mitochondria-targeting) 2350, 3652, 3657, 3660, 3664; (Peroxisome-targeting) 2030, 2032, 2038, 2353

Translation analysis (T2): Further analysis of protein expression in T2 (second generation) plants descended from selected plant lines tested above was conducted by Western blot using the same procedure as described above. Lines 2007, 2023, 2335, 2350, 2353 and 3652 were submitted for analysis. Pools of 10-day old T2 seedlings previously grown were subsequently frozen and stored at −80 C. Total protein was extracted under native conditions and Western blots performed to visualize transgene-encoded protein in the T2 generation. 75 μg of total extract was loaded for all T2 samples.

Presence of the transgene-encoded protein was confirmed in all lines tested except 2335, which was inconclusive. These results demonstrate that the transgene is heritable across generations and may be successfully translated in T2 generation transgenic plants.

Thus, successful transformation of Arabidopsis plants with vectors as described has been achieved. It has further been shown that transgenic Arabidopsis plants express the transgene of interest, and that mature protein encoded by the transgene has been detected by Western blot. It is therefore expected that plants expressing VHPO encoded by the transgene will produce bromoform when grown.

### Example 3—Liquid Culture of Arabidopsis Seedlings

Whilst it is expected that transgenic Arabidopsis plants of the invention, such as those described in Example 2 may produce bromoform without the addition of any additional substrates or cofactors, the plants may be grown in liquid culture with the addition of substrates and cofactors (such as KBr, Sodium orthovanadate, pentane-2,4-dione, and H₂O₂) to encourage and/or increase bromoform production further.

For each transgenic plant sample prepare approximately 100 seeds in a 1.5 mL Eppendorf tube, add 1 mL of sterile water and mix well. Put the tube upright at room temperature (23° C.-25° C.) for 10 min. Remove the water and add 1 mL of fresh sterilisation solution (5% (v/v) bleach, 0.01% (v/v) Tween 20 in sterile water). Gently mix on a shaker for 5-10 min. Discard the sterilisation solution and wash with 1 mL sterile water for 5 min. Repeat this step 3 times. This step should be performed in a laminar flow hood to avoid contamination.

Keep the surface-sterilized seeds in the closed tube with 1 mL sterile water at 4° C. in the dark for 2-4 days to promote uniformed germination prior to using the seeds in experiments. Prepare half strength Murashige and Skoog (½ MS) liquid growth medium (Sigma-Aldrich) containing 0.5% w/v glucose (or sucrose) and 0.1% w/v MES buffer. Adjust pH to 5.7 using 5M KOH. Autoclave to sterilise. For each sample transfer the 100 sterile seeds into a sterile 250 ml Erlenmeyer flask containing 50 ml of sterile ½ MS medium. Add lids and incubate the flasks on an orbital shaker at ~150 rpm placed in a growth chamber set to standard Arabidopsis growth conditions (20-22° C., 16 h light [100 μmol m-2 s-1 PPFD], 8 h dark).

After 5-7 days there should be clumps of young seedlings growing in the flasks. Pour off the ½ MS media, then replace with fresh ½ MS media containing appropriate substrates and cofactors for bromoform production (50 mM KBr, 10 μM sodium orthovanadate, 1 mM pentane-2,4-dione and 2 mM H₂O₂) and incubate for up to 5 to 7 days. The seedlings can then be rinsed with sterile water, blotted dry on paper towel, weighed, frozen in liquid nitrogen and stored at −80° C. for analysis. Note the number of seeds per sample and medium incubation volumes can be scaled down to do cultures in smaller vessels such as multi-well plates—dependent on how much tissue is required for later extraction.

### Example 4—Soil Culture of Arabidopsis with Leaf Spraying

Whilst it is expected that transgenic Arabidopsis plants of the invention, such as those described in Example 2, may produce bromoform without the addition of any additional substrates or cofactors, the plants may be grown in soil culture and additionally provided with substrates and cofactors (such as KBr, sodium orthovanadate, pentane-2,4-dione, and H₂O₂) to encourage and/or increase bromoform production further.

Transgenic Arabidopsis plants approximately 4-6 weeks old were grown in soil for 3 weeks. Substrates and cofactors were provided by spraying a solution of 50 mM KBr, 10 μM sodium orthovanadate, 1 mM pentane-2,4-dione, and 2 mM H₂O₂ directly onto the rosette leaves of the plants. The leaves were sprayed with the solution three times per day for three days. A small amount of surfactant such as Tween 20 may be added to the solution to aid uptake as the leaf surface is hydrophobic.

### Example 5—In Vivo Production of Bromoform by Transgenic Arabidopsis Lines

Seedlings or rosette leaf tissue from T2 Arabidopsis lines expressing AtMbb1 were tested for the in vivo production of bromoform. Bromoform was detected by GC-MS as described by Thapa et al (2020). Leaf tissue samples collected from transgenic plants were extracted with MeOH overnight. Following incubation, the samples were vigorously agitated on a vortex mixer, centrifuged at 16,000×g for 30 min to remove debris, and an aliquot of the supernatant was subsequently analyzed by GC-MS (1260G with 7890a MS; Agilent Technologies) in electron ionization (70 eV) mode using a DF-5 ms ultra inert GC column (30 m length, 0.25 mm width and 0.5 μM film thickness). Frozen tissue samples were extracted in 300 ul/100 mg tissue, the extract was centrifuged and then 50 ul used for GC/MS analysis. Bromoform production was quantified based on calibration curves generated from a bromoform standard. The column temperature conditions will be as follows: 40° C. for 3 min, increased to 200° C. at 10° C./min and held for 1 min with a total run time of 20 min. Injection port, interface and ion source will be kept at 250° C., 300° C. and 230° C., respectively. Helium will be used as carrier gas at a flow rate of 0.9 m/min.

Bromoform standards were tested against a known concentration of naphthalene (0.5 μg/mL, retention time of 10.263 minutes), and were detectable between 0.25 and 5 μg/mL with a retention time of 7.905 minutes. 0.5 μg/mL naphthalene was included in all test samples as an internal standard. Each sample was extracted in chloroform spiked with 0.5 μg/mL naphthalene, centrifuged, and 50 μL of the supernatant run through the GCMS.

T2 leaf samples: T2 plants for each of 19 different Arabidopsis events expressing AtMbb1 were grown in soil for three weeks. Leaves from these T2 plants, grown according to Example 4 were harvested, frozen and analyzed for the presence of bromoform by gas chromatography-mass spectrometry (GCMS). Samples underwent a double extraction and concentration process. They were initially extracted with chloroform as described above. Samples were then re-extracted with 150 μL saturated brine and 50 μL methanol, vortexed to separate the phases and the upper chloroform and methanol phase was transferred to a clean tube. This lower phase was reextracted with 300 μL of methyl tert-butyl ether and the top layer transferred to the chloroform/methanol. Samples were then dried under nitrogen gas and immediately redissolved in 50 μL methanol for GCMS injection. This resulted in the detection of bromoform from a total of 4 events (2007, 2011, 2012, and 2023) whereas no bromoform was detected from wild-type Arabidopsis plants. Peaks were detected at retention times of 7.922 (2007), 7.913 (2011), and 7.918 (2012 and 2023) minutes, consistent with bromoform elution. Some samples were reinjected in a different in order to confirm that positive results were not due to reagent contamination or carryover. Lines 2007, 2011, and 2012 express cytosolic AtMbb1 (no signal sequence) whereas the AtMbb1 is directed to the chloroplasts in the event 2023.

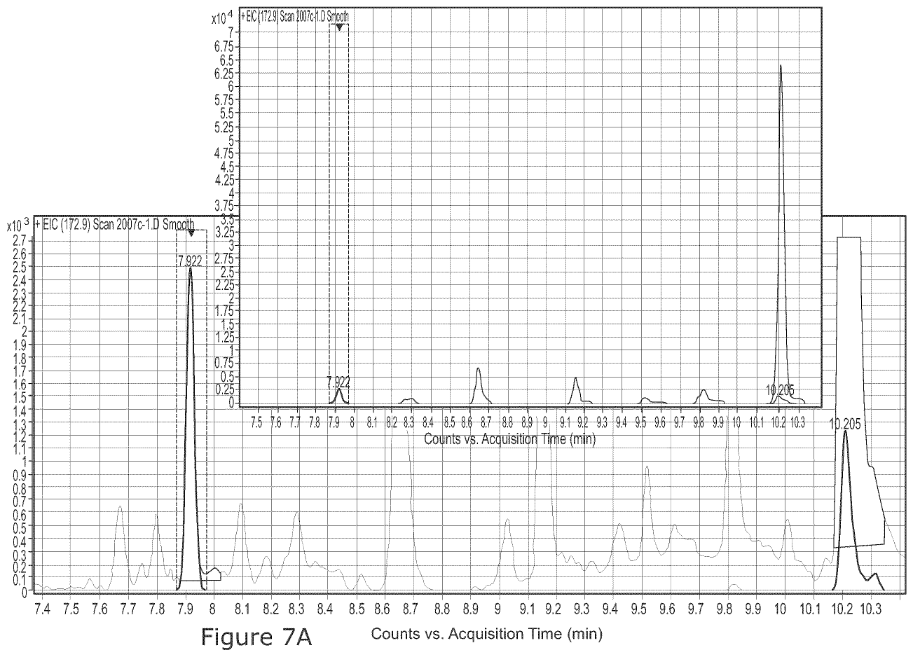

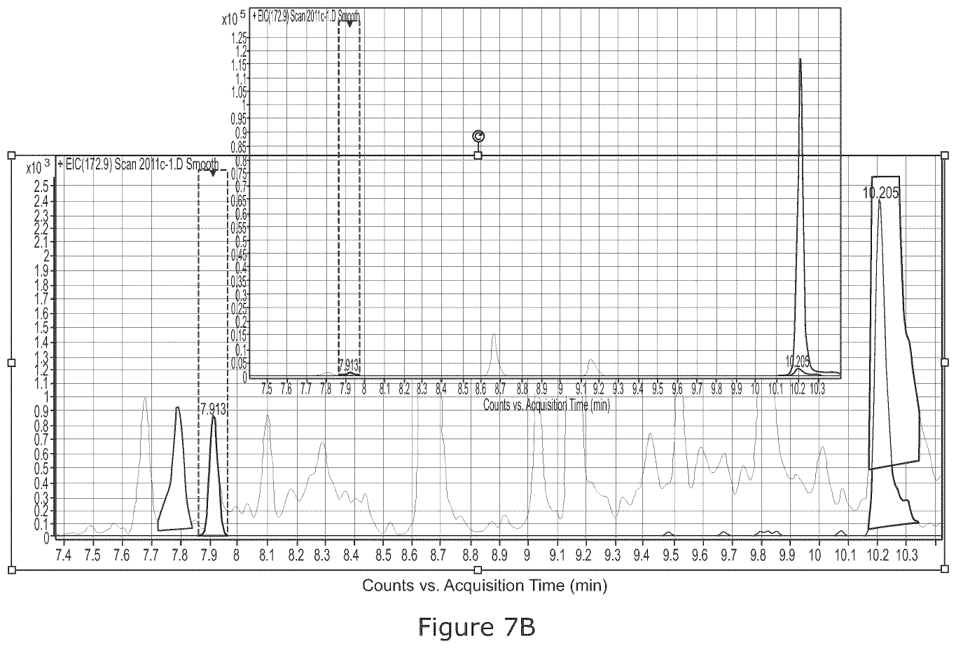

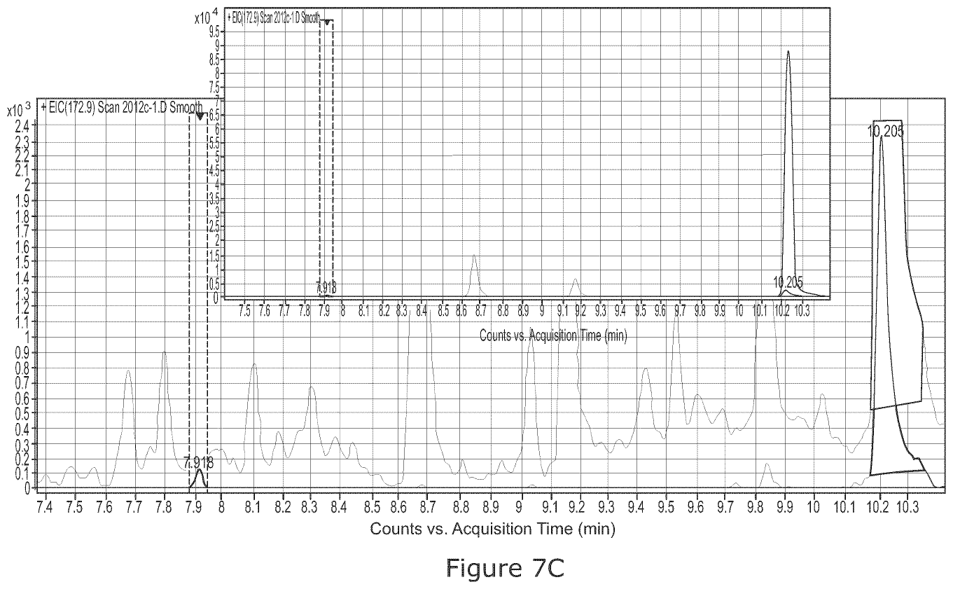

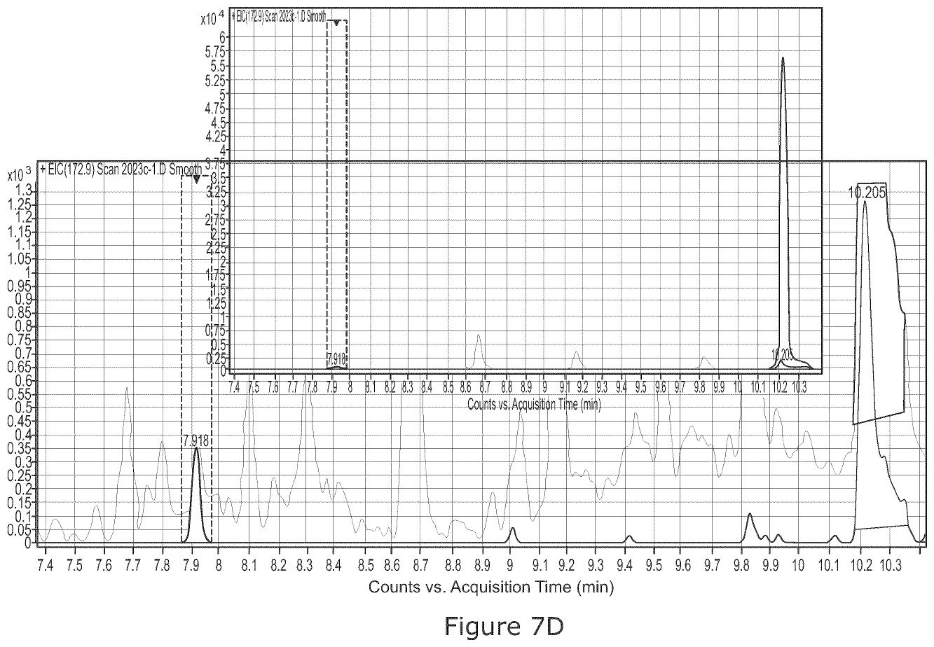

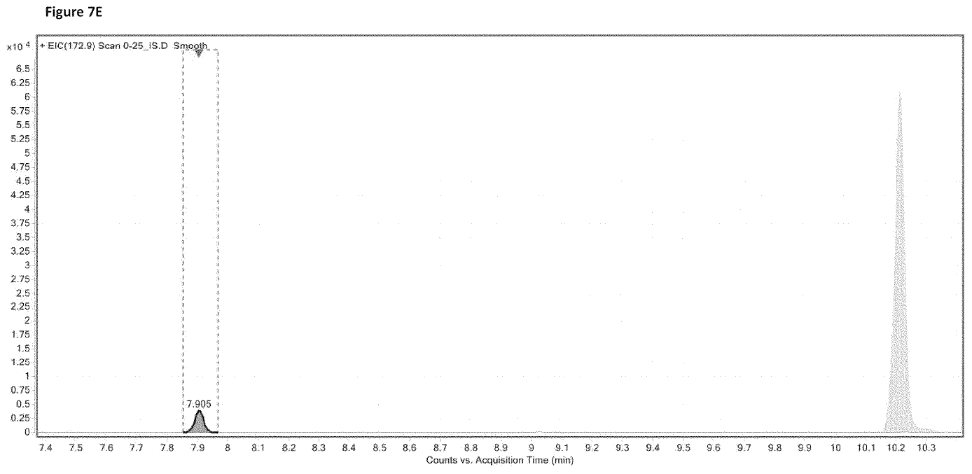

FIGS. 7A-D are gas chromatograms of T2 leaf samples; for each sample two chromatograms are presented with x-axes of different scales in order to better visualize the data. Sample material was spiked with internal standard and as such peaks at elution times for bromoform and naphthalene are visible in each sample chromatogram. The peaks labelled with elution time relate to the sample material of the combined extracted ion chromatogram. FIG. 7E is a chromatogram of the positive control: 0.25 μg/mL bromoform standard. FIG. 7A is the chromatogram for the 2007 sample. FIG. 7B is the chromatogram for the 2011 sample. FIG. 7C is the chromatogram for the 2012 sample. FIG. 7D is the gas chromatogram for the 2023 sample.

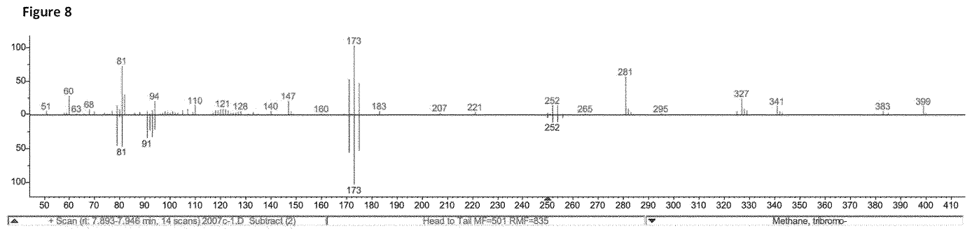

The mass spectra of samples were then analyzed to confirm the identity of the bromoform peak. As an example, the mass spectrum from event 2007 is shown in FIG. 8, along with the NIST reference spectrum for bromoform. The 2007 sample mass spectrum is shown above, the NIST reference spectrum below. The 2007 sample spectrum shows a strong match to the expected spectrum for bromoform, with corresponding peaks for full and partial bromoform ions. This confirms the presence of bromoform in the 2007 sample, initially detected by gas chromatography.

T2 seedling samples: Bromoform was also detected in plant material taken from T2 seedlings grown according to Example 3 and tested according to the same protocol as described above in Example 5. In addition to the transgenic T2 seedlings, wild-type (non-transgenic) Arabidopsis seedlings were analyzed in parallel as a negative control. An extract of A. taxiformis algae was used as a positive control. Commercial bromoform was used as a standard. Bromoform peaks were observed by gas chromatography and peaks matching full bromoform ions detected by mass spectrometry in T2 seedling samples.

Taken together, in these examples we have shown that algal VHPO enzymes can be expressed in functional form in plant cells, and that transgenic plants expressing such VHPO enzymes are able to produce bromoform in vivo.

TABLE 6
List of Identified Sequences

| SEQ ID | | |
|---|---|---|
| NO | Description | Sequence |
| 1 | A. taxiformis | MTDTQNPNRAEVAFKVRVSAAELARARGSPTHLSNNSESR |
| | Vanadium- | FRNPDGTRSLLANFTKGLPHTKETALVVSAIDYDSFVRAI |
| | dependent | DSGDPRDFADLPLGPQGVEPRFTSGIASDPEVGTRAWESG |
| | haloperoxidase | GAGLVFDLEGPDAQAVTMPPAPELDSDELVTEVTECYWMS |
| | Mbb1 | LLRDVPFPTFDSDPHIQSAAESINNTQWIQFKNNPPAHLT |
| | [QKI80117.1] | AAERSRLRGPITTANVFRGITPGDEVGPYLSQFLLVGTTG |
| | | IANGNEVGDGFIQYGGMRMDQRVRVAKSHIDYMTTFGAYL |
| | | DVQNAANVSGRELYNEEEPRFRFIHTPRDLATYVHFDALY |
| | | QAYLNACIILLDIGAPFDSGIPFQLDNDIDKQQGFATFGG |
| | | PHILSLVTEVATRALKAVRFQKFNVHRRLRPEAIGARVDR |
| | | YCATKAPEFAGAAKLSEALDKKLLQKVHDHNKKQNLLSDR |
| | | GNPRANDFNPDGDVSKGNLLMPMAFPEGSPMHPAYGAGHA |
| | | TVAGACVTVLKAFFDGGYRLPFCYITDENGTGLKAVEIDE |
| | | PLTVDGELNKICSNISIGRNWAGVHYFTDYIESIRIGEEI |
| | | AIGILQEQKLTFSENFSMTLNKFDGSTIRI |
| 2 | A. taxiformis | MAKQKSLFTLLSATLESHLSTHAFQLLALIIYALANALMF |
| | NAD(P)H | VWGAHDEFHHHTDANNLRWYICIARGAGYTLNLNTALVIL |
| | oxidase Mbb2 | LAARLFATYLRETPLQHILPLDKSFPAFHIVVAYTIAAAV |
| | [QKI80118.1] | VIHASFHLAWLVAYDMWETGMWGFTMSAATGVVLLVVFIV |
| | | MFISAMPKYRKKHFRIFYLIHSVGALLFFGLLVFHGMYNR |
| | | VPETYKWIAAPLIIYTIDRVLRRYKISTAELELTGEHSSL |
| | | KGSDILELRVPKPFDYQAGQYAEVCVKSINSEWHPFTIAS |
| | | SPHEDSMCFYIKALGDWTTNLRDAFEARVENDLYEPLKVQ |
| | | IRGPFGAPAQHVSGYCRVVLISGGVGSTPFAAICKHLHHL |
| | | NKSENHSKALESQVSSKRQSQVQWRIRDAISILFDVSLDD |
| | | SRNDEAANQQRRQQLADMLNMSPKGDVHNVSFKRTQSKRS |
| | | DDFDAVEDSKQMNFSLCRQDSKSSFNLDDCSDAIRYYNRP |
| | | SFQFFSSSPRHVINWYEYRTRLLAFLHTTRFTFALLLTLV |
| | | ARIVILCIVSIFNLGHIGLYNSHIDATWVIATNSILGLIL |
| | | GVALLTTILLEISFMRVRFFYRVWRCVDFFIFLPITFLCN |
| | | ISNFASWNGHAQPKFLIFLDLIIVLPIMLLLLCHRMYRSV |
| | | GSRNLLDDTAECRQGCKCNKTIPDVDFVWTTPRDSDDEWL |
| | | RNELYPLATGTELRLHRYVTRENMADLEDPERFITTANAG |
| | | RPQWDEIFAQIAEQTPSHSKIGVFFCGPHPMGAAVQKSMR |
| | | KVEVMSNLRGSYLRKTESAVLVDDLILRDEGEVKLLREYG |
| | | CNIRFVFREENFS |
| 3 | A. taxiformis | MTPPTRRQTALVKRIAAADLAANRHHPLHVRNADETTYKL |
| | Vanadium- | PNHVCSYISAFTKGLPHHLDGRLVSPHHFQLFVHAVQTGH |
| | dependent | VTDIAAIPLGPPHSSSFRSGIAKGAAVRAWESMAAGNTFD |
| | haloperoxidase | LQGPDAQAVTMPPCPRLNSHELVTEMTELYYMALLRDVPF |
| | Mbb3 | THFNKHHLVQSAVHDMNHTHWTRSNRCCLPRPERRRHRSK |
| | [QKI80119.1] | FTTDNIFRGVTPGDITGPYISQFLLVGTKGLGDAHEETEG |
| | | YVQYGATRIDQRVRTVEPKIDYMTTWAAYLDVQNGADLRR |
| | | HSTYAKPPSYRFISTPRDLATYVHYDALYQAYLNACIILL |
| | | DINAPYDGGLPFTADDAVDKQQGFATFGAPHVLTLVTEVA |
| | | TRALKAVRFQKYAVHRRLRPEAVGGLLEQYRRYGGGHSDL |
| | | AYIIRPIRHLADSLSSDLMFMVAKENGRQNSSITDNGHPR |
| | | SHDEGSDDTHLLSMAYAEGSPMHPSYGAGHATVAGACVTI |
| | | LKAFFDHTFKLPFAYVSSSDGRKLKTVKLSRKLTVEDELN |
| | | KLAANISIGRSWAGVHYYSDYVESIRLGEEVAIGMLKEQK |
| | | LTYSEKFTMTIPKFDGSVIEI |
| 4 | A. taxiformis | MAEERRQNALDIRIQAARLAKKRDHPTHEANGDEDRYPET |
| | Vanadium- | LIGSFTKGLPHEKETGLLSNPADFADFVRAINTGAIKDIR |
| | dependent | RLKLGIDEDPRFISGIASKGDKHPFADTRAWESMAAGLTY |
| | haloperoxidase | DLEGPDAQAVTMPPAPKLDSDELVTEITESYWMALIRDVP |
| | Mbb4 | FTEFERDGNTAAAAASISRTRWVQFNEQPSQRPSTLTDEE |
| | [QKI80120.1] | IARLRGPYTKKNVFRGVTIGENVGPYLSQFLLVGTKGIGD |
| | | VQQISDGYIQYGSMRMDQRVRVAAPKRDYMTTWASWLDVQ |
| | | NAGDLRGREIYDDDTPFRFITTPRDLATWVHFDALYQAYL |
| | | NACIILLDIKAPFDPHIPFQADDDVDKQQGFATFGGPHIL |
| | | SLCTEVATRALKAVRFQKYNVHRRLRPEAIGGLVERFKKT |
| | | NGDAKFAPVKKLVNDLDGDMLRRIEQHNCEQNKLSDDGHA |
| | | RRDDYSPEGESNQTYLLPMAFPEGSPMHPSYGAGHATVAG |
| | | ACVTVLKAFFDHEYELDFCYVPTTDGKRLEKVNINEKLTV |
| | | EGELNKLCANICIGRNWAGVHYYSDYFESIKVGEEIAIGI |
| | | LQEQKLTYGEDFFMTLSKFDGEKIRI |
| 5 | XP_00571423 | MSAARRQEAFQTRVRAARLARDRDHQVFEPNGEEDRYFRP |
| | 7.1 vanadium- | NSQIFSYLGSYTKGLPHNRRTALITSPNHFVRFVRAIDSG |
| | dependent | EPADFIKVPLGPRPALQFESEEECRAANLFKSGIAGRNNT |
| | bromoperoxidase, | FPSSCLRAWESMGAGLVFDLEGPDAQAVGMPPAPTLDSDE |
| | vBPO | LVAEISEVYWMALLRDVRFTDFTNPGPIGRAINTLNKTAW |
| | [Chondrus | IRAARDPPDSLTPQERARLRGPFNPSNIFRGTLPGDDVGP |
| | crispus] | YLSQFLLVGTKGIGGAQDMADGFISYGAHRIDQRVRHARK |
| | | ELDYMTSWEAFLDVQNGADVTGRDTFESGSDAFRFITTPR |
| | | DLATYVHFDALYQAYLNACLIMLDSGVRFDKGIPFGEPDF |
| | | KDHQRGFAHFGGPHILSLVTEVATRALKAVRFQKFNTHRR |
| | | LRPEAVGGLIERFNSNPDDPQFQDVKPLFEALDEDMMRRV |
| | | ARHNREQNQGSDFGMPRADDFNPAGDTLETMLLPMAFPEG |
| | | SPMHPSYGAGHATVAGACVTVLKAFFQHDTELDFCFVPSD |
| | | DGSRLVDASHNMNKKLTVEGELNKVCSNISIGRNWAGVHY |
| | | FTDYIESILLGEQIALGILEEQMLTFPETFTMTVPLFSGE |
| | | LKVLRST |
| 6 | XP_00571943 | MATPCFAQAPAPTLRRSPRRLPLTPTSFLPRPFLPKPFPR |
| | 5.1 vanadium- | SPRHPHPRLNAASARTSAAQPPDRAATPCATRQQQALELR |
| | dependent | RAAAQLAHDRAHPAHVSNGSEELHLHPKTGAPLLLANFTK |
| | bromoperoxidase, | GLPHERDTGLAADAAHYDLFVAAVHSGLPDDIRAIPLGPQ |
| | vBPO | AAPGVRLPRSLRFRSGIARSSHLFPHTDVRAWESMAAGNA |
| | [Chondrus | FDLQGPDAQAVTMPPAPALASPELALEMTENYWMALLRDV |
| | crispus] | RFSQFAADPTVAAAMESMNATEWVALACGDASAVAEEERK |
| | | RLRGPFTAQNVFRGNTQGDCKGPYISQFLLSGSAGLGQVN |
| | | SGADGYIQYGAMRMDQRVRVALEGLDYMTTWESWLDVQNG |
| | | ADLRGRELYEDGERFRFICTPRDLATYVHYDALYQAYLNA |
| | | CIIMLNKKIPFDKGLPFQKDDDIDKQQGFALFGPPHILTL |
| | | CTEVATRALKGIRFQKYNVHRRLRPEAVGGRVERYHHNCE |
| | | DPLFADVKPLYDALDKDMLERVAAHNAEQNAKDDFGNSRF |
| | | EDYSPTGSSGRTYLLPMAFPEGSPMHPAYGAGHAAVAGAC |
| | | TTILKAFFDHEHELDFAYVPTADGSKLEDVVDSLGEKLTV |
| | | EGELNKVCSNISVGRNWAGVHYFTDYRESIILGEKIALGL |
| | | LEEQKLTYAEEFSMTIPLFDGSTVTI |
| 7 | XP_00571089 | MSDMSTEISPRQQEAYDIRVAAATLALARDRKRKVFEPNG |
| | 4.1 vanadium- | EEDRYFRPNSQILSYLGSYTKGLPHNRSTALVTSPNHFVR |
| | dependent | FVQAIDSGEPADFIKVPLGPRPALEFTTEEECIAANLFKS |
| | bromoperoxidase, | GIATRNDSFDPACLRAWESMGAGLVFDLEGPDAQAVGMPP |
| | vBPO | APTLDSDELVAEISELYWMALLRDVRFTDFTNPGPIRRAI |
| | [Chondrus | NTLNRTTWIRAARDPPDSLTPQERARLRGPFNPSSIFRGT |
| | crispus] | LPGDDVGPYLSQFLLVGTEGIGGAQDRADGFISYGAHRID |
| | | QRVRHATKELDYMTSWEAFLDVQNGADVAGRDTFESGSDA |
| | | FRFITTPRDLATYVHFDALYQAYLNACLILLDNKVKFDQG |
| | | IPMGEPDFNGNQGIPFQDPDFKDHQRGFAHFGGPHILSLV |
| | | TEVATRALKAVRFQKFNTHRRLRPEAVGGLIERFNSNPDD |
| | | PQFQDVKPLFEALDEDMLRRVASHNREQNERSDFGMPRAD |
| | | DFNPAGDTLETMLLPMAFPEGSPMHPSYGAGHATVAGACV |
| | | TVLKAFFQHDATLDFCYVPSDDGSRLDDASHTLNKKLTVE |
| | | GELNKVCSNISIGRNWAGVHYFTDYIESILLGEQIALGIL |
| | | EEQMLTFPETFTMTVPLFSGGFRTLISTSDP |
| 8 | Acaryochloris | MTDRRQDSYAVRVEAAELARSRNHPDHQANGDEERYAGAK |
| | marina | YFMSFTKGLPHNEETGLLGDPKDFVEFRRAIDDGFIDPFS |
| | vanadium- | SDLIRHGALYKVEGGEVKRVDREVDGDPPNNFRQWEAPTA |
| | dependent | GVVFDLEGPDAQAVTMPPAPPLLDGNGNPNPELVLEMAEV |
| | bromoperoxidase | YELAILRDQPFNDFENGRSNAAITQSIDRLNALSYISNQT |
| | | GRPRKVNTSGQLDAQTVFRGSSPGVEAGPYLSQFLLIGNT |
| | | DLNRDADGIGGGKVEDGLITYGALQINQRVPIAEPGKDYM |
| | | RSMSEYVEVQRGIRPPRETYGSTNGGSVAQPNRPPRRFIS |
| | | TPRDLSTYVHYDALYEAYLNACIILLEMGTPFDPGFDHLS |
| | | GVGAAASSFESRRNAGGFALYGGPHILTLVTEVATRALKA |
| | | VRFQKFNNHIRLRPEALAARIELVNSFDGLSQADKDTVPE |
| | | VLKKYIGLFSNALKPTLNALGGNYLLPMAFPEGSPMHPAY |
| | | GAGHATVAGACVTILKAFFDTSAVLAKSGQSIAFKRLENG |
| | | DKPIAFRAPDLPGSGPGEGVPKDDLKPFKPNHELTLEGEL |
| | | NKLAANISIGRDMAGVHYFTDYYDSLRMGEEIAIGILEEQ |
| | | ALTYSTDPFVLSVPTFDGDVRRIGRR |
| 9 | Alteromonas | MNETQNORSKDSLDYRIRIASLNRHQSIHSQVMHINNGDE |
| | naphthalenivorans | KSSINYAMSFTKGLEHNPETGLVQNAETCEALIDAINGGE |
| | vanadium- | ISPFHAIDVPTAEGKRRLWEAPTAGFVFDVQGPDAQEVTM |
| | dependent | APPPGLGSSELTFEMAEVYELAVLRDTALNKFSELSDEIA |
| | bromoperoxidase | GPCERLNKLSYAMRGFCGRPRLTAKNACGDKVLTPGTVFR |
| | | GSSPGVGIGPYLSCFMLMGGGNTKSSVESGLINYGAQTIN |
| | | QKVPKASDKNFMLEFDEWVAVQNGYSRNNTPKTGTGNYEE |
| | | NSGELKLTFIKTPRDLATYVHFDALYQAYLNACLILLSQK |
| | | ESQDFAPFDSNFVNLSGEFPNSRAGGFALFGGPHILSLVT |
| | | EVATRALKAVRYQKFQVHRRLRPEVLAARIAKSDNDAFKA |
| | | AVGGAASTNFENMKADLSDTLAKINELFHTEDLLPMAFAE |
| | | GSPMHPSYGAGHSTVAGACVTVLKAFFDTNAQLARKGESV |
| | | CFVKPEIAERDNWQPVKIVATDNDDTLTIEDSDEVITLEA |
| | | ELNKLAANISIGRNMGGVHFFSDYYDSLRMGEEIALGILK |
| | | DQAESYPYDAFTLSVKTFDGDEKLITRS |
| 10 | Ascophyllum | MMKASGGIITALLAAFPPCLSQTVNELADMQTRPLLSGSV |
| | nodosum | CRVRDTVDFLSPTKRAKITFKRRIGIAAGELAVGPTCHLN |
| | vanadium- | NGDEANVPLFDGQFHKSLPHDDVGRVNPEAYQLLLDCIES |
| | dependent | NDINVCDQVPSGVESDGRKLVNPLGGGGHQVDGADSDNIF |
| | bromoperoxidase | IKQPDNLLSERLAAQQAEVYWMALLRDIPFSQFGTNNTVQ |
| | | MAAANLQGFDAFNGLSISRDADGNIDPMQDLFRTDWPGVS |
| | | SGPMVSQFMLANFDIDGIVVEPKAKTLVPEMEYMTGVDTW |
| | | LNIQNGGPPEDTLFVDEPLFIRNGRDLAALSFNDVLYTEA |
| | | FRTILILFNESILAEAGPYGSSTRQEGFTTLGTSHYIHAM |
| | | AAGSSSTRHAWYAKWQVHRVLRPEAYGGLLHFVINNLIDD |
| | | VPLPASIVSNTELLNAVESLNQAQNGGTNQVFLLPMAVGE |
| | | GSPVHPAYPSGHAINLGAYLTVLKAFLGFELGQRCFPSPM |
| | | ISNDAGTDRIPFVPSDGDRVGTCINEDGEEEVGLTYEGEL |
| | | NKVTSNVAIGRSHLGVHWRMDGVFGAEMGEAGAIRRLQQE |
| | | LGGLPEARDTEGPIPPASYKFRLYSGTMIELFPDNRYMLG |
| | | DQMCKGFFTGDDFCVPADEEETAELEDLVLGSVVDQTCAE |
| | | L |
| 11 | Corallina | MGIPADNLQSRAKASFDTRVSAAELALARGVVPSLANGEE |
| | officinalis | LLYRNPDPENGDPSFIVSFTKGLPHDDNGAIIDPDDFLAF |
| | vanadium- | VRAINSGDEKEIADLTLGPARDPDTGLPIWRSDLANSLEL |
| | dependent | EVRGWENSSAGLTFDLEGPDAQSIAMPPAPVLTSPELIAE |
| | bromoperoxidase | IAELYLMALGREIEFSEFDSPKNAEYIQFAIDQLNGLEWE |
| | | NTPAMLGDPPAEIRRRRGEVTVGNLFRGILPGSEVGPYLS |
| | | QYIIVGSKQIGSATGGNKTLVSPNAADEFDGEIAYGSITI |
| | | SQRVRIATPGRDFMTDLKVFLDVQDAADFRGFESYEPGAR |
| | | LIRTIRDLATWVHFDALYEAYLNACLILLANRVPFDPNIP |
| | | FQQEDKLDNQDVFVNFGDAHVLSLVTEVATRALKAVRYQK |
| | | FNIHRRLRPEATGGLISVNKIAAEKGESVFPEVDLAVEEL |
| | | EDILEKAEISNRKQNIADGDPDPDPSFLLPQAFAEGSPFH |
| | | PSYGSGHAVVAGACVTILKAFFDSNFQIDQVFEVDKDEDK |
| | | LVKSSFKGTLTVAGELNKLADNIAIGRNMAGVHYFSDQFE |
| | | SILLGEQVAIGILEEQSLTYGENFFFNLPKFDGTTIQI |
| 12 | Corallina | MGIPADNLQSRAKASFDTRVAAAELALARGVVPSFANGEE |
| | pilulifera | LLYRNCETGDPSFIASFTKGLPHDDNGAIIDPDDFLAFVR |
| | vanadium- | AINSGDEKEIADLTLGPARDPETGLPIWRSDLANSLELEV |
| | dependent | RGWENSSAGLTFDLEGPDAQSVAMPPAPVLMSPELIAEMA |
| | bromoperoxidase | ELYLMALGRDIEFSEFESPKNAAFIRSAIERLNGLEWENT |
| | | PAKLGDPPAEIRRRRGEVTVGNLFRGILPGSEVGPYLSQY |
| | | IIVGSKQIGSATVGNKTFVSPNAADEFDGEIAYGSITISQ |
| | | RVRIATPGRDFMTDLKVFLDVQDGADFRGFESYEPGARLI |
| | | RTIRDLATWVHFDALYEAYLNACLILLANGVPFDPNLPFQ |
| | | QEDKLDNHDVFVNFGSAHVLSLVTEVATRALKAVRYQKFN |
| | | IHRRLRPEATGGLISVNKKSFLAGSDIIFPEVSELVEELS |
| | | SILDDVAESNEKONRADGIVSPDKSFLLPMAFAEGSPFHP |
| | | SYGSGHAVVAGACVTILKAFFDANFQIDKVFEVDTDEDKL |
| | | VKSSFKGTLTVAGELNKLADNVAIGRNMAGVHYFSDQFES |
| | | LLLGEQIAIGILEEQSLTYGENFFFNLPKFDGTTIQI |
| 13 | Gracilaria | MASHIPRFTKAYAVREQAALMAKSRPHPLHVSNNEELRYR |
| | changii | IPTSGEPSHIGSYTKGLPHDRNTGVLSNPDDFQSFIRAMD |
| | vanadium- | AGDQFSIRDVPLGPQNEQQAFQSGIAKGVSARAWESMAAG |
| | dependent | LTYDLEGPDAQSVTMPPAPALDSDELLTEVTELYWMALLR |
| | bromoperoxidase | DVPFSEFNGDKNNDIQEAVDSLNSTPWVQIAKNGVVPTTL |
| | | TDAERVRLRGPFTVDNVFRGTTRGDRDGPYLSQFMLIGNT |
| | | GIGNGNRIEDGFIRYGAHRVDQRVRVAEMGRDFMTTWSAF |
| | | VDVQNGADVRGREVYNNDVRYRFISTPRDLATYVHFDALY |
| | | QAYLNACLILLGMRVPFDKGLPFQREDDIDHQQTFATFGA |
| | | PHILSLVTEVATRALKAVRFQKFNVHRRLRPEAVGGLLHR |
| | | YMQETTNDLYRGAKQLAENLDANLLDKVKRHNAKQNMKSD |
| | | GDAPRANDLGECEDADEITLLPMAFPEGSPMHPSYGAGHA |
| | | TVAGACVTILKAFFDHGYELRMLDEDGETMIPFAFVPTAD |
| | | GSGLENRIGEYRNLTALTVEGELNKVCSNICIGRNWGGVH |
| | | YFSDYRESIRVGEQVAIGLLEEQKLTYGENFSMTVPLFDG |
| | | SVHRI |
| 14 | Laminaria | MKISSLGLTALLAAFAPCLGYEEPPEPTQPLLSGNVCRVR |
| | digitata | DSLDFLDPVPRAKVTLLKRLAIAKDEISVGPTCHINNGDE |
| | vanadium- | ENVPLFAGQYHKTLPHDKFGQVDEDAYKKLLECVFTSDIN |
| | dependent | ECEKVPSGAGRRGGAKLTNPLGGTAHQVTGADSDNVFITT |
| | bromoperoxidase | PDSLLSERLAAQQAEVYWMALLRDIPFGEFAKNDYVRLAA |
| | | ENLQGLPAFKGLNIPRSEGGKIDPVTDLFRTTWPGVTTGP |
| | | VVSQFMLSDFLIDSIKVTPKADPLTPGVDYMTAFQPWLDV |
| | | QNGASKLETTFDEENPRFIRNGRDLATIALRDQLYTEAFR |
| | | AALILFTEGALGGEVGPYAEAERQQGFATFGEPHILTAMA |
| | | SASSVTRHAWYAKWQVHRMLRPEAYGALVHNTLMRDVITP |
| | | LPDSILRNTELLNRVEVHNQRMNPDGEKTFLLPMAAAQGS |
| | | PTHPAYPSGHAINNGAYITALKAFLGYEAGQKCFPNPVVS |
| | | NDEGTKRIKYKPSGREIVGECVNEKGKLVEGLTYEGELNK |
| | | ISANVLLGRSHIGVHWRMDGVYGALMGETSCVRRLQQELP |
| | | GLPEAREVEGKKRRGDIPPATYKFRLYSGKILELYGRNLY |
| | | KLDGKLCEGAFTGDDFCDPIDEDDYSSFDDIVEEHAQFSL |
| | | HGHTEL |
| 15 | Asparagopsis | MATPDTQNPNRAEASFKIRLSAAELARSRGSPTHISNNSE |
| | armata Azores | ARFRNPDGTRSLLANFTKGLPHINETGLVQSALDYDQFVR |
| | - Mbb1_like | AIDSGDARDFAALPLGPQNVTPRFTSGIATNPEQSGVRAW |
| | | ESMGAGRTFDLEGPDAQAVTMPPAPELDSEELVTEVTECY |
| | | WMSLLRDVPFPTFHCDEDVAAAAKSINDTQWIKFKENPPT |
| | | HLTSAERSRLRGPIDASNVFRGITPGDDVGPYLSQFLLVG |
| | | TTGIANGNEVSDGFIQYGGMRMDQRVRVASPKVDYMTTFG |
| | | SWLDVQNAADVTGRERYESDPGFRFIHTPRDLATYVHFDA |
| | | LYQAYLNACIILLDIGAPFDSGVPFQLDNDIDKQQAFATF |
| | | GGPHILSLVTEVATRALKAVRFQKFNVHRRLRPETVGGRV |
| | | DRYYKTKAPEFSGAAKLVDSLDSDLLDRVYYHNKKQNKLS |
| | | DNGAPRSADFNPDGGLSEGNLLMPMAFPEGSPMHPAYGAG |
| | | HATVAGACVTVLKAFFDGGFPLPFAYVTNENGCGLEPVEV |
| | | SEPLTVDGELNKICSNISIGRNWAGVHYFTDYIESIRIGE |
| | | EIAIGILQEQKLTFSENFSMTLNKFDGSTIRI |
| 16 | Asparagopsis | MNTAFPFRRKWSPSLWSARLESYLSTHAFELLTVITYITA |
| | armata Azores | LSLMFVWGAHDEFHNQSPDTNLRWYISIARGAGYTLNLNT |
| | - Mbb2-2_like | ALVILLASRLFFTFIRDTPLHKILPVDKSFPAFHILVGYT |
| | | IAASLFIHVPFHFTWIIAYDKWSAGLWDFTMSAVTGVVLL |
| | | LIFIIMFMFALPFYRKKYFRLFYISHMVGALLFFPLLIFH |
| | | GMYRRVPETYKWVALPLVIYILDRIARRFKISTVQLKLTS |
| | | EHSALKGKDILELRIPKPFTYRAGQYAEIRVPSLSREWHP |
| | | FTIASAPHEKCMCFFIKSLGNWTKELRGAFQNRMEGEDPN |
| | | PLLVQVRGPFGAPAQHVSSYERVVLISGGVGSTPFAAISK |
| | | SLHFLNKNKTCPSTDFSKTNLSEMKHIEQRISETISAVYD |
| | | IDLANSSDEPESEEQRKQYITDMMNLTAQNSEELDVSTSR |
| | | TSEQCVSSRSCPNIGDGLRILDVENQLEESSVSSMSTCDD |
| | | EEDYSSDFSEEMRLEKELAARKRKMARQKIARLYERRSIL |
| | | FSFLHTTRVTLALQLSLIARIVLVCIVSIFNIGEFNFGDS |
| | | RIGAKWVIGADAVLSVLHAVTLTLTILLEISFMGPRFFRS |
| | | SWQFMDFFVFVPLSILCSVSGVLSWTGTEMSGTLVFLHFI |
| | | VLLPIMLIFMSARLYRSIGSLNLFHDRKCNPNCACGKKIP |
| | | DVDFIWTTRHTVDDQWLREELFPLEDGTKLRLHRYITGKE |
| | | SEDFESAETFITTSNIGRPPWDEIFAKIAQKSPSHAEIGV |
| | | FFCGPHSMGAAVRKSMRKTEIRSNLRGAYLETTNVVDLVA |
| | | DLLLRNTDHAAELRRHGCNIRFVFREENFG |
| 17 | Asparagopsis | MPSPSSSTCPKSTQDLLTARLESHLSTHAFELLALVVYAT |
| | armata Azores | ALLLMFLWGAHDEFHHHTSDDNMRWYISIARAAGYTLNLN |
| | - Mbb2_like | TALVILLAARLFSTYIRETPLHLIIPLDKSFPAFHIIVAY |
| | | AIAISLVLHVAFHFAWIIAYDKWEPGLWGYTMSASTGSVL |
| | | LIVFLVMFITALPTFRKKRFRFFYLTHMFGVILFFPLLIF |
| | | HGMYNRVPETYKWITAPLIIYIIDRLLRRFKVSTVPLQLT |
| | | SDHSSLKSADILELRVPKPFHYRAGQYAEVCVPSISSEWH |
| | | PFTIASAPHEETMTFFIKTLGNWTQALRHQFQTRMDSDDC |
| | | KPLTVQIRGPYGAPAQHVGRYQRVVLVSGGVGATPFAAIS |
| | | KHLHHLNVAEHHKKTLDVDIPSKHTIQLGNRIRETISNMY |
| | | GLGLGESGCAEDSNQQRRQHLADMLNMSPDGAVQKLGNEI |
| | | SHPSPTGSSDVSDTCSSENVDLSFSSSIGSQMATLGTYEK |
| | | ECSSRKGGREDKMGRSAYSKHNLMFLNGKRSRLLSLLHTT |
| | | RITLLLLFTLIARLVLTCVVSIFDLGHFSTTSGHIEAKWV |
| | | IVTNVVLGLIVGLTLFTIIVLEVSFMGTRFFHRNWRCVDF |
| | | LVFVPLSFLCNLYGIVTWAGRDQSSFLTFVDYIIITPIIF |
| | | LLLCHRLYRSIGTRNLFDDNGSCRGSEHSVPEVDFIWTVA |
| | | HEGDDEWLREDLYTLATGTELRLHRYVTREKEVEVEGGEE |
| | | FITSARAGRPKWDEIFLKIAEESRSCSEIGVFFCGPSGMG |
| | | KQVRESLRRAEVFSSLRGSYLRKTGGGEVGDEVRLLKRFG |
| | | SNVRFVFREENFS |
| 18 | Asparagopsis | MPPPTRRQSALVTRIAAADEAANRSHPPHLRNGDETLYKT |
| | armata Azores | TSNALSYLATFTKGLPHHPDGRLVSAQHFQLFARAVQTGT |
| | - Mbb3_like | PQHIADIPLGPPSTFSTFRSGIAKDAPLRAWESMAAGNTF |
| | | DLQGPDPQSVTIPPCPRLESNELVTEMTELYYMALLRDVP |
| | | FSHFKSHHLVAAAVDDINHTHWIQSHKCCIPELERHRHRE |
| | | PFSSSNVFRGTTPGERNGPYISQFLIVGTKGLGEIHDEAD |
| | | GYIQYGAIRIDQRVRSVETEIDYMTTWAAFLDVQNAADLR |
| | | RHSTYAKSSSYRFLYTPRDLATYVHHDSLYQAYLNACIIL |
| | | LDMKVPFDSGLPFTTEDHIDKQQGFVNFGGPHILTLVTEV |
| | | ATRALKAVRFQKYAVHRRLRPEAVGGLLEQYRRYGGGYSE |
| | | LANTIRPIRHLARSLSSNLMSLVAAANGHQNATMSDNGFP |
| | | RSHDTGSGETHLLPMAYPEGSPMHPSYGAGHATVAGACVT |
| | | ILKGFFDHTYKLPFAYVSSCNGKMLKKAKVAKKLSVEGEL |
| | | NKLASNISIGRSWAGVHYYSDYVESIRLGEEVAIGILKEQ |
| | | KLTYSEKFSMTIPKFDGTTIKI |
| 19 | Asparagopsis | MSEERHASALRVRSEAADLAASRPLPTHLANGDEHRYHDT |
| | armata Azores | HLASYTKGLPHEKDTGLSSSPDHFAAFVRAIETGAQEDIT |
| | - Mbb4-2_like | SLPLGIDEDPRFISAIASEGSDHPIADTRAWESMAAGNTY |
| | | DLEGPDAQAVTMPPAPKIDSEELLTEITECYWMALLRDVP |
| | | FTEFERDGTTAAAAASISRTRWVQFQEQPGQRPCDLSDAE |
| | | IARLRGPFRKTNIFRGVTPGEQIGPYLSQFLLVGTKGIAN |
| | | IQEISDGFIQYGGMRMDQRVRVATPNRDYMTTWASWLDVQ |
| | | NAGDVRGRETYDNDTPFRFITTPRDLATYVHFDALYQAYL |
| | | NACLILLDTKAPFDPEIPFVEDDDIDKQQGFATFGGPHIL |
| | | SLVTEVATRALKAVRFQKFGTHRRLRPEAVGGLIERYRKT |
| | | EGAEPFEPIQKIATEIDNDLMTRISQHNAAQNELSDNGGA |
| | | RSGDYNPEGCESAETYLLPMAFPEGSPMHPSYGAGHAAVA |
| | | GACVTILKAFFDHTHELEFCYVPSTDGQNLEEVEINEPLT |
| | | VEHELNKLCANISIGRNWAGVHYYSDYIESARLGEEVAIG |
| | | ILEEQSLTYGEEFSMTIPKFDGTTIKISNSK |
| 20 | Asparagopsis | MSTTRQAAALQVRTDAAKLAASRKLPTHTPNGDETRFSDS |
| | armata Azores | YIASYTKGLPHDKKTGLLSSTDDFTAFVRAINTGAQEDIR |
| | - Mbb4_like | SLRLGIDETPRFISGIASPALDLPIADTRAWESMAAGNTF |
| | | DLEGPDAQAVTMPPAPKIDSEELLTEITECYWMALLRDVP |
| | | FSEFHRNGTVAAAAASISRTRWVQFQEQPGQRPSDLTNAE |
| | | IQRLRGPFNKSNVFRGDMPGEQVGPYLSQFLLVGTQGIGK |
| | | MHQPEEGFIQYGGMRMDQRVRFATPNRDYMTTWASWLDVQ |
| | | NAGDVRSRETYDKDTDYRFITTPRDLATYVHFDALYQAYL |
| | | NACLILLDIKAPFDHGLPFVQDDDIDKQQGFATFGGPHIL |
| | | SLVTEVATRALKAVRFQKFGTHRRLRPEAVGGLIERYRKT |
| | | NGAAPFAAAKKLATEIDNELMTRISRHNAKQNEFSDNGKA |
| | | RRNDFNPEGGASAETYLLPMAFPEGSPMHPSYGAGHAAVA |
| | | GACVTILKAFFDHKYKLKFCFEPSTDGRKLEKVNISEPLT |
| | | VEHELNKLCANISIGRNWAGVHYYSDYEESAKLGEEVAIG |
| | | ILQEQSLTYGEKFSMTLPKFDGTCITISN |
| 21 | Asparagopsis | MATPDTQNPNRAEASFKIRLSAAELARSRGSPTHISNNSE |
| | armata Ireland | ARFRNPDGTRSLLANFTKGLPHINETGLVQSALDYDQFVR |
| | - Mbb1_like | AIDSGDARDFAALPLGPQNVTPRFTSGIATNPEQSGVRAW |
| | | ESMGAGRTFDLEGPDAQAVTMPPAPELDSEELVTEVTECY |
| | | WMSLLRDVPFPTFHCDEDVAAAAKSINDTQWIKFKENPPT |
| | | HLTSAERSRLRGPIDASNVFRGITPGDDVGPYLSQFLLVG |
| | | TTGIANGNEVSDGFIQYGGMRMDQRVRVASPKVDYMTTFG |
| | | SWLDVQNAADVTGRERYESDPGFRFIHTPRDLATYVHFDA |
| | | LYQAYLNACIILLDIGAPFDSGVPFQLDNDIDKQQAFATF |
| | | GGPHILSLVTEVATRALKAVRFQKFNVHRRLRPETVGGRV |
| | | DRYYKTKAPEFSGAAKLVDSLDSDLLDRVYYHNKKQNKLS |
| | | DNGAPRSADFNPDGGLSEGNLLMPMAFPEGSPMHPAYGAG |
| | | HATVAGACVTVLKAFFDGGFPLPFAYVTNENGCGLEPVEV |
| | | SEPLTVDGELNKICSNISIGRNWAGVHYFTDYIESIRIGE |
| | | EIAIGILQEQKLTFSENFSMTLNKFDGSTIRI |
| 22 | Asparagopsis | MNTAFPFRRKWSPSLWSARLESYLSTHAFELLTVITYITA |
| | armata Ireland | LSLMFVWGAHDEFHNQSPDTNLRWYISIARGAGYTLNLNT |
| | - Mbb2-2_like | ALVILLASRLFFTFIRDTPLHKILPVDKSFPAFHILVGYT |
| | | IAASLFIHVPFHFTWIIAYDKWSAGLWDFTMSAVTGVVLL |
| | | LIFIIMFMFALPFYRKKYFRLFYISHMVGALLFFPLLIFH |
| | | GMYRRVPETYKWVALPLVIYILDRIARRFKISTVQLKLTS |
| | | EHSALKGKDILELRIPKPFTYRAGQYAEIRVPSLSREWHP |
| | | FTIASAPHEKCMCFFIKSLGNWTKELRGAFQNRMEGEDPN |
| | | PLLVQVRGPFGAPAQHVSSYERVVLISGGVGSTPFAAISK |
| | | SLHFLNKNKTCPSTDFSKTNLSEMKHIEQRISETISAVYD |
| | | IDLANSSDEPESEEQRKQYITDMMNLTAQNSEELDVSTSR |
| | | TSEQCVSSRSCPNIGDGLRILDVENQLEESSVSSMSTCDD |
| | | EEDYSSDFSEEMRLEKELAARKRKMARQKIARLYERRSIL |
| | | FSFLHTTRVTLALQLSLIARIVLVCIVSIFNIGEFNFGDS |
| | | RIGAKWVIGADAVLSVLHAVTLTLTILLEISFMGPRFFRS |
| | | SWQFMDFFVFVPLSILCSVSGVLSWTGTEMSGTLVFLHFI |
| | | VLLPIMLIFMSARLYRSIGSLNLFHDRKCNPNCACGKKIP |
| | | DVDFIWTTRHTVDDQWLREELFPLEDGTKLRLHRYITGKE |
| | | SEDFESAETFITTSNIGRPPWDEIFAKIAQKSPSHAEIGV |
| | | FFCGPHSMGAAVRKSMRKTEIRSNLRGAYLETTNVVDLVA |
| | | DLLLRNTDHAAELRRHGCNIRFVFREENFG |
| 23 | Asparagopsis | MAPRSGKQRCSPTSLTLLSARLESYLSTNAFSLLALFIYS |
| | armata Ireland | LANILMFVWGAHDEFHHHTSQKNLRWYICIARGAGYTLNL |
| | - Mbb2-3_like | NTALVILLAARLFATYIRETPLQHILPIDKSFPAFHIIVA |
| | | YTIAAALFIHVSFHLVWIIAYDKWELGMWGFTMSATTGFA |
| | | LLTVFVVMFVSALPRFRKKQFRTFYLIHMVGALLFFTLLI |
| | | FHGMYNRVPETYKWIAGPLAIYTIDRVLRRFKISTAKLEL |
| | | TGEHSSLKGSDILELRVPKTFEYRAGQYAEVCVPSINNEW |
| | | HPFTIASAPHEKSMCFYIKAIGDWTRHLRDAFEARIENDL |
| | | YEPLKVQIRGPYGAPAQHVRGYRRVVLISGGVGSTPFAAI |
| | | CKNLHHLNKSENHSKALESHGSSKRQSQIHWHIRDAISNL |
| | | FDVCLDDSRSDAASNQQRIQQVADMLNMSPKGNVHNVSFK |
| | | RNLSKTSDDCSSVEEITKQIRADEEISLSQKEKQSSYNLD |
| | | ACSESIRYYNRPSANVFNVAQRRIFNFYEYRSRLLAFLHT |
| | | TRFTFALLLTLVARIVILCVVSIFNLGHIGISDSHIDAEW |
| | | VIATNSLLGLVLGIALLTTVLLEISFMRLRFFYRGWRCVD |
| | | FFIFLPITFLCNISNFMSWSGRQQAKFLIFLDLIIVVPIM |
| | | FLLLCHRLYRSIGARNLLDDTQCRQSCTCNKTIPDVDFVW |
| | | TTPRDSEDEWLRSELYPLATGTDLRLHRYVTREKDLDLEE |
| | | PEHFITTANTGRPQWDEIFAQIAEQTPSHNEVGVFFCGPH |
| | | PMGAAVQKSMRKVEVMSNLRGSYLRKTDSELLVEDLSLRD |
| | | EKEVKLLREHGCNIRFVFREENFS |
| 24 | Asparagopsis | MPSPSSSTCPKSTQDLLTARLESHLSTHAFELLALVVYAT |
| | armata Ireland | ALLLMFLWGAHDEFHHHTSDDNMRWYISIARAAGYTLNLN |
| | - Mbb2_like | TALVILLAARLFSTYIRETPLHLIIPLDKSFPAFHIIVAY |
| | | AIAISLVLHVAFHFAWIIAYDKWEPGLWGYTMSASTGSVL |
| | | LIVFLVMFITALPTFRKKRFRFFYLTHMFGVILFFPLLIF |
| | | HGMYNRVPETYKWITAPLIIYIIDRLLRRFKVSTVPLQLT |
| | | SDHSSLKSADILELRVPKPFHYRAGQYAEVCVPSISSEWH |
| | | PFTIASAPHEETMTFFIKTLGNWTQALRHQFQTRMDSDDC |
| | | KPLTVQIRGPYGAPAQHVGRYQRVVLVSGGVGATPFAAIS |
| | | KHLHHLNVAEHHKKTLDVDIPSKHTIQLGNRIRETISNMY |
| | | GLGLGESGCAEDSNQQRRQHLADMLNMSPDGAVQKLGNEI |
| | | SHPSPTGSSDVSDTCSSENVDLSFSSSIGSQMATLGTYEK |
| | | ECSSRKGGREDMLGRSAYSKNNLMFLKGKRSRLLSLLHTT |
| | | RITLLLLFTLIARLVLTCVVSIFDLGHFSTTSGHIEAKWV |
| | | IVTNVVLGLIVGLTLFTIIVLEVSFMGTRFFHRNWRCVDF |
| | | LVFVPLSFLCNLYGIVTWAGRDQSSFLTFVDYIIITPIIF |
| | | LLLCHRLYRSIGTRNLFDDNGSCRGSEHSVPEVDFIWTVA |
| | | HEGDDEWLREDLYTLATGTELRLHRYVTREKEVEVEGGEE |
| | | FITSARAGRPKWDEIFLKIAEESRSCSEIGVFFCGPSGMG |
| | | KQVRESLRRAEVFSSLRGSYLRKTGGGEVGDEVRLLKRFG |
| | | SNVRFVFREENFS |
| 25 | Asparagopsis | MPPPTRRQSALVTRIAAADEAANRSHPPHLRNGDETLYKT |
| | armata Ireland | TSNALSYLATFTKGLPHHPDGRLVSAQHFQLFARAVQTGT |
| | - Mbb3_like | PQHIADIPLGPPSTFSTFRSGIAKDAPLRAWESMAAGNTF |
| | | DLQGPDPQSVTIPPCPRLESNELVTEMTELYYMALLRDVP |
| | | FSHFKSHHLVAAAVDDINHTHWIQSHKCCIPELERHRHRE |
| | | PFSSSNVFRGTTPGERNGPYISQFLIVGTKGLGEIHDEAD |
| | | GYIQYGAIRIDQRVRSVETEIDYMTTWAAFLDVQNAADLR |
| | | RHSTYAKSSSYRFLYTPRDLATYVHHDSLYQAYLNACIIL |
| | | LDMKVPFDSGLPFTTEDHIDKQQGFVNFGGPHILTLVTEV |
| | | ATRALKAVRFQKYAVHRRLRPEAVGGLLEQYRRYGGGYSE |
| | | LANTIRPIRHLARSLSSNLMSLVAAANGHQNATMSDNGFP |
| | | RSHDTGSGETHLLPMAYPEGSPMHPSYGAGHATVAGACVT |
| | | ILKGFFDHTYKLPFAYVSSCNGKMLKKAKVAKKLSVEGEL |
| | | NKLASNISIGRSWAGVHYYSDYVESIRLGEEVAIGILKEQ |
| | | KLTYSEKFSMTIPKFDGTTIKI |
| 26 | Asparagopsis | MSEERHASALRVRSEAADLAASRPLPTHLANGDEHRYHDT |
| | armata Ireland | HLASYTKGLPHEKDTGLSSSPDHFAAFVRAIETGAQEDIT |
| | - Mbb4-2_like | SLPLGIDEDPRFISAIASEGSDHPIADTRAWESMAAGNTY |
| | | DLEGPDAQAVTMPPAPKIDSEELLTEITECYWMALLRDVP |
| | | FTEFERDGTTAAAAASISRTRWVQFQEQPGQRPCDLSDAE |
| | | IARLRGPFRKTNIFRGVTPGEQIGPYLSQFLLVGTKGIAN |
| | | IQEISDGFIQYGGMRMDQRVRVATPNRDYMTTWASWLDVQ |
| | | NAGDVRGRETYDNDTPFRFITTPRDLATYVHFDALYQAYL |
| | | NACLILLDTKAPFDPEIPFVEDDDIDKQQGFATFGGPHIL |
| | | SLVTEVATRALKAVRFQKFGTHRRLRPEAVGGLIERYRKT |
| | | EGAEPFEPIQKIATEIDNDLMTRISQHNAAQNELSDNGGA |
| | | RSGDYNPEGCESAETYLLPMAFPEGSPMHPSYGAGHAAVA |
| | | GACVTILKAFFDHTHELEFCYVPSTDGQNLEEVEINEPLT |
| | | VEHELNKLCANISIGRNWAGVHYYSDYIESARLGEEVAIG |
| | | ILEEQSLTYGEEFSMTIPKFDGTTIKISNSK |
| 27 | Asparagopsis | MSTTRQAAALQVRTDAAKLAASRKLPTHTPNGDETRFSDS |
| | armata Ireland | YIASYTKGLPHDKKTGLLSSTDDFTAFVRAINTGAQEDIR |
| | - Mbb4_like | SLRLGIDETPRFISGIASPALDLPIADTRAWESMAAGNTF |
| | | DLEGPDAQAVTMPPAPKIDSEELLTEITECYWMALLRDVP |
| | | FSEFHRNGTVAAAAASISRTRWVQFQEQPGQRPSDLTNAE |
| | | IQRLRGPFNKSNVFRGDMPGEQVGPYLSQFLLVGTQGIGK |
| | | MHQPEEGFIQYGGMRMDQRVRFATPNRDYMTTWASWLDVQ |
| | | NAGDVRSRETYDKDTDYRFITTPRDLATYVHFDALYQAYL |
| | | NACLILLDIKAPFDHGLPFVQDDDIDKQQGFATFGGPHIL |
| | | SLVTEVATRALKAVRFQKFGTHRRLRPEAVGGLIERYRKT |
| | | NGAAPFAAAKKLATEIDNELMTRISRHNAKQNEFSDNGKA |
| | | RRNDFNPEGGASAETYLLPMAFPEGSPMHPSYGAGHAAVA |
| | | GACVTILKAFFDHKYKLKFCFEPSTDGRKLEKVNISEPLT |
| | | VEHELNKLCANISIGRNWAGVHYYSDYEESAKLGEEVAIG |
| | | ILQEQSLTYGEKFSMTLPKFDGTCITISN |
| 28 | Asparagopsis | MATPDTQNPNRAEASFKIRLSAAELARSRGSPTHISNNSE |
| | armata Ireland | ARFRNPDGTRSLLANFTKGLPQINETGLVQSALDYDQFVR |
| | 2 - Mbb1_like | AIDSGDARDFAALPLGPQNVTPRFTSGIATNPEQSGVRAW |
| | | ESMGAGRTFDLEGPDAQAVTMPPAPELDSEELVTEVTECY |
| | | WMSLLRDVPFPTFHCDEDVAAAAKSINDTQWIKFKENPPT |
| | | HLTSAERSRLRGPIDASNVVRGITPGDDVGPYLSQFLLVG |
| | | TTGIANGNEVSDGFIQYGGMRMDQRVRVASPKVDYMTTFG |
| | | SWLDVQNAADVTGRERYESDPGFRFIHTPRDLATYVHFDA |
| | | LYQAYLNACIILLDIGAPFDSGVPFQLDNDIDKQQAFATF |
| | | GGPHILSLVTEVATRALKAVRFQKFNVHRRLRPETVGGRV |
| | | DRYYKTKAPEFSGAAKLVDSLDSDLLDRVYYHNKKQNKLS |
| | | DNGAPRSADFNPDGGLSEGNLLMPMAFPEGSPMHPAYGAG |
| | | HATVAGACVTVLKAFFDGGFPLPFAYVTNENGCGLEPVEV |
| | | SEPLTVDGELNKICSNISIGRNWAGVHYFTDYIESIRIGE |
| | | EIAIGILQEQKLTFSENFSMTLNKFDGSTIRI |
| 29 | Asparagopsis | MNTAFPFRRKWSPSLWSARLESYLSTHAFELLTVITYITA |
| | armata Ireland | LSLMFVWGAHDEFHNQSPDTNLRWYISIARGAGYTLNLNT |
| | 2 - Mbb2- | ALVILLASRLFFTFIRDTPLHKILPVDKSFPAFHILVGYT |
| | 2_like | IAASLFIHVPFHFTWIIAYDKWSAGLWDFTMSAVTGVVLL |
| | | LIFIIMFMFALPFYRKKYFRLFYISHMVGALLFFPLLIFH |
| | | GMYRRVPETYKWVALPLVIYILDRIARRFKISTVQLKLTS |
| | | EHSALKGKDILELRIPKPFTYRAGQYAEIRVPSLSREWHP |
| | | FTIASAPHEKCMCFFIKSLGNWTKELRGAFQNRMEGEDPN |
| | | PLLVQVRGPFGAPAQHVSSYERVVLISGGVGSTPFAAISK |
| | | SLHFLNKNKTCPSTDFSKTNLSEMKHIEQRISETISAVYD |
| | | IDLANSSDEPESEEQRKQYITDMMNLTAQNSEELDVSTSR |
| | | TSEQCVSSRSCPNIGDGLRILDVENQLEESSVSSMSTCDD |
| | | EEDYSSDFSEEMRLEKELAARKRKMARQKIARLYERRSIL |
| | | FSFLHTTRVTLALQLSLIARIVLVCIVSIFNIGEFNFGDS |
| | | RIGAKWVIGADAVLSVLHAVTLTLTILLEISFMGPRFFRS |
| | | SWQFMDFFVFVPLSILCSVSGVLSWTGTEMSGTLVFLHFI |
| | | VLLPIMLIFMSARLYRSIGSLNLFHDRKCNPNCACGKKIP |
| | | DVDFIWTTRHTVDDQWLREELFPLEDGTKLRLHRYITGKE |
| | | SEDFESAETFITTSNIGRPPWDEIFAKIAQKSPSHAEIGV |
| | | FFCGPHSMGAAVRKSMRKTEIRSNLRGAYLETTNVVDLVA |
| | | DLLLRNTDHAAELRRHGCNIRFVFREENFG |
| 30 | Asparagopsis | MAPRSGKQRCSPTSLTLLSARLESYLSTNAFSLLALFIYS |
| | armata Ireland | LANILMFVWGAHDEFHHHTSQKNLRWYICIARGAGYTLNL |
| | 2 - Mbb2- | NTALVILLAARLFATYIRETPLQHILPIDKSFPAFHIIVA |
| | 3_like | YTIAAALFIHVSFHLVWIIAYDKWELGMWGFTMSATTGFA |
| | | LLTVFVVMFVSALPRFRKKQFRTFYLIHMVGALLFFTLLI |
| | | FHGMYNRVPETYKWIAGPLAIYTIDRVLRRFKISTAKLEL |
| | | TGEHSSLKGSDILELRVPKTFEYRAGQYAEVCVPSINNEW |
| | | HPFTIASAPHEKSMCFYIKAIGDWTRHLRDAFEARIENDL |
| | | YEPLKVQIRGPYGAPAQHVRGYRRVVLISGGVGSTPFAAI |
| | | CKNLHHLNKSENHSKALESHGSSKRQSQIHWHIRDAISNL |
| | | FDVCLDDSRSDAASNQQRIQQVADMLNMSPKGNVHNVSFK |
| | | RNLSKTSDDCSSVEEITKQIRADEEISLSQKEKQSSYNLD |
| | | ACSESIRYYNRPSANVFNVAQRRIFNFYEYRSRLLAFLHT |
| | | TRFTFALLLTLVARIVILCVVSIFNLGHIGISDSHIDAEW |
| | | VIATNSLLGLVLGIALLTTVLLEISFMRLRFFYRGWRCVD |
| | | FFIFLPITFLCNISNFMSWSGRQQAKFLIFLDLIIVVPIM |
| | | FLLLCHRLYRSIGARNLLDDTQCRQSCTCNKTIPDVDFVW |
| | | TTPRDSEDEWLRSELYPLATGTDLRLHRYVTREKDLDLEE |
| | | PEHFITTANTGRPQWDEIFAQIAEQTPSHNEVGVFFCGPH |
| | | PMGAAVQKSMRKVEVMSNLRGSYLRKTDSELLVEDLSLRD |
| | | EKEVKLLREHGCNIRFVFREENFS |
| 31 | Asparagopsis | MPSPSSSTCPKSTQDLLTARLESHLSTHAFELLALVVYAT |
| | armata Ireland | ALLLMFLWGAHDEFHHHTSDDNMRWYISIARAAGYTLNLN |
| | 2 - Mbb2_like | TALVILLAARLFSTYIRETPLHLIIPLDKSFPAFHIIVAY |
| | | AIAISLVLHVAFHFAWIIAYDKWEPGLWGYTMSASTGSVL |
| | | LIVFLVMFITALPTFRKKRFRFFYLTHMFGVILFFPLLIF |
| | | HGMYNRVPETYKWITAPLIIYIIDRLLRRFKVSTVPLQLT |
| | | SDHSSLKSADILELRVPKPFHYRAGQYAEVCVPSISSEWH |
| | | PFTIASAPHEETMTFFIKTLGNWTQALRHQFQTRMDSDDC |
| | | KPLTVQIRGPYGAPAQHVGRYQRVVLVSGGVGATPFAAIS |
| | | KHLHHLNVAEHHKKTLDVDIPSKHTIQLGNRIRETISNMY |
| | | GLGLGESGCAEDSNQQRRQHLADMLNMSPDGAVQKLGNEI |
| | | SHPSPTGSSDVSDTCSSENVDLSFSSSIGSQMATLGTYEK |
| | | ECSSRKGGREDKMGRSAYSKHNLMFLNGKRSRLLSLLHTT |
| | | RITLLLLFTLIARLVLTCVVSIFDLGHFSTTSGHIEAKWV |
| | | IVTNVVLGLIVGLTLFTIIVLEVSFMGTRFFHRNWRCVDF |
| | | LVFVPLSFLCNLYGIVTWAGRDQSSFLTFVDYIIITPIIF |
| | | LLLCHRLYRSIGTRNLFDDNGSCRGSEHSVPEVDFIWTVA |
| | | HEGDDEWLREDLYTLATGTELRLHRYVTREKEVEVEGGEE |
| | | FITSARAGRPKWDEIFLKIAEESRSCSEIGVFFCGPSGMG |
| | | KQVRESLRRAEVFSSLRGSYLRKTGGGEVGDEVRLLKRFG |
| | | SNVRFVFREENFS |
| 32 | Asparagopsis | MPPPTRRQSALVTRIAAADEAANRSHPPHLRNGDETLYKT |
| | armata Ireland | TSNALSYLATFTKGLPHHPDGRLVSAQHFQLFARAVQTGT |
| | 2 - Mbb3_like | PQHIADIPLGPPSTFSTFRSGIAKDAPLRAWESMAAGNTF |
| | | DLQGPDPQSVTIPPCPRLESNELVTEMTELYYMALLRDVP |
| | | FSHFKSHHLVAAAVDDINHTHWIQSHKCCIPELERHRHRE |
| | | PFSSSNVFRGTTPGERNGPYISQFLIVGTKGLGEIHDEAD |
| | | GYIQYGAIRIDQRVRSVETEIDYMTTWAAFLDVQNAADLR |
| | | RHSTYAKSSSYRFLYTPRDLATYVHHDSLYQAYLNACIIL |
| | | LDMKVPFDSGLPFTTEDHIDKQQGFVNFGGPHILTLVTEV |
| | | ATRALKAVRFQKYAVHRRLRPEAVGGLLEQYRRYGGGYSE |
| | | LANTIRPIRHLARSLSSNLMSLVAAANGHQNATMSDNGFP |
| | | RSHDTGSGETHLLPMAYPEGSPMHPSYGAGHATVAGACVT |
| | | ILKGFFDHTYKLPFAYVSSCNGKMLKKAKVAKKLSVEGEL |
| | | NKLASNISIGRSWAGVHYYSDYVESIRLGEEVAIGILKEQ |
| | | KLTYSEKFSMTIPKFDGTTIKI |
| 33 | Asparagopsis | MSEERHASALRVRSEAADLAASRPLPTHLANGDEHRYHDT |
| | armata Ireland | HLASYTKGLPHEKDTGLSSSPDHFAAFVRAIETGAQEDIT |
| | 2 - Mbb4- | SLPLGIDEDPRFISAIASEGSDHPIADTRAWESMAAGNTY |
| | 2_like | DLEGPDAQAVTMPPAPKIDSEELLTEITECYWMALLRDVP |
| | | FTEFERDGTTAAAAASISRTRWVQFQEQPGQRPCDLSDAE |
| | | IARLRGPFRKTNIFRGVTPGEQIGPYLSQFLLVGTKGIAN |
| | | IQEISDGFIQYGGMRMDQRVRVATPNRDYMTTWASWLDVQ |
| | | NAGDVRGRETYDNDTPFRFITTPRDLATYVHFDALYQAYL |
| | | NACLILLDTKAPFDPEIPFVEDDDIDKQQGFATFGGPHIL |
| | | SLVTEVATRALKAVRFQKFGTHRRLRPEAVGGLIERYRKT |
| | | EGAEPFEPIQKIATEIDNDLMTRISQHNAAQNELSDNGGA |
| | | RSGDYNPEGCESAETYLLPMAFPEGSPMHPSYGAGHAAVA |
| | | GACVTILKAFFDHTHELEFCYVPSTDGQNLEEVEINEPLT |
| | | VEHELNKLCANISIGRNWAGVHYYSDYIESARLGEEVAIG |
| | | ILEEQSLTYGEEFSMTIPKFDGTTIKISNSK |
| 34 | Asparagopsis | MSTTRQAAALQVRTDAAKLAASRKLPTHTPNGDETRFSDS |
| | armata Ireland | YIASYTKGLPHDKKTGLLSSTDDFTAFVRAINTGAQEDIR |
| | 2 - Mbb4_like | SLRLGIDETPRFISGIASPALDLPIADTRAWESMAAGNTF |
| | | DLEGPDAQAVTMPPAPKIDSEELLTEITECYWMALLRDVP |
| | | FSEFHRNGTVAAAAASISRTRWVQFQEQPGQRPSDLTNAE |
| | | IQRLRGPFNKSNVFRGDMPGEQVGPYLSQFLLVGTQGIGK |
| | | MHQPEEGFIQYGGMRMDQRVRFATPNRDYMTTWASWLDVQ |
| | | NAGDVRSRETYDKDTEYRFITTPRDLATYVHFDALYQAYL |
| | | NACLILLDIKAPFDHGLPFVQDDDIDKQQGFATFGGPHIL |
| | | SLVTEVATRALKAVRFQKFGTHRRLRPEAVGGLIERYRKI |
| | | NGAAPFAAAKKLATEIDNELMTRISRHNAKQNEFSDNGKA |
| | | RRNDFNPEGGASAETYLLPMAFPEGSPMHPSYAAGHAAVA |
| | | GACVTILKAFFDHKYKLKFCFEPSTDGRKLEKVNISEPLT |
| | | VEHELNKLCANISIGRNWAGVHYYSDYEESAKLGEEVAIG |
| | | ILQEQSLTYGEKFSMTLPKFDGTCITISN |
| 35 | Asparagopsis | MATPDTQNPNRAEASFKIRLSAAELARSRGSPTHISNNSE |
| | armata Ireland | ARFRNPDGTRSLLANFTKGLPHINETGLVQSALDYDQFVR |
| | 3 - Mbb1_like | AIDSGDARDFAALPLGPQNVTPRFTSGIATNPEQSGVRAW |
| | | ESMGAGRTFDLEGPDAQAVTMPPAPELDSEELVTEVTECY |
| | | WMSLLRDVPFPTFHCDEDVAAAAKSINDTQWIKFKENPPT |
| | | HLTSAERSRLRGPIDASNVFRGITPGDDVGPYLSQFLLVG |
| | | TTGIANGNEVSDGFIQYGGMRMDQRVRVASPKVDYMTTFG |
| | | SWLDVQNAADVTGRERYESDPGFRFIHTPRDLATYVHFDA |
| | | LYQAYLNACIILLDIGAPFDSGVPFQLDNDIDKQQAFATF |
| | | GGPHILSLVTEVATRALKAVRFQKFNVHRRLRPETVGGRV |
| | | DRYYKTKAPEFSGAAKLVDSLDSDLLDRVYYHNKKQNKLS |
| | | DNGAPRSADFNPDGGLSEGNLLMPMAFPEGSPMHPAYGAG |
| | | HATVAGACVTVLKAFFDGGFPLPFAYVTNENGCGLEPVEV |
| | | SEPLTVDGELNKICSNISIGRNWAGVHYFTDYIESIRIGE |
| | | EIAIGILQEQKLTFSENFSMTLNKFDGSTIRI |
| 36 | Asparagopsis | MNTAFPFRRKWSPSLWSARLESYLSTHAFELLTVITYITA |
| | armata Ireland | LSLMFVWGAHDEFHNQSPDTNLRWYISIARGAGYTLNLNT |
| | 3 - Mbb2- | ALVILLASRLFFTFIRDTPLHKILPVDKSFPAFHILVGYT |
| | 2_like | IAASLFIHVPFHFTWIIAYDKWSAGLWDFTMSAVTGVVLL |
| | | LIFIIMFMFALPFYRKKYFRLFYISHMVGALLFFPLLIFH |
| | | GMYRRVPETYKWVALPLVIYILDRIARRFKISTVQLKLTS |
| | | EHSALKGKDILELRIPKPFTYRAGQYAEIRVPSLSREWHP |
| | | FTIASAPHEKCMCFFIKSLGNWTKELRGAFQNRMEGEDPN |
| | | PLLVQVRGPFGAPAQHVSSYERVVLISGGVGSTPFAAISK |
| | | SLHFLNKNKTCPSTDFSKTNLSEMKHIEQRISETISAVYD |
| | | IDLANSSDEPESEEQRKQYITDMMNLTAQNSEELDVSTSR |
| | | TSEQCVSSRSCPNIGDGLRILDVENQLEESSVSSMSTCDD |
| | | EEDYSSDFSEEMRLEKELAARKRKMARQKIARLYERRSIL |
| | | FSFLHTTRVTLALQLSLIARIVLVCIVSIFNIGEFNFGDS |
| | | RIGAKWVIGADAVLSVLHAVTLTLTILLEISFMGPRFFRS |
| | | SWQFMDFFVFVPLSILCSVSGVLSWTGTEMSGTLVFLHFI |
| | | VLLPIMLIFMSARLYRSIGSLNLFHDRKCNPNCACGKKIP |
| | | DVDFIWTTRHTVDDQWLREELFPLEDGTKLRLHRYITGKE |
| | | SEDFESAETFITTSNIGRPPWDEIFAKIAQKSPSHAEIGV |
| | | FFCGPHSMGAAVRKSMRKTEIRSNLRGAYLETTNVVDLVA |
| | | DLLLRNTDHAAELRRHGCNIRFVFREENFG |
| 37 | Asparagopsis | MPSPSSSTCPKSTQDLLTARLESHLSTHAFELLALVVYAT |
| | armata Ireland | ALLLMFLWGAHDEFHHHTSDDNMRWYISIARAAGYTLNLN |
| | 3 - Mbb2_like | TALVILLAARLFSTYIRETPLHLIIPLDKSFPAFHIIVAY |
| | | AIAISLVLHVAFHFAWIIAYDKWEPGLWGYTMSASTGSVL |
| | | LIVFLVMFITALPTFRKKRFRFFYLTHMFGVILFFPLLIF |
| | | HGMYNRVPETYKWITAPLIIYIIDRLLRRFKVSTVPLQLT |
| | | SDHSSLKSADILELRVPKPFHYRAGQYAEVCVPSISSEWH |
| | | PFTIASAPHEETMTFFIKTLGNWTQALRHQFQTRMDSDDC |
| | | KPLTVQIRGPYGAPAQHVGRYQRVVLVSGGVGATPFAAIS |
| | | KHLHHLNVAEHHKKTLDVDIPSKHTIQLGNRIRETISNMY |
| | | GLGLGESGCAEDSNQQRRQHLADMLNMSPDGAVQKLGNEI |
| | | SHPSPTGSSDVSDTCSSENVDLSFSSSIGSQMATLGTYEK |
| | | ECSSRKGGREDKMGRSAYSKHNLMFLNGKRSRLLSLLHTT |
| | | RITLLLLFTLIARLVLTCVVSIFDLGHFSTTSGHIEAKWV |
| | | IVTNVVLGLIVGLTLFTIIVLEVSFMGTRFFHRNWRCVDF |
| | | LVFVPLSFLCNLYGIVTWAGRDQSSFLTFVDYIIITPIIF |
| | | LLLCHRLYRSIGTRNLFDDNGSCRGSEHSVPEVDFIWTVA |
| | | HEGDDEWLREDLYTLATGTELRLHRYVTREKEVEVEGGEE |
| | | FITSARAGRPKWDEIFLKIAEESRSCSEIGVFFCGPSGMG |
| | | KQVRESLRRAEVFSSLRGSYLRKTGGGEVGDEVRLLKRFG |
| | | SNVRFVFREENFS |
| 38 | Asparagopsis | MPPPTRRQSALVTRIAAADEAANRSHPPHLRNGDETLYKT |
| | armata Ireland | TSNALSYLATFTKGLPHHPDGRLVSAQHFQLFARAVQTGT |
| | 3 - Mbb3_like | PQHIADIPLGPPSTFSTFRSGIAKDAPLRAWESMAAGNTF |
| | | DLQGPDPQSVTIPPCPRLESNELVTEMTELYYMALLRDVP |
| | | FSHFKSHHLVAAAVDDINHTHWIQSHKCCIPELERHRHRE |
| | | PFSSSNVFRGTTPGERNGPYISQFLIVGTKGLGEIHDEAD |
| | | GYIQYGAIRIDQRVRSVETEIDYMTTWAAFLDVQNAADLR |
| | | RHSTYAKSSSYRFLYTPRDLATYVHHDSLYQAYLNACIIL |
| | | LDMKVPFDSGLPFTTEDHIDKQQGFVNFGGPHILTLVTEV |
| | | ATRALKAVRFQKYAVHRRLRPEAVGGLLEQYRRYGGGYSE |
| | | LANTIRPIRHLARSLSSNLMSLVAAANGHQNATMSDNGFP |
| | | RSHDTGSGETHLLPMAYPEGSPMHPSYGAGHATVAGACVT |
| | | ILKGFFDHTYKLPFAYVSSCNGKMLKKAKVAKKLSVEGEL |
| | | NKLASNISIGRSWAGVHYYSDYVESIRLGEEVAIGILKEQ |
| | | KLTYSEKFSMTIPKFDGTTIKI |
| 39 | Asparagopsis | MSEERHASALRVRSEAADLAASRPLPTHLANGDEHRYHDT |
| | armata Ireland | HLASYTKGLPHEKDTGLSSSPDHFAAFVRAIETGAQEDIT |
| | 3 - Mbb4- | SLPLGIDEDPRFISAIASEGSDHPIADTRAWESMAAGNTY |
| | 2_like | DLEGPDAQAVTMPPAPKIDSEELLTEITECYWMALLRDVP |
| | | FTEFERDGTTAAAAASISRTRWVQFQEQPGQRPCDLSDAE |
| | | IARLRGPFRKTNIFRGVTPGEQIGPYLSQFLLVGTKGIAN |
| | | IQEISDGFIQYGGMRMDQRVRVATPNRDYMTTWASWLDVQ |
| | | NAGDVRGRETYDNDTPFRFITTPRDLATYVHFDALYQAYL |
| | | NACLILLDTKAPFDPEIPFVEDDDIDKQQGFATFGGPHIL |
| | | SLVTEVATRALKAVRFQKFGTHRRLRPEAVGGLIERYRKT |
| | | EGAEPFEPIQKIATEIDNDLMTRISQHNAAQNELSDNGGA |
| | | RSGDYNPEGCESAETYLLPMAFPEGSPMHPSYGAGHAAVA |
| | | GACVTILKAFFDHTHELEFCYVPSTDGQNLEEVEINEPLT |
| | | VEHELNKLCANISIGRNWAGVHYYSDYIESARLGEEVAIG |
| | | ILEEQSLTYGEEFSMTIPKFDGTTIKISNSK |
| 4 | Asparagopsis | MSTTRQAAALQVRTDAAKLAASRKLPTHTPNGDETRFSDS |
| | armata Ireland | YIASYTKGLPHDKKTGLLSSTDDFTAFVRAINTGAQEDIR |
| | 3 - Mbb4_like | SLRLGIDETPRFISGIASPALDLPIADTRAWESMAAGNTF |
| | | DLEGPDAQAVTMPPAPKIDSEELLTEITECYWMALLRDVP |
| | | FSEFHRNGTVAAAAASISRTRWVQFQEQPGQRPSDLTNAE |
| | | IQRLRGPFNKSNVFRGDMPGEQVGPYLSQFLLVGTQGIGK |
| | | MHQPEEGFIQYGGMRMDQRVRFATPNRDYMTTWASWLDVQ |
| | | NAGDVRSRETYDKDTDYRFITTPRDLATYVHFDALYQAYL |
| | | NACLILLDIKAPFDHGLPFVQDDDIDKQQGFATFGGPHIL |
| | | SLVTEVATRALKAVRFQKFGTHRRLRPEAVGGLIERYRKT |
| | | NGAAPFAAAKKLATEIDNELMTRISRHNAKQNEFSDNGKA |
| | | RRNDFNPEGGASAETYLLPMAFPEGSPMHPSYGAGHAAVA |
| | | GACVTILKAFFDHKYKLKFCFEPSTDGRKLEKVNISEPLT |
| | | VEHELNKLCANISIGRNWAGVHYYSDYEESAKLGEEVAIG |
| | | ILQEQSLTYGEKFSMTLPKFDGTCITISN |
| 41 | Asparagopsis | MTDTQNPNRAEVAFKVRVSAAELARARGSPTHLSNNSESR |
| | taxiformis | FRNPDGTRSLLANFTKGLPHTKETALVVSAIDYDSFVRAI |
| | Azores - | DSGDPRDFADLPLGPQGVEPRFTSGIASDPEVGTRAWESG |
| | Mbb1_like | GAGLVFDLEGPDAQAVTMPPAPELDSDELVTEVTECYWMS |
| | | LLRDVPFPTFDSDPHIQSAAESINNTQWIQFKNNPPAHLT |
| | | AAERSRLRGPITTANVFRGITPGDEVGPYLSQFLLVGTTG |
| | | IANGNEVGDGFIQYGGMRMDQRVRVAKSHIDYMTTFGAYL |
| | | DVQNAANVSGRELYNEEEPRFRFIHTPRDLATYVHFDALY |
| | | QAYLNACIILLDIGAPFDSGIPFQLDNDIDKQQGFATFGG |
| | | PHILSLVTEVATRALKAVRFQKFNVHRRLRPEAIGARVDR |
| | | YCATKAPEFAGAAKLSEALDKKLLQKVHDHNKKQNLLSDR |
| | | GNPRANDFNPDGDVSKGNLLMPMAFPEGSPMHPAYGAGHA |
| | | TVAGACVTVLKAFFDGGYRLPFCYITDENGTGLKAVEIDE |
| | | PLTVDGELNKICSNISIGRNWAGVHYFTDYIESIRIGEEI |
| | | AIGILQEQKLTFSENFSMTLNKFDGSTIRI |
| 42 | Asparagopsis | MDTVSPGDSRQKAQGKMRPMRSFAILSANLEAFLSTHAFS |
| | taxiformis | LLFLIFYLTCTALMFVWGFTTQWKKTDNEAMIYFISIARG |
| | Azores - | AGYSLNLNTSLVILIAARLTFTLIRETPLQYILPLDKIFP |
| | Mbb2_like | LFHMFVGYMIAFLVLIHGTFHMTWIIRFQGWNSGLWGFTM |
| | | SFVTGFIILPVFVVMLITSLPHFRKNKFRLFYLFHLYGAA |
| | | LFFSILIFHGMYFERPETYKWITPSLIIYLLDRFIRRMKI |
| | | SSHDLDIALEKSILKDGDILELRVPKPFDYRPGHYAEVMV |
| | | PSINKEWHPFSIASAPHEETMCFYIKVLGDWTTEVRNAFK |
| | | ARLDGTITEPLKVRVRGPYGAPCQDVDGFKRVVLVSGGVG |
| | | ATPFSAIVKELHYLKTQAKNKNDGPGRIARDIDVRIRDSI |
| | | ANLYDVEVQDMEKATHIAQQKSIYVTEMLELTARRTSSLH |
| | | RVRSSVMDRMHGSSSTKTVLSTDAAIPSLEDGPVGGGQPM |
| | | SRMQRLSSSLSKAGNIRRVMSTLSENPVTWDQVMSAQNDP |
| | | LTNTTDLRRQMARLYDKRGMLLAFMHTARFTFFLLLTLIV |
| | | RIIIISVAATLDSPYFQLNDQFDGYTRLWPVVVDALFSVP |
| | | VAAALVVSITLEISFMRMRYFDTAARTFDALVLMPAAVIG |
| | | CVLAILTWARQDPESVLVKVIEYVLFIPLLFLGLSWRLYR |
| | | ITRSRSLLSDPACRCDNSDHIPDVDFIWSFPYEQSDQWLQ |
| | | DELGPLAKGTELRLHRYVTREKELDVEAAKSFITSANTGR |
| | | PKWDDIFQTIAEETPSNGAIGVFFCGPHVMGNQVRDAMLK |
| | | VEVLSNLRAAYLRNTPDEVLMTDLNLPNAEAVERLRSLGC |
| | | SVHFVYREENFG |
| 43 | Asparagopsis | MTPPTRRQTALVKRIAAADLAANRHHPHHVRNADESTYKL |
| | taxiformis | PNHVCSYISAFTKGLPHHLDGRLVSPHHFQLFVHAVQTGH |
| | Azores - | VTDIAAIPLGPPHSSSFRSGIAKGAAVRAWESMAAGNTFD |
| | Mbb3_like | LQGPDAQAVTMPPCPRLNSHELVTEMTELYYMALLRDVPF |
| | | THFNKHHLVQSAVHDMNHTHWTRSNRCCLPRPERRRHRSK |
| | | FTTDNIFRGVTPGDITGPYISQFLLVGTKGLGDAHEETEG |
| | | YVQYGATRIDQRVRTVEPKIDYMTTWAAYLDVQNGADLRR |
| | | HSTYAKPPSYRFISTPRDLATYVHYDALYQAYLNACIILL |
| | | DINAPYDGGLPFTADDAVDKQQGFATFGAPHVLTLVTEVA |
| | | TRALKAVRFQKYAVHRRLRPEAVGGLLEQYRRYGGGHSDL |
| | | AYIIRPIRHLADSLSSDLMFMVAKENGRQNSSITDNGHPR |
| | | SHDEGSDDTHLLSMAYAEGSPMHPSYGAGHATVAGACVTI |
| | | LKAFFDHTFKLPFAYVSSSDGRKLKTVKLSRKLTVEDELN |
| | | KLAANISIGRSWAGVHYYSDYVESIRLGEEVAIGMLKEQK |
| | | LTYSEKFTMTIPKFDGSVIEI |
| 44 | Asparagopsis | MAEERRQNALDIRIQAARLAKKRDHPTHEANGDEDRYPET |
| | taxiformis | LIGSFTKGLPHEKETGLLSNPADFADFVRAINTGAIKDIR |
| | Azores - | RLKLGIDEDPRFISGIASKGDKHPFADTRAWESMAAGLTY |
| | Mbb4_like | DLEGPDAQAVTMPPAPKLDSDELVTEITESYWMALIRDVP |
| | | FTEFERDGNTAAAAASISRTRWVQFNEQPSQRPSTLTDEE |
| | | IARLRGPYTKKNVFRGVTIGENVGPYLSQFLLVGTKGIGD |
| | | VQQISDGYIQYGSMRMDQRVRVAAPKRDYMTTWASWLDVQ |
| | | NAGDLRGREIYDDDTPFRFITTPRDLATWVHFDALYQAYL |
| | | NACIILLDIKAPFDPHIPFQADDDVDKQQGFATFGGPHIL |
| | | SLCTEVATRALKAVRFQKYNVHRRLRPEAIGGLVERFKKT |
| | | NGDAKFAPVKKLVNDLDGDMLRRIEQHNCEQNKLSDDGHA |
| | | RRDDYSPEGESNQTYLLPMAFPEGSPMHPSYGAGHATVAG |
| | | ACVTVLKAFFDHEYELDFCYVPTTDGKRLEKVNINEKLTV |
| | | EGELNKLCANICIGRNWAGVHYYSDYFESIKVGEEIAIGI |
| | | LQEQKLTYGEDFFMTLSKFDGEKIRI |
| 45 | Asparagopsis | MFHNAFVASAPVGVARIRGASITTKHSPFNAFRRQSSAAS |
| | taxiformis | VKMAQDNSDQEQRRKDAKQVRINAANLAFNRGVRPHKSNG |
| | Azores - | DESDFKETLIGTFTKGLRHNTETGQIFSRDLFDPFRASLA |
| | Mbb_like | TGDPADFKKIRLGPKLPKDTPGRWQSTLANGAPGLPFESQ |
| | | VKARAWESAGAGLAFDLEGPDAQAVTMPPAPKLASAELEF |
| | | EIAELYFMALARDIPFSLWDSPDTKAFFDNAIATLSALPW |
| | | VSEEFGKGLTDEERFRREGKLNLTTGNIFRGYGAGVRDGP |
| | | YISQYLIRGSQGLGTDGTATGQVMYGSLGLDLRVRSIEPR |
| | | KDWMTTWEAFIDVQDGADVRQGEFPDYKCAPDGSPIFRFI |
| | | ADGRDLSTYVHYDALYEAYLNACFALLADGVGVDTGLPFR |
| | | DDDEHDKQTAFATFGGPHILSLVCEVATRALKAVRYQKFQ |
| | | IHLRARPEAVGGLIHAQKSERCDFGEEVRALVARLDGCGL |
| | | LDLVADKNMQQNQTFADRVKDRSASSPSYLLPMAFPEGSP |
| | | MHPSYGAGHGTVAGACTIILKAFFNTVQFDGKCAVKDAKP |
| | | SNVEFTPLPNEIFEADAHGCLQPVCLPDGMTLTVDGELNK |
| | | IANNVAFGRSLAGVHWSSDNIESLILGEEVARGILEEQVL |
| | | TFAEQHSFTYVGFDGHLNVIRSQPKHELNVDKRTVEVLRS |
| | | DDPVA |
| 46 | Corallina | MGIPADNLQSRAKASFDTRVAAAELALARGAVPSFANGEE |
| | officinalis | LLYRNSETGDPSFIGSFTKGLPHDDNGAIIDPDDFLAFVR |
| | halo | AINSGDEKEIAALTLGPARDPETGLPIWRSDLANSLDLEV |
| | peroxidase | RGWENSSAGLTFDLEGPDAQSVAMPPAPVLTSPELIAEMA |
| | pdb\|lQHB\|A | ELYLMALGRDIEFSEFDSPKNAAFIRSAIERLNGLEWENT |
| | Chain A | PAKLGDPPAEIRRRRGEVTVGNLFRGILPGSEVGPYLSQF |
| | | IIVGSKQIGSATVGNKTLVSPNAADEFDGEIAYGSITISQ |
| | | RVRIATPGRDFMTDLKVFLDVQDAADFRGFESYEPGARLI |
| | | RTIRDLATWVHFDSLYEAYLNACLILLANGVPFDPNLPFQ |
| | | QEDKLDNQDVFVNFGSAHVLSLVTEVATRALKAVRYQKFN |
| | | IHRRLRPEATGGLISVNKNAFLKSESVFPEVDVLVEELSS |
| | | ILDDSASSNEKQNIADGDVSPGKSFLLPMAFAEGSPFHPS |
| | | YGSGHAVVAGACVTILKAFFDANFQIDQVFEVDTDEDKLV |
| | | KSSFPGPLTVAGELNKLADNVAIGRNMAGVHYFSDQFESL |
| | | LLGEQIAIGILEEQSLTYGENFFFNLPKFDGTTIQI |
| 47 | A. taxiformis | ATGACCGACACACAGAATCCCAATCGTGCAGAAGTGGCCT |
| | Vanadium- | TCAAAGTCCGCGTATCAGCAGCTGAGCTGGCACGCGCTCG |
| | dependent | TGGCTCCCCAACCCATCTATCCAACAACTCGGAGAGTCGC |
| | haloperoxidase | TTCAGAAACCCTGATGGTACCCGTTCGCTGCTCGCCAATT |
| | Mbb1 Coding | TCACCAAGGGTCTCCCGCATACCAAGGAAACCGCTCTAGT |
| | sequence | GGTGTCCGCAATCGACTACGATAGCTTTGTTCGCGCAATT |
| | | GACAGCGGTGATCCTCGTGATTTCGCAGACCTCCCGCTCG |
| | | GTCCGCAGGGTGTTGAGCCCAGATTCACCTCCGGTATCGC |
| | | TAGCGACCCAGAGGTCGGAACCCGTGCCTGGGAAAGTGGT |
| | | GGTGCTGGTCTTGTCTTCGATCTCGAAGGTCCCGATGCTC |
| | | AGGCTGTCACTATGCCGCCCGCGCCCGAGCTGGACAGCGA |
| | | TGAATTGGTTACTGAAGTCACAGAATGCTACTGGATGTCT |
| | | CTGCTGAGAGACGTTCCCTTCCCGACATTTGACAGTGATC |
| | | CCCATATTCAGTCAGCTGCAGAGTCCATCAATAACACCCA |
| | | ATGGATTCAATTCAAGAACAACCCACCAGCACATCTCACA |
| | | GCTGCTGAGCGTTCCCGTCTCAGAGGCCCCATCACCACTG |
| | | CCAACGTCTTCAGAGGTATCACACCGGGAGACGAAGTCGG |
| | | ACCCTATCTGTCACAGTTCCTCCTTGTCGGTACCACCGGT |
| | | ATTGCCAATGGCAATGAAGTTGGCGACGGATTCATCCAGT |
| | | ACGGTGGCATGCGTATGGACCAGCGCGTGCGTGTCGCCAA |
| | | GTCACACATTGATTACATGACCACATTCGGCGCCTATCTC |
| | | GACGTGCAAAATGCCGCCAACGTCTCCGGTCGCGAATTGT |
| | | ACAATGAAGAGGAACCCCGTTTCCGCTTCATTCACACTCC |
| | | TCGTGATCTGGCCACTTACGTGCATTTCGACGCCTTGTAT |
| | | CAGGCCTACCTCAACGCTTGCATCATCTTGCTCGACATCG |
| | | GTGCGCCATTCGACTCTGGAATTCCCTTCCAGCTCGATAA |
| | | CGACATTGATAAGCAGCAAGGATTTGCCACTTTCGGCGGT |
| | | CCGCACATCCTCTCTCTAGTTACCGAAGTGGCCACCCGAG |
| | | CCCTCAAGGCTGTTCGCTTCCAGAAATTCAACGTCCATCG |
| | | TCGTCTGCGTCCGGAGGCCATTGGTGCCCGAGTCGATCGA |
| | | TACTGCGCAACAAAGGCACCTGAATTTGCTGGCGCTGCCA |
| | | AGCTCTCCGAAGCGCTGGACAAGAAGTTGTTGCAGAAGGT |
| | | GCACGATCATAACAAGAAACAAAACTTGTTGAGCGATCGA |
| | | GGAAACCCGCGTGCAAACGACTTCAACCCCGACGGAGATG |
| | | TGAGCAAAGGTAATCTGCTGATGCCCATGGCGTTCCCAGA |
| | | GGGAAGTCCTATGCATCCCGCGTACGGCGCTGGACATGCG |
| | | ACTGTTGCAGGCGCTTGTGTCACAGTGTTGAAGGCGTTCT |
| | | TCGATGGTGGTTACAGGTTGCCGTTCTGCTACATCACCGA |
| | | CGAGAATGGAACAGGTCTTAAGGCAGTGGAAATTGACGAG |
| | | CCGTTGACCGTCGACGGCGAGTTGAATAAGATTTGCTCCA |
| | | ATATTTCGATTGGTCGCAATTGGGCTGGCGTGCATTACTT |
| | | CACTGATTACATCGAGAGTATTCGCATTGGTGAGGAGATT |
| | | GCCATTGGTATTCTGCAGGAGCAAAAGCTGACGTTCAGTG |
| | | AAAACTTCTCAATGACGCTGAACAAGTTTGACGGTTCGAC |
| | | CATCCGTATCTAG |
| 48 | A. taxiformis | ATGGCTAAGCAGAAGTCTCTCTTCACGCTGCTCAGCGCCA |
| | NAD(P)H | CACTAGAATCTCATCTCTCCACCCACGCTTTCCAACTGCT |
| | oxidase Mbb2 | CGCGCTGATCATCTATGCACTCGCCAACGCGCTCATGTTT |
| | Coding | GTCTGGGGCGCCCATGATGAATTCCACCATCACACCGACG |
| | sequence | CCAATAATCTGCGATGGTACATCTGTATCGCCCGCGGCGC |
| | | AGGTTACACACTCAATCTCAACACGGCGCTCGTTATTCTG |
| | | CTCGCCGCCCGACTCTTTGCAACTTATCTTCGAGAAACGC |
| | | CCCTACAGCATATTCTGCCCCTCGACAAGAGCTTTCCAGC |
| | | TTTTCACATCGTTGTAGCCTACACTATTGCCGCCGCCGTT |
| | | GTTATTCACGCCTCGTTTCATCTAGCCTGGCTCGTCGCCT |
| | | ATGACATGTGGGAAACTGGCATGTGGGGATTTACGATGAG |
| | | CGCCGCGACCGGAGTCGTCCTGCTCGTTGTCTTTATTGTC |
| | | ATGTTTATTTCAGCGATGCCCAAATACCGGAAAAAACATT |
| | | TTCGCATCTTTTATCTTATTCATTCCGTTGGTGCTCTGCT |
| | | CTTTTTCGGTCTGCTCGTATTTCATGGAATGTACAACAGA |
| | | GTACCGGAGACGTACAAATGGATTGCTGCACCGCTTATCA |
| | | TCTATACCATTGATAGAGTACTTCGACGGTACAAGATATC |
| | | CACCGCTGAGCTCGAATTAACTGGAGAACATTCCTCGCTG |
| | | AAAGGGAGTGATATTCTGGAATTGCGAGTACCAAAGCCGT |
| | | TTGATTATCAAGCTGGACAGTATGCAGAAGTTTGCGTCAA |
| | | ATCAATTAACAGTGAGTGGCATCCGTTTACAATTGCCAGT |
| | | TCGCCTCATGAGGACTCAATGTGCTTCTATATTAAAGCGC |
| | | TTGGTGATTGGACAACAAATTTACGAGATGCTTTTGAAGC |
| | | CCGAGTTGAGAATGATCTGTACGAACCACTTAAGGTACAG |
| | | ATTCGAGGCCCATTCGGAGCTCCAGCTCAACATGTGAGTG |
| | | GGTATTGTCGAGTCGTGCTTATTTCCGGTGGTGTCGGCTC |
| | | CACCCCATTCGCAGCTATTTGCAAGCATCTGCATCATTTG |
| | | AACAAGTCGGAAAATCATTCCAAAGCTCTCGAATCGCAAG |
| | | TTTCCTCCAAAAGGCAGTCTCAAGTGCAATGGCGTATTCG |
| | | AGATGCCATTTCCATACTTTTCGACGTCAGTCTGGATGAT |
| | | AGTCGAAATGATGAAGCCGCAAATCAGCAAAGACGACAGC |
| | | AACTGGCTGATATGCTCAACATGTCGCCAAAGGGTGATGT |
| | | GCATAACGTTTCATTCAAACGCACTCAATCCAAGAGGTCA |
| | | GATGACTTTGATGCCGTGGAGGACTCGAAACAAATGAATT |
| | | TTTCATTGTGTCGCCAAGATAGCAAATCGAGTTTCAATCT |
| | | TGATGACTGTTCTGATGCCATCCGATACTACAATCGTCCT |
| | | TCCTTTCAGTTCTTCAGTTCATCCCCAAGGCATGTAATCA |
| | | ATTGGTATGAATACCGTACAAGGCTACTCGCATTTCTGCA |
| | | CACTACTCGGTTTACGTTTGCACTTTTGCTCACGCTCGTC |
| | | GCAAGAATTGTTATCTTGTGCATAGTGTCCATATTCAATC |
| | | TCGGACACATTGGACTTTACAATAGTCATATCGATGCTAC |
| | | GTGGGTGATTGCCACTAACTCCATATTGGGACTCATACTT |
| | | GGAGTCGCTCTGTTAACAACTATTCTATTGGAAATCAGTT |
| | | TTATGCGCGTGCGCTTCTTCTACCGAGTCTGGCGCTGTGT |
| | | AGACTTTTTCATCTTTCTACCTATAACTTTCCTGTGCAAC |
| | | ATCAGCAATTTCGCCAGCTGGAACGGTCATGCACAGCCGA |
| | | AATTCCTAATATTCCTGGATTTGATCATTGTACTGCCCAT |
| | | CATGCTGCTTTTGTTATGCCATCGTATGTACCGGAGCGTC |
| | | GGGTCACGAAACCTTCTGGACGACACGGCCGAATGTCGCC |
| | | AGGGCTGTAAATGCAACAAAACGATCCCGGATGTTGATTT |
| | | TGTGTGGACGACGCCACGCGACAGCGACGACGAATGGCTG |
| | | CGAAATGAACTGTATCCACTGGCCACGGGGACGGAGCTTC |
| | | GGTTGCATCGGTATGTGACTCGCGAGAATATGGCCGACTT |
| | | GGAGGACCCAGAGCGCTTCATTACAACGGCAAACGCTGGA |
| | | CGACCACAGTGGGACGAGATTTTCGCACAGATTGCAGAGC |
| | | AAACGCCAAGTCACAGCAAGATCGGGGTGTTCTTTTGCGG |
| | | GCCGCATCCGATGGGTGCTGCCGTGCAAAAGAGTATGCGC |
| | | AAAGTGGAGGTCATGAGTAATCTGCGTGGGTCGTACCTGC |
| | | GCAAGACAGAGAGTGCGGTTCTGGTCGATGATTTGATACT |
| | | GAGGGATGAGGGGGAAGTGAAGTTATTGAGGGAATATGGG |
| | | TGTAATATTCGATTTGTTTTCCGCGAGGAAAACTTTAGCT |
| | | AG |
| 49 | A. taxiformis | ATGACGCCGCCAACACGCCGCCAAACAGCGCTCGTCAAGC |
| | Vanadium- | GCATTGCCGCCGCAGACCTGGCCGCCAATCGTCACCATCC |
| | dependent | GCTTCATGTGCGCAATGCCGACGAAACCACCTACAAATTG |
| | haloperoxidase | CCAAACCATGTCTGTTCCTACATATCCGCTTTCACCAAGG |
| | Mbb3 Coding | GTTTGCCCCATCATCTCGACGGCCGCCTCGTTTCCCCACA |
| | sequence | CCACTTTCAACTCTTCGTCCACGCCGTGCAAACTGGCCAT |
| | | GTCACAGACATTGCCGCCATTCCACTCGGACCCCCACACT |
| | | CCTCCTCCTTCCGTTCCGGCATCGCCAAGGGCGCCGCCGT |
| | | GCGCGCATGGGAGTCCATGGCCGCGGGTAACACCTTTGAT |
| | | TTGCAAGGGCCTGATGCACAGGCCGTTACCATGCCCCCTT |
| | | GTCCGCGTCTCAATTCACACGAGCTCGTCACAGAGATGAC |
| | | AGAGCTTTATTATATGGCACTGTTGCGCGACGTGCCGTTT |
| | | ACGCATTTCAACAAACATCATCTCGTTCAGTCCGCTGTGC |
| | | ACGACATGAATCATACCCATTGGACTCGGTCCAACAGATG |
| | | CTGTTTACCTCGCCCCGAGCGCCGTCGACATCGCTCCAAA |
| | | TTCACCACTGATAACATCTTCCGCGGTGTAACGCCCGGTG |
| | | ACATAACCGGTCCCTACATTTCCCAGTTTCTCCTTGTCGG |
| | | CACAAAGGGTCTCGGCGATGCACACGAAGAAACAGAAGGC |
| | | TACGTTCAATACGGCGCCACAAGAATCGACCAGCGAGTGC |
| | | GCACTGTCGAACCCAAAATTGACTACATGACGACATGGGC |
| | | CGCTTATCTCGACGTGCAGAATGGTGCCGACTTACGCCGT |
| | | CACTCCACCTATGCCAAACCACCGTCTTACCGCTTCATTA |
| | | GCACCCCGCGCGACCTCGCAACCTATGTGCATTACGACGC |
| | | GTTGTATCAAGCGTATCTCAACGCGTGCATCATCCTGCTC |
| | | GACATCAACGCGCCATACGATGGCGGACTGCCCTTCACCG |
| | | CCGACGATGCCGTCGACAAGCAGCAGGGCTTTGCGACATT |
| | | TGGCGCGCCACATGTGCTGACACTTGTGACAGAAGTGGCG |
| | | ACACGCGCGCTCAAAGCTGTGCGCTTTCAAAAGTATGCCG |
| | | TGCATCGGCGTCTGCGGCCGGAAGCTGTCGGCGGTCTATT |
| | | GGAGCAGTATAGACGCTACGGTGGTGGACATTCGGATCTC |
| | | GCATACATCATTCGTCCGATTCGTCATCTCGCCGACTCGC |
| | | TCAGTTCCGATCTGATGTTCATGGTCGCCAAGGAGAACGG |
| | | ACGCCAGAATAGCTCGATTACGGATAACGGCCATCCGCGC |
| | | TCGCATGACGAAGGTTCGGATGATACGCATCTGCTTTCCA |
| | | TGGCGTATGCGGAGGGAAGCCCGATGCATCCTAGTTATGG |
| | | CGCGGGACATGCAACGGTTGCGGGCGCTTGTGTAACAATC |
| | | CTGAAAGCGTTCTTCGATCATACGTTCAAACTGCCGTTTG |
| | | CGTACGTGTCGTCGAGCGATGGGAGGAAATTGAAGACGGT |
| | | CAAGTTGAGCAGGAAATTGACCGTGGAGGATGAGTTGAAC |
| | | AAGTTGGCAGCGAATATAAGCATTGGACGCAGTTGGGCTG |
| | | GCGTCCATTACTATTCGGATTATGTTGAGAGCATTCGGTT |
| | | GGGCGAGGAGGTGGCCATCGGGATGCTCAAGGAGCAGAAG |
| | | TTAACTTATTCGGAGAAATTCACGATGACAATACCCAAAT |
| | | TTGACGGCTCCGTCATTGAAATTTAG |
| 50 | A. taxiformis | ATGGCAGAGGAACGTCGCCAAAACGCTCTAGACATCCGCA |
| | Vanadium- | TCCAAGCCGCCAGGCTTGCGAAGAAACGTGACCATCCAAC |
| | dependent | CCATGAGGCAAACGGCGATGAGGATCGTTACCCAGAAACA |
| | haloperoxidase | CTCATCGGCTCCTTCACTAAAGGTCTTCCACATGAGAAAG |
| | Mbb4 Coding | AAACCGGTCTCCTCTCCAACCCCGCCGATTTCGCCGACTT |
| | sequence | TGTTCGCGCTATCAACACAGGCGCGATCAAAGACATAAGA |
| | | CGCCTCAAACTTGGAATCGATGAGGATCCTCGCTTCATAT |
| | | CTGGTATTGCCTCCAAGGGCGACAAGCATCCTTTCGCCGA |
| | | TACGCGCGCCTGGGAATCCATGGCCGCCGGTCTCACATAC |
| | | GATCTAGAAGGCCCTGATGCCCAAGCTGTCACCATGCCGC |
| | | CGGCGCCGAAACTCGACAGTGATGAACTCGTCACAGAGAT |
| | | TACGGAGAGCTATTGGATGGCTCTTATTCGTGATGTGCCG |
| | | TTCACAGAGTTTGAACGTGATGGAAATACAGCCGCAGCCG |
| | | CAGCTTCCATCAGTCGCACTCGTTGGGTACAATTCAATGA |
| | | GCAGCCTTCACAGCGTCCGTCAACATTGACCGATGAAGAG |
| | | ATTGCTAGATTGAGGGGTCCGTATACCAAGAAAAATGTAT |
| | | TCCGTGGTGTGACGATCGGTGAGAACGTCGGCCCGTATCT |
| | | GTCTCAGTTCTTACTGGTAGGCACGAAAGGTATCGGCGAT |
| | | GTACAGCAAATTTCGGATGGTTACATTCAGTACGGAAGTA |
| | | TGCGAATGGATCAGCGTGTCCGCGTCGCAGCGCCGAAACG |
| | | CGATTATATGACAACTTGGGCATCTTGGCTAGATGTGCAG |
| | | AACGCTGGTGATTTACGTGGACGTGAAATTTACGATGATG |
| | | ATACACCGTTCCGCTTCATTACAACCCCACGCGACCTTGC |
| | | GACATGGGTTCATTTCGACGCGTTATACCAGGCATATTTG |
| | | AATGCTTGCATCATCCTTCTCGACATCAAGGCGCCGTTCG |
| | | ATCCACACATCCCGTTCCAGGCTGATGACGATGTAGATAA |
| | | GCAGCAAGGATTTGCAACGTTTGGCGGCCCGCACATCCTA |
| | | TCATTGTGCACGGAAGTGGCTACGAGAGCACTCAAGGCAG |
| | | TTCGCTTCCAAAAGTACAACGTGCATCGTCGTCTTCGTCC |
| | | GGAGGCTATCGGCGGGCTTGTGGAACGCTTCAAGAAGACA |
| | | AACGGAGATGCCAAGTTTGCCCCAGTGAAGAAGCTGGTGA |
| | | ATGACCTGGACGGCGATATGCTGCGACGCATTGAGCAGCA |
| | | CAACTGTGAGCAGAATAAGCTTTCAGATGATGGACATGCA |
| | | CGCCGAGACGACTATTCACCGGAGGGTGAGAGTAACCAGA |
| | | CGTACCTTCTGCCCATGGCGTTCCCGGAGGGGAGCCCGAT |
| | | GCACCCGTCATATGGTGCAGGTCACGCCACAGTTGCCGGT |
| | | GCGTGTGTGACGGTGCTCAAGGCATTCTTCGACCATGAAT |
| | | ACGAGCTGGACTTCTGCTACGTACCGACCACGGATGGCAA |
| | | GCGTCTGGAGAAGGTAAATATCAATGAAAAATTGACAGTG |
| | | GAAGGCGAACTGAACAAGCTGTGCGCTAATATCTGTATTG |
| | | GTCGAAACTGGGCTGGCGTGCATTACTACTCGGACTACTT |
| | | TGAGAGTATCAAAGTTGGTGAGGAGATTGCAATTGGTATT |
| | | TTGCAAGAACAGAAGCTTACGTATGGTGAAGACTTTTTCA |
| | | TGACGCTGTCCAAGTTTGACGGTGAAAAGATCCGTATCTA |
| | | A |
| 51 | Chondrus | ATGTCGGCCGCCCGTAGACAGGAAGCCTTCCAGACCCGTG |
| | crispus | TCCGTGCCGCCCGCCTGGCTCGCGACCGTGACCACCAAGT |
| | vanadium- | CTTCGAACCCAACGGTGAGGAGGACCGCTACTTCCGCCCC |
| | dependent | AACTCCCAGATCTTCAGCTACCTCGGCTCCTACACCAAGG |
| | bromoperoxidase, | GCTTGCCTCACAACCGCCGCACGGCCCTCATCACCTCGCC |
| | vBPO_01 | GAACCACTTCGTCCGCTTCGTCCGAGCCATCGACTCGGGC |
| | Coding | GAGCCAGCCGACTTCATCAAGGTCCCGCTCGGCCCGCGCC |
| | sequence | CGGCCCTCCAGTTCGAGTCCGAGGAAGAGTGCCGCGCCGC |
| | | CAACCTCTTCAAATCCGGCATCGCCGGCCGCAACAACACC |
| | | TTCCCGTCCTCCTGCCTGCGCGCATGGGAGAGCATGGGCG |
| | | CCGGCCTCGTCTTCGACCTGGAGGGCCCGGACGCCCAGGC |
| | | CGTCGGCATGCCGCCCGCCCCCACGCTGGACAGCGACGAG |
| | | CTCGTCGCCGAGATCTCCGAGGTCTACTGGATGGCCCTGC |
| | | TGCGCGACGTCCGCTTCACTGATTTCACCAACCCCGGCCC |
| | | GATCGGCAGGGCCATCAACACCCTCAACAAGACCGCCTGG |
| | | ATCCGGGCGGCACGGGACCCCCCCGACTCGCTCACGCCGC |
| | | AGGAGCGCGCCCGCCTCCGCGGCCCGTTCAACCCGAGCAA |
| | | CATCTTCCGCGGAACCCTCCCCGGAGATGACGTCGGCCCG |
| | | TACCTCTCCCAGTTCTTGCTCGTGGGAACTAAGGGCATTG |
| | | GCGGCGCACAGGACATGGCTGACGGGTTTATTAGCTACGG |
| | | CGCCCACCGCATCGACCAGCGCGTGCGTCACGCGAGGAAG |
| | | GAGCTCGACTACATGACCTCTTGGGAGGCCTTCTTGGACG |
| | | TCCAGAACGGCGCCGATGTCACCGGTCGCGATACCTTTGA |
| | | GTCGGGCTCTGACGCCTTCCGCTTCATCACAACCCCCCGT |
| | | GATCTCGCCACGTACGTCCACTTTGACGCGCTCTACCAGG |
| | | CCTACCTCAATGCCTGCCTGATCATGCTTGACAGCGGCGT |
| | | GAGGTTTGATAAGGGTATTCCGTTTGGTGAGCCAGACTTC |
| | | AAGGATCATCAGAGAGGCTTCGCTCATTTCGGAGGACCGC |
| | | ATATTCTTTCCCTCGTCACAGAAGTTGCCACAAGGGCTTT |
| | | GAAGGCTGTTCGCTTCCAAAAGTTCAACACGCATCGCCGT |
| | | CTGCGTCCCGAGGCCGTTGGTGGCCTGATCGAGCGCTTCA |
| | | ACAGCAACCCTGACGACCCTCAGTTCCAGGATGTGAAGCC |
| | | GTTGTTCGAAGCGCTGGATGAGGATATGATGCGCCGCGTT |
| | | GCTCGTCACAACAGGGAACAGAACCAAGGCTCTGACTTTG |
| | | GCATGCCGCGTGCCGACGACTTCAACCCCGCCGGTGACAC |
| | | TCTCGAGACGATGCTGTTGCCCATGGCTTTCCCCGAGGGA |
| | | AGTCCGATGCATCCGTCGTACGGTGCCGGCCATGCCACTG |
| | | TGGCCGGGGCTTGTGTTACCGTGTTGAAGGCATTCTTCCA |
| | | GCACGACACCGAGCTCGATTTCTGCTTCGTTCCCAGTGAT |
| | | GACGGAAGCCGTCTTGTCGACGCCAGCCACAACATGAATA |
| | | AGAAATTGACTGTAGAGGGAGAGTTGAACAAAGTCTGCTC |
| | | CAACATCAGCATTGGCCGCAACTGGGCTGGCGTTCATTAC |
| | | TTTACCGATTACATTGAGTCGATTCTCCTCGGGGAACAGA |
| | | TCGCGCTAGGCATCTTGGAGGAGCAGATGTTGACTTTCCC |
| | | CGAAACCTTCACAATGACCGTCCCTCTCTTCAGCGGCGAA |
| | | TTGAAGGTCCTCCGCTCGACCTGA |
| 52 | Chondrus | ATGGCAACCCCGTGCTTCGCCCAGGCGCCGGCCCCCACCC |
| | crispus | TACGACGATCGCCGCGCCGTCTCCCCCTCACCCCGACCTC |
| | vanadium- | CTTCCTGCCGAGGCCGTTCCTGCCAAAGCCCTTCCCCCGC |
| | dependent | TCCCCCCGCCACCCGCATCCGCGCCTCAACGCCGCCTCCG |
| | bromoperoxidase, | CGCGCACCTCCGCCGCCCAACCCCCCGACCGCGCAGCCAC |
| | vBPO_02 | GCCGTGCGCTACCCGCCAGCAGCAAGCGCTGGAGCTGCGC |
| | Coding | CGCGCGGCGGCGCAGCTCGCGCACGACCGTGCGCACCCCG |
| | sequence | CGCACGTGTCGAACGGGTCGGAGGAGCTTCACCTGCACCC |
| | | GAAGACGGGCGCGCCGCTGCTGCTGGCCAACTTCACGAAG |
| | | GGGCTGCCGCACGAGCGCGACACGGGGCTGGCAGCCGACG |
| | | CCGCGCACTACGACCTCTTCGTGGCGGCGGTCCATTCCGG |
| | | CTTGCCGGACGACATCCGCGCCATCCCGCTCGGCCCGCAG |
| | | GCCGCGCCCGGCGTCCGCCTCCCGCGCTCGCTCCGCTTCC |
| | | GCTCCGGCATCGCACGCAGCTCGCACCTCTTCCCGCACAC |
| | | TGACGTCCGCGCCTGGGAGAGCATGGCCGCGGGGAACGCG |
| | | TTCGACCTGCAGGGGCCGGACGCGCAGGCCGTCACCATGC |
| | | CGCCCGCGCCCGCCCTCGCCTCCCCCGAGCTCGCGCTGGA |
| | | GATGACGGAGAACTACTGGATGGCCCTGCTGCGCGACGTC |
| | | CGCTTCTCGCAGTTTGCCGCGGACCCCACCGTCGCCGCCG |
| | | CCATGGAGAGCATGAACGCTACCGAGTGGGTCGCCCTCGC |
| | | GTGCGGCGACGCGTCGGCCGTCGCGGAGGAGGAGCGCAAG |
| | | CGCCTGCGCGGGCCGTTTACCGCGCAGAACGTGTTCCGTG |
| | | GGAATACGCAAGGCGATTGCAAGGGGCCCTATATCTCCCA |
| | | GTTTCTGCTGTCTGGAAGCGCGGGGCTGGGCCAAGTGAAC |
| | | AGCGGCGCGGATGGATACATCCAGTACGGAGCAATGCGTA |
| | | TGGATCAGCGCGTTCGTGTCGCCCTCGAGGGGCTTGACTA |
| | | TATGACCACGTGGGAGAGCTGGCTGGATGTGCAGAACGGG |
| | | GCCGACTTGCGGGGAAGGGAGTTGTATGAGGACGGCGAAC |
| | | GCTTTAGATTTATTTGCACCCCGAGGGACTTGGCCACTTA |
| | | TGTCCATTATGATGCCCTGTATCAGGCTTACTTGAACGCC |
| | | TGCATTATCATGCTCAACAAGAAGATCCCCTTTGACAAGG |
| | | GCCTGCCGTTCCAGAAGGATGACGACATCGACAAGCAGCA |
| | | GGGCTTCGCCTTATTCGGCCCGCCGCACATTCTCACCCTG |
| | | TGCACGGAGGTCGCCACCAGGGCCCTCAAGGGCATCCGCT |
| | | TCCAAAAGTACAACGTCCACCGCCGTCTGCGTCCGGAGGC |
| | | TGTCGGCGGCCGCGTGGAGCGCTACCACCACAACTGCGAG |
| | | GACCCGCTCTTCGCCGACGTCAAGCCGCTGTACGACGCCC |
| | | TGGATAAGGACATGCTCGAGCGTGTTGCCGCGCACAACGC |
| | | CGAGCAGAACGCCAAGGACGACTTTGGGAACAGCCGCTTC |
| | | GAAGACTATTCCCCGACGGGTAGCTCCGGCAGGACCTATC |
| | | TGCTGCCCATGGCGTTTCCGGAGGGCAGCCCCATGCACCC |
| | | GGCGTACGGCGCAGGGCATGCGGCAGTGGCCGGCGCGTGC |
| | | ACCACTATCCTCAAGGCGTTCTTCGACCACGAGCACGAGC |
| | | TTGACTTTGCGTACGTGCCCACCGCGGATGGCAGCAAATT |
| | | GGAGGATGTTGTTGACTCTTTGGGGGAGAAGCTGACCGTC |
| | | GAGGGAGAGCTCAACAAGGTCTGCTCGAATATCAGCGTCG |
| | | GGCGCAACTGGGCTGGTGTGCACTACTTTACAGACTATCG |
| | | CGAGTCCATCATCCTTGGGGAAAAGATTGCCCTCGGCCTG |
| | | CTGGAGGAGCAGAAGCTTACGTATGCTGAGGAGTTCTCCA |
| | | TGACGATTCCTCTCTTTGATGGTTCCACTGTCACCATTTA |
| | | G |
| 53 | Chondrus | ATGTCGGACATGTCGACTGAGATCAGCCCTAGACAACAGG |
| | crispus | AAGCCTACGATATCCGTGTCGCCGCCGCCACCCTGGCTCT |
| | vanadium- | GGCTCGCGACCGTAAACGCAAAGTCTTCGAACCCAACGGT |
| | dependent | GAGGAGGACCGCTACTTCCGCCCCAACTCCCAGATCCTCA |
| | bromoperoxidase, | GCTACCTCGGCTCCTACACCAAGGGCCTGCCTCACAACCG |
| | vBPO_03 | CAGCACGGCCCTCGTCACCTCGCCGAACCACTTCGTCCGC |
| | Coding | TTCGTCCAAGCCATCGACTCGGGCGAGCCAGCCGACTTCA |
| | sequence | TCAAGGTCCCGCTCGGCCCGCGCCCGGCCCTCGAGTTCAC |
| | | GACCGAGGAAGAGTGCATCGCCGCCAACCTCTTCAAGTCC |
| | | GGCATCGCCACCCGCAACGACAGCTTCGATCCCGCCTGCC |
| | | TGCGCGCATGGGAGAGCATGGGCGCCGGTCTCGTCTTCGA |
| | | CCTGGAGGGCCCGGACGCCCAGGCCGTCGGCATGCCGCCC |
| | | GCCCCCACGCTGGACAGCGACGAGCTCGTCGCCGAGATCT |
| | | CCGAGCTGTACTGGATGGCCCTGCTGCGCGACGTCCGCTT |
| | | CACTGACTTCACTAACCCCGGCCCGATCCGCAGGGCCATC |
| | | AACACCCTCAACAGGACCACCTGGATCCGCGCGGCACGGG |
| | | ACCCCCCTGACTCGCTCACGCCGCAGGAGCGCGCCCGCCT |
| | | CCGCGGCCCGTTCAACCCGAGCAGCATCTTCCGCGGAACA |
| | | CTCCCCGGAGATGACGTCGGCCCGTACCTCTCCCAGTTCC |
| | | TGCTCGTGGGAACTGAGGGTATTGGCGGCGCGCAGGACAG |
| | | GGCTGACGGGTTTATTAGCTACGGTGCCCACCGCATCGAC |
| | | CAGCGCGTGCGTCACGCGACAAAGGAGCTCGACTACATGA |
| | | CCTCTTGGGAGGCCTTTTTGGACGTCCAGAACGGCGCTGA |
| | | CGTTGCCGGTCGCGATACCTTTGAGTCGGGCTCTGACGCC |
| | | TTCCGCTTCATTACGACCCCCCGTGATCTCGCCACGTATG |
| | | TCCACTTTGACGCGCTCTACCAGGCCTACCTCAATGCCTG |
| | | CCTGATCCTGCTCGACAACAAAGTGAAATTTGATCAGGGT |
| | | ATTCCGATGGGTGAGCCAGACTTCAACGGTAATCAGGGTA |
| | | TTCCGTTTCAAGATCCAGACTTCAAGGATCATCAGCGAGG |
| | | CTTTGCTCATTTCGGAGGACCGCATATTCTCTCCCTCGTC |
| | | ACAGAAGTTGCCACAAGGGCTTTGAAGGCCGTTCGCTTCC |
| | | AAAAGTTCAACACGCATCGCCGTCTGCGTCCCGAAGCCGT |
| | | TGGTGGCCTGATCGAGCGCTTCAACAGCAACCCTGACGAC |
| | | CCTCAGTTCCAGGATGTGAAGCCGTTGTTCGAAGCGCTGG |
| | | ATGAGGATATGCTGCGCCGCGTTGCTAGTCACAACCGTGA |
| | | ACAGAACGAACGCTCTGACTTTGGCATGCCGCGGGCCGAC |
| | | GACTTCAACCCCGCCGGTGACACTCTCGAGACGATGCTGT |
| | | TGCCCATGGCTTTCCCCGAGGGAAGTCCGATGCATCCGTC |
| | | GTACGGTGCCGGCCATGCCACTGTGGCCGGGGCTTGTGTT |
| | | ACCGTGTTGAAGGCATTCTTCCAACATGACGCCACGCTCG |
| | | ACTTCTGCTACGTTCCCAGTGATGACGGAAGCCGTCTTGA |
| | | CGACGCCAGCCACACCTTGAATAAGAAATTGACTGTAGAG |
| | | GGAGAGTTGAACAAGGTCTGCTCCAACATCAGCATTGGCC |
| | | GCAACTGGGCTGGCGTTCATTACTTTACCGACTACATTGA |
| | | GTCGATTCTGCTCGGCGAACAGATCGCGCTAGGCATCTTG |
| | | GAGGAGCAGATGTTGACTTTCCCCGAAACCTTCACAATGA |
| | | CCGTCCCTCTCTTCAGTGGCGGCTTCAGGACCCTCATCTC |
| | | GACCTCTGATCCTTGA |
| 54 | Asparagopsis | TTAAATGCGAATTGTGCTTCCATCAAACTTGTTGAGGGTC |
| | armata Azores | ATCGAGAAATTCTCACTGAAGGTCAACTTCTGTTCCTGAA |
| | Mbb1_like | GAATGCCAATAGCAATTTCCTCACCAATGCGGATACTTTC |
| | coding | AATGTAGTCAGTGAAGTAATGCACACCAGCCCAGTTACGT |
| | sequence | CCAATGGAAATATTAGAGCAAATCTTGTTCAGCTCTCCGT |
| | | CAACTGTGAGCGGCTCAGACACTTCAACCGGCTCGAGGCC |
| | | GCAGCCATTTTCGTTAGTGACGTATGCAAACGGCAGGGGG |
| | | AATCCACCATCAAAGAATGCCTTGAGAACAGTTACACAGG |
| | | CTCCTGCAACAGTGGCATGTCCAGCACCATAAGCCGGATG |
| | | CATCGGGCTTCCCTCTGGGAATGCCATTGGCATGAGAAGG |
| | | TTGCCTTCGCTCAGGCCGCCATCAGGATTGAAATCTGCAC |
| | | TACGCGGAGCACCGTTGTCGCTCAACTTATTTTGCTTCTT |
| | | GTTATGGTAATACACTCGATCCAACAGGTCAGAATCCAGG |
| | | GAATCTACCAATTTAGCAGCTCCGGAAAATTCGGGTGCTT |
| | | TGGTTTTGTAGTATCGGTCGACACGACCGCCGACAGTCTC |
| | | CGGGCGTAGACGGCGATGGACGTTGAACTTCTGGAAGCGC |
| | | ACTGCCTTGAGAGCGCGAGTGGCCACCTCAGTAACGAGAG |
| | | AGAGAATATGCGGTCCGCCAAACGTGGCAAATGCCTGCTG |
| | | CTTATCTATATCATTGTCGAGCTGGAACGGGACTCCGGAA |
| | | TCGAACGGTGCACCAATGTCGAGCAGAATGATGCAGGCGT |
| | | TAAGATAGGCCTGGTACAGAGCGTCGAAATGGACGTATGT |
| | | CGCCAGATCACGCGGTGTGTGAATGAAGCGGAAACCAGGG |
| | | TCTGACTCGTATCGCTCACGCCCGGTGACATCCGCAGCGT |
| | | TCTGCACGTCAAGCCAGGATCCAAAGGTGGTCATGTAGTC |
| | | AACCTTGGGGGATGCCACGCGTACCCGCTGGTCCATGCGC |
| | | ATACCACCGTACTGGATGAAACCATCGCTGACCTCGTTTC |
| | | CGTTTGCGATTCCTGTGGTACCGACCAAGAGGAACTGGGA |
| | | CAAATATGGGCCTACATCATCTCCAGGAGTGATGCCCCTG |
| | | AAGACATTGGAGGCATCGATGGGGCCTCTGAGACGGGAGC |
| | | GTTCGGCAGATGTGAGATGGGTGGGAGGGTTTTCCTTGAA |
| | | TTTAATCCATTGAGTGTCGTTGATACTCTTGGCAGCGGCT |
| | | GCGACATCTTCGTCGCAATGGAATGTTGGGAAGGGTACAT |
| | | CCCTGAGCAGGGACATCCAATAACATTCGGTGACTTCAGT |
| | | GACCAACTCCTCGCTGTCTAGCTCTGGTGCGGGCGGCATA |
| | | GTGACGGCCTGTGCATCAGGCCCTTCGAGATCGAATGTGC |
| | | GTCCAGCACCCATACTTTCCCAGGCACGAACTCCCGACTG |
| | | CTCTGGGTTTGTGGCGATGCCGGATGTGAATCTGGGGGTG |
| | | ACATTCTGGGGGCCGAGTGGGAGAGCTGCGAAATCGCGGG |
| | | CATCTCCGCTGTCAATAGCGCGTACAAATTGGTCATAATC |
| | | GAGGGCAGATTGAACCAGACCTGTTTCGTTGATATGGGGG |
| | | AGACCTTTCGTGAAGTTAGCCAAGAGAGATCGTGTACCAT |
| | | CTGGGTTTCTGAAGCGAGCCTCTGAATTGTTGGAGATGTG |
| | | AGTTGGAGAACCACGGGAGCGAGCAAGCTCGGCGGCAGAG |
| | | AGGCGAATCTTGAAGGAGGCCTCTGCGCGATTGGGATTCT |
| | | GTGTGTCGGGGGTTGCCAT |
| 55 | Asparagopsis | ATGAACACAGCATTTCCCTTCAGAAGAAAATGGTCCCCAA |
| | armata Azores | GTCTTTGGAGTGCTCGCCTCGAGTCCTATCTCTCCACTCA |
| | - Mbb2-2_like | CGCCTTCGAGCTTCTCACAGTTATCACATACATAACTGCA |
| | coding | CTCTCTCTCATGTTCGTATGGGGAGCTCACGATGAGTTCC |
| | sequence | ACAATCAATCTCCCGACACAAATCTCCGCTGGTACATTTC |
| | | TATTGCTCGCGGAGCAGGTTACACACTCAACCTCAACACA |
| | | GCACTTGTCATTCTACTTGCATCTCGCCTCTTCTTCACAT |
| | | TCATCCGTGATACTCCCCTGCACAAGATTCTTCCTGTTGA |
| | | CAAAAGCTTCCCCGCCTTCCATATCCTTGTTGGATATACG |
| | | ATAGCCGCATCTCTCTTCATTCATGTCCCGTTCCATTTCA |
| | | CATGGATCATTGCTTACGATAAATGGAGTGCAGGACTTTG |
| | | GGATTTCACCATGAGCGCTGTCACAGGCGTCGTATTGTTA |
| | | CTTATCTTCATAATCATGTTCATGTTCGCTCTACCATTCT |
| | | ACAGAAAAAAATATTTCCGGCTCTTCTACATTTCCCACAT |
| | | GGTTGGAGCACTGCTGTTCTTTCCTCTTCTCATCTTCCAT |
| | | GGTATGTACCGTCGTGTTCCGGAGACTTACAAGTGGGTTG |
| | | CTCTACCTCTCGTGATCTACATTCTGGACAGAATCGCCCG |
| | | TCGCTTCAAAATCTCCACCGTTCAGCTGAAGCTCACTTCC |
| | | GAGCATTCAGCTCTCAAAGGAAAAGACATTTTGGAACTTC |
| | | GTATTCCGAAACCCTTCACATATCGCGCTGGTCAGTATGC |
| | | AGAGATCCGTGTACCATCCTTGAGTAGGGAGTGGCATCCC |
| | | TTCACAATTGCTAGTGCACCCCATGAAAAATGCATGTGCT |
| | | TTTTCATCAAATCACTCGGGAATTGGACCAAGGAGCTTCG |
| | | AGGGGCATTTCAAAACCGTATGGAAGGAGAAGATCCGAAC |
| | | CCATTGCTAGTTCAAGTCCGTGGTCCATTTGGCGCTCCCG |
| | | CTCAACATGTCTCTTCGTATGAAAGAGTGGTCCTCATATC |
| | | TGGCGGAGTTGGTTCAACGCCCTTCGCAGCCATCTCCAAA |
| | | AGTTTGCATTTCTTGAACAAGAACAAAACCTGCCCTTCTA |
| | | CTGATTTTTCGAAAACCAATTTGTCTGAAATGAAGCATAT |
| | | TGAGCAGCGTATCTCGGAAACTATTTCTGCTGTATATGAT |
| | | ATCGATTTGGCAAACAGCTCCGATGAACCGGAATCTGAAG |
| | | AACAAAGGAAGCAGTATATTACTGACATGATGAACTTGAC |
| | | GGCCCAAAATTCCGAAGAACTCGATGTTTCCACTTCAAGA |
| | | ACAAGTGAACAATGTGTATCATCGAGGAGCTGCCCAAATA |
| | | TTGGAGATGGTCTCCGGATATTGGATGTCGAAAATCAATT |
| | | AGAAGAATCTTCAGTGAGCAGCATGTCTACATGCGACGAT |
| | | GAAGAAGATTATTCATCAGATTTCAGTGAAGAAATGAGAC |
| | | TTGAAAAGGAACTTGCCGCCAGGAAACGTAAGATGGCTCG |
| | | CCAAAAAATTGCCCGTCTCTATGAGAGACGTTCAATATTA |
| | | TTCTCTTTTCTTCATACCACTCGGGTAACTCTGGCACTTC |
| | | AACTCTCTCTAATTGCACGCATTGTTCTTGTGTGCATTGT |
| | | ATCAATCTTCAACATTGGTGAATTCAACTTTGGGGACAGC |
| | | CGCATAGGTGCCAAATGGGTTATAGGTGCCGATGCAGTGT |
| | | TGAGTGTTTTGCATGCAGTCACTCTAACTTTGACCATACT |
| | | CTTGGAAATTAGCTTCATGGGACCCCGCTTCTTCCGTTCT |
| | | TCTTGGCAGTTTATGGACTTTTTCGTTTTCGTCCCGCTCA |
| | | GTATTCTGTGCAGCGTCAGTGGTGTACTATCTTGGACTGG |
| | | GACGGAAATGTCAGGAACATTAGTTTTTCTTCACTTCATT |
| | | GTATTGCTGCCAATTATGCTTATTTTCATGAGTGCTCGTC |
| | | TGTATCGGAGTATTGGGTCCCTGAATCTATTTCATGATCG |
| | | GAAGTGTAACCCAAACTGCGCATGCGGAAAGAAAATCCCA |
| | | GACGTTGACTTTATCTGGACAACACGTCACACCGTAGATG |
| | | ACCAATGGTTACGAGAAGAACTCTTTCCTCTAGAGGACGG |
| | | CACAAAACTCCGTCTTCACCGATATATTACAGGCAAGGAA |
| | | AGTGAAGACTTTGAAAGTGCTGAGACCTTTATTACTACAA |
| | | GCAACATTGGAAGACCGCCCTGGGACGAAATATTCGCCAA |
| | | AATTGCCCAAAAGTCACCAAGTCACGCAGAAATCGGAGTG |
| | | TTTTTCTGCGGACCACACTCAATGGGAGCAGCTGTGAGGA |
| | | AAAGCATGCGAAAAACTGAAATACGGAGTAACTTGCGTGG |
| | | GGCATATCTGGAGACAACTAATGTTGTCGATCTTGTTGCC |
| | | GATCTTCTTCTGAGAAATACTGACCATGCAGCAGAGCTTC |
| | | GGAGGCATGGGTGCAATATTCGATTTGTATTTCGTGAAGA |
| | | GAACTTCGGTTGA |
| 56 | Asparagopsis | TCAGCTAAAGTTCTCCTCCCGAAATACGAATCGAACATTG |
| | armata Azores- | CTTCCAAACCGCTTCAGTAATCTTACCTCGTCCCCAACCT |
| | Mbb2_like | CCCCTCCACCCGTTTTCCTCAAGTATGATCCCCTCAAACT |
| | coding | ACTGAAAACTTCCGCCCGCCGCAAACTCTCCCGAACCTGC |
| | sequence | TTCCCCATCCCACTGGGTCCACAGAAGAACACCCCGATCT |
| | | CACTGCAGCTCCGCGACTCCTCCGCGATCTTCAAAAATAT |
| | | TTCATCCCACTTCGGACGCCCCGCCCTAGCAGATGTGATA |
| | | AACTCCTCCCCTCCCTCCACCTCCACTTCCTTCTCCCGTG |
| | | TCACGTACCGATGCAGTCGCAGCTCCGTCCCCGTCGCAAG |
| | | CGTGTACAGATCTTCGCGAAGCCACTCGTCGTCCCCCTCG |
| | | TGCGCGACCGTCCAGATAAAGTCCACTTCCGGAACGCTGT |
| | | GTTCTGACCCCCGGCAACTCCCGTTATCGTCGAAAAGGTT |
| | | TCGCGTCCCAATGCTTCGGTATAATCGATGACAGAGAAGT |
| | | AGGAAGATAATCGGTGTGATAATAATATAGTCGACAAATG |
| | | TCAGGAAGGACGATTGGTCTCGTCCAGCCCACGTTACAAT |
| | | TCCGTATAGATTGCACAGAAATGACAGAGGAACGAACACC |
| | | AAAAAGTCAACGCATCGCCAATTCCTGTGGAAGAATCGCG |
| | | TCCCCATAAAACTCACTTCCAGCACAATTATTGTGAATAG |
| | | AGTCAATCCAACAATTAGTCCCAGAACGACGTTTGTCACG |
| | | ATAACCCACTTAGCCTCTATGTGTCCGCTGGTTGTGCTGA |
| | | AATGACCAAGATCGAAGATAGATACAACACACGTCAGCAC |
| | | TAATCGCGCAATTAGTGTGAACAGCAGCAGCAATGTTATT |
| | | CGAGTCGTGTGAAGTAAGGAAAGAAGTCTAGAGCGCTTTC |
| | | CATTCAGAAACATCAAATTATGCTTCGAGTACGCAGATCG |
| | | CCCCATCTTATCTTCTCTCCCGCCTTTTCTAGAGCTGCAC |
| | | TCTTTCTCGTATGTTCCCAGCGTGGCCATTTGACTCCCAA |
| | | TACTTGATGAAAACGAGAGATCGACATTTTCCGAACTGCA |
| | | CGTATCACTCACATCAGAAGAGCCTGTCGGAGACGGATGA |
| | | GAGATTTCATTTCCTAATTTCTGCACCGCTCCATCCGGGG |
| | | ACATGTTCAGCATATCCGCCAGATGCTGTCTTCTTTGTTG |
| | | GTTGGAATCCTCTGCGCAGCCGCTTTCACCCAACCCCAAG |
| | | CCGTACATATTGGAGATCGTTTCTCTGATCCGGTTCCCGA |
| | | GTTGTATTGTATGCTTAGATGGAATGTCAACATCGAGCGT |
| | | CTTTTTGTGATGTTCAGCGACATTCAGGTGGTGCAGATGT |
| | | TTCGAGATGGCGGCAAAAGGGGTGGCGCCAACACCTCCTG |
| | | ACACCAACACTACCCGTTGATATCTGCCGACGTGCTGAGC |
| | | GGGAGCGCCGTACGGTCCGCGGATCTGTACCGTAAGTGGT |
| | | TTGCAGTCGTCTGAATCCATGCGTGTTTGGAATTGGTGGC |
| | | GCAGTGCTTGAGTCCAATTGCCAAGAGTTTTGATGAAGAA |
| | | GGTCATGGTTTCTTCGTGGGGTGCGCTTGCGATTGTGAAT |
| | | GGATGCCACTCTGAACTGATCGAAGGGACACAGACCTCTG |
| | | CATATTGCCCCGCTCTGTAATGGAAGGGCTTTGGGACTCG |
| | | GAGTTCGAGAATGTCAGCACTCTTGAGAGAAGAATGATCG |
| | | CTGGTGAGCTGCAAGGGAACTGTGGACACTTTGAAACGAC |
| | | GAAGAAGTCTATCAATAATGTAGATGATGAGTGGGGCGGT |
| | | GATCCATTTGTAAGTTTCTGGAACGCGGTTGTACATTCCA |
| | | TGAAAGATTAGTAGAGGGAAGAAGAGTATCACTCCGAACA |
| | | TATGTGTGAGATAGAAGAATCGGAAGCGTTTCTTCCGAAA |
| | | TGTTGGGAGGGCAGTTATGAACATGACTAGAAACACAATG |
| | | AGGAGGACAGATCCTGTCGATGCGCTCATAGTGTATCCCC |
| | | AAAGGCCTGGTTCCCATTTATCGTAGGCAATAATCCACGC |
| | | GAAATGGAAAGCGACATGGAGGACGAGGGAGATGGCAATA |
| | | GCGTAAGCGACAATAATATGGAAGGCTGGGAAGCTTTTGT |
| | | CAAGAGGAATGATTAGATGCAATGGGGTCTCGCGAATATA |
| | | AGTTGAAAAGAGTCTGGCAGCGAGAAGAATGACCAATGCA |
| | | GTATTGAGATTTAGGGTGTAACCTGCTGCGCGGGCGATGG |
| | | AGATGTACCATCGCATGTTATCATCGGAAGTGTGGTGATG |
| | | GAATTCGTCATGGGCTCCCCATAGGAACATGAGGAGGAGT |
| | | GCGGTGGCATACACGACGAGAGCGAGAAGTTCGAAGGCAT |
| | | GTGTGGAGAGGTGGGACTCGAGTCGAGCGGTGAGGAGATC |
| | | TTGAGTGGACTTTGGACATGTGGATGAGGATGGAGAGGGC |
| | | AT |
| 57 | Asparagopsis | ATGCCTCCCCCAACTCGTCGTCAATCCGCTCTCGTCACTC |
| | armata Azores | GCATTGCAGCTGCAGATGAAGCCGCAAACCGTTCACATCC |
| | - Mbb3_like | TCCTCATCTAAGAAATGGCGATGAGACGCTGTACAAGACT |
| | coding | ACCTCAAATGCTCTCTCCTACCTCGCGACCTTCACAAAGG |
| | sequence | GTCTCCCTCATCATCCCGACGGTCGTCTAGTCTCCGCACA |
| | | GCATTTTCAACTCTTCGCCCGTGCTGTCCAAACAGGAACT |
| | | CCACAGCACATTGCAGATATTCCCCTCGGACCTCCATCCA |
| | | CCTTTTCCACATTCCGCTCCGGAATTGCAAAGGATGCCCC |
| | | ACTCCGCGCTTGGGAATCCATGGCCGCTGGAAATACATTC |
| | | GACCTTCAAGGGCCTGATCCCCAATCAGTCACAATACCCC |
| | | CATGTCCACGTCTCGAATCCAATGAGCTCGTCACAGAGAT |
| | | GACAGAGCTTTACTACATGGCACTCCTCAGGGATGTCCCA |
| | | TTCTCTCATTTCAAAAGTCATCACCTCGTCGCCGCTGCCG |
| | | TCGACGATATCAATCACACCCATTGGATTCAATCCCATAA |
| | | ATGTTGCATCCCAGAACTGGAACGTCACAGACACAGAGAA |
| | | CCCTTCTCATCCTCTAATGTCTTCCGTGGCACCACACCAG |
| | | GCGAGCGCAATGGTCCTTACATCTCACAATTCCTCATTGT |
| | | TGGAACCAAAGGGCTCGGAGAAATCCACGACGAGGCAGAT |
| | | GGCTACATCCAATACGGTGCCATCCGAATCGATCAGAGAG |
| | | TTCGATCAGTCGAGACAGAAATCGACTACATGACCACCTG |
| | | GGCCGCCTTCCTTGACGTCCAGAATGCCGCAGATTTGCGC |
| | | AGACATTCTACATATGCCAAATCAAGCTCCTATCGCTTCC |
| | | TGTACACACCACGCGACCTCGCTACCTACGTTCATCATGA |
| | | CTCGCTCTACCAAGCCTATCTCAACGCATGCATCATCCTC |
| | | CTCGACATGAAAGTCCCATTCGACTCCGGTCTCCCCTTCA |
| | | CCACGGAAGACCACATCGACAAACAACAGGGCTTTGTAAA |
| | | CTTTGGAGGCCCCCATATCCTCACACTCGTCACAGAGGTT |
| | | GCTACCCGAGCCCTCAAGGCAGTCCGTTTCCAAAAATACG |
| | | CCGTCCACAGACGTCTTCGCCCAGAGGCTGTCGGCGGCCT |
| | | CTTAGAACAATACAGACGATACGGTGGCGGATATTCAGAG |
| | | CTAGCAAATACTATTCGTCCCATACGCCATCTCGCCCGTT |
| | | CTCTAAGTTCAAACCTCATGTCGCTCGTCGCAGCAGCGAA |
| | | CGGCCATCAGAACGCAACAATGAGCGACAACGGATTTCCA |
| | | CGCTCCCATGATACAGGCTCCGGTGAGACTCATTTGCTGC |
| | | CAATGGCCTATCCGGAGGGAAGTCCGATGCACCCTAGCTA |
| | | TGGTGCCGGGCATGCCACAGTGGCCGGGGCGTGCGTAACG |
| | | ATATTGAAAGGCTTTTTCGATCACACCTACAAGCTCCCGT |
| | | TCGCCTATGTCTCATCCTGCAATGGGAAGATGCTGAAGAA |
| | | GGCAAAGGTCGCGAAGAAACTTTCGGTGGAAGGAGAGCTG |
| | | AATAAACTGGCGTCTAATATCAGTATTGGACGTAGCTGGG |
| | | CTGGCGTACACTACTACTCCGACTATGTGGAGAGTATTCG |
| | | ATTGGGAGAGGAAGTGGCGATTGGAATCCTGAAAGAGCAA |
| | | AAGTTAACTTATTCGGAAAAATTCTCAATGACAATTCCCA |
| | | AATTTGATGGCACAACCATCAAAATATAG |
| 58 | Asparagopsis | ATGTCCGAAGAACGCCATGCATCCGCCTTGAGAGTCCGTA |
| | armata Azores | GTGAAGCCGCAGATCTCGCTGCTTCACGCCCCCTCCCCAC |
| | - Mbb4-2_like | CCACCTTGCAAACGGCGATGAGCATCGCTACCACGACACC |
| | coding | CACCTCGCCTCCTACACGAAAGGTCTTCCACACGAAAAGG |
| | sequence | ATACCGGCCTCTCCTCCTCTCCAGATCACTTCGCAGCCTT |
| | | TGTACGCGCCATCGAAACAGGCGCTCAAGAAGATATTACA |
| | | AGTCTCCCCCTAGGTATCGATGAAGACCCCCGTTTCATAT |
| | | CCGCCATCGCTTCAGAGGGCTCCGACCATCCCATAGCCGA |
| | | CACTAGAGCGTGGGAATCCATGGCAGCTGGTAATACATAC |
| | | GATCTGGAGGGACCGGACGCTCAGGCAGTCACCATGCCGC |
| | | CTGCCCCAAAAATCGATAGTGAAGAACTCTTGACGGAGAT |
| | | CACCGAGTGCTACTGGATGGCCCTTCTTCGCGACGTCCCG |
| | | TTCACAGAGTTCGAAAGGGATGGCACTACCGCTGCCGCAG |
| | | CCGCTTCGATCAGCCGAACGCGCTGGGTGCAGTTCCAAGA |
| | | GCAGCCCGGGCAACGCCCCTGCGATCTGTCTGATGCAGAA |
| | | ATTGCACGTTTGCGAGGTCCATTCAGGAAAACAAACATCT |
| | | TCCGCGGCGTGACCCCTGGTGAACAAATCGGTCCCTATCT |
| | | CTCTCAGTTCCTTCTCGTTGGCACAAAGGGTATCGCGAAC |
| | | ATTCAGGAAATTTCAGATGGATTCATTCAATACGGAGGTA |
| | | TGCGTATGGACCAACGTGTTCGTGTGGCAACCCCAAATAG |
| | | GGACTACATGACCACCTGGGCATCCTGGCTCGACGTGCAG |
| | | AACGCAGGTGATGTACGAGGACGTGAAACGTACGACAACG |
| | | ACACTCCATTCCGTTTCATTACTACACCGCGCGATCTTGC |
| | | CACCTACGTCCATTTCGATGCACTTTACCAGGCATACCTG |
| | | AACGCATGTCTCATTCTTCTCGACACCAAGGCCCCTTTCG |
| | | ATCCAGAAATCCCTTTTGTGGAAGATGATGACATCGATAA |
| | | ACAACAGGGATTCGCTACCTTTGGAGGTCCCCATATCCTC |
| | | TCGCTTGTCACAGAGGTCGCAACTAGGGCATTGAAGGCTG |
| | | TGCGCTTCCAGAAGTTTGGTACCCACCGCCGTCTACGCCC |
| | | CGAGGCTGTCGGCGGTCTTATCGAGCGATACCGAAAGACG |
| | | GAGGGAGCAGAGCCCTTTGAGCCCATCCAGAAAATCGCCA |
| | | CTGAGATCGACAATGACCTTATGACACGAATCTCGCAGCA |
| | | CAACGCCGCGCAGAATGAACTTTCCGACAACGGTGGTGCC |
| | | CGAAGCGGCGATTATAATCCTGAGGGCTGCGAAAGCGCAG |
| | | AAACTTATCTTCTCCCGATGGCATTCCCGGAGGGTAGCCC |
| | | AATGCATCCCAGCTATGGTGCAGGGCATGCTGCTGTTGCG |
| | | GGTGCATGTGTGACGATCCTAAAGGCGTTCTTCGACCACA |
| | | CTCACGAGCTTGAATTCTGTTATGTGCCCAGCACAGACGG |
| | | CCAGAATCTTGAGGAGGTGGAAATCAATGAACCGCTCACA |
| | | GTGGAGCATGAGCTCAACAAGCTTTGTGCCAACATCAGTA |
| | | TTGGTAGGAATTGGGCTGGTGTGCACTATTACTCGGATTA |
| | | CATAGAGAGCGCAAGGTTGGGTGAAGAGGTTGCAATTGGT |
| | | ATTCTAGAAGAACAGAGTCTGACGTACGGTGAGGAGTTCT |
| | | CCATGACTATTCCCAAGTTTGATGGTACGACTATCAAGAT |
| | | TTCAAACTCAAAGTAA |
| 59 | Asparagopsis | TCAGTTGGAGATCGTAATGCACGTTCCATCAAACTTGGGG |
| | armata Azores | AGGGTCATGGAGAACTTCTCACCATACGTCAAGCTCTGTT |
| | - Mbb4_like | CTTGCAAAATACCGATGGCAACCTCTTCACCCAACTTTGC |
| | coding | ACTCTCTTCGTAATCCGAGTAGTAGTGCACACCGGCCCAG |
| | sequence | TTTCTTCCAATACTGATGTTGGCACACAACTTGTTCAGCT |
| | | CGTGTTCAACTGTGAGCGGTTCACTGATGTTAACCTTCTC |
| | | TAGCTTCCTGCCGTCTGTGCTGGGTTCAAAGCAGAACTTT |
| | | AACTTGTATTTGTGATCAAAGAAGGCCTTGAGGATCGTGA |
| | | CACAGGCGCCAGCGACAGCGGCGTGTCCAGCACCGTAGCT |
| | | GGGATGCATTGGGCTTCCCTCCGGGAACGCCATTGGAAGA |
| | | AGGTAGGTTTCTGCGCTTGCGCCACCCTCGGGGTTGAAAT |
| | | CATTTCTTCGTGCTTTACCGTTGTCGGAAAACTCATTCTG |
| | | CTTGGCGTTGTGACGCGAGATGCGCGTCATAAGCTCATTG |
| | | TCGATCTCGGTAGCGAGCTTCTTAGCGGCTGCGAATGGCG |
| | | CTGCGCCGTTCGTCTTGCGGTAGCGCTCGATGAGTCCGCC |
| | | GACAGCTTCGGGGCGTAGGCGACGATGCGTACCGAACTTC |
| | | TGGAAGCGCACAGCCTTAAGTGCTCTGGTTGCAACCTCTG |
| | | TGACAAGAGAAAGGATGTGAGGCCCGCCAAAGGTGGCAAA |
| | | TCCCTGTTGTTTATCGATGTCGTCGTCCTGCACAAAAGGA |
| | | AGGCCATGATCGAAGGGAGCCTTTATGTCGAGAAGAATGA |
| | | GACAGGCATTGAGGTATGCTTGATAGAGGGCGTCGAAATG |
| | | GACGTAGGTTGCGAGATCGCGCGGGGTGGTAATGAAACGG |
| | | TAATCAGTATCTTTGTCGTATGTTTCACGTGAGCGGACGT |
| | | CCCCTGCATTCTGTACGTCGAGCCAGGAGGCCCAAGTAGT |
| | | CATATAGTCGCGGTTGGGAGTCGCAAAGCGAACGCGCTGG |
| | | TCCATTCGCATACCTCCGTATTGAATGAAACCTTCTTCTG |
| | | GCTGGTGCATTTTCCCGATACCCTGTGTACCGACAAGCAA |
| | | GAACTGGGAGAGGTAGGGGCCTACCTGTTCACCAGGCATG |
| | | TCGCCGCGGAAGACGTTTGATTTGTTGAAGGGGCCGCGCA |
| | | GACGTTGGATTTCAGCATTTGTGAGATCGGATGGGCGCTG |
| | | GCCGGGCTGCTCCTGAAACTGTACCCAGCGTGTACGACTA |
| | | ATCGATGCGGCTGCGGCAGCAACTGTTCCGTTTCGATGAA |
| | | ATTCTGAGAAGGGCACGTCGCGAAGAAGGGCCATCCAATA |
| | | GCATTCTGTGATCTCTGTCAAGAGCTCTTCGCTGTCGATT |
| | | TTAGGTGCAGGAGGCATGGTGACTGCCTGTGCATCAGGCC |
| | | CTTCGAGATCGAAGGTATTGCCTGCGGCCATGGATTCCCA |
| | | GGCGCGAGTGTCAGCGATGGGGAGGTCGAGTGCTGGGGAT |
| | | GCGATGCCGGAGATGAAACGAGGTGTTTCATCAATACCTA |
| | | GGCGGAGGCTCCTGATGTCTTCCTGTGCGCCTGTGTTGAT |
| | | AGCACGCACAAAGGCAGTGAAGTCATCTGTAGATGAGAGA |
| | | AGGCCTGTCTTTTTGTCGTGTGGTAAACCCTTTGTGTAGG |
| | | ATGCGATGTATGAATCTGAGAAACGGGTCTCATCGCCATT |
| | | GGGGGTGTGGGTAGGGAGTTTGCGGGAGGCTGCTAGTTTT |
| | | GCGGCATCTGTGCGTACTTGTAGTGCGGCTGCTTGACGTG |
| | | TGGTAGACAT |
| 60 | Asparagopsis | TTAAATGCGAATTGTGCTTCCATCAAACTTGTTGAGGGTC |
| | armata Ireland | ATCGAGAAATTCTCACTGAAGGTCAACTTCTGTTCCTGAA |
| | -Mbb1_like | GAATGCCAATAGCAATTTCCTCACCAATGCGGATACTTTC |
| | coding | AATGTAGTCAGTGAAGTAATGCACACCAGCCCAGTTACGT |
| | sequence | CCAATGGAAATATTAGAGCAAATCTTGTTCAGCTCTCCGT |
| | | CAACTGTGAGCGGCTCAGACACTTCAACCGGCTCGAGGCC |
| | | GCAGCCATTTTCGTTAGTGACGTATGCAAACGGCAGGGGG |
| | | AATCCACCATCAAAGAATGCCTTGAGAACAGTTACACAGG |
| | | CTCCTGCAACAGTGGCATGTCCAGCACCATAAGCCGGATG |
| | | CATCGGGCTTCCCTCTGGGAATGCCATTGGCATGAGAAGG |
| | | TTGCCTTCGCTCAGGCCGCCATCAGGATTGAAATCTGCAC |
| | | TACGCGGAGCACCGTTGTCGCTCAACTTATTTTGCTTCTT |
| | | GTTATGGTAATACACTCGATCCAACAGGTCAGAATCCAGG |
| | | GAATCTACCAATTTAGCAGCTCCGGAAAATTCGGGTGCTT |
| | | TGGTTTTGTAGTATCGGTCGACACGACCGCCGACAGTCTC |
| | | CGGGCGTAGACGGCGATGGACGTTGAACTTCTGGAAGCGC |
| | | ACTGCCTTGAGAGCGCGAGTGGCCACCTCAGTAACGAGAG |
| | | AGAGAATATGCGGTCCGCCAAACGTGGCAAATGCCTGCTG |
| | | CTTATCTATATCATTGTCGAGCTGGAACGGGACTCCGGAA |
| | | TCGAACGGTGCACCAATGTCGAGCAGAATGATGCAGGCGT |
| | | TAAGATAGGCCTGGTACAGAGCGTCGAAATGGACGTATGT |
| | | CGCCAGATCACGCGGTGTGTGAATGAAGCGGAAACCAGGG |
| | | TCTGACTCGTATCGCTCACGCCCGGTGACATCCGCAGCGT |
| | | TCTGCACGTCAAGCCAGGATCCAAAGGTGGTCATGTAGTC |
| | | AACCTTGGGGGATGCCACGCGTACCCGCTGGTCCATGCGC |
| | | ATACCACCGTACTGGATGAAACCATCGCTGACCTCGTTTC |
| | | CGTTTGCGATTCCTGTGGTACCGACCAAGAGGAACTGGGA |
| | | CAAATATGGGCCTACATCATCTCCAGGAGTGATGCCCCTG |
| | | AAGACATTGGAGGCATCGATGGGGCCTCTGAGACGGGAGC |
| | | GTTCGGCAGATGTGAGATGGGTGGGAGGGTTTTCCTTGAA |
| | | TTTAATCCATTGAGTGTCGTTGATACTCTTGGCAGCGGCT |
| | | GCGACATCTTCGTCGCAATGGAATGTTGGGAAGGGTACAT |
| | | CCCTGAGCAGGGACATCCAATAACATTCGGTGACTTCAGT |
| | | GACCAACTCCTCGCTGTCTAGCTCTGGTGCGGGCGGCATA |
| | | GTGACGGCCTGTGCATCAGGCCCTTCGAGATCGAATGTGC |
| | | GTCCAGCACCCATACTTTCCCAGGCACGAACTCCCGACTG |
| | | CTCTGGGTTTGTGGCGATGCCGGATGTGAATCTGGGGGTG |
| | | ACATTCTGGGGGCCGAGTGGGAGAGCTGCGAAATCGCGGG |
| | | CATCTCCGCTGTCAATAGCGCGTACAAATTGGTCATAATC |
| | | GAGGGCAGATTGAACCAGACCTGTTTCGTTGATATGGGGG |
| | | AGACCTTTCGTGAAGTTAGCCAAGAGAGATCGTGTACCAT |
| | | CTGGGTTTCTGAAGCGAGCCTCTGAATTGTTGGAGATGTG |
| | | AGTTGGAGAACCACGGGAGCGAGCAAGCTCGGCGGCAGAG |
| | | AGGCGAATCTTGAAGGAGGCCTCTGCGCGATTGGGATTCT |
| | | GTGTGTCGGGGGTTGCCAT |
| 61 | Asparagopsis | ATGAACACAGCATTTCCCTTCAGAAGAAAATGGTCCCCAA |
| | armata Ireland | GTCTTTGGAGTGCTCGCCTCGAGTCCTATCTCTCCACTCA |
| | - Mbb2-2_like | CGCCTTCGAGCTTCTCACAGTTATCACATACATAACTGCA |
| | coding | CTCTCTCTCATGTTCGTATGGGGAGCTCACGATGAGTTCC |
| | sequence | ACAATCAATCTCCCGACACAAATCTCCGCTGGTACATTTC |
| | | TATTGCTCGCGGAGCAGGTTACACACTCAACCTCAACACA |
| | | GCACTTGTCATTCTACTTGCATCTCGCCTCTTCTTCACAT |
| | | TCATCCGTGATACTCCCCTGCACAAGATTCTTCCTGTTGA |
| | | CAAAAGCTTCCCCGCCTTCCATATCCTTGTTGGATATACG |
| | | ATAGCCGCATCTCTCTTCATTCATGTCCCGTTCCATTTCA |
| | | CATGGATCATTGCTTACGATAAATGGAGTGCAGGACTTTG |
| | | GGATTTCACCATGAGCGCTGTCACAGGCGTCGTATTGTTA |
| | | CTTATCTTCATAATCATGTTCATGTTCGCTCTACCATTCT |
| | | ACAGAAAAAAATATTTCCGGCTCTTCTACATTTCCCACAT |
| | | GGTTGGAGCACTGCTGTTCTTTCCTCTTCTCATCTTCCAT |
| | | GGTATGTACCGTCGTGTTCCGGAGACTTACAAGTGGGTTG |
| | | CTCTACCTCTCGTGATCTACATTCTGGACAGAATCGCCCG |
| | | TCGCTTCAAAATCTCCACCGTTCAGCTGAAGCTCACTTCC |
| | | GAGCATTCAGCTCTCAAAGGAAAAGACATTTTGGAACTTC |
| | | GTATTCCGAAACCCTTCACATATCGCGCTGGTCAGTATGC |
| | | AGAGATCCGTGTACCATCCTTGAGTAGGGAGTGGCATCCC |
| | | TTCACAATTGCTAGTGCACCCCATGAAAAATGCATGTGCT |
| | | TTTTCATCAAATCACTCGGGAATTGGACCAAGGAGCTTCG |
| | | AGGGGCATTTCAAAACCGTATGGAAGGAGAAGATCCGAAC |
| | | CCATTGCTAGTTCAAGTCCGTGGTCCATTTGGCGCTCCCG |
| | | CTCAACATGTCTCTTCGTATGAAAGAGTGGTCCTCATATC |
| | | TGGCGGAGTTGGTTCAACGCCCTTCGCAGCCATCTCCAAA |
| | | AGTTTGCATTTCTTGAACAAGAACAAAACCTGCCCTTCTA |
| | | CTGATTTTTCGAAAACCAATTTGTCTGAAATGAAGCATAT |
| | | TGAGCAGCGTATCTCGGAAACTATTTCTGCTGTATATGAT |
| | | ATCGATTTGGCAAACAGCTCCGATGAACCGGAATCTGAAG |
| | | AACAAAGGAAGCAGTATATTACTGACATGATGAACTTGAC |
| | | GGCCCAAAATTCCGAAGAACTCGATGTTTCCACTTCAAGA |
| | | ACAAGTGAACAATGTGTATCATCGAGGAGCTGCCCAAATA |
| | | TTGGAGATGGTCTCCGGATATTGGATGTCGAAAATCAATT |
| | | AGAAGAATCTTCAGTGAGCAGCATGTCTACATGCGACGAT |
| | | GAAGAAGATTATTCATCAGATTTCAGTGAAGAAATGAGAC |
| | | TTGAAAAGGAACTTGCCGCCAGGAAACGTAAGATGGCTCG |
| | | CCAAAAAATTGCCCGTCTCTATGAGAGACGTTCAATATTA |
| | | TTCTCTTTTCTTCATACCACTCGGGTAACTCTGGCACTTC |
| | | AACTCTCTCTAATTGCACGCATTGTTCTTGTGTGCATTGT |
| | | ATCAATCTTCAACATTGGTGAATTCAACTTTGGGGACAGC |
| | | CGCATAGGTGCCAAATGGGTTATAGGTGCCGATGCAGTGT |
| | | TGAGTGTTTTGCATGCAGTCACTCTAACTTTGACCATACT |
| | | CTTGGAAATTAGCTTCATGGGACCCCGCTTCTTCCGTTCT |
| | | TCTTGGCAGTTTATGGACTTTTTCGTTTTCGTCCCGCTCA |
| | | GTATTCTGTGCAGCGTCAGTGGTGTACTATCTTGGACTGG |
| | | GACGGAAATGTCAGGAACATTAGTTTTTCTTCACTTCATT |
| | | GTATTGCTGCCAATTATGCTTATTTTCATGAGTGCTCGTC |
| | | TGTATCGGAGTATTGGGTCCCTGAATCTATTTCATGATCG |
| | | GAAGTGTAACCCAAACTGCGCATGCGGAAAGAAAATCCCA |
| | | GACGTTGACTTTATCTGGACAACACGTCACACCGTAGATG |
| | | ACCAATGGTTACGAGAAGAACTCTTTCCTCTAGAGGACGG |
| | | CACAAAACTCCGTCTTCACCGATATATTACAGGCAAGGAA |
| | | AGTGAAGACTTTGAAAGTGCTGAGACCTTTATTACTACAA |
| | | GCAACATTGGAAGACCGCCCTGGGACGAAATATTCGCCAA |
| | | AATTGCCCAAAAGTCACCAAGTCACGCAGAAATCGGAGTG |
| | | TTTTTCTGCGGACCACACTCAATGGGAGCAGCTGTGAGGA |
| | | AAAGCATGCGAAAAACTGAAATACGGAGTAACTTGCGTGG |
| | | GGCATATCTGGAGACAACTAATGTTGTCGATCTTGTTGCC |
| | | GATCTTCTTCTGAGAAATACTGACCATGCAGCAGAGCTTC |
| | | GGAGGCATGGGTGCAATATTCGATTTGTATTTCGTGAAGA |
| | | GAACTTCGGTTGA |
| 62 | Asparagopsis | CTAACTAAAGTTTTCCTCTCGAAAGACAAACCGAATATTG |
| | armata Ireland | CATCCGTGTTCTCTCAATAACTTAACCTCCTTTTCATCCC |
| | - Mbb2-3_like | TCAATGACAAGTCTTCCACTAACAATTCACTGTCAGTCTT |
| | coding | TCGCAAATACGATCCTCGGAGGTTGCTCATCACTTCAACT |
| | sequence | TTCCTCATACTCTTCTGAACTGCTGCACCCATAGGATGTG |
| | | GCCCGCAGAAGAACACTCCAACTTCATTGTGGCTAGGAGT |
| | | TTGTTCGGCAATTTGTGCGAAAATCTCATCCCACTGAGGC |
| | | CGTCCAGTATTCGCAGTAGTGATGAAATGTTCAGGTTCTT |
| | | CCAGGTCCAAGTCCTTTTCACGTGTTACATATCGGTGCAA |
| | | TCGTAGATCAGTTCCTGTGGCCAGCGGGTACAGCTCACTC |
| | | CGCAGCCACTCATCCTCACTATCACGAGGAGTAGTCCATA |
| | | CAAAGTCAACGTCTGGAATAGTTTTGTTACAGGTGCAACT |
| | | CTGACGACATTGTGTGTCATCCAAAAGGTTGCGTGCGCCA |
| | | ATACTTCGGTACAGGCGATGGCAAAGCAAGAGGAACATGA |
| | | TTGGAACGACAATAATCAAGTCCAGAAAAATAAGAAATTT |
| | | CGCTTGTTGACGTCCACTCCAACTCATGAAATTGCTGATA |
| | | TTACAGAGAAAGGTAATAGGGAGAAAGATAAAGAAGTCGA |
| | | CGCAACGCCAACCTCTGTAGAAGAAGCGCAGTCGCATGAA |
| | | ACTGATTTCCAGTAGAACGGTAGTCAACAGCGCAATCCCG |
| | | AGTACGAGTCCCAATAAGGAATTAGTGGCAATGACCCATT |
| | | CGGCATCAATATGACTGTCACTGATTCCAATATGCCCAAG |
| | | ATTGAAAATAGATACAACGCACAAAATAACAATTCTTGCA |
| | | ACCAGAGTGAGAAGAAGAGCAAATGTGAATCTTGTTGTAT |
| | | GCAGAAAAGCGAGCAGTCTTGAGCGGTACTCATAGAAATT |
| | | GAAAATCCTCCGCTGCGCAACATTGAAGACGTTGGCTGAA |
| | | GGTCGATTATAATATCGAATAGATTCAGAACATGCATCAA |
| | | GATTATAACTGCTTTGCTTCTCTTTCTGAGACAGCGATAT |
| | | TTCCTCATCCGCTCTGATCTGCTTGGTTATTTCTTCAACC |
| | | GAACTGCAATCATCAGACGTCTTTGATAGATTGCGTTTAA |
| | | AAGAAACGTTATGGACGTTTCCCTTTGGAGACATATTGAG |
| | | CATATCGGCGACTTGTTGTATTCGCTGTTGGTTCGATGCA |
| | | GCATCACTGCGGCTATCATCCAGACATACATCGAAGAGAT |
| | | TGGAAATGGCATCACGGATGTGCCAATGAATTTGAGACTG |
| | | TCTTTTCGAAGATCCATGTGATTCCAAAGCTTTCGAATGG |
| | | TTTTCAGACTTGTTGAGATGGTGTAGGTTTTTGCAGATTG |
| | | CTGCGAAAGGTGTTGAACCGACCCCTCCGGAGATAAGAAC |
| | | GACGCGACGATATCCTCTCACGTGCTGGGCTGGAGCACCA |
| | | TATGGTCCGCGGATCTGGACTTTGAGCGGTTCATATAGGT |
| | | CATTCTCAATTCGAGCTTCGAACGCATCTCGAAGATGTCT |
| | | AGTCCAATCACCAATCGCCTTGATGTAGAAGCACATAGAT |
| | | TTCTCATGAGGTGCACTTGCAATTGTAAAAGGATGCCACT |
| | | CATTATTTATTGAAGGCACGCATACCTCTGCATATTGACC |
| | | AGCACGGTATTCGAACGTTTTTGGGACTCGCAGCTCCAGA |
| | | ATATCACTTCCCTTCAGGGAAGAGTGCTCACCAGTGAGTT |
| | | CGAGCTTCGCTGTTGAAATCTTGAATCGTCTCAGAACCCT |
| | | ATCAATTGTGTAGATAGCGAGAGGTCCGGCAATCCATTTG |
| | | TACGTTTCGGGGACTCTGTTGTACATGCCGTGAAAGATAA |
| | | GCAGAGTGAAGAATAGTAATGCACCAACCATATGGATGAG |
| | | GTAGAAGGTACGGAATTGCTTCTTTCGAAATCTAGGAAGA |
| | | GCAGAGACGAACATGACGACAAATACTGTGAGAAGAGCAA |
| | | AACCTGTAGTGGCACTCATAGTAAACCCCCACATGCCTAA |
| | | TTCCCATTTATCATAGGCAATGATCCACACGAGATGGAAG |
| | | GAAACATGTATGAAGAGTGCGGCTGCAATGGTGTAGGCCA |
| | | CGATGATGTGGAAAGCTGGGAAACTTTTGTCGATTGGGAG |
| | | GATGTGTTGCAGAGGAGTTTCACGGATGTAGGTCGCGAAA |
| | | AGTCGGGCGGCGAGAAGGATGACGAGAGCGGTATTGAGAT |
| | | TGAGTGTGTATCCAGCACCACGGGCAATGCAGATGTACCA |
| | | TCGTAGATTTTTTTGAGAAGTATGGTGATGAAATTCGTCA |
| | | TGGGCACCCCAGACGAACATGAGAATGTTTGCGAGTGAGT |
| | | AGATGAAAAGTGCGAGAAGCGAGAATGCATTTGTTGATAG |
| | | ATAGGATTCTAGGCGTGCGCTGAGGAGAGTGAGAGAGGTG |
| | | GGAGAGCATCGTTGCTTGCCGGAGCGGGGAGCCAT |
| 63 | Asparagopsis | ATGCCCTCTCCATCCTCATCCACATGTCCAAAGTCCACTC |
| | armata Ireland- | AAGATCTCCTCACCGCTCGACTCGAGTCCCACCTCTCCAC |
| | Mbb2_like | ACATGCCTTCGAACTTCTCGCTCTCGTCGTGTATGCCACC |
| | coding | GCACTCCTCCTCATGTTCCTATGGGGAGCCCATGACTACA |
| | sequence | TCTCCATCGCCCGCGCAGCAGGTTACACCCTAAATGAATT |
| | | CCATCACCACACTTCCGATGATAACATGCGATGGCTCAAT |
| | | ACTGCATTGGTCATTCTTCTCGCTGCCAGACTCTTTTCAA |
| | | CTTATATTCGCGAGACCCCATTGCATCTAATCATTCCTCT |
| | | TGACAAAAGCTTCCCAGCCTTCCATATTATTGTCGCTTAC |
| | | GCTATTGCCATCTCCCTCGTCCTCCATGTCGCTTTCCATT |
| | | TCGCGTGGATTATTGCCTACGATAAATGGGAACCAGGCCT |
| | | TTGGGGATACACTATGAGCGCATCGACAGGATCTGTCCTC |
| | | CTCATTGTGTTTCTAGTCATGTTCATAACTGCCCTCCCAA |
| | | CATTTCGGAAGAAACGCTTCCGATTCTTCTATCTCACACA |
| | | TATGTTCGGAGTGATACTCTTCTTCCCTCTACTAATCTTT |
| | | CATGGAATGTACAACCGCGTTCCAGAAACTTACAAATGGA |
| | | TCACCGCCCCACTCATCATCTACATTATTGATAGACTTCT |
| | | TCGTCGTTTCAAAGTGTCCACAGTTCCCTTGCAGCTCACC |
| | | AGCGATCATTCTTCTCTCAAGAGTGCTGACATTCTCGAAC |
| | | TCCGAGTCCCAAAGCCCTTCCATTACAGAGCGGGGCAATA |
| | | TGCAGAGGTCTGTGTCCCTTCGATCAGTTCAGAGTGGCAT |
| | | CCATTCACAATCGCAAGCGCACCCCACGAAGAAACCATGA |
| | | CCTTCTTCATCAAAACTCTTGGCAATTGGACTCAAGCACT |
| | | GCGCCACCAATTCCAAACACGCATGGATTCAGACGACTGC |
| | | AAACCACTTACGGTACAGATCCGCGGACCGTACGGCGCTC |
| | | CCGCTCAGCACGTCGGCAGATATCAACGGGTAGTGTTGGT |
| | | GTCAGGAGGTGTTGGCGCCACCCCTTTTGCCGCCATCTCG |
| | | AAACATCTGCACCACCTGAATGTCGCTGAACATCACAAAA |
| | | AGACGCTCGATGTTGACATTCCATCTAAGCATACAATACA |
| | | ACTCGGGAACCGGATCAGAGAAACGATCTCCAATATGTAC |
| | | GGCTTGGGGTTGGGTGAAAGCGGCTGCGCAGAGGATTCCA |
| | | ACCAACAAAGAAGACAGCATCTGGCGGATATGCTGAACAT |
| | | GTCCCCGGATGGAGCGGTGCAGAAATTAGGAAATGAAATC |
| | | TCTCATCCGTCTCCGACAGGCTCTTCTGATGTGAGTGATA |
| | | CGTGCAGTTCGGAAAATGTCGATCTCTCGTTTTCATCAAG |
| | | TATTGGGAGTCAAATGGCCACGCTGGGAACATACGAGAAA |
| | | GAGTGCAGCTCTAGAAAAGGCGGGAGAGAAGATATGCTGG |
| | | GGCGATCTGCGTACTCGAAGAATAATTTGATGTTTCTTAA |
| | | GGGAAAGCGCTCTAGACTTCTTTCCTTACTTCACACGACT |
| | | CGAATAACATTGCTGCTGCTGTTCACACTAATTGCGCGAT |
| | | TAGTGCTGACGTGTGTTGTATCTATCTTCGATCTTGGTCA |
| | | TTTCAGCACAACCAGCGGACACATAGAGGCTAAGTGGGTT |
| | | ATCGTGACAAACGTCGTTCTGGGACTAATTGTTGGATTGA |
| | | CTCTATTCACAATAATTGTGCTGGAAGTGAGTTTTATGGG |
| | | GACGCGATTCTTCCACAGGAATTGGCGATGCGTTGACTTT |
| | | TTGGTGTTCGTTCCTCTGTCATTTCTGTGCAATCTATACG |
| | | GAATTGTAACGTGGGCTGGACGAGACCAATCGTCCTTCCT |
| | | GACATTTGTCGACTATATTATTATCACACCGATTATCTTC |
| | | CTACTTCTCTGTCATCGATTATACCGAAGCATTGGGACGC |
| | | GAAACCTTTTCGACGATAACGGGAGTTGCCGGGGGTCAGA |
| | | ACACAGCGTTCCGGAAGTGGACTTTATCTGGACGGTCGCG |
| | | CACGAGGGGGACGACGAGTGGCTTCGCGAAGATCTGTACA |
| | | CGCTTGCGACGGGGACGGAGCTGCGACTGCATCGGTACGT |
| | | GACACGGGAGAAGGAAGTGGAGGTGGAGGGAGGGGAGGAG |
| | | TTTATCACATCTGCTAGGGCGGGGCGTCCGAAGTGGGATG |
| | | AAATATTTTTGAAGATCGCGGAGGAGTCGCGGAGCTGCAG |
| | | TGAGATCGGGGTGTTCTTCTGTGGACCCAGTGGGATGGGG |
| | | AAGCAGGTTCGGGAGAGTTTGCGGCGGGCGGAAGTTTTCA |
| | | GTAGTTTGAGGGGATCATACTTGAGGAAAACGGGTGGAGG |
| | | GGAGGTTGGGGACGAGGTAAGATTACTGAAGCGGTTTGGA |
| | | AGCAATGTTCGATTCGTATTTCGGGAGGAGAACTTTAGCT |
| | | GA |
| 64 | Asparagopsis | ATGCCTCCCCCAACTCGTCGTCAATCCGCTCTCGTCACTC |
| | armata Ireland | GCATTGCAGCTGCAGATGAAGCCGCAAACCGTTCACATCC |
| | Mbb3_like | TCCTCATCTAAGAAATGGCGATGAGACGCTGTACAAGACT |
| | coding | ACCTCAAATGCTCTCTCCTACCTCGCGACCTTCACAAAGG |
| | sequence | GTCTCCCTCATCATCCCGACGGTCGTCTAGTCTCCGCACA |
| | | GCATTTTCAACTCTTCGCCCGTGCTGTCCAAACAGGAACT |
| | | CCACAGCACATTGCAGATATTCCCCTCGGACCTCCATCCA |
| | | CCTTTTCCACATTCCGCTCCGGAATTGCAAAGGATGCCCC |
| | | ACTCCGCGCTTGGGAATCCATGGCCGCTGGAAATACATTC |
| | | GACCTTCAAGGGCCTGATCCCCAATCAGTCACAATACCCC |
| | | CATGTCCACGTCTCGAATCCAATGAGCTCGTCACAGAGAT |
| | | GACAGAGCTTTACTACATGGCACTCCTCAGGGATGTCCCA |
| | | TTCTCTCATTTCAAAAGTCATCACCTCGTCGCCGCTGCCG |
| | | TCGACGATATCAATCACACCCATTGGATTCAATCCCATAA |
| | | ATGTTGCATCCCAGAACTGGAACGTCACAGACACAGAGAA |
| | | CCCTTCTCATCCTCTAATGTCTTCCGTGGCACCACACCAG |
| | | GCGAGCGCAATGGTCCTTACATCTCACAATTCCTCATTGT |
| | | TGGAACCAAAGGGCTCGGAGAAATCCACGACGAGGCAGAT |
| | | GGCTACATCCAATACGGTGCCATCCGAATCGATCAGAGAG |
| | | TTCGATCAGTCGAGACAGAAATCGACTACATGACCACCTG |
| | | GGCCGCCTTCCTTGACGTCCAGAATGCCGCAGATTTGCGC |
| | | AGACATTCTACATATGCCAAATCAAGCTCCTATCGCTTCC |
| | | TGTACACACCACGCGACCTCGCTACCTACGTTCATCATGA |
| | | CTCGCTCTACCAAGCCTATCTCAACGCATGCATCATCCTC |
| | | CTCGACATGAAAGTCCCATTCGACTCCGGTCTCCCCTTCA |
| | | CCACGGAAGACCACATCGACAAACAACAGGGCTTTGTAAA |
| | | CTTTGGAGGCCCCCATATCCTCACACTCGTCACAGAGGTT |
| | | GCTACCCGAGCCCTCAAGGCAGTCCGTTTCCAAAAATACG |
| | | CCGTCCACAGACGTCTTCGCCCAGAGGCTGTCGGCGGCCT |
| | | CTTAGAACAATACAGACGATACGGTGGCGGATATTCAGAG |
| | | CTAGCAAATACTATTCGTCCCATACGCCATCTCGCCCGTT |
| | | CTCTAAGTTCAAACCTCATGTCGCTCGTCGCAGCAGCGAA |
| | | CGGCCATCAGAACGCAACAATGAGCGACAACGGATTTCCA |
| | | CGCTCCCATGATACAGGCTCCGGTGAGACTCATTTGCTGC |
| | | CAATGGCCTATCCGGAGGGAAGTCCGATGCACCCTAGCTA |
| | | TGGTGCCGGGCATGCCACAGTGGCCGGGGCGTGCGTAACG |
| | | ATATTGAAAGGCTTTTTCGATCACACCTACAAGCTCCCGT |
| | | TCGCCTATGTCTCATCCTGCAATGGGAAGATGCTGAAGAA |
| | | GGCAAAGGTCGCGAAGAAACTTTCGGTGGAAGGAGAGCTG |
| | | AATAAACTGGCGTCTAATATCAGTATTGGACGTAGCTGGG |
| | | CTGGCGTACACTACTACTCCGACTATGTGGAGAGTATTCG |
| | | ATTGGGAGAGGAAGTGGCGATTGGAATCCTGAAAGAGCAA |
| | | AAGTTAACTTATTCGGAAAAATTCTCAATGACAATTCCCA |
| | | AATTTGATGGCACAACCATCAAAATATAG |
| 65 | Asparagopsis | TTACTTTGAGTTTGAAATCTTGATAGTCGTACCATCAAAC |
| | armata Ireland | TTGGGAATAGTCATGGAGAACTCCTCACCGTACGTCAGAC |
| | - Mbb4-2_like | TCTGTTCTTCTAGAATACCAATTGCAACCTCTTCACCCAA |
| | coding | CCTTGCGCTCTCTATGTAATCCGAGTAATAGTGCACACCA |
| | sequence | GCCCAATTCCTACCAATACTGATGTTGGCACAAAGCTTGT |
| | | TGAGCTCATGCTCCACTGTGAGCGGTTCATTGATTTCCAC |
| | | CTCCTCAAGATTCTGGCCGTCTGTGCTGGGCACATAACAG |
| | | AATTCAAGCTCGTGAGTGTGGTCGAAGAACGCCTTTAGGA |
| | | TCGTCACACATGCACCCGCAACAGCAGCATGCCCTGCACC |
| | | ATAGCTGGGATGCATTGGGCTACCCTCCGGGAATGCCATC |
| | | GGGAGAAGATAAGTTTCTGCGCTTTCGCAGCCCTCAGGAT |
| | | TATAATCGCCGCTTCGGGCACCACCGTTGTCGGAAAGTTC |
| | | ATTCTGCGCGGCGTTGTGCTGCGAGATTCGTGTCATAAGG |
| | | TCATTGTCGATCTCAGTGGCGATTTTCTGGATGGGCTCAA |
| | | AGGGCTCTGCTCCCTCCGTCTTTCGGTATCGCTCGATAAG |
| | | ACCGCCGACAGCCTCGGGGCGTAGACGGCGGTGGGTACCA |
| | | AACTTCTGGAAGCGCACAGCCTTCAATGCCCTAGTTGCGA |
| | | CCTCTGTGACAAGCGAGAGGATATGGGGACCTCCAAAGGT |
| | | AGCGAATCCCTGTTGTTTATCGATGTCATCATCTTCCACA |
| | | AAAGGGATTTCTGGATCGAAAGGGGCCTTGGTGTCGAGAA |
| | | GAATGAGACATGCGTTCAGGTATGCCTGGTAAAGTGCATC |
| | | GAAATGGACGTAGGTGGCAAGATCGCGCGGTGTAGTAATG |
| | | AAACGGAATGGAGTGTCGTTGTCGTACGTTTCACGTCCTC |
| | | GTACATCACCTGCGTTCTGCACGTCGAGCCAGGATGCCCA |
| | | GGTGGTCATGTAGTCCCTATTTGGGGTTGCCACACGAACA |
| | | CGTTGGTCCATACGCATACCTCCGTATTGAATGAATCCAT |
| | | CTGAAATTTCCTGAATGTTCGCGATACCCTTTGTGCCAAC |
| | | GAGAAGGAACTGAGAGAGATAGGGACCGATTTGTTCACCA |
| | | GGGGTCACGCCGCGGAAGATGTTTGTTTTCCTGAATGGAC |
| | | CTCGCAAACGTGCAATTTCTGCATCAGACAGATCGCAGGG |
| | | GCGTTGCCCGGGCTGCTCTTGGAACTGCACCCAGCGCGTT |
| | | CGGCTGATCGAAGCGGCTGCGGCAGCGGTAGTGCCATCCC |
| | | TTTCGAACTCTGTGAACGGGACGTCGCGAAGAAGGGCCAT |
| | | CCAGTAGCACTCGGTGATCTCCGTCAAGAGTTCTTCACTA |
| | | TCGATTTTTGGGGCAGGCGGCATGGTGACTGCCTGAGCGT |
| | | CCGGTCCCTCCAGATCGTATGTATTACCAGCTGCCATGGA |
| | | TTCCCACGCTCTAGTGTCGGCTATGGGATGGTCGGAGCCC |
| | | TCTGAAGCGATGGCGGATATGAAACGGGGGTCTTCATCGA |
| | | TACCTAGGGGGAGACTTGTAATATCTTCTTGAGCGCCTGT |
| | | TTCGATGGCGCGTACAAAGGCTGCGAAGTGATCTGGAGAG |
| | | GAGGAGAGGCCGGTATCCTTTTCGTGTGGAAGACCTTTCG |
| | | TGTAGGAGGCGAGGTGGGTGTCGTGGTAGCGATGCTCATC |
| | | GCCGTTTGCAAGGTGGGTGGGGAGGGGGCGTGAAGCAGCG |
| | | AGATCTGCGGCTTCACTACGGACTCTCAAGGCGGATGCAT |
| | | GGCGTTCTTCGGACAT |
| 66 | Asparagopsis | TCAGTTGGAGATCGTAATGCACGTTCCATCAAACTTGGGG |
| | armata Ireland | AGGGTCATGGAGAACTTCTCACCATACGTCAAGCTCTGTT |
| | - Mbb4_like | CTTGCAAAATACCGATGGCAACCTCTTCACCCAACTTTGC |
| | coding | ACTCTCTTCGTAATCCGAGTAGTAGTGCACACCGGCCCAG |
| | sequence | TTTCTTCCAATACTGATGTTGGCACACAACTTGTTCAGCT |
| | | CGTGTTCAACTGTGAGCGGTTCACTGATGTTAACCTTCTC |
| | | TAGCTTCCTGCCGTCTGTGCTGGGTTCAAAGCAGAACTTT |
| | | AACTTGTATTTGTGATCAAAGAAGGCCTTGAGGATCGTGA |
| | | CACAGGCGCCAGCGACAGCGGCGTGTCCAGCACCGTAGCT |
| | | GGGATGCATTGGGCTTCCCTCCGGGAACGCCATTGGAAGA |
| | | AGGTAGGTTTCTGCGCTTGCGCCACCCTCGGGGTTGAAAT |
| | | CATTTCTTCGTGCTTTACCGTTGTCGGAAAACTCATTCTG |
| | | CTTGGCGTTGTGACGCGAGATGCGCGTCATAAGCTCATTG |
| | | TCGATCTCGGTAGCGAGCTTCTTAGCGGCTGCGAATGGCG |
| | | CTGCGCCGTTCGTCTTGCGGTAGCGCTCGATGAGTCCGCC |
| | | GACAGCTTCGGGGCGTAGGCGACGATGCGTACCGAACTTC |
| | | TGGAAGCGCACAGCCTTAAGTGCTCTGGTTGCAACCTCTG |
| | | TGACAAGAGAAAGGATGTGAGGCCCGCCAAAGGTGGCAAA |
| | | TCCCTGTTGTTTATCGATGTCGTCGTCCTGCACAAAAGGA |
| | | AGGCCATGATCGAAGGGAGCCTTTATGTCGAGAAGAATGA |
| | | GACAGGCATTGAGGTATGCTTGATAGAGGGCGTCGAAATG |
| | | GACGTAGGTTGCGAGATCGCGCGGGGTGGTAATGAAACGG |
| | | TAATCAGTATCTTTGTCGTATGTTTCACGTGAGCGGACGT |
| | | CCCCTGCATTCTGTACGTCGAGCCAGGAGGCCCAAGTAGT |
| | | CATATAGTCGCGGTTGGGAGTCGCAAAGCGAACGCGCTGG |
| | | TCCATTCGCATACCTCCGTATTGAATGAAACCTTCTTCTG |
| | | GCTGGTGCATTTTCCCGATACCCTGTGTACCGACAAGCAA |
| | | GAACTGGGAGAGGTAGGGGCCTACCTGTTCACCAGGCATG |
| | | TCGCCGCGGAAGACGTTTGATTTGTTGAAGGGGCCGCGCA |
| | | GACGTTGGATTTCAGCATTTGTGAGATCGGATGGGCGCTG |
| | | GCCGGGCTGCTCCTGAAACTGTACCCAGCGTGTACGACTA |
| | | ATCGATGCGGCTGCGGCAGCAACTGTTCCGTTTCGATGAA |
| | | ATTCTGAGAAGGGCACGTCGCGAAGAAGGGCCATCCAATA |
| | | GCATTCTGTGATCTCTGTCAAGAGCTCTTCGCTGTCGATT |
| | | TTAGGTGCAGGAGGCATGGTGACTGCCTGTGCATCAGGCC |
| | | CTTCGAGATCGAAGGTATTGCCTGCGGCCATGGATTCCCA |
| | | GGCGCGAGTGTCAGCGATGGGGAGGTCGAGTGCTGGGGAT |
| | | GCGATGCCGGAGATGAAACGAGGTGTTTCATCAATACCTA |
| | | GGCGGAGGCTCCTGATGTCTTCCTGTGCGCCTGTGTTGAT |
| | | AGCACGCACAAAGGCAGTGAAGTCATCTGTAGATGAGAGA |
| | | AGGCCTGTCTTTTTGTCGTGTGGTAAACCCTTTGTGTAGG |
| | | ATGCGATGTATGAATCTGAGAAACGGGTCTCATCGCCATT |
| | | GGGGGTGTGGGTAGGGAGTTTGCGGGAGGCTGCTAGTTTT |
| | | GCGGCATCTGTGCGTACTTGTAGTGCGGCTGCTTGACGTG |
| | | TGGTAGACAT |
| 67 | Asparagopsis | ATGGCAACCCCCGACACACAGAATCCCAATCGCGCAGAGG |
| | armata Ireland | CCTCCTTCAAGATTCGCCTCTCTGCCGCCGAGCTTGCTCG |
| | 2 - Mbb1_like | CTCCCGTGGTTCTCCAACTCACATCTCCAACAATTCAGAG |
| | coding | GCTCGCTTCAGAAACCCAGATGGTACACGATCTCTCTTGG |
| | sequence | CTAACTTCACGAAAGGTCTCCCCCAGATCAACGAAACAGG |
| | | TCTGGTTCAATCTGCCCTCGATTATGACCAATTTGTACGC |
| | | GCTATTGACAGCGGAGATGCCCGCGATTTCGCAGCTCTCC |
| | | CACTCGGCCCCCAGAATGTCACCCCCAGATTCACATCCGG |
| | | CATCGCCACAAACCCAGAGCAGTCGGGAGTTCGTGCCTGG |
| | | GAAAGTATGGGTGCTGGACGCACATTCGATCTCGAAGGGC |
| | | CTGATGCACAGGCCGTCACTATGCCGCCCGCACCAGAGCT |
| | | AGACAGCGAGGAGTTGGTCACTGAAGTCACCGAATGTTAT |
| | | TGGATGTCCCTGCTCAGGGATGTACCCTTCCCAACATTCC |
| | | ATTGCGACGAAGATGTCGCAGCCGCTGCCAAGAGTATCAA |
| | | CGACACTCAATGGATTAAATTCAAGGAAAACCCTCCCACC |
| | | CATCTCACATCTGCCGAACGCTCCCGTCTCAGAGGCCCCA |
| | | TCGATGCCTCCAATGTCGTCAGGGGCATCACTCCTGGAGA |
| | | TGATGTAGGCCCATATTTGTCCCAGTTCCTCTTGGTCGGT |
| | | ACCACAGGAATCGCAAACGGAAACGAGGTCAGCGATGGTT |
| | | TCATCCAGTACGGTGGTATGCGCATGGACCAGCGGGTACG |
| | | CGTGGCATCCCCCAAGGTTGACTACATGACCACCTTTGGA |
| | | TCCTGGCTTGACGTGCAGAACGCTGCGGATGTCACCGGGC |
| | | GTGAGCGATACGAGTCAGACCCTGGTTTCCGCTTCATTCA |
| | | CACACCGCGTGATCTGGCGACATACGTCCATTTCGACGCT |
| | | CTGTACCAGGCCTATCTTAACGCCTGCATCATTCTGCTCG |
| | | ACATTGGTGCACCGTTCGATTCCGGAGTCCCGTTCCAGCT |
| | | CGACAATGATATAGATAAGCAGCAGGCATTTGCCACGTTT |
| | | GGCGGACCGCATATTCTCTCTCTCGTTACTGAGGTGGCCA |
| | | CTCGCGCTCTCAAGGCAGTGCGCTTCCAGAAGTTCAACGT |
| | | CCATCGCCGTCTACGCCCGGAGACTGTCGGCGGTCGTGTC |
| | | GACCGATACTACAAAACCAAAGCACCCGAATTTTCCGGAG |
| | | CTGCTAAATTGGTAGATTCCCTGGATTCTGACCTGTTGGA |
| | | TCGAGTGTATTACCATAACAAGAAGCAAAATAAGTTGAGC |
| | | GACAACGGTGCTCCGCGTAGTGCAGATTTCAATCCTGATG |
| | | GCGGCCTGAGCGAAGGCAACCTTCTCATGCCAATGGCATT |
| | | CCCAGAGGGAAGCCCGATGCATCCGGCTTATGGTGCTGGA |
| | | CATGCCACTGTTGCAGGAGCCTGTGTAACTGTTCTCAAGG |
| | | CATTCTTTGATGGTGGATTCCCCCTGCCGTTTGCATACGT |
| | | CACTAACGAAAATGGCTGCGGCCTCGAGCCGGTTGAAGTG |
| | | TCTGAGCCGCTCACAGTTGACGGAGAGCTGAACAAGATTT |
| | | GCTCTAATATTTCCATTGGACGTAACTGGGCTGGTGTGCA |
| | | TTACTTCACTGACTACATTGAAAGTATCCGCATTGGTGAG |
| | | GAAATTGCTATTGGCATTCTTCAGGAACAGAAGTTGACCT |
| | | TCAGTGAGAATTTCTCGATGACCCTCAACAAGTTTGATGG |
| | | AAGCACAATTCGCATTTAA |
| 68 | Asparagopsis | ATGAACACAGCATTTCCCTTCAGAAGAAAATGGTCCCCAA |
| | armata Ireland | GTCTTTGGAGTGCTCGCCTCGAGTCCTATCTCTCCACTCA |
| | 2 - Mbb2- | CGCCTTCGAGCTTCTCACAGTTATCACATACATAACTGCA |
| | 2_like coding | CTCTCTCTCATGTTCGTATGGGGAGCTCACGATGACATTT |
| | sequence | CTATTGCTCGCGGAGCAGGTTACACACTCAACCTGTTCCA |
| | | CAATCAATCTCCCGACACAAATCTCCGCTGGTACAACACA |
| | | GCACTTGTCATTCTACTTGCATCTCGCCTCTTCTTCACAT |
| | | TCATCCGTGATACTCCCCTGCACAAGATTCTTCCTGTTGA |
| | | CAAAAGCTTCCCCGCCTTCCATATCCTTGTTGGATATACG |
| | | ATAGCCGCATCTCTCTTCATTCATGTCCCGTTCCATTTCA |
| | | CATGGATCATTGCTTACGATAAATGGAGTGCAGGACTTTG |
| | | GGATTTCACCATGAGCGCTGTCACAGGCGTCGTATTGTTA |
| | | CTTATCTTCATAATCATGTTCATGTTCGCTCTACCATTCT |
| | | ACAGAAAAAAATATTTCCGGCTCTTCTACATTTCCCACAT |
| | | GGTTGGAGCACTGCTGTTCTTTCCTCTTCTCATCTTCCAT |
| | | GGTATGTACCGTCGTGTTCCGGAGACTTACAAGTGGGTTG |
| | | CTCTACCTCTCGTGATCTACATTCTGGACAGAATCGCCCG |
| | | TCGCTTCAAAATCTCCACCGTTCAGCTGAAGCTCACTTCC |
| | | GAGCATTCAGCTCTCAAAGGAAAAGACATTTTGGAACTTC |
| | | GTATTCCGAAACCCTTCACATATCGCGCTGGTCAGTATGC |
| | | AGAGATCCGTGTACCATCCTTGAGTAGGGAGTGGCATCCC |
| | | TTCACAATTGCTAGTGCACCCCATGAAAAATGCATGTGCT |
| | | TTTTCATCAAATCACTCGGGAATTGGACCAAGGAGCTTCG |
| | | AGGGGCATTTCAAAACCGTATGGAAGGAGAAGATCCGAAC |
| | | CCATTGCTAGTTCAAGTCCGTGGTCCATTTGGCGCTCCCG |
| | | CTCAACATGTCTCTTCGTATGAAAGAGTGGTCCTCATATC |
| | | TGGCGGAGTTGGTTCAACGCCCTTCGCAGCCATCTCCAAA |
| | | AGTTTGCATTTCTTGAACAAGAACAAAACCTGCCCTTCTA |
| | | CTGATTTTTCGAAAACCAATTTGTCTGAAATGAAGCATAT |
| | | TGAGCAGCGTATCTCGGAAACTATTTCTGCTGTATATGAT |
| | | ATCGATTTGGCAAACAGCTCCGATGAACCGGAATCTGAAG |
| | | AACAAAGGAAGCAGTATATTACTGACATGATGAACTTGAC |
| | | GGCCCAAAATTCCGAAGAACTCGATGTTTCCACTTCAAGA |
| | | ACAAGTGAACAATGTGTATCATCGAGGAGCTGCCCAAATA |
| | | TTGGAGATGGTCTCCGGATATTGGATGTCGAAAATCAATT |
| | | AGAAGAATCTTCAGTGAGCAGCATGTCTACATGCGACGAT |
| | | GAAGAAGATTATTCATCAGATTTCAGTGAAGAAATGAGAC |
| | | TTGAAAAGGAACTTGCCGCCAGGAAACGTAAGATGGCTCG |
| | | CCAAAAAATTGCCCGTCTCTATGAGAGACGTTCAATATTA |
| | | TTCTCTTTTCTTCATACCACTCGGGTAACTCTGGCACTTC |
| | | AACTCTCTCTAATTGCACGCATTGTTCTTGTGTGCATTGT |
| | | ATCAATCTTCAACATTGGTGAATTCAACTTTGGGGACAGC |
| | | CGCATAGGTGCCAAATGGGTTATAGGTGCCGATGCAGTGT |
| | | TGAGTGTTTTGCATGCAGTCACTCTAACTTTGACCATACT |
| | | CTTGGAAATTAGCTTCATGGGACCCCGCTTCTTCCGTTCT |
| | | TCTTGGCAGTTTATGGACTTTTTCGTTTTCGTCCCGCTCA |
| | | GTATTCTGTGCAGCGTCAGTGGTGTACTATCTTGGACTGG |
| | | GACGGAAATGTCAGGAACATTAGTTTTTCTTCACTTCATT |
| | | GTATTGCTGCCAATTATGCTTATTTTCATGAGTGCTCGTC |
| | | TGTATCGGAGTATTGGGTCCCTGAATCTATTTCATGATCG |
| | | GAAGTGTAACCCAAACTGCGCATGCGGAAAGAAAATCCCA |
| | | GACGTTGACTTTATCTGGACAACACGTCACACCGTAGATG |
| | | ACCAATGGTTACGAGAAGAACTCTTTCCTCTAGAGGACGG |
| | | CACAAAACTCCGTCTTCACCGATATATTACAGGCAAGGAA |
| | | AGTGAAGACTTTGAAAGTGCTGAGACCTTTATTACTACAA |
| | | GCAACATTGGAAGACCGCCCTGGGACGAAATATTCGCCAA |
| | | AATTGCCCAAAAGTCACCAAGTCACGCAGAAATCGGAGTG |
| | | TTTTTCTGCGGACCACACTCAATGGGAGCAGCTGTGAGGA |
| | | AAAGCATGCGAAAAACTGAAATACGGAGTAACTTGCGTGG |
| | | GGCATATCTGGAGACAACTAATGTTGTCGATCTTGTTGCC |
| | | GATCTTCTTCTGAGAAATACTGACCATGCAGCAGAGCTTC |
| | | GGAGGCATGGGTGCAATATTCGATTTGTATTTCGTGAAGA |
| | | GAACTTCGGTTGA |
| 69 | Asparagopsis | ATGGCTCCCCGCTCCGGCAAGCAACGATGCTCTCCCACCT |
| | armata Ireland | CTCTCACTCTCCTCAGCGCACGCCTAGAATCCTATCTATC |
| | 2 - Mbb2- | AACAAATGCATTCTCGCTTCTCGCACTTTTCATCTACTCA |
| | 3_like coding | CTCGCAAACATTCTCATGTTCGTCTGGGGTGCCCATGACG |
| | sequence | AATTTCATCACCATACTTCTCAAAAAAATCTACGATGGTA |
| | | CATCTGCATTGCCCGTGGTGCTGGATACACACTCAATCTC |
| | | AATACCGCTCTCGTCATCCTTCTCGCCGCCCGACTTTTCG |
| | | CGACCTACATCCGTGAAACTCCTCTGCAACACATCCTCCC |
| | | AATCGACAAAAGTTTCCCAGCTTTCCACATCATCGTGGCC |
| | | TACACCATTGCAGCCGCACTCTTCATACATGTTTCCTTCC |
| | | ATCTCGTGTGGATCATTGCCTATGATAAATGGGAATTAGG |
| | | CATGTGGGGGTTTACTATGAGTGCCACTACAGGTTTTGCT |
| | | CTTCTCACAGTATTTGTCGTCATGTTCGTCTCTGCTCTTC |
| | | CTAGATTTCGAAAGAAGCAATTCCGTACCTTCTACCTCAT |
| | | CCATATGGTTGGTGCATTACTATTCTTCACTCTGCTTATC |
| | | TTTCACGGCATGTACAACAGAGTCCCCGAAACGTACAAAT |
| | | GGATTGCCGGACCTCTCGCTATCTACACAATTGATAGGGT |
| | | TCTGAGACGATTCAAGATTTCAACAGCGAAGCTCGAACTC |
| | | ACTGGTGAGCACTCTTCCCTGAAGGGAAGTGATATTCTGG |
| | | AGCTGCGAGTCCCAAAAACGTTCGAATACCGTGCTGGTCA |
| | | ATATGCAGAGGTATGCGTGCCTTCAATAAATAATGAGTGG |
| | | CATCCTTTTACAATTGCAAGTGCACCTCATGAGAAATCTA |
| | | TGTGCTTCTACATCAAGGCGATTGGTGATTGGACTAGACA |
| | | TCTTCGAGATGCGTTCGAAGCTCGAATTGAGAATGACCTA |
| | | TATGAACCGCTCAAAGTCCAGATCCGCGGACCATATGGTG |
| | | CTCCAGCCCAGCACGTGAGAGGATATCGTCGCGTCGTTCT |
| | | TATCTCCGGAGGGGTCGGTTCAACACCTTTCGCAGCAATC |
| | | TGCAAAAACCTACACCATCTCAACAAGTCTGAAAACCATT |
| | | CGAAAGCTTTGGAATCACATGGATCTTCGAAAAGACAGTC |
| | | TCAAATTCATTGGCACATCCGTGATGCCATTTCCAATCTC |
| | | TTCGATGTATGTCTGGATGATAGCCGCAGTGATGCTGCAT |
| | | CGAACCAACAGCGAATACAACAAGTCGCCGATATGCTCAA |
| | | TATGTCTCCAAAGGGAAACGTCCATAACGTTTCTTTTAAA |
| | | CGCAATCTATCAAAGACGTCTGATGATTGCAGTTCGGTTG |
| | | AAGAAATAACCAAGCAGATCAGAGCGGATGAGGAAATATC |
| | | GCTGTCTCAGAAAGAGAAGCAAAGCAGTTATAATCTTGAT |
| | | GCATGTTCTGAATCTATTCGATATTATAATCGACCTTCAG |
| | | CCAACGTCTTCAATGTTGCGCAGCGGAGGATTTTCAATTT |
| | | CTATGAGTACCGCTCAAGACTGCTCGCTTTTCTGCATACA |
| | | ACAAGATTCACATTTGCTCTTCTTCTCACTCTGGTTGCAA |
| | | GAATTGTTATTTTGTGCGTTGTATCTATTTTCAATCTTGG |
| | | GCATATTGGAATCAGTGACAGTCATATTGATGCCGAATGG |
| | | GTCATTGCCACTAATTCCTTATTGGGACTCGTACTCGGGA |
| | | TTGCGCTGTTGACTACCGTTCTACTGGAAATCAGTTTCAT |
| | | GCGACTGCGCTTCTTCTACAGAGGTTGGCGTTGCGTCGAC |
| | | TTCTTTATCTTTCTCCCTATTACCTTTCTCTGTAATATCA |
| | | GCAATTTCATGAGTTGGAGTGGACGTCAACAAGCGAAATT |
| | | TCTTATTTTTCTGGACTTGATTATTGTCGTTCCAATCATG |
| | | TTCCTCTTGCTTTGCCATCGCCTGTACCGAAGTATTGGCG |
| | | CACGCAACCTTTTGGATGACACACAATGTCGTCAGAGTTG |
| | | CACCTGTAACAAAACTATTCCAGACGTTGACTTTGTATGG |
| | | ACTACTCCTCGTGATAGTGAGGATGAGTGGCTGCGGAGTG |
| | | AGCTGTACCCGCTGGCCACAGGAACTGATCTACGATTGCA |
| | | CCGATATGTAACACGTGAAAAGGACTTGGACCTGGAAGAA |
| | | CCTGAACATTTCATCACTACTGCGAATACTGGACGGCCTC |
| | | AGTGGGATGAGATTTTCGCACAAATTGCCGAACAAACTCC |
| | | TAGCCACAATGAAGTTGGAGTGTTCTTCTGCGGGCCACAT |
| | | CCTATGGGTGCAGCAGTTCAGAAGAGTATGAGGAAAGTTG |
| | | AAGTGATGAGCAACCTCCGAGGATCGTATTTGCGAAAGAC |
| | | TGACAGTGAATTGTTAGTGGAAGACTTGTCATTGAGGGAT |
| | | GAAAAGGAGGTTAAGTTATTGAGAGAACACGGATGCAATA |
| | | TTCGGTTTGTCTTTCGAGAGGAAAACTTTAGTTAG |
| 70 | Asparagopsis | ATGCCCTCTCCATCCTCATCCACATGTCCAAAGTCCACTC |
| | armata Ireland | AAGATCTCCTCACCGCTCGACTCGAGTCCCACCTCTCCAC |
| | 2 - Mbb2_like | ACATGCCTTCGAACTTCTCGCTCTCGTCGTGTATGCCACC |
| | coding | GCACTCCTCCTCATGTTCCTATGGGGAGCCCATGACTACA |
| | sequence | TCTCCATCGCCCGCGCAGCAGGTTACACCCTAAATGAATT |
| | | CCATCACCACACTTCCGATGATAACATGCGATGGCTCAAT |
| | | ACTGCATTGGTCATTCTTCTCGCTGCCAGACTCTTTTCAA |
| | | CTTATATTCGCGAGACCCCATTGCATCTAATCATTCCTCT |
| | | TGACAAAAGCTTCCCAGCCTTCCATATTATTGTCGCTTAC |
| | | GCTATTGCCATCTCCCTCGTCCTCCATGTCGCTTTCCATT |
| | | TCGCGTGGATTATTGCCTACGATAAATGGGAACCAGGCCT |
| | | TTGGGGATACACTATGAGCGCATCGACAGGATCTGTCCTC |
| | | CTCATTGTGTTTCTAGTCATGTTCATAACTGCCCTCCCAA |
| | | CATTTCGGAAGAAACGCTTCCGATTCTTCTATCTCACACA |
| | | TATGTTCGGAGTGATACTCTTCTTCCCTCTACTAATCTTT |
| | | CATGGAATGTACAACCGCGTTCCAGAAACTTACAAATGGA |
| | | TCACCGCCCCACTCATCATCTACATTATTGATAGACTTCT |
| | | TCGTCGTTTCAAAGTGTCCACAGTTCCCTTGCAGCTCACC |
| | | AGCGATCATTCTTCTCTCAAGAGTGCTGACATTCTCGAAC |
| | | TCCGAGTCCCAAAGCCCTTCCATTACAGAGCGGGGCAATA |
| | | TGCAGAGGTCTGTGTCCCTTCGATCAGTTCAGAGTGGCAT |
| | | CCATTCACAATCGCAAGCGCACCCCACGAAGAAACCATGA |
| | | CCTTCTTCATCAAAACTCTTGGCAATTGGACTCAAGCACT |
| | | GCGCCACCAATTCCAAACACGCATGGATTCAGACGACTGC |
| | | AAACCACTTACGGTACAGATCCGCGGACCGTACGGCGCTC |
| | | CCGCTCAGCACGTCGGCAGATATCAACGGGTAGTGTTGGT |
| | | GTCAGGAGGTGTTGGCGCCACCCCTTTTGCCGCCATCTCG |
| | | AAACATCTGCACCACCTGAATGTCGCTGAACATCACAAAA |
| | | AGACGCTCGATGTTGACATTCCATCTAAGCATACAATACA |
| | | ACTCGGGAACCGGATCAGAGAAACGATCTCCAATATGTAC |
| | | GGCTTGGGGTTGGGTGAAAGCGGCTGCGCAGAGGATTCCA |
| | | ACCAACAAAGAAGACAGCATCTGGCGGATATGCTGAACAT |
| | | GTCCCCGGATGGAGCGGTGCAGAAATTAGGAAATGAAATC |
| | | TCTCATCCGTCTCCGACAGGCTCTTCTGATGTGAGTGATA |
| | | CGTGCAGTTCGGAAAATGTCGATCTCTCGTTTTCATCAAG |
| | | TATTGGGAGTCAAATGGCCACGCTGGGAACATACGAGAAA |
| | | GAGTGCAGCTCTAGAAAAGGCGGGAGAGAAGATAAGATGG |
| | | GGCGATCTGCGTACTCGAAGCATAATTTGATGTTTCTGAA |
| | | TGGAAAGCGCTCTAGACTTCTTTCCTTACTTCACACGACT |
| | | CGAATAACATTGCTGCTGCTGTTCACACTAATTGCGCGAT |
| | | TAGTGCTGACGTGTGTTGTATCTATCTTCGATCTTGGTCA |
| | | TTTCAGCACAACCAGCGGACACATAGAGGCTAAGTGGGTT |
| | | ATCGTGACAAACGTCGTTCTGGGACTAATTGTTGGATTGA |
| | | CTCTATTCACAATAATTGTGCTGGAAGTGAGTTTTATGGG |
| | | GACGCGATTCTTCCACAGGAATTGGCGATGCGTTGACTTT |
| | | TTGGTGTTCGTTCCTCTGTCATTTCTGTGCAATCTATACG |
| | | GAATTGTAACGTGGGCTGGACGAGACCAATCGTCCTTCCT |
| | | GACATTTGTCGACTATATTATTATCACACCGATTATCTTC |
| | | CTACTTCTCTGTCATCGATTATACCGAAGCATTGGGACGC |
| | | GAAACCTTTTCGACGATAACGGGAGTTGCCGGGGGTCAGA |
| | | ACACAGCGTTCCGGAAGTGGACTTTATCTGGACGGTCGCG |
| | | CACGAGGGGGACGACGAGTGGCTTCGCGAAGATCTGTACA |
| | | CGCTTGCGACGGGGACGGAGCTGCGACTGCATCGGTACGT |
| | | GACACGGGAGAAGGAAGTGGAGGTGGAGGGAGGGGAGGAG |
| | | TTTATCACATCTGCTAGGGCGGGGCGTCCGAAGTGGGATG |
| | | AAATATTTTTGAAGATCGCGGAGGAGTCGCGGAGCTGCAG |
| | | TGAGATCGGGGTGTTCTTCTGTGGACCCAGTGGGATGGGG |
| | | AAGCAGGTTCGGGAGAGTTTGCGGCGGGCGGAAGTTTTCA |
| | | GTAGTTTGAGGGGATCATACTTGAGGAAAACGGGTGGAGG |
| | | GGAGGTTGGGGACGAGGTAAGATTACTGAAGCGGTTTGGA |
| | | AGCAATGTTCGATTCGTATTTCGGGAGGAGAACTTTAGCT |
| | | GA |
| 71 | Asparagopsis | CTATATTTTGATGGTTGTGCCATCAAATTTGGGAATTGTC |
| | armata Ireland | ATTGAGAATTTTTCCGAATAAGTTAACTTTTGCTCTTTCA |
| | 2 - Mbb3_like | GGATTCCAATCGCCACTTCCTCTCCCAATCGAATACTCTC |
| | coding | CACATAGTCGGAGTAGTAGTGTACGCCAGCCCAGCTACGT |
| | sequence | CCAATACTGATATTAGACGCCAGTTTATTCAGCTCTCCTT |
| | | CCACCGAAAGTTTCTTCGCGACCTTTGCCTTCTTCAGCAT |
| | | CTTCCCATTGCAGGATGAGACATAGGCGAACGGGAGCTTG |
| | | TAGGTGTGATCGAAAAAGCCTTTCAATATCGTTACGCACG |
| | | CCCCGGCCACTGTGGCATGCCCGGCACCATAGCTAGGGTG |
| | | CATCGGACTTCCCTCCGGATAGGCCATTGGCAGCAAATGA |
| | | GTCTCACCGGAGCCTGTATCATGGGAGCGTGGAAATCCGT |
| | | TGTCGCTCATTGTTGCGTTCTGATGGCCGTTCGCTGCTGC |
| | | GACGAGCGACATGAGGTTTGAACTTAGAGAACGGGCGAGA |
| | | TGGCGTATGGGACGAATAGTATTTGCTAGCTCTGAATATC |
| | | CGCCACCGTATCGTCTGTATTGTTCTAAGAGGCCGCCGAC |
| | | AGCCTCTGGGCGAAGACGTCTGTGGACGGCGTATTTTTGG |
| | | AAACGGACTGCCTTGAGGGCTCGGGTAGCAACCTCTGTGA |
| | | CGAGTGTGAGGATATGGGGGCCTCCAAAGTTTACAAAGCC |
| | | CTGTTGTTTGTCGATGTGGTCTTCCGTGGTGAAGGGGAGA |
| | | CCGGAGTCGAATGGGACTTTCATGTCGAGGAGGATGATGC |
| | | ATGCGTTGAGATAGGCTTGGTAGAGCGAGTCATGATGAAC |
| | | GTAGGTAGCGAGGTCGCGTGGTGTGTACAGGAAGCGATAG |
| | | GAGCTTGATTTGGCATATGTAGAATGTCTGCGCAAATCTG |
| | | CGGCATTCTGGACGTCAAGGAAGGCGGCCCAGGTGGTCAT |
| | | GTAGTCGATTTCTGTCTCGACTGATCGAACTCTCTGATCG |
| | | ATTCGGATGGCACCGTATTGGATGTAGCCATCTGCCTCGT |
| | | CGTGGATTTCTCCGAGCCCTTTGGTTCCAACAATGAGGAA |
| | | TTGTGAGATGTAAGGACCATTGCGCTCGCCTGGTGTGGTG |
| | | CCACGGAAGACATTAGAGGATGAGAAGGGTTCTCTGTGTC |
| | | TGTGACGTTCCAGTTCTGGGATGCAACATTTATGGGATTG |
| | | AATCCAATGGGTGTGATTGATATCGTCGACGGCAGCGGCG |
| | | ACGAGGTGATGACTTTTGAAATGAGAGAATGGGACATCCC |
| | | TGAGGAGTGCCATGTAGTAAAGCTCTGTCATCTCTGTGAC |
| | | GAGCTCATTGGATTCGAGACGTGGACATGGGGGTATTGTG |
| | | ACTGATTGGGGATCAGGCCCTTGAAGGTCGAATGTATTTC |
| | | CAGCGGCCATGGATTCCCAAGCGCGGAGTGGGGCATCCTT |
| | | TGCAATTCCGGAGCGGAATGTGGAAAAGGTGGATGGAGGT |
| | | CCGAGGGGAATATCTGCAATGTGCTGTGGAGTTCCTGTTT |
| | | GGACAGCACGGGCGAAGAGTTGAAAATGCTGTGCGGAGAC |
| | | TAGACGACCGTCGGGATGATGAGGGAGACCCTTTGTGAAG |
| | | GTCGCGAGGTAGGAGAGAGCATTTGAGGTAGTCTTGTACA |
| | | GCGTCTCATCGCCATTTCTTAGATGAGGAGGATGTGAACG |
| | | GTTTGCGGCTTCATCTGCAGCTGCAATGCGAGTGACGAGA |
| | | GCGGATTGACGACGAGTTGGGGGAGGCAT |
| 72 | Asparagopsis | TTACTTTGAGTTTGAAATCTTGATAGTCGTACCATCAAAC |
| | armata Ireland | TTGGGAATAGTCATGGAGAACTCCTCACCGTACGTCAGAC |
| | 2 - Mbb4- | TCTGTTCTTCTAGAATACCAATTGCAACCTCTTCACCCAA |
| | 2_like coding | CCTTGCGCTCTCTATGTAATCCGAGTAATAGTGCACACCA |
| | sequence | GCCCAATTCCTACCAATACTGATGTTGGCACAAAGCTTGT |
| | | TGAGCTCATGCTCCACTGTGAGCGGTTCATTGATTTCCAC |
| | | CTCCTCAAGATTCTGGCCGTCTGTGCTGGGCACATAACAG |
| | | AATTCAAGCTCGTGAGTGTGGTCGAAGAACGCCTTTAGGA |
| | | TCGTCACACATGCACCCGCAACAGCAGCATGCCCTGCACC |
| | | ATAGCTGGGATGCATTGGGCTACCCTCCGGGAATGCCATC |
| | | GGGAGAAGATAAGTTTCTGCGCTTTCGCAGCCCTCAGGAT |
| | | TATAATCGCCGCTTCGGGCACCACCGTTGTCGGAAAGTTC |
| | | ATTCTGCGCGGCGTTGTGCTGCGAGATTCGTGTCATAAGG |
| | | TCATTGTCGATCTCAGTGGCGATTTTCTGGATGGGCTCAA |
| | | AGGGCTCTGCTCCCTCCGTCTTTCGGTATCGCTCGATAAG |
| | | ACCGCCGACAGCCTCGGGGCGTAGACGGCGGTGGGTACCA |
| | | AACTTCTGGAAGCGCACAGCCTTCAATGCCCTAGTTGCGA |
| | | CCTCTGTGACAAGCGAGAGGATATGGGGACCTCCAAAGGT |
| | | AGCGAATCCCTGTTGTTTATCGATGTCATCATCTTCCACA |
| | | AAAGGGATTTCTGGATCGAAAGGGGCCTTGGTGTCGAGAA |
| | | GAATGAGACATGCGTTCAGGTATGCCTGGTAAAGTGCATC |
| | | GAAATGGACGTAGGTGGCAAGATCGCGCGGTGTAGTAATG |
| | | AAACGGAATGGAGTGTCGTTGTCGTACGTTTCACGTCCTC |
| | | GTACATCACCTGCGTTCTGCACGTCGAGCCAGGATGCCCA |
| | | GGTGGTCATGTAGTCCCTATTTGGGGTTGCCACACGAACA |
| | | CGTTGGTCCATACGCATACCTCCGTATTGAATGAATCCAT |
| | | CTGAAATTTCCTGAATGTTCGCGATACCCTTTGTGCCAAC |
| | | GAGAAGGAACTGAGAGAGATAGGGACCGATTTGTTCACCA |
| | | GGGGTCACGCCGCGGAAGATGTTTGTTTTCCTGAATGGAC |
| | | CTCGCAAACGTGCAATTTCTGCATCAGACAGATCGCAGGG |
| | | GCGTTGCCCGGGCTGCTCTTGGAACTGCACCCAGCGCGTT |
| | | CGGCTGATCGAAGCGGCTGCGGCAGCGGTAGTGCCATCCC |
| | | TTTCGAACTCTGTGAACGGGACGTCGCGAAGAAGGGCCAT |
| | | CCAGTAGCACTCGGTGATCTCCGTCAAGAGTTCTTCACTA |
| | | TCGATTTTTGGGGCAGGCGGCATGGTGACTGCCTGAGCGT |
| | | CCGGTCCCTCCAGATCGTATGTATTACCAGCTGCCATGGA |
| | | TTCCCACGCTCTAGTGTCGGCTATGGGATGGTCGGAGCCC |
| | | TCTGAAGCGATGGCGGATATGAAACGGGGGTCTTCATCGA |
| | | TACCTAGGGGGAGACTTGTAATATCTTCTTGAGCGCCTGT |
| | | TTCGATGGCGCGTACAAAGGCTGCGAAGTGATCTGGAGAG |
| | | GAGGAGAGGCCGGTATCCTTTTCGTGTGGAAGACCTTTCG |
| | | TGTAGGAGGCGAGGTGGGTGTCGTGGTAGCGATGCTCATC |
| | | GCCGTTTGCAAGGTGGGTGGGGAGGGGGCGTGAAGCAGCG |
| | | AGATCTGCGGCTTCACTACGGACTCTCAAGGCGGATGCAT |
| | | GGCGTTCTTCGGACAT |
| 73 | Asparagopsis | ATGTCTACCACACGTCAAGCAGCCGCACTACAAGTACGCA |
| | armata Ireland | CAGATGCCGCAAAACTAGCAGCCTCCCGCAAACTCCCTAC |
| | 2 - Mbb4_like | CCACACCCCCAATGGCGATGAGACCCGTTTCTCAGATTCA |
| | coding | TACATCGCATCCTACACAAAGGGTTTACCACACGACAAAA |
| | sequence | AGACAGGCCTTCTCTCATCTACAGATGACTTCACTGCCTT |
| | | TGTGCGTGCTATCAACACAGGCGCACAGGAAGACATCAGG |
| | | AGCCTCCGCCTAGGTATTGATGAAACACCTCGTTTCATCT |
| | | CCGGCATCGCATCCCCAGCACTCGACCTCCCCATCGCTGA |
| | | CACTCGCGCCTGGGAATCCATGGCCGCAGGCAATACCTTC |
| | | GATCTCGAAGGGCCTGATGCACAGGCAGTCACCATGCCTC |
| | | CTGCACCTAAAATCGACAGCGAAGAGCTCTTGACAGAGAT |
| | | CACAGAATGCTATTGGATGGCCCTTCTTCGCGACGTGCCC |
| | | TTCTCAGAATTTCATCGAAACGGAACAGTTGCTGCCGCAG |
| | | CCGCATCGATTAGTCGTACACGCTGGGTACAGTTTCAGGA |
| | | GCAGCCCGGCCAGCGCCCATCCGATCTCACAAATGCTGAA |
| | | ATCCAACGTCTGCGCGGCCCCTTCAACAAATCAAACGTCT |
| | | TCCGCGGCGACATGCCTGGTGAACAGGTAGGCCCCTACCT |
| | | CTCCCAGTTCTTGCTTGTCGGTACACAGGGTATCGGGAAA |
| | | ATGCACCAGCCAGAAGAAGGTTTCATTCAATACGGAGGTA |
| | | TGCGAATGGACCAGCGCGTTCGCTTTGCGACTCCCAACCG |
| | | CGACTATATGACTACTTGGGCCTCCTGGCTCGACGTACAG |
| | | AATGCAGGGGACGTCCGCTCACGTGAAACATACGACAAAG |
| | | ATACTGAGTACCGTTTCATTACCACCCCGCGCGATCTCGC |
| | | AACCTACGTCCATTTCGACGCCCTCTATCAAGCATACCTC |
| | | AATGCCTGTCTCATTCTTCTCGACATAAAGGCTCCCTTCG |
| | | ATCATGGCCTTCCTTTTGTGCAGGACGACGACATCGATAA |
| | | ACAACAGGGATTTGCCACCTTTGGCGGGCCTCACATCCTT |
| | | TCTCTTGTCACAGAGGTTGCAACCAGAGCACTTAAGGCTG |
| | | TGCGCTTCCAGAAGTTCGGTACGCATCGTCGCCTACGCCC |
| | | CGAAGCTGTCGGCGGACTCATCGAGCGCTACCGCAAGACG |
| | | AACGGCGCAGCGCCATTCGCAGCCGCTAAGAAGCTCGCTA |
| | | CCGAGATCGACAATGAGCTTATGACGCGCATCTCGCGTCA |
| | | CAACGCCAAGCAGAATGAGTTTTCCGACAACGGTAAAGCA |
| | | CGAAGAAATGATTTCAACCCCGAGGGTGGCGCAAGCGCAG |
| | | AAACCTACCTTCTTCCAATGGCGTTCCCGGAGGGAAGCCC |
| | | AATGCATCCCAGCTACGCTGCTGGACACGCCGCTGTCGCT |
| | | GGCGCCTGTGTCACGATCCTCAAGGCCTTCTTTGATCACA |
| | | AATACAAGTTAAAGTTCTGCTTTGAACCCAGCACAGACGG |
| | | CAGGAAGCTAGAGAAGGTTAACATCAGTGAACCGCTCACA |
| | | GTTGAACACGAGCTGAACAAGTTGTGTGCCAACATCAGTA |
| | | TTGGAAGAAACTGGGCCGGTGTGCACTACTACTCGGATTA |
| | | CGAAGAGAGTGCAAAGTTGGGTGAAGAGGTTGCCATCGGT |
| | | ATTTTGCAAGAACAGAGCTTGACGTATGGTGAGAAGTTCT |
| | | CCATGACCCTCCCCAAGTTTGATGGAACGTGCATTACGAT |
| | | CTCCAACTGATTAAATGCGAATTGTGCTTCCATCAAACTT |
| | | GTTGAGGGT |
| 74 | Asparagopsis | CATCGAGAAATTCTCACTGAAGGTCAACTTCTGTTCCTGA |
| | armata Ireland | AGAATGCCAATAGCAATTTCCTCACCAATGCGGATACTTT |
| | 3 - Mbb1_like | CAATGTAGTCAGTGAAGTAATGCACACCAGCCCAGTTACG |
| | coding | TCCAATGGAAATATTAGAGCAAATCTTGTTCAGCTCTCCG |
| | sequence | TCAACTGTGAGCGGCTCAGACACTTCAACCGGCTCGAGGC |
| | | CGCAGCCATTTTCGTTAGTGACGTATGCAAACGGCAGGGG |
| | | GAATCCACCATCAAAGAATGCCTTGAGAACAGTTACACAG |
| | | GCTCCTGCAACAGTGGCATGTCCAGCACCATAAGCCGGAT |
| | | GCATCGGGCTTCCCTCTGGGAATGCCATTGGCATGAGAAG |
| | | GTTGCCTTCGCTCAGGCCGCCATCAGGATTGAAATCTGCA |
| | | CTACGCGGAGCACCGTTGTCGCTCAACTTATTTTGCTTCT |
| | | TGTTATGGTAATACACTCGATCCAACAGGTCAGAATCCAG |
| | | GGAATCTACCAATTTAGCAGCTCCGGAAAATTCGGGTGCT |
| | | TTGGTTTTGTAGTATCGGTCGACACGACCGCCGACAGTCT |
| | | CCGGGCGTAGACGGCGATGGACGTTGAACTTCTGGAAGCG |
| | | CACTGCCTTGAGAGCGCGAGTGGCCACCTCAGTAACGAGA |
| | | GAGAGAATATGCGGTCCGCCAAACGTGGCAAATGCCTGCT |
| | | GCTTATCTATATCATTGTCGAGCTGGAACGGGACTCCGGA |
| | | ATCGAACGGTGCACCAATGTCGAGCAGAATGATGCAGGCG |
| | | TTAAGATAGGCCTGGTACAGAGCGTCGAAATGGACGTATG |
| | | TCGCCAGATCACGCGGTGTGTGAATGAAGCGGAAACCAGG |
| | | GTCTGACTCGTATCGCTCACGCCCGGTGACATCCGCAGCG |
| | | TTCTGCACGTCAAGCCAGGATCCAAAGGTGGTCATGTAGT |
| | | CAACCTTGGGGGATGCCACGCGTACCCGCTGGTCCATGCG |
| | | CATACCACCGTACTGGATGAAACCATCGCTGACCTCGTTT |
| | | CCGTTTGCGATTCCTGTGGTACCGACCAAGAGGAACTGGG |
| | | ACAAATATGGGCCTACATCATCTCCAGGAGTGATGCCCCT |
| | | GAAGACATTGGAGGCATCGATGGGGCCTCTGAGACGGGAG |
| | | CGTTCGGCAGATGTGAGATGGGTGGGAGGGTTTTCCTTGA |
| | | ATTTAATCCATTGAGTGTCGTTGATACTCTTGGCAGCGGC |
| | | TGCGACATCTTCGTCGCAATGGAATGTTGGGAAGGGTACA |
| | | TCCCTGAGCAGGGACATCCAATAACATTCGGTGACTTCAG |
| | | TGACCAACTCCTCGCTGTCTAGCTCTGGTGCGGGCGGCAT |
| | | AGTGACGGCCTGTGCATCAGGCCCTTCGAGATCGAATGTG |
| | | CGTCCAGCACCCATACTTTCCCAGGCACGAACTCCCGACT |
| | | GCTCTGGGTTTGTGGCGATGCCGGATGTGAATCTGGGGGT |
| | | GACATTCTGGGGGCCGAGTGGGAGAGCTGCGAAATCGCGG |
| | | GCATCTCCGCTGTCAATAGCGCGTACAAATTGGTCATAAT |
| | | CGAGGGCAGATTGAACCAGACCTGTTTCGTTGATATGGGG |
| | | GAGACCTTTCGTGAAGTTAGCCAAGAGAGATCGTGTACCA |
| | | TCTGGGTTTCTGAAGCGAGCCTCTGAATTGTTGGAGATGT |
| | | GAGTTGGAGAACCACGGGAGCGAGCAAGCTCGGCGGCAGA |
| | | GAGGCGAATCTTGAAGGAGGCCTCTGCGCGATTGGGATTC |
| | | TGTGTGTCGGGGGTTGCCAT |
| 75 | Asparagopsis | TCTCAGAAGAAGATCGGCAACAAGATCGACAACATTAGGC |
| | armata Ireland | ACCCATGCCTCCGAAGCTCTGCTGCATGGTCAGTATTTTG |
| | 3 - Mbb2- | TCTCCAGATATGCCCCACGCAAGTTACTCCGTATTTCAGT |
| | 2_like coding | TTTTCGCATGCTTTTCCTCACAGCTGCTCCCATTGAGTGT |
| | sequence | GGTCCGCAGAAAAACACTCCGATTTCTGCGTGACTTGGTG |
| | | ACTTTTGGGCAATTTTGGCGAATATTTCGTCCCAGGGCGG |
| | | TCTTCCAATGTTGCTTGTAGTAATAAAGGTCTCAGCACTT |
| | | TCAAAGTCTTCACTTTCCTTGCCTGTAATATATCGGTGAA |
| | | GACGGAGTTTTGTGCCGTCCTCTAGAGGAAAGAGTTCTTC |
| | | TCGTAACCATTGGTCATCTACGGTGTGACGTGTTGTCCAG |
| | | ATAAAGTCAACGTCTGGGATTTTCTTTCCGCATGCGCAGT |
| | | TTGGGTTACACTTCCGATCATGAAATAGATTCAGGGACCC |
| | | AATACTCCGATACAGACGAGCACTCATGAAAATAAGCATA |
| | | ATTGGCAGCAATACAATGAAGTGAAGAAAAACTAATGTTC |
| | | CTGACATTTCCGTCCCAGTCCAAGATAGTACACCACTGAC |
| | | GCTGCACAGAATACTGAGCGGGACGAAAACGAAAAAGTCC |
| | | ATAAACTGCCAAGAAGAACGGAAGAAGCGGGGTCCCATGA |
| | | AGCTAATTTCCAAGAGTATGGTCAAAGTTAGAGTGACTGC |
| | | ATGCAAAACACTCAACACTGCATCGGCACCTATAACCCAT |
| | | TTGGCACCTATGCGGCTGTCCCCAAAGTTGAATTCACCAA |
| | | TGTTGAAGATTGATACAATGCACACAAGAACAATGCGTGC |
| | | AATTAGAGAGAGTTGAAGTGCCAGAGTTACCCGAGTGGTA |
| | | TGAAGAAAAGAGAATAATATTGAACGTCTCTCATAGAGAC |
| | | GGGCAATTTTTTGGCGAGCCATCTTACGTTTCCTGGCGGC |
| | | AAGTTCCTTTTCAAGTCTCATTTCTTCACTGAAATCTGAT |
| | | GAATAATCTTCTTCATCGTCGCATGTAGACATGCTGCTCA |
| | | CTGAAGATTCTTCTAATTGATTTTCGACATCCAATATCCG |
| | | GAGACCATCTCCAATATTTGGGCAGCTCCTCGATGATACA |
| | | CATTGTTCACTTGTTCTTGAAGTGGAAACATCGAGTTCTT |
| | | CGGAATTTTGGGCCGTCAAGTTCATCATGTCAGTAATATA |
| | | CTGCTTCCTTTGTTCTTCAGATTCCGGTTCATCGGAGCTG |
| | | TTTGCCAAATCGATATCATATACAGCAGAAATAGTTTCCG |
| | | AGATACGCTGCTCAATATGCTTCATTTCAGACAAATTGGT |
| | | TTTCGAAAAATCAGTAGAAGGGCAGGTTTTGTTCTTGTTC |
| | | AAGAAATGCAAACTTTTGGAGATGGCTGCGAAGGGCGTTG |
| | | AACCAACTCCGCCAGATATGAGGACCACTCTTTCATACGA |
| | | AGAGACATGTTGAGCGGGAGCGCCAAATGGACCACGGACT |
| | | TGAACTAGCAATGGGTTCGGATCTTCTCCTTCCATACGGT |
| | | TTTGAAATGCCCCTCGAAGCTCCTTGGTCCAATTCCCGAG |
| | | TGATTTGATGAAAAAGCACATGCATTTTTCATGGGGTGCA |
| | | CTAGCAATTGTGAAGGGATGCCACTCCCTACTCAAGGATG |
| | | GTACACGGATCTCTGCATACTGACCAGCGCGATATGTGAA |
| | | GGGTTTCGGAATACGAAGTTCCAAAATGTCTTTTCCTTTG |
| | | AGAGCTGAATGCTCGGAAGTGAGCTTCAGCTGAACGGTGG |
| | | AGATTTTGAAGCGACGGGCGATTCTGTCCAGAATGTAGAT |
| | | CACGAGAGGTAGAGCAACCCACTTGTAAGTCTCCGGAACA |
| | | CGACGGTACATACCATGGAAGATGAGAAGAGGAAAGAACA |
| | | GCAGTGCTCCAACCATGTGGGAAATGTAGAAGAGCCGGAA |
| | | ATATTTTTTTCTGTAGAATGGTAGAGCGAACATGAACATG |
| | | ATTATGAAGATAAGTAACAATACGACGCCTGTGACAGCGC |
| | | TCATGGTGAAATCCCAAAGTCCTGCACTCCATTTATCGTA |
| | | AGCAATGATCCATGTGAAATGGAACGGGACATGAATGAAG |
| | | AGAGATGCGGCTATCGTATATCCAACAAGGATATGGAAGG |
| | | CGGGGAAGCTTTTGTCAACAGGAAGAATCTTGTGCAGGGG |
| | | AGTATCACGGATGAATGTGAAGAAGAGGCGAGATGCAAGT |
| | | AGAATGACAAGTGCTGTGTTGAGGTTGAGTGTGTAACCTG |
| | | CTCCGCGAGCAATAGAAATGTACCAGCGGAGATTTGTGTC |
| | | GGGAGATTGATTGTGGAACTCATCGTGAGCTCCCCATACG |
| | | AACATGAGAGAGAGTGCAGTTATGTATGTGATAACTGTGA |
| | | GAAGCTCGAAGGCGTGAGTGGAGAGATAGGACTCGAGGCG |
| | | AGCACTCCAAAGACTTGGGGACCATTTTCTTCTGAAGGGA |
| | | AATGCTGTGTTCAT |
| 76 | Asparagopsis | ATGCCCTCTCCATCCTCATCCACATGTCCAAAGTCCACTC |
| | armata Ireland | AAGATCTCCTCACCGCTCGACTCGAGTCCCACCTCTCCAC |
| | 3 - Mbb2_like | ACATGCCTTCGAACTTCTCGCTCTCGTCGTGTATGCCACC |
| | coding | GCACTCCTCCTCATGTTCCTATGGGGAGCCCATGACGAAT |
| | sequence | TCCATCACCACACTTCCGATGATAACATGCGATGGTACAT |
| | | CTCCATCGCCCGCGCAGCAGGTTACACCCTAAATCTCAA |
| | | TACTGCATTGGTCATTCTTCTCGCTGCCAGACTCATTCCT |
| | | CTTGACAAAAGCTTCCCAGCCTTCCATATTATTTTTTCAA |
| | | CTTATATTCGCGAGACCCCATTGCATCTAATCGTCGCTTA |
| | | CGCTATTGCCATCTCCCTCGTCCTCCATGTCGCTTTCCAT |
| | | TTCGCGTGGATTATTGCCTACGATAAATGGGAACCAGGCC |
| | | TTTGGGGATACACTATGAGCGCATCGACAGGATCTGTCCT |
| | | CCTCATTGTGTTTCTAGTCATGTTCATAACTGCCCTCCCA |
| | | ACATTTCGGAAGAAACGCTTCCGATTCTTCTATCTCACAC |
| | | ATATGTTCGGAGTGATACTCTTCTTCCCTCTACTAATCTT |
| | | TCATGGAATGTACAACCGCGTTCCAGAAACTTACAAATGG |
| | | ATCACCGCCCCACTCATCATCTACATTATTGATAGACTTC |
| | | TTCGTCGTTTCAAAGTGTCCACAGTTCCCTTGCAGCTCAC |
| | | CAGCGATCATTCTTCTCTCAAGAGTGCTGACATTCTCGAA |
| | | CTCCGAGTCCCAAAGCCCTTCCATTACAGAGCGGGGCAAT |
| | | ATGCAGAGGTCTGTGTCCCTTCGATCAGTTCAGAGTGGCA |
| | | TCCATTCACAATCGCAAGCGCACCCCACGAAGAAACCATG |
| | | ACCTTCTTCATCAAAACTCTTGGCAATTGGACTCAAGCAC |
| | | TGCGCCACCAATTCCAAACACGCATGGATTCAGACGACTG |
| | | CAAACCACTTACGGTACAGATCCGCGGACCGTACGGCGCT |
| | | CCCGCTCAGCACGTCGGCAGATATCAACGGGTAGTGTTGG |
| | | TGTCAGGAGGTGTTGGCGCCACCCCTTTTGCCGCCATCTC |
| | | GAAACATCTGCACCACCTGAATGTCGCTGAACATCACAAA |
| | | AAGACGCTCGATGTTGACATTCCATCTAAGCATACAATAC |
| | | AACTCGGGAACCGGATCAGAGAAACGATCTCCAATATGTA |
| | | CGGCTTGGGGTTGGGTGAAAGCGGCTGCGCAGAGGATTCC |
| | | AACCAACAAAGAAGACAGCATCTGGCGGATATGCTGAACA |
| | | TGTCCCCGGATGGAGCGGTGCAGAAATTAGGAAATGAAAT |
| | | CTCTCATCCGTCTCCGACAGGCTCTTCTGATGTGAGTGAT |
| | | ACGTGCAGTTCGGAAAATGTCGATCTCTCGTTTTCATCAA |
| | | GTATTGGGAGTCAAATGGCCACGCTGGGAACATACGAGAA |
| | | AGAGTGCAGCTCTAGAAAAGGCGGGAGAGAAGATAAGATG |
| | | GGGCGATCTGCGTACTCGAAGCATAATTTGATGTTTCTGA |
| | | ATGGAAAGCGCTCTAGACTTCTTTCCTTACTTCACACGAC |
| | | TCGAATAACATTGCTGCTGCTGTTCACACTAATTGCGCGA |
| | | TTAGTGCTGACGTGTGTTGTATCTATCTTCGATCTTGGTC |
| | | ATTTCAGCACAACCAGCGGACACATAGAGGCTAAGTGGGT |
| | | TATCGTGACAAACGTCGTTCTGGGACTAATTGTTGGATTG |
| | | ACTCTATTCACAATAATTGTGCTGGAAGTGAGTTTTATGG |
| | | GGACGCGATTCTTCCACAGGAATTGGCGATGCGTTGACTT |
| | | TTTGGTGTTCGTTCCTCTGTCATTTCTGTGCAATCTATAC |
| | | GGAATTGTAACGTGGGCTGGACGAGACCAATCGTCCTTCC |
| | | TGACATTTGTCGACTATATTATTATCACACCGATTATCTT |
| | | CCTACTTCTCTGTCATCGATTATACCGAAGCATTGGGACG |
| | | CGAAACCTTTTCGACGATAACGGGAGTTGCCGGGGGTCAG |
| | | AACACAGCGTTCCGGAAGTGGACTTTATCTGGACGGTCGC |
| | | GCACGAGGGGGACGACGAGTGGCTTCGCGAAGATCTGTAC |
| | | ACGCTTGCGACGGGGACGGAGCTGCGACTGCATCGGTACG |
| | | TGACACGGGAGAAGGAAGTGGAGGTGGAGGGAGGGGAGGA |
| | | GTTTATCACATCTGCTAGGGCGGGGCGTCCGAAGTGGGAT |
| | | GAAATATTTTTGAAGATCGCGGAGGAGTCGCGGAGCTGCA |
| | | GTGAGATCGGGGTGTTCTTCTGTGGACCCAGTGGGATGGG |
| | | GAAGCAGGTTCGGGAGAGTTTGCGGCGGGCGGAAGTTTTC |
| | | AGTAGTTTGAGGGGATCATACTTGAGGAAAACGGGTGGAG |
| | | GGGAGGTTGGGGACGAGGTAAGATTACTGAAGCGGTTTGG |
| | | AAGCAATGTTCGATTCGTATTTCGGGAGGAGAACTTTAGC |
| | | TGA |
| 77 | Asparagopsis | ATGCCTCCCCCAACTCGTCGTCAATCCGCTCTCGTCACTC |
| | armata Ireland | GCATTGCAGCTGCAGATGAAGCCGCAAACCGTTCACATCC |
| | 3 - Mbb3_like | TCCTCATCTAAGAAATGGCGATGAGACGCTGTACAAGACT |
| | coding | ACCTCAAATGCTCTCTCCTACCTCGCGACCTTCACAAAGG |
| | sequence | GTCTCCCTCATCATCCCGACGGTCGTCTAGTCTCCGCACA |
| | | GCATTTTCAACTCTTCGCCCGTGCTGTCCAAACAGGAACT |
| | | CCACAGCACATTGCAGATATTCCCCTCGGACCTCCATCCA |
| | | CCTTTTCCACATTCCGCTCCGGAATTGCAAAGGATGCCCC |
| | | ACTCCGCGCTTGGGAATCCATGGCCGCTGGAAATACATTC |
| | | GACCTTCAAGGGCCTGATCCCCAATCAGTCACAATACCCC |
| | | CATGTCCACGTCTCGAATCCAATGAGCTCGTCACAGAGAT |
| | | GACAGAGCTTTACTACATGGCACTCCTCAGGGATGTCCCA |
| | | TTCTCTCATTTCAAAAGTCATCACCTCGTCGCCGCTGCCG |
| | | TCGACGATATCAATCACACCCATTGGATTCAATCCCATAA |
| | | ATGTTGCATCCCAGAACTGGAACGTCACAGACACAGAGAA |
| | | CCCTTCTCATCCTCTAATGTCTTCCGTGGCACCACACCAG |
| | | GCGAGCGCAATGGTCCTTACATCTCACAATTCCTCATTGT |
| | | TGGAACCAAAGGGCTCGGAGAAATCCACGACGAGGCAGAT |
| | | GGCTACATCCAATACGGTGCCATCCGAATCGATCAGAGAG |
| | | TTCGATCAGTCGAGACAGAAATCGACTACATGACCACCTG |
| | | GGCCGCCTTCCTTGACGTCCAGAATGCCGCAGATTTGCGC |
| | | AGACATTCTACATATGCCAAATCAAGCTCCTATCGCTTCC |
| | | TGTACACACCACGCGACCTCGCTACCTACGTTCATCATGA |
| | | CTCGCTCTACCAAGCCTATCTCAACGCATGCATCATCCTC |
| | | CTCGACATGAAAGTCCCATTCGACTCCGGTCTCCCCTTCA |
| | | CCACGGAAGACCACATCGACAAACAACAGGGCTTTGTAAA |
| | | CTTTGGAGGCCCCCATATCCTCACACTCGTCACAGAGGTT |
| | | GCTACCCGAGCCCTCAAGGCAGTCCGTTTCCAAAAATACG |
| | | CCGTCCACAGACGTCTTCGCCCAGAGGCTGTCGGCGGCCT |
| | | CTTAGAACAATACAGACGATACGGTGGCGGATATTCAGAG |
| | | CTAGCAAATACTATTCGTCCCATACGCCATCTCGCCCGTT |
| | | CTCTAAGTTCAAACCTCATGTCGCTCGTCGCAGCAGCGAA |
| | | CGGCCATCAGAACGCAACAATGAGCGACAACGGATTTCCA |
| | | CGCTCCCATGATACAGGCTCCGGTGAGACTCATTTGCTGC |
| | | CAATGGCCTATCCGGAGGGAAGTCCGATGCACCCTAGCTA |
| | | TGGTGCCGGGCATGCCACAGTGGCCGGGGCGTGCGTAACG |
| | | ATATTGAAAGGCTTTTTCGATCACACCTACAAGCTCCCGT |
| | | TCGCCTATGTCTCATCCTGCAATGGGAAGATGCTGAAGAA |
| | | GGCAAAGGTCGCGAAGAAACTTTCGGTGGAAGGAGAGCTG |
| | | AATAAACTGGCGTCTAATATCAGTATTGGACGTAGCTGGG |
| | | CTGGCGTACACTACTACTCCGACTATGTGGAGAGTATTCG |
| | | ATTGGGAGAGGAAGTGGCGATTGGAATCCTGAAAGAGCAA |
| | | AAGTTAACTTATTCGGAAAAATTCTCAATGACAATTCCCA |
| | | AATTTGATGGCACAACCATCAAAATATAG |
| 78 | Asparagopsis | TTACTTTGAGTTTGAAATCTTGATAGTCGTACCATCAAAC |
| | armata Ireland | TTGGGAATAGTCATGGAGAACTCCTCACCGTACGTCAGAC |
| | 3 - Mbb4- | TCTGTTCTTCTAGAATACCAATTGCAACCTCTTCACCCAA |
| | 2_like coding | CCTTGCGCTCTCTATGTAATCCGAGTAATAGTGCACACCA |
| | sequence | GCCCAATTCCTACCAATACTGATGTTGGCACAAAGCTTGT |
| | | TGAGCTCATGCTCCACTGTGAGCGGTTCATTGATTTCCAC |
| | | CTCCTCAAGATTCTGGCCGTCTGTGCTGGGCACATAACAG |
| | | AATTCAAGCTCGTGAGTGTGGTCGAAGAACGCCTTTAGGA |
| | | TCGTCACACATGCACCCGCAACAGCAGCATGCCCTGCACC |
| | | ATAGCTGGGATGCATTGGGCTACCCTCCGGGAATGCCATC |
| | | GGGAGAAGATAAGTTTCTGCGCTTTCGCAGCCCTCAGGAT |
| | | TATAATCGCCGCTTCGGGCACCACCGTTGTCGGAAAGTTC |
| | | ATTCTGCGCGGCGTTGTGCTGCGAGATTCGTGTCATAAGG |
| | | TCATTGTCGATCTCAGTGGCGATTTTCTGGATGGGCTCAA |
| | | AGGGCTCTGCTCCCTCCGTCTTTCGGTATCGCTCGATAAG |
| | | ACCGCCGACAGCCTCGGGGCGTAGACGGCGGTGGGTACCA |
| | | AACTTCTGGAAGCGCACAGCCTTCAATGCCCTAGTTGCGA |
| | | CCTCTGTGACAAGCGAGAGGATATGGGGACCTCCAAAGGT |
| | | AGCGAATCCCTGTTGTTTATCGATGTCATCATCTTCCACA |
| | | AAAGGGATTTCTGGATCGAAAGGGGCCTTGGTGTCGAGAA |
| | | GAATGAGACATGCGTTCAGGTATGCCTGGTAAAGTGCATC |
| | | GAAATGGACGTAGGTGGCAAGATCGCGCGGTGTAGTAATG |
| | | AAACGGAATGGAGTGTCGTTGTCGTACGTTTCACGTCCTC |
| | | GTACATCACCTGCGTTCTGCACGTCGAGCCAGGATGCCCA |
| | | GGTGGTCATGTAGTCCCTATTTGGGGTTGCCACACGAACA |
| | | CGTTGGTCCATACGCATACCTCCGTATTGAATGAATCCAT |
| | | CTGAAATTTCCTGAATGTTCGCGATACCCTTTGTGCCAAC |
| | | GAGAAGGAACTGAGAGAGATAGGGACCGATTTGTTCACCA |
| | | GGGGTCACGCCGCGGAAGATGTTTGTTTTCCTGAATGGAC |
| | | CTCGCAAACGTGCAATTTCTGCATCAGACAGATCGCAGGG |
| | | GCGTTGCCCGGGCTGCTCTTGGAACTGCACCCAGCGCGTT |
| | | CGGCTGATCGAAGCGGCTGCGGCAGCGGTAGTGCCATCCC |
| | | TTTCGAACTCTGTGAACGGGACGTCGCGAAGAAGGGCCAT |
| | | CCAGTAGCACTCGGTGATCTCCGTCAAGAGTTCTTCACTA |
| | | TCGATTTTTGGGGCAGGCGGCATGGTGACTGCCTGAGCGT |
| | | CCGGTCCCTCCAGATCGTATGTATTACCAGCTGCCATGGA |
| | | TTCCCACGCTCTAGTGTCGGCTATGGGATGGTCGGAGCCC |
| | | TCTGAAGCGATGGCGGATATGAAACGGGGGTCTTCATCGA |
| | | TACCTAGGGGGAGACTTGTAATATCTTCTTGAGCGCCTGT |
| | | TTCGATGGCGCGTACAAAGGCTGCGAAGTGATCTGGAGAG |
| | | GAGGAGAGGCCGGTATCCTTTTCGTGTGGAAGACCTTTCG |
| | | TGTAGGAGGCGAGGTGGGTGTCGTGGTAGCGATGCTCATC |
| | | GCCGTTTGCAAGGTGGGTGGGGAGGGGGCGTGAAGCAGCG |
| | | AGATCTGCGGCTTCACTACGGACTCTCAAGGCGGATGCAT |
| | | GGCGTTCTTCGGACAT |
| 79 | Asparagopsis | TCAGTTGGAGATCGTAATGCACGTTCCATCAAACTTGGGG |
| | armata Ireland | AGGGTCATGGAGAACTTCTCACCATACGTCAAGCTCTGTT |
| | 3 - Mbb4_like | CTTGCAAAATACCGATGGCAACCTCTTCACCCAACTTTGC |
| | coding | ACTCTCTTCGTAATCCGAGTAGTAGTGCACACCGGCCCAG |
| | sequence | TTTCTTCCAATACTGATGTTGGCACACAACTTGTTCAGCT |
| | | CGTGTTCAACTGTGAGCGGTTCACTGATGTTAACCTTCTC |
| | | TAGCTTCCTGCCGTCTGTGCTGGGTTCAAAGCAGAACTTT |
| | | AACTTGTATTTGTGATCAAAGAAGGCCTTGAGGATCGTGA |
| | | CACAGGCGCCAGCGACAGCGGCGTGTCCAGCACCGTAGCT |
| | | GGGATGCATTGGGCTTCCCTCCGGGAACGCCATTGGAAGA |
| | | AGGTAGGTTTCTGCGCTTGCGCCACCCTCGGGGTTGAAAT |
| | | CATTTCTTCGTGCTTTACCGTTGTCGGAAAACTCATTCTG |
| | | CTTGGCGTTGTGACGCGAGATGCGCGTCATAAGCTCATTG |
| | | TCGATCTCGGTAGCGAGCTTCTTAGCGGCTGCGAATGGCG |
| | | CTGCGCCGTTCGTCTTGCGGTAGCGCTCGATGAGTCCGCC |
| | | GACAGCTTCGGGGCGTAGGCGACGATGCGTACCGAACTTC |
| | | TGGAAGCGCACAGCCTTAAGTGCTCTGGTTGCAACCTCTG |
| | | TGACAAGAGAAAGGATGTGAGGCCCGCCAAAGGTGGCAAA |
| | | TCCCTGTTGTTTATCGATGTCGTCGTCCTGCACAAAAGGA |
| | | AGGCCATGATCGAAGGGAGCCTTTATGTCGAGAAGAATGA |
| | | GACAGGCATTGAGGTATGCTTGATAGAGGGCGTCGAAATG |
| | | GACGTAGGTTGCGAGATCGCGCGGGGTGGTAATGAAACGG |
| | | TAATCAGTATCTTTGTCGTATGTTTCACGTGAGCGGACGT |
| | | CCCCTGCATTCTGTACGTCGAGCCAGGAGGCCCAAGTAGT |
| | | CATATAGTCGCGGTTGGGAGTCGCAAAGCGAACGCGCTGG |
| | | TCCATTCGCATACCTCCGTATTGAATGAAACCTTCTTCTG |
| | | GCTGGTGCATTTTCCCGATACCCTGTGTACCGACAAGCAA |
| | | GAACTGGGAGAGGTAGGGGCCTACCTGTTCACCAGGCATG |
| | | TCGCCGCGGAAGACGTTTGATTTGTTGAAGGGGCCGCGCA |
| | | GACGTTGGATTTCAGCATTTGTGAGATCGGATGGGCGCTG |
| | | GCCGGGCTGCTCCTGAAACTGTACCCAGCGTGTACGACTA |
| | | ATCGATGCGGCTGCGGCAGCAACTGTTCCGTTTCGATGAA |
| | | ATTCTGAGAAGGGCACGTCGCGAAGAAGGGCCATCCAATA |
| | | GCATTCTGTGATCTCTGTCAAGAGCTCTTCGCTGTCGATT |
| | | TTAGGTGCAGGAGGCATGGTGACTGCCTGTGCATCAGGCC |
| | | CTTCGAGATCGAAGGTATTGCCTGCGGCCATGGATTCCCA |
| | | GGCGCGAGTGTCAGCGATGGGGAGGTCGAGTGCTGGGGAT |
| | | GCGATGCCGGAGATGAAACGAGGTGTTTCATCAATACCTA |
| | | GGCGGAGGCTCCTGATGTCTTCCTGTGCGCCTGTGTTGAT |
| | | AGCACGCACAAAGGCAGTGAAGTCATCTGTAGATGAGAGA |
| | | AGGCCTGTCTTTTTGTCGTGTGGTAAACCCTTTGTGTAGG |
| | | ATGCGATGTATGAATCTGAGAAACGGGTCTCATCGCCATT |
| | | GGGGGTGTGGGTAGGGAGTTTGCGGGAGGCTGCTAGTTTT |
| | | GCGGCATCTGTGCGTACTTGTAGTGCGGCTGCTTGACGTG |
| | | TGGTAGACAT |
| 80 | Asparagopsis | CTAGATACGGATGGTCGAACCGTCAAACTTGTTCAGCGTC |
| | taxiformis | ATTGAGAAGTTTTCACTGAACGTCAGCTTTTGCTCCTGCA |
| | Azores | GAATACCAATGGCAATCTCCTCACCAATGCGAATACTCTC |
| | Mbb1_like | GATGTAATCAGTGAAGTAATGCACGCCAGCCCAATTGCGA |
| | coding | CCAATCGAAATATTGGAGCAAATCTTATTCAACTCGCCGT |
| | sequence | CGACGGTCAACGGCTCGTCAATTTCCACTGCCTTAAGACC |
| | | TGTTCCATTCTCGTCGGTGATGTAGCAGAACGGCAACCTG |
| | | TAACCACCATCGAAGAACGCCTTCAACACTGTGACACAAG |
| | | CGCCTGCAACAGTCGCATGTCCAGCGCCGTACGCGGGATG |
| | | CATAGGACTTCCCTCTGGGAACGCCATGGGCATCAGCAGA |
| | | TTACCTTTGCTCACATCTCCGTCGGGGTTGAAGTCGTTTG |
| | | CACGCGGGTTTCCTCGATCGCTCAACAAGTTTTGTTTCTT |
| | | GTTATGATCGTGCACCTTCTGCAACAACTTCTTGTCCAGC |
| | | GCTTCGGAGAGCTTGGCAGCGCCAGCAAATTCAGGTGCCT |
| | | TTGTTGCGCAGTATCGATCGACTCGGGCACCAATGGCCTC |
| | | CGGACGCAGACGACGATGGACGTTGAATTTCTGGAAGCGA |
| | | ACAGCCTTGAGGGCTCGAGTGGCCACTTCGGTAACTAGAG |
| | | AGAGGATGTGCGGACCGCCGAAAGTGGCAAATCCTTGCTG |
| | | CTTATCAATGTCGTTATCGAGCTGGAAGGGAATTCCAGAG |
| | | TCGAATGGCGCACCGATGTCGAGCAAGATGATGCAAGCGT |
| | | TGAGGTAGGCCTGATACAAGGCGTCGAAATGCACGTAAGT |
| | | GGCCAGATCACGAGGAGTGTGAATGAAGCGGAAACGGGGT |
| | | TCCTCTTCATTGTACAATTCGCGACCGGAGACGTTGGCGG |
| | | CATTTTGCACGTCGAGATAGGCGCCGAATGTGGTCATGTA |
| | | ATCAATGTGTGACTTGGCGACACGCACGCGCTGGTCCATA |
| | | CGCATGCCACCGTACTGGATGAATCCGTCGCCAACTTCAT |
| | | TGCCATTGGCAATACCGGTGGTACCGACAAGGAGGAACTG |
| | | TGACAGATAGGGTCCGACTTCGTCTCCCGGTGTGATACCT |
| | | CTGAAGACGTTGGCAGTGGTGATGGGGCCTCTGAGACGGG |
| | | AACGCTCAGCAGCTGTGAGATGTGCTGGTGGGTTGTTCTT |
| | | GAATTGAATCCATTGGGTGTTATTGATGGACTCTGCAGCT |
| | | GACTGAATATGGGGATCACTGTCAAATGTCGGGAAGGGAA |
| | | CGTCTCTCAGCAGAGACATCCAGTAGCATTCTGTGACTTC |
| | | AGTAACCAATTCATCGCTGTCCAGCTCGGGCGCGGGCGGC |
| | | ATAGTGACAGCCTGAGCATCGGGACCTTCGAGATCGAAGA |
| | | CAAGACCAGCACCGCCACTTTCCCAGGCACGGGTTCCGAC |
| | | CTCTGGGTCGCTAGCGATACCGGAGGTGAATCTGGGCTCA |
| | | ACGCCCTGCGGACCGAGCGGGAGGTCTGCGAAATCACGAG |
| | | GATCACCGCTGTCAATTGCGCGAACAAAGCTATCGTAGTC |
| | | GATTGCGGACACCACTAGAGCGGTTTCCTTGGTATGCGGG |
| | | AGACCCTTGGTGAAATTGGCGAGCAGCGAACGGGTACCAT |
| | | CAGGGTTTCTGAAGCGACTCTCCGAGTTGTTGGACAGATG |
| | | GGTTGGGGAGCCACGAGCGCGTGCCAGCTCAGCTGCTGAT |
| | | ACGCGGACTTTGAAGGCCACTTCTGCACGATTGGGATTCT |
| | | GTGTGTCGGTCAT |
| 81 | Asparagopsis | ATGGATACCGTTTCCCCCGGAGACTCTCGCCAGAAAGCAC |
| | taxiformis | AGGGAAAGATGCGCCCCATGCGCTCCTTCGCCATCCTCTC |
| | Azores- | GGCTAATCTCGAAGCTTTCCTCTCTACACATGCCTTCTCC |
| | Mbb2_like | CTCCTCTTCCTCATCTTCTACCTCACCTGTACAGCCCTCA |
| | coding | TGTTCGTTTGGGGCTTCACCACACAGTGGAAGAAGACCGA |
| | sequence | CAACGAAGCCATGATCTATTTCATTTCCATTGCCAGAGGT |
| | | GCTGGTTACAGTCTCAATCTCAACACAAGCCTCGTCATCC |
| | | TCATTGCCGCTAGACTCACTTTCACTCTTATCCGCGAAAC |
| | | TCCCCTCCAATACATTCTCCCACTCGATAAGATTTTCCCG |
| | | CTTTTCCACATGTTTGTCGGCTACATGATTGCATTCCTCG |
| | | TTCTCATCCACGGCACTTTCCATATGACATGGATCATCCG |
| | | CTTCCAAGGATGGAACTCGGGTCTCTGGGGTTTCACAATG |
| | | AGTTTCGTCACCGGTTTCATCATCCTTCCAGTCTTTGTCG |
| | | TCATGCTCATCACTTCCCTTCCTCACTTCCGCAAGAACAA |
| | | GTTCCGCCTCTTCTACCTCTTCCATCTTTACGGCGCCGCT |
| | | CTCTTCTTCTCCATCCTCATCTTCCACGGTATGTATTTTG |
| | | AGAGGCCAGAGACCTACAAATGGATTACACCCTCCCTCAT |
| | | CATCTATCTCCTCGATCGTTTCATCAGACGTATGAAGATC |
| | | AGCAGTCATGATCTGGACATTGCCCTGGAAAAATCCATCC |
| | | TCAAGGACGGTGATATTCTCGAACTCCGTGTTCCCAAACC |
| | | CTTCGACTATCGTCCCGGCCATTATGCAGAGGTTATGGTT |
| | | CCGTCCATTAACAAGGAATGGCATCCATTCTCCATCGCTA |
| | | GTGCACCACACGAAGAAACCATGTGCTTCTACATCAAGGT |
| | | GCTCGGTGATTGGACCACCGAAGTTCGTAATGCTTTCAAG |
| | | GCTCGTCTCGATGGTACAATCACTGAACCACTCAAAGTAC |
| | | GTGTTCGTGGCCCGTACGGAGCTCCATGCCAGGATGTCGA |
| | | CGGTTTCAAGCGTGTCGTCCTCGTCTCCGGAGGTGTTGGT |
| | | GCCACACCATTTTCCGCCATTGTTAAGGAGCTGCACTACC |
| | | TCAAGACGCAGGCTAAGAACAAGAACGATGGCCCGGGACG |
| | | CATTGCCCGCGACATTGATGTTCGCATTCGTGACAGTATC |
| | | GCTAATCTCTACGATGTCGAAGTGCAGGACATGGAGAAGG |
| | | CTACTCACATTGCGCAGCAGAAGAGCATCTACGTTACTGA |
| | | AATGCTTGAGCTCACCGCCAGGAGGACCTCTTCCCTGCAC |
| | | CGAGTGAGGTCCAGTGTCATGGATCGCATGCATGGATCGT |
| | | CATCTACAAAGACAGTGCTCAGTACCGATGCTGCTATTCC |
| | | CAGCTTGGAGGATGGCCCTGTTGGTGGTGGCCAACCGATG |
| | | AGCAGGATGCAGCGTTTGTCATCTTCATTGAGCAAGGCTG |
| | | GAAACATTCGACGAGTGATGAGCACCTTGTCGGAGAACCC |
| | | GGTTACTTGGGATCAAGTGATGTCTGCGCAGAACGATCCA |
| | | TTGACCAACACGACCGATCTTCGTCGTCAGATGGCGCGCC |
| | | TTTACGATAAGAGAGGAATGCTCTTGGCGTTCATGCACAC |
| | | AGCTCGCTTCACCTTTTTCCTGCTCCTCACTTTGATTGTA |
| | | CGAATTATTATTATTTCCGTTGCCGCCACTCTGGATTCGC |
| | | CCTATTTCCAGCTCAACGATCAATTTGATGGCTACACGCG |
| | | CTTGTGGCCTGTCGTTGTCGACGCCCTCTTCTCCGTTCCA |
| | | GTTGCTGCTGCACTGGTGGTCTCCATTACACTTGAGATTA |
| | | GTTTCATGCGCATGCGATACTTTGACACTGCGGCGAGAAC |
| | | CTTTGACGCCTTAGTCCTGATGCCAGCGGCCGTCATCGGC |
| | | TGTGTGCTTGCCATCCTTACTTGGGCACGACAGGACCCGG |
| | | AATCTGTTCTTGTCAAGGTTATTGAATATGTACTGTTCAT |
| | | CCCGCTATTGTTCCTAGGTCTGAGCTGGCGCTTGTACCGC |
| | | ATCACACGGTCGAGGAGTCTTCTCTCGGATCCGGCATGCC |
| | | GATGCGATAACTCGGACCACATTCCCGATGTTGACTTTAT |
| | | CTGGTCGTTCCCCTACGAGCAGAGTGATCAATGGTTGCAG |
| | | GATGAGCTCGGTCCGCTAGCAAAGGGTACTGAACTACGAC |
| | | TTCATAGGTATGTGACTAGGGAGAAGGAACTTGATGTGGA |
| | | GGCGGCGAAATCATTTATTACCAGTGCCAATACTGGACGT |
| | | CCAAAGTGGGATGATATTTTCCAGACAATCGCCGAGGAAA |
| | | CGCCAAGTAATGGAGCAATTGGTGTGTTCTTCTGTGGACC |
| | | CCACGTGATGGGCAATCAAGTTCGCGATGCCATGCTCAAG |
| | | GTCGAGGTGCTGAGCAATTTGCGAGCTGCTTACTTGCGAA |
| | | ATACGCCCGACGAGGTGTTGATGACCGACTTGAATTTGCC |
| | | AAATGCTGAAGCAGTGGAGAGGCTGCGATCGTTGGGATGC |
| | | AGCGTTCATTTCGTCTATAGGGAAGAAAACTTTGGTTGA |
| 82 | Asparagopsis | ATGACGCCGCCAACACGCCGCCAAACAGCGCTCGTCAAGC |
| | taxiformis | GCATTGCCGCCGCAGACCTGGCCGCCAATCGTCACCATCC |
| | Azores | GCATCATGTGCGCAATGCCGACGAATCCACCTACAAATTG |
| | Mbb3_like | CCAAACCATGTCTGTTCCTACATATCCGCTTTCACCAAGG |
| | coding | GTTTGCCCCATCATCTCGACGGCCGCCTCGTTTCCCCACA |
| | sequence | CCACTTTCAACTCTTCGTCCACGCCGTGCAAACTGGCCAT |
| | | GTCACAGACATTGCCGCCATTCCACTCGGACCCCCACACT |
| | | CCTCCTCCTTCCGTTCCGGCATCGCCAAGGGCGCCGCCGT |
| | | GCGCGCATGGGAGTCCATGGCCGCGGGTAACACCTTTGAT |
| | | TTGCAAGGGCCTGATGCACAGGCCGTTACCATGCCCCCTT |
| | | GTCCGCGTCTCAATTCACACGAGCTCGTCACAGAGATGAC |
| | | AGAGCTTTATTATATGGCACTGTTGCGCGACGTGCCGTTT |
| | | ACGCATTTCAACAAACATCATCTCGTTCAGTCCGCTGTGC |
| | | ACGACATGAATCATACCCATTGGACTCGGTCCAACAGATG |
| | | CTGTTTACCTCGCCCCGAGCGCCGTCGACATCGCTCCAAA |
| | | TTCACCACTGATAACATCTTCCGCGGTGTAACGCCCGGTG |
| | | ACATAACCGGTCCCTACATTTCCCAGTTTCTCCTTGTCGG |
| | | CACAAAGGGTCTCGGCGATGCACACGAAGAAACAGAAGGC |
| | | TACGTTCAATACGGCGCCACAAGAATCGACCAGCGAGTGC |
| | | GCACTGTCGAACCCAAAATTGACTACATGACGACATGGGC |
| | | CGCTTATCTCGACGTGCAGAATGGTGCCGACTTACGCCGT |
| | | CACTCCACCTATGCCAAACCACCGTCCTACCGCTTCATTA |
| | | GCACCCCGCGCGACCTCGCAACCTATGTGCATTACGACGC |
| | | GTTGTATCAAGCGTATCTCAACGCGTGCATCATCCTGCTC |
| | | GACATCAACGCGCCATACGATGGCGGACTGCCCTTCACCG |
| | | CCGACGATGCCGTCGACAAGCAGCAGGGCTTTGCGACATT |
| | | TGGCGCGCCACATGTGCTGACACTTGTGACAGAAGTGGCG |
| | | ACACGCGCGCTCAAAGCTGTGCGCTTTCAAAAGTATGCCG |
| | | TGCATCGGCGTCTGCGGCCGGAAGCTGTCGGCGGTCTATT |
| | | GGAGCAGTATAGACGCTACGGTGGTGGACATTCGGATCTG |
| | | GCATACATCATTCGTCCGATTCGTCATCTCGCCGACTCGC |
| | | TCAGTTCCGATCTGATGTTCATGGTCGCCAAGGAGAACGG |
| | | ACGCCAGAATAGCTCGATTACGGATAACGGCCATCCGCGC |
| | | TCGCATGACGAAGGTTCGGATGATACGCATCTGCTTTCCA |
| | | TGGCGTATGCGGAGGGAAGCCCGATGCATCCTAGTTATGG |
| | | CGCGGGACATGCAACGGTTGCGGGCGCTTGTGTAACAATC |
| | | CTGAAAGCGTTCTTCGATCATACGTTCAAACTGCCGTTTG |
| | | CGTACGTGTCGTCGAGCGATGGGAGGAAATTGAAGACGGT |
| | | CAAGTTGAGCAGGAAATTGACCGTGGAGGATGAGTTGAAC |
| | | AAGTTGGCAGCGAATATAAGCATTGGACGCAGTTGGGCTG |
| | | GCGTCCATTACTATTCGGATTATGTTGAGAGCATTCGGTT |
| | | GGGCGAGGAGGTGGCCATCGGGATGCTCAAGGAGCAGAAG |
| | | TTAACTTATTCGGAGAAATTCACGATGACAATACCCAAAT |
| | | TTGACGGCTCCGTCATTGAAATTTAG |
| 83 | Asparagopsis | ATGGCAGAGGAACGTCGCCAAAACGCTCTAGACATCCGCA |
| | taxiformis | TCCAAGCCGCCAGGCTTGCGAAGAAACGTGACCATCCAAC |
| | Azores | CCATGAGGCAAACGGCGATGAGGATCGTTACCCAGAAACA |
| | Mbb4_like | CTCATCGGCTCCTTCACTAAAGGTCTTCCACATGAGAAAG |
| | coding | AAACCGGTCTCCTCTCCAACCCCGCCGATTTCGCCGACTT |
| | sequence | TGTTCGCGCTATCAACACAGGCGCGATCAAAGACATAAGA |
| | | CGCCTCAAACTTGGAATCGATGAGGATCCTCGCTTCATAT |
| | | CTGGTATTGCCTCCAAGGGCGACAAGCATCCTTTCGCCGA |
| | | TACGCGCGCCTGGGAATCCATGGCCGCCGGTCTCACGTAC |
| | | GATCTAGAAGGCCCTGATGCCCAAGCTGTCACCATGCCGC |
| | | CGGCGCCGAAACTCGACAGTGATGAACTCGTCACAGAGAT |
| | | TACGGAGAGCTATTGGATGGCTCTTATTCGTGATGTGCCG |
| | | TTCACAGAGTTTGAACGTGATGGAAATACAGCCGCAGCCG |
| | | CAGCTTCCATCAGTCGCACTCGTTGGGTACAGTTCAATGA |
| | | GCAGCCTTCACAGCGTCCGTCAACATTGACCGATGAAGAG |
| | | ATTGCTAGATTGAGGGGTCCGTATACCAAGAAAAATGTAT |
| | | TCCGTGGTGTGACGATCGGTGAGAACGTCGGCCCGTATCT |
| | | GTCTCAGTTCTTACTGGTAGGCACGAAAGGTATCGGCGAT |
| | | GTACAGCAAATTTCGGATGGTTACATTCAGTACGGAAGTA |
| | | TGCGAATGGATCAGCGTGTCCGCGTCGCAGCGCCGAAACG |
| | | CGATTATATGACAACTTGGGCATCTTGGCTAGATGTGCAG |
| | | AACGCTGGTGATTTACGTGGACGTGAAATTTACGATGATG |
| | | ATACACCGTTCCGCTTCATTACAACCCCACGCGACCTTGC |
| | | GACATGGGTTCATTTCGACGCGTTATACCAGGCATATTTG |
| | | AATGCTTGCATCATCCTTCTCGACATCAAGGCGCCGTTCG |
| | | ATCCACACATCCCGTTCCAGGCTGATGACGATGTAGATAA |
| | | GCAGCAAGGATTTGCAACGTTTGGCGGCCCGCACATCCTA |
| | | TCATTGTGCACGGAAGTGGCTACGAGAGCACTCAAGGCAG |
| | | TTCGCTTCCAAAAGTACAACGTGCATCGTCGTCTTCGTCC |
| | | GGAGGCTATCGGCGGGCTTGTGGAACGCTTCAAGAAGACA |
| | | AACGGAGATGCCAAGTTTGCCCCAGTGAAGAAGCTGGTGA |
| | | ATGACCTGGACGGCGATATGCTGCGACGCATTGAGCAGCA |
| | | CAACTGTGAGCAGAATAAGCTTTCAGATGATGGACATGCA |
| | | CGCCGAGACGACTATTCACCGGAGGGTGAGAGTAACCAGA |
| | | CGTACCTTCTGCCCATGGCGTTCCCGGAGGGGAGCCCGAT |
| | | GCACCCGTCATATGGTGCAGGTCACGCCACAGTTGCCGGT |
| | | GCGTGTGTGACGGTGCTCAAGGCATTCTTCGACCATGAAT |
| | | ACGAGCTGGACTTCTGCTACGTACCGACCACGGATGGCAA |
| | | GCGTCTGGAGAAGGTAAATATCAATGAAAAATTGACAGTG |
| | | GAAGGCGAACTGAACAAGCTGTGCGCTAATATCTGTATTG |
| | | GTCGAAACTGGGCTGGCGTGCATTACTACTCGGACTACTT |
| | | TGAGAGTATCAAAGTTGGTGAGGAGATTGCAATTGGTATT |
| | | TTGCAAGAACAGAAGCTTACGTATGGTGAAGACTTTTTCA |
| | | TGACGCTGTCCAAGTTTGACGGTGAAAAGATCCGTATCTA |
| | | A |
| 84 | Asparagopsis | TCAAGCAACAGGATCATCACTTCGCAACACCTCCACAGTG |
| | taxiformis | CGCTTGTCAACATTCAGCTCGTGCTTTGGCTGGCTACGAA |
| | Azores | TGACATTAAGGTGGCCATCGAAGCCGACGTAAGTGAAGCT |
| | Mbb_like | ATGTTGCTCCGCGAAGGTCAGCACCTGCTCCTCAAGAATT |
| | coding | CCCCTCGCGACTTCCTCTCCGAGGATCAGAGACTCAATGT |
| | sequence | TGTCGGACGACCAGTGTACTCCTGCAAGTGATCGACCAAA |
| | | TGCGACATTGTTTGCAATCTTGTTCAGCTCACCATCAACA |
| | | GTCAGCGTCATACCGTCCGGAAGACACACGGGCTGCAGAC |
| | | ATCCATGCGCGTCGGCCTCAAAGATCTCATTCGGCAAGGG |
| | | GGTAAACTCCACATTAGACGGCTTCGCGTCCTTCACAGCG |
| | | CACTTTCCGTCGAACTGCACCGTGTTAAAGAATGCCTTCA |
| | | GAATAATCGTGCAAGCGCCAGCCACTGTCCCGTGTCCTGC |
| | | CCCATACGAGGGATGCATAGGAGAACCTTCGGGGAACGCC |
| | | ATTGGCAGTAAGTACGACGGAGAAGACGCGCTGCGGTCTT |
| | | TCACACGGTCAGCAAACGTCTGGTTCTGCTGCATGTTCTT |
| | | GTCAGCAACAAGGTCGAGGAGACCGCATCCGTCGAGCCGA |
| | | GCGACAAGAGCGCGAACTTCCTCGCCGAAGTCGCATCGCT |
| | | CACTCTTCTGCGCGTGGATCAGACCGCCGACTGCTTCAGG |
| | | CCGGGCCCGGAGATGAATCTGGAACTTCTGGTAACGGACT |
| | | GCCTTGAGAGCCCTGGTCGCAACCTCACAGACAAGGGACA |
| | | GGATGTGCGGACCGCCGAAAGTCGCGAACGCTGTTTGTTT |
| | | GTCGTGCTCGTCGTCATCGCGGAACGGTAAGCCCGTATCG |
| | | ACCCCGACACCGTCCGCCAGCAGAGCAAAGCATGCATTCA |
| | | AGTAGGCTTCGTACAACGCATCGTAATGGACATATGTCGA |
| | | AAGGTCGCGTCCATCTGCAATAAACCGGAATATCGGACTC |
| | | CCATCAGGAGCACACTTGTAGTCGGGGAATTCACCCTGCC |
| | | TGACATCCGCCCCGTCCTGAACATCGATGAAAGCCTCCCA |
| | | CGTTGTCATCCAATCTTTTCTTGGCTCAATTGATCTCACT |
| | | CGCAAGTCAAGCCCAAGCGAACCGTACATAACCTGTCCTG |
| | | TTGCAGTACCATCCGTTCCAAGGCCCTGGCTACCGCGGAT |
| | | CAGATATTGTGATATGTACGGACCATCTCGGACCCCAGCT |
| | | CCGTAGCCTCTGAAAATATTTCCAGTAGTCAAGTTAAGCT |
| | | TCCCCTCCCGTCTGAACCTCTCCTCATCAGTGAGCCCCTT |
| | | CCCGAACTCTTCAGAGACCCACGGCAACGCGCTCAAGGTA |
| | | GCAATCGCATTATCAAAGAAGGCCTTCGTATCGGGAGAGT |
| | | CCCACAAACTGAAAGGAATATCCCGCGCAAGCGCCATGAA |
| | | GTAAAGCTCTGCAATTTCAAACTCAAGCTCCGCGGACGCG |
| | | AGCTTCGGCGCGGGCGGCATAGTGACAGCCTGCGCGTCGG |
| | | GTCCTTCCAGGTCAAACGCGAGGCCTGCGCCTGCGCTTTC |
| | | CCACGCGCGCGCCTTGACCTGCGACTCGAACGGCAGGCCG |
| | | GGCGCGCCGTTTGCAAGCGTGGATTGCCAGCGGCCGGGAG |
| | | TGTCTTTTGGCAGCTTCGGGCCCAGCCGGATTTTCTTGAA |
| | | GTCCGCGGGATCGCCCGTGGCCAGCGACGCGCGGAAGGGG |
| | | TCGAAAAGATCACGGCTGAAAATCTGACCGGTCTCGGTGT |
| | | TGTGCCGCAGACCCTTCGTGAAAGTGCCGATGAGGGTTTC |
| | | CTTGAAATCTGACTCATCTCCGTTGCTCTTGTGTGGGCGA |
| | | ACTCCGCGGTTGAATGCCAGGTTTGCTGCGTTGATCCTGA |
| | | CCTGCTTGGCGTCTTTGCGACGCTGCTCCTGGTCGGAGTT |
| | | GTCCTGGGCCATTTTCACGGAGGCGGCGGAAGACTGTCGA |
| | | CGGAAAGCATTGAAAGGGGAATGCTTCGTGGTGATTGATG |
| | | CGCCTCTGATACGTGCAACCCCAACTGGCGCAGAGGCTAC |
| | | GAAAGCGTTATGGAACAT |
| 85 | cDNA for | ATGACCGACACACAGAATCCCAATCGTGCAGAAGTGGCCT |
| | Asparagopsis | TCAAAGTCCGCGTATCAGCAGCTGAGCTGGCACGCGCTCG |
| | taxiformis | TGGCTCCCCAACCCATCTGTCCAACAACTCGGAGAGTCGC |
| | Mbb1 | TTCAGAAACCCTGATGGTACCCGTTCGCTGCTCGCCAATT |
| | | TCACCAAGGGTCTCCCGCATACCAAGGAAACCGCTCTAGT |
| | | GGTGTCCGCAATCGACTACGATAGCTTTGTTCGCGCAATT |
| | | GACAGCGGTGATCCTCGTGATTTCGCAGACCTCCCGCTCG |
| | | GTCCGCAGGGCGTTGAGCCCAGATTCACCTCCGGTATCGC |
| | | TAGCGACCCAGAGGTCGGAACCCGTGCCTGGGAAAGTGGC |
| | | GGTGCTGGTCTTGTCTTCGATCTCGAAGGTCCCGATGCTC |
| | | AGGCTGTCACTATGCCGCCCGCGCCCGAGCTGGACAGCGA |
| | | TGAATTGGTTACTGAAGTCACAGAATGCTACTGGATGTCT |
| | | CTGCTGAGAGACGTTCCCTTCCCGACATTTGACAGTGATC |
| | | CCCATATTCAGTCAGCTGCAGAGTCCATCAATAACACCCA |
| | | ATGGATTCAATTCAAGAACAACCCACCAGCACATCTCACA |
| | | GCTGCTGAGCGTTCCCGTCTCAGAGGCCCCATCACCACTG |
| | | CCAACGTCTTCAGAGGTATCACACCGGGAGACGAAGTCGG |
| | | ACCCTATCTGTCACAGTTCCTCCTTGTCGGTACCACCGGT |
| | | ATTGCCAATGGCAATGAAGTTGGCGACGGATTCATCCAGT |
| | | ACGGTGGCATGCGTATGGACCAGCGCGTGCGTGTCGCCAA |
| | | GTCACACATTGATTACATGACCACATTCGGCGCCTATCTC |
| | | GACGTGCAAAATGCCGCCAACGTCTCCGGTCGCGAATTGT |
| | | ACAATGAAGAGGAACCCCGTTTCCGCTTCATTCACACTCC |
| | | TCGTGATCTGGCCACTTACGTGCATTTCGACGCCTTGTAT |
| | | CAGGCCTACCTCAACGCTTGCATCATCTTGCTCGACATCG |
| | | GTGCGCCATTCGACTCTGGAATTCCCTTCCAGCTCGATAA |
| | | CGACATTGATAAGCAGCAAGGATTTGCCACTTTCGGCGGT |
| | | CCGCACATCCTCTCTCTAGTTACCGAAGTGGCCACTCGAG |
| | | CCCTCAAGGCTGTTCGCTTCCAGAAATTCAACGTCCATCG |
| | | TCGTCTGCGTCCGGAGGCCATTGGTGCCCGAGTCGATCGA |
| | | TACTGCGCAACAAAGGCACCTGAATTTGCTGGCGCTGCCA |
| | | AGCTCTCCGAAGCGCTGGACAAGAAGTTGTTGCAGAAGGT |
| | | GCACGATCATAACAAGAAACAAAACTTGTTGAGCGATCGA |
| | | GGAAACCCGCGTGCAAACGACTTCAACCCCGACGGAGATG |
| | | TGAGCAAAGGTAATCTGCTGATGCCCATGGCGTTCCCAGA |
| | | GGGAAGTCCTATGCATCCCGCGTACGGCGCTGGACATGCG |
| | | ACTGTTGCAGGCGCTTGTGTCACAGTGTTGAAGGCGTTCT |
| | | TCGATGGTGGTTACAGGTTGCCGTTCTGCTACATCACCGA |
| | | CGAGAATGGAACAGGTCTTAAGGCAGTGGAAATTGACGAG |
| | | CCGTTGACCGTCGACGGCGAGTTGAATAAGATTTGCTCCA |
| | | ATATTTCGATTGGTCGCAATTGGGCTGGCGTGCATTACTT |
| | | CACTGATTACATCGAGAGTATTCGCATTGGTGAGGAGATT |
| | | GCCATTGGTATTCTGCAGGAGCAAAAGCTGACGTTCAGTG |
| | | AAAACTTCTCAATGACGCTGAACAAGTTTGACGGTTCGAC |
| | | CATCCGTATCTAG |
| 86 | CDNA for | ATGTCCGAAGAACGCCATGCATCCGCCTTGAGAGTCCGTA |
| | Asparagopsis | GTGAAGCCGCAGATCTCGCTGCTTCACGCCCCCTCCCCAC |
| | armata Mbb4- | CCACCTTGCAAACGGCGATGAGCATCGCTACCACGACACC |
| | 2 | CACCTCGCCTCCTACACGAAAGGTCTTCCACACGAAAAGG |
| | | ATACCGGCCTCTCCTCCTCTCCAGATCACTTCGCAGCCTT |
| | | TGTACGCGCCATCGAAACAGGCGCTCAAGAAGATATTACA |
| | | AGTCTCCCCCTAGGTATCGATGAAGACCCCCGTTTCATAT |
| | | CCGCCATCGCTTCAGAGGGCTCCGACCATCCCATAGCCGA |
| | | CACTAGAGCGTGGGAATCCATGGCAGCTGGTAATACATAC |
| | | GATCTGGAGGGACCGGACGCTCAGGCAGTCACCATGCCGC |
| | | CTGCCCCAAAAATCGATAGTGAAGAACTCTTGACGGAGAT |
| | | CACCGAGTGCTACTGGATGGCCCTTCTTCGCGACGTCCCG |
| | | TTCACAGAGTTCGAAAGGGATGGCACTACCGCTGCCGCAG |
| | | CCGCTTCGATCAGCCGAACGCGCTGGGTGCAGTTCCAAGA |
| | | GCAGCCCGGGCAACGCCCCTGCGATCTGTCTGATGCAGAA |
| | | ATTGCACGTTTGCGAGGTCCATTCAGGAAAACAAACATCT |
| | | TCCGCGGCGTGACCCCTGGTGAACAAATCGGTCCCTATCT |
| | | CTCTCAGTTCCTTCTCGTTGGCACAAAGGGTATCGCGAAC |
| | | ATTCAGGAAATTTCAGATGGATTCATTCAATACGGAGGTA |
| | | TGCGTATGGACCAACGTGTTCGTGTGGCAACCCCAAATAG |
| | | GGACTACATGACCACCTGGGCATCCTGGCTCGACGTGCAG |
| | | AACGCAGGTGATGTACGAGGACGTGAAACGTACGACAACG |
| | | ACACTCCATTCCGTTTCATTACTACACCGCGCGATCTTGC |
| | | CACCTACGTCCATTTCGATGCACTTTACCAGGCATACCTG |
| | | AACGCATGTCTCATTCTTCTCGACACCAAGGCCCCTTTCG |
| | | ATCCAGAAATCCCTTTTGTGGAAGATGATGACATCGATAA |
| | | ACAACAGGGATTCGCTACCTTTGGAGGTCCCCATATCCTC |
| | | TCGCTTGTCACAGAGGTCGCAACTAGGGCATTGAAGGCTG |
| | | TGCGCTTCCAGAAGTTTGGTACCCACCGCCGTCTACGCCC |
| | | CGAGGCTGTCGGCGGTCTTATCGAGCGATACCGAAAGACG |
| | | GAGGGAGCAGAGCCCTTTGAGCCCATCCAGAAAATCGCCA |
| | | CTGAGATCGACAATGACCTTATGACACGAATCTCGCAGCA |
| | | CAACGCCGCGCAGAATGAACTTTCCGACAACGGTGGTGCC |
| | | CGAAGCGGCGATTATAATCCTGAGGGCTGCGAAAGCGCAG |
| | | AAACTTATCTTCTCCCGATGGCATTCCCGGAGGGTAGCCC |
| | | AATGCATCCCAGCTATGGTGCAGGGCATGCTGCTGTTGCG |
| | | GGTGCATGTGTGACGATCCTAAAGGCGTTCTTCGACCACA |
| | | CTCACGAGCTTGAATTCTGTTATGTGCCCAGCACAGACGG |
| | | CCAGAATCTTGAGGAGGTGGAAATCAATGAACCGCTCACA |
| | | GTGGAGCATGAGCTCAACAAGCTTTGTGCCAACATCAGTA |
| | | TTGGTAGGAATTGGGCTGGTGTGCACTATTACTCGGATTA |
| | | CATAGAGAGCGCAAGGTTGGGTGAAGAGGTTGCAATTGGT |
| | | ATTCTAGAAGAACAGAGTCTGACGTACGGTGAGGAGTTCT |
| | | CCATGACTATTCCCAAGTTTGATGGTACGACTATCAAGAT |
| | | TTCAAACTCAAAGTAA |
| 87 | CDNA for | ATGGCAACCCCGTGCTTCGCCCAGGCGCCGGCCCCCACCC |
| | Chondrus | TACGACGATCGCCGCGCCGTCTCCCCCTCACCCCGACCTC |
| | crispus Mbb3 | CTTCCTGCCGAGGCCGTTCCTGCCAAAGCCCTTCCCCCGC |
| | (XM_0057193 | TCCCCCCGCCACCCGCATCCGCGCCTCAACGCCGCCTCCG |
| | 78.1) | CGCGCACCTCCGCCGCCCAACCCCCCGACCGCGCAGCCAC |
| | | GCCGTGCGCTACCCGCCAGCAGCAAGCGCTGGAGCTGCGC |
| | | CGCGCGGCGGCGCAGCTCGCGCACGACCGTGCGCACCCCG |
| | | CGCACGTGTCGAACGGGTCGGAGGAGCTTCACCTGCACCC |
| | | GAAGACGGGCGCGCCGCTGCTGCTGGCCAACTTCACGAAG |
| | | GGGCTGCCGCACGAGCGCGACACGGGGCTGGCAGCCGACG |
| | | CCGCGCACTACGACCTCTTCGTGGCGGCGGTCCATTCCGG |
| | | CTTGCCGGACGACATCCGCGCCATCCCGCTCGGCCCGCAG |
| | | GCCGCGCCCGGCGTCCGCCTCCCGCGCTCGCTCCGCTTCC |
| | | GCTCCGGCATCGCACGCAGCTCGCACCTCTTCCCGCACAC |
| | | TGACGTCCGCGCCTGGGAGAGCATGGCCGCGGGGAACGCG |
| | | TTCGACCTGCAGGGGCCGGACGCGCAGGCCGTCACCATGC |
| | | CGCCCGCGCCCGCCCTCGCCTCCCCCGAGCTCGCGCTGGA |
| | | GATGACGGAGAACTACTGGATGGCCCTGCTGCGCGACGTC |
| | | CGCTTCTCGCAGTTTGCCGCGGACCCCACCGTCGCCGCCG |
| | | CCATGGAGAGCATGAACGCTACCGAGTGGGTCGCCCTCGC |
| | | GTGCGGCGACGCGTCGGCCGTCGCGGAGGAGGAGCGCAAG |
| | | CGCCTGCGCGGGCCGTTTACCGCGCAGAACGTGTTCCGTG |
| | | GGAATACGCAAGGCGATTGCAAGGGGCCCTATATCTCCCA |
| | | GTTTCTGCTGTCTGGAAGCGCGGGGCTGGGCCAAGTGAAC |
| | | AGCGGCGCGGATGGATACATCCAGTACGGAGCAATGCGTA |
| | | TGGATCAGCGCGTTCGTGTCGCCCTCGAGGGGCTTGACTA |
| | | TATGACCACGTGGGAGAGCTGGCTGGATGTGCAGAACGGG |
| | | GCCGACTTGCGGGGAAGGGAGTTGTATGAGGACGGCGAAC |
| | | GCTTTAGATTTATTTGCACCCCGAGGGACTTGGCCACTTA |
| | | TGTCCATTATGATGCCCTGTATCAGGCTTACTTGAACGCC |
| | | TGCATTATCATGCTCAACAAGAAGATCCCCTTTGACAAGG |
| | | GCCTGCCGTTCCAGAAGGATGACGACATCGACAAGCAGCA |
| | | GGGCTTCGCCTTATTCGGCCCGCCGCACATTCTCACCCTG |
| | | TGCACGGAGGTCGCCACCAGGGCCCTCAAGGGCATCCGCT |
| | | TCCAAAAGTACAACGTCCACCGCCGTCTGCGTCCGGAGGC |
| | | TGTCGGCGGCCGCGTGGAGCGCTACCACCACAACTGCGAG |
| | | GACCCGCTCTTCGCCGACGTCAAGCCGCTGTACGACGCCC |
| | | TGGATAAGGACATGCTCGAGCGTGTTGCCGCGCACAACGC |
| | | CGAGCAGAACGCCAAGGACGACTTTGGGAACAGCCGCTTC |
| | | GAAGACTATTCCCCGACGGGTAGCTCCGGCAGGACCTATC |
| | | TGCTGCCCATGGCGTTTCCGGAGGGCAGCCCCATGCACCC |
| | | GGCGTACGGCGCAGGGCATGCGGCAGTGGCCGGCGCGTGC |
| | | ACCACTATCCTCAAGGCGTTCTTCGACCACGAGCACGAGC |
| | | TTGACTTTGCGTACGTGCCCACCGCGGATGGCAGCAAATT |
| | | GGAGGATGTTGTTGACTCTTTGGGGGAGAAGCTGACCGTC |
| | | GAGGGAGAGCTCAACAAGGTCTGCTCGAATATCAGCGTCG |
| | | GGCGCAACTGGGCTGGTGTGCACTACTTTACAGACTATCG |
| | | CGAGTCCATCATCCTTGGGGAAAAGATTGCCCTCGGCCTG |
| | | CTGGAGGAGCAGAAGCTTACGTATGCTGAGGAGTTCTCCA |
| | | TGACGATTCCTCTCTTTGATGGTTCCACTGTCACCATTTA |
| | | G |
| 88 | Maize RUBS | ATGGCGCCCACCGTGATGATGGCCTCGTCGGCAACCGCCG |
| | chloroplast | TCGCCCCGTTCCAGGGTCTCAAGTCCGCCGCCAGCCTCCC |
| | signal peptide | CGTCGCCCGCCGCAGCACCAGGAGCCTCGGCAACGTCAGC |
| | | AACGGCGGAAGGATCCGGTGCATG |
| 89 | Maize SOD3 | ATGGCTCTCCGCACCCTGGCATCGAAGAAGGTCCTATCCT |
| | mitochondrial | TCCCGTTCGGCGGCGCGGGCCGGCCGTTGGCGGCGGCGGC |
| | signal peptide | GTCTGCGAGGGGGGTG |
| 90 | Peroxisome | CCGCTGGTGGTGCAACTATACAGTGTCTTG |
| 91 | signal peptide | CTGCAGTGCAGCGTGACCCGGTCGTGCCCCTCTCTAGAGA |
| | from Zea mays | TAATGAGCATTGCATGTCTAAGTTATAAAAAATTACCACA |
| | SK1 gene | TATTTTTTTTGTCACACTTGTTTGAAGTGCAGTTTATCTA |
| | Maize Ubi-1 | TCTTTATACATATATTTAAACTTTACTCTACGAATAATAT |
| | promoter | AATCTATAGTACTACAATAATATCAGTGTTTTAGAGAATC |
| | including an | ATATAAATGAACAGTTAGACATGGTCTAAAGGACAATTGA |
| | intron | GTATTTTGACAACAGGACTCTACAGTTTTATCTTTTTAGT |
| | | GTGCATGTGTTCTCCTTTTTTTTTGCAAATAGCTTCACCT |
| | | ATATAATACTTCATCCATTTTATTAGTACATCCATTTAGG |
| | | GTTTAGGGTTAATGGTTTTTATAGACTAATTTTTTTAGTA |
| | | CATCTATTTTATTCTATTTTAGCCTCTAAATTAAGAAAAC |
| | | TAAAACTCTATTTTAGTTTTTTTATTTAATAATTTAGATA |
| | | TAAAATAGAATAAAATAAAGTGACTAAAAATTAAACAAAT |
| | | ACCCTTTAAGAAATTAAAAAAACTAAGGAAACATTTTTCT |
| | | TGTTTCGAGTAGATAATGCCAGCCTGTTAAACGCCGTCGA |
| | | CGAGTCTAACGGACACCAACCAGCGAACCAGCAGCGTCGC |
| | | GTCGGGCCAAGCGAAGCAGACGGCACGGCATCTCTGTCGC |
| | | TGCCTCTGGACCCCTCTCGAGAGTTCCGCTCCACCGTTGG |
| | | ACTTGCTCCGCTGTCGGCATCCAGAAATTGCGTGGCGGAG |
| | | CGGCAGACGTGAGCCGGCACGGCAGGCGGCCTCCTCCTCC |
| | | TCTCACGGCACCGGCAGCTACGGGGGATTCCTTTCCCACC |
| | | GCTCCTTCGCTTTCCCTTCCTCGCCCGCCGTAATAAATAG |
| | | ACACCCCCTCCACACCCTCTTTCCCCAACCTCGTGTTGTT |
| | | CGGAGCGCACACACACACAACCAGATCTCCCCCAAATCCA |
| | | CCCGTCGGCACCTCCGCTTCAAGGTACGCCGCTCGTCCTC |
| | | CCCCCCCCCCCCTCTCTACCTTCTCTAGATCGGCGTTCCG |
| | | GTCCATGGTTAGGGCCCGGTAGTTCTACTTCTGTTCATGT |
| | | TTGTGTTAGATCCGTGTTTGTGTTAGATCCGTGCTGCTAG |
| | | CGTTCGTACACGGATGCGACCTGTACGTCAGACACGTTCT |
| | | GATTGCTAACTTGCCAGTGTTTCTCTTTGGGGAATCCTGG |
| | | GATGGCTCTAGCCGTTCCGCAGACGGGATCGATTTCATGA |
| | | TTTTTTTTGTTTCGTTGCATAGGGTTTGGTTTGCCCTTTT |
| | | CCTTTATTTCAATATATGCCGTGCACTTGTTTGTCGGGTC |
| | | ATCTTTTCATGCTTTTTTTTGTCTTGGTTGTGATGATGTG |
| | | GTCTGGTTGGGCGGTCGTTCTAGATCGGAGTAGAATTCTG |
| | | TTTCAAACTACCTGGTGGATTTATTAATTTTGGATCTGTA |
| | | TGTGTGTGCCATACATATTCATAGTTACGAATTGAAGATG |
| | | ATGGATGGAAATATCGATCTAGGATAGGTATACATGTTGA |
| | | TGCGGGTTTTACTGATGCATATACAGAGATGCTTTTTGTT |
| | | CGCTTGGTTGTGATGATGTGGTGTGGTTGGGCGGTCGTTC |
| | | ATTCGTTCTAGATCGGAGTAGAATACTGTTTCAAACTACC |
| | | TGGTGTATTTATTAATTTTGGAACTGTATGTGTGTGTCAT |
| | | ACATCTTCATAGTTACGAGTTTAAGATGGATGGAAATATC |
| | | GATCTAGGATAGGTATACATGTTGATGTGGGTTTTACTGA |
| | | TGCATATACATGATGGCATATGCAGCATCTATTCATATGC |
| | | TCTAACCTTGAGTACCTATCTATTATAATAAACAAGTATG |
| | | TTTTATAATTATTTTGATCTTGATATACTTGGATGATGGC |
| | | ATATGCAGCAGCTATATGTGGATTTTTTTAGCCCTGCCTT |
| | | CATACGCTATTTATTTGCTTGGTACTGTTTCTTTTGTCGA |
| | | TGCTCACCCTGTTGTTTGGTGTTACTTCTGCAG |
| 92 | enhanced 35S | TGAGACTTTTCAACAAAGGGTAATATCGGGAAACCTCCTC |
| | promoter | GGATTCCATTGCCCAGCTATCTGTCACTTCATCAAAAGGA |
| | | CAGTAGAAAAGGAAGGTGGCACCTACAAATGCCATCATTG |
| | | CGATAAAGGAAAGGCTATCGTTCAAGATGCCTCTGCCGAC |
| | | AGTGGTCCCAAAGATGGACCCCCACCCACGAGGAGCATCG |
| | | TGGAAAAAGAAGACGTTCCAACCACGTCTTCAAAGCAAGT |
| | | GGATTGATGTGAACATGGTGGAGCACGACACTCTCGTCTA |
| | | CTCCAAGAATATCAAAGATACAGTCTCAGAAGACCAAAGG |
| | | GCTATTGAGACTTTTCAACAAAGGGTAATATCGGGAAACC |
| | | TCCTCGGATTCCATTGCCCAGCTATCTGTCACTTCATCAA |
| | | AAGGACAGTAGAAAAGGAAGGTGGCACCTACAAATGCCAT |
| | | CATTGCGATAAAGGAAAGGCTATCGTTCAAGATGCCTCTG |
| | | CCGACAGTGGTCCCAAAGATGGACCCCCACCCACGAGGAG |
| | | CATCGTGGAAAAAGAAGACGTTCCAACCACGTCTTCAAAG |
| | | CAAGTGGATTGATGTGATATCTCCACTGACGTAAGGGATG |
| | | ACGCACAATCCCACTATCCTTCGCAAGACCCTTCCTCTAT |
| | | ATAAGGAAGTTCATTTCATTTGGAGAGGACACGCTGA |
| 93 | Linker 1 | ATTGCTGCAGCGGCCGAATTCAAG |
| 94 | 3X (G4S) | GGCGGAGGTGGTTCTGGCGGAGGTGGTTCTGGCGGAGGTG |
| | linker | GTTCTGGCGGAGGTGGTTCT |
| 95 | 3x HA | TATCCGTACGACGTGCCAGACTATGCGGGCTATCCCTATG |
| | | ACGTCCCGGACTATGCAGGATACCCTTACGATGTTCCTGA |
| | | TTACGCT |
| 96 | NOS | GATCGTTCAAACATTTGGCAATAAAGTTTCTTAAGATTGA |
| | terminator | ATCCTGTTGCCGGTCTTGCGATGATTATCATATAATTTCT |
| | | GTTGAATTACGTTAAGCATGTAATAATTAACATGTAATGC |
| | | ATGACGTTATTTATGAGATGGGTTTTTATGATTAGAGTCC |
| | | CGCAATTATACATTTAATACGCGATAGAAAACAAAATATA |
| | | GCGCGCAAACTAGGATAAATTATCGCGCGCGGTGTCATCT |
| | | ATGTTACTAGATCCGATGATAAGCTGTCAAACATGA |
| 97 | eGFP | ATGGTGAGCAAGGGCGAGGAGCTGTTCACCGGGGTGGTGC |
| | | CCATCCTGGTCGAGCTGGACGGCGACGTAAACGGCCACAA |
| | | GTTCAGCGTGTCCGGCGAGGGCGAGGGCGATGCCACCTAC |
| | | GGCAAGCTGACCCTGAAGTTCATCTGCACCACCGGCAAGC |
| | | TGCCCGTGCCCTGGCCCACCCTCGTGACCACCTTCACCTA |
| | | CGGCGTGCAGTGCTTCAGCCGCTACCCCGACCACATGAAG |
| | | CAGCACGACTTCTTCAAGTCCGCCATGCCCGAAGGCTACG |
| | | TCCAGGAGCGCACCATCTTCTTCAAGGACGACGGCAACTA |
| | | CAAGACCCGCGCCGAGGTGAAGTTCGAGGGCGACACCCTG |
| | | GTGAACCGCATCGAGCTGAAGGGCATCGACTTCAAGGAGG |
| | | ACGGCAACATCCTGGGGCACAAGCTGGAGTACAACTACAA |
| | | CAGCCACAACGTCTATATCATGGCCGACAAGCAGAAGAAC |
| | | GGCATCAAGGTGAACTTCAAGATCCGCCACAACATCGAGG |
| | | ACGGCAGCGTGCAGCTCGCCGACCACTACCAGCAGAACAC |
| | | CCCCATCGGCGACGGCCCCGTGCTGCTGCCCGACAACCAC |
| | | TACCTGAGCACCCAGTCCGCCCTGAGCAAAGACCCCAACG |
| | | AGAAGCGCGATCACATGGTCCTGCTGGAGTTCGTGACCGC |
| | | CGCCGGGATCACTCACGGCATGGACGAGCTGTACAAGTAA |

Example expressions of the inventive concept are set out in the following clauses.

1. A nucleic acid construct for inducing expression of a polypeptide in a plant cell, the nucleic acid construct comprising a coding sequence encoding a polypeptide comprising a vanadate-dependent haloperoxidase (VHPO), or a functional fragment or homologue thereof;
wherein the coding sequence is operably linked to one or more regulatory elements, suitable to drive expression of the polypeptide in a plant cell.
   2. A modified plant cell comprising a nucleic acid coding sequence encoding a polypeptide comprising a vanadate-dependent haloperoxidase (VHPO), or a functional fragment or homologue thereof.
   3. The modified plant cell of clause 2, wherein the modified plant cell comprises the construct of clause 1.
   4. A plant comprising the modified plant cell of clause 2.
   5. The plant of clause 4, wherein the plant expresses VHPO in parts which are edible by livestock, suitably in its leaves or grains.
   6. The plant or plant cell of any of clauses 2 to 5, wherein the plant or plant cell produces a brominated hydrocarbon, suitably bromoform.
   7. The nucleic acid construct, plant cell or plant of any preceding clause, wherein the VHPO polypeptide is derived from a species selected from Asparagopsis spp. (e.g. A. taxiformis, A. armata), Acaryochloris marina, Alaria esculenta, Ascophyllum nodosum, Alteromonas naphthalenivorans; Caulerpa spp. (e.g. Caulerpa taxifolia); Chaetomorpha spp. (e.g. Chaetomorpha linum), Chondrus crispus, Colpomenia sinuosa, Corallina officinalis, Corallina pilulifera, Cystoseira trinodis, Furcellaria spp. Gracilaria spp. (e.g. Gracilaria changii, Gracilaria vermiculophylla); Hormophysa triquetra, Hypnea pannosa, Laminaria spp. (e.g. L. digitata, L. saccharina), Laurencia filiformis, Macrocystis pyrifera, Sargassum flavicans, Zonaria farlowii, Cladophora patentiramea, Dictyota bartayresii, Gigartina spp. (e.g. Gigartina stellata), Oedogonium spp., Padina spp. (e.g. Padina australis), Pterocladia capillacea and Ulva spp. (e.g. U. intestinalis, U. linza, U. lactuca).
   8. The nucleic acid construct, plant cell or plant of any preceding clause, wherein the VHPO polypeptide is derived from a microalgal or macroalgal species.
   9. The nucleic acid construct, plant cell or plant of clause 8, wherein the microalgal species is a member of the genus Emiliana, Calcidiscus or Chaetocerus.
   10. The nucleic acid construct, plant cell or plant of clause 8, wherein the macroalgal species is a member of the genus Asparagopsis or Chondrus.
   11. The nucleic acid construct, plant cell or plant of any preceding clause, wherein the VHPO polypeptide is derived from Asparagopsis armata or Asparagopsis taxiformis.
   12. The nucleic acid construct, plant cell or plant of clause 11, wherein the VHPO polypeptide is derived from Asparagopsis taxiformis Azores, Asparagopsis armata Azores, or Asparagopsis armata Ireland.
   13. The nucleic acid construct, plant cell or plant of any preceding clause, wherein the VHPO is a vanadate-dependent bromoperoxidase (VBPO).
   14. The nucleic acid construct, plant cell or plant of any preceding clause, wherein the VBPO is selected from Mbb1, Mbb3 and Mbb4 of A. taxiformis.
   15. The nucleic acid construct, plant cell or plant of any preceding clause, wherein the nucleic acid is DNA.
   16. The nucleic acid construct, plant cell or plant of clause 15, wherein the DNA is in the form of a plasmid.
   17. The nucleic acid construct, plant cell or plant of any preceding clause, wherein the coding sequence comprises a sequence at least 80%, at least 85%, at least 90%, at least 95%, at least 97%, at least 99%, or 100% identical to any one of SEQ ID NOs: 47, 49 to 54, 57 to 60, 64 to 67, 71 to 74, 77 to 80, and 82 to 87.
   17a. The nucleic acid construct, plant cell or plant of any preceding clause, wherein the construct comprises a sequence at least 80%, at least 85%, at least 90%, at least 95%, at least 97%, at least 99%, or 100% identical to any one of SEQ ID NOs: 52, 58, or 80.
   18. The nucleic acid construct, plant cell or plant of any preceding clause, wherein the coding sequence comprises a sequence at least 80%, at least 85%, at least 90%, at least 95%, at least 97%, at least 99%, or 100% identical to any one of SEQ ID NOs: 47, 49 and 50 (Mbb1, Mbb3 or Mbb4 of A. taxiformis).
   19. The nucleic acid construct, plant cell or plant of any preceding clause, wherein the coding sequence comprises a sequence at least 80%, at least 85%, at least 90%, at least 95%, at least 97%, at least 99%, or 100% identical to any one of SEQ ID NOs: 54, 57 to 60, 64 to 67, 71 to 74, and 77 to 79 (VHPO of A. armata).
   20. The nucleic acid construct, plant cell or plant of any preceding clause, wherein the polypeptide comprising the VHPO comprises an amino acid sequence at least 80%, at least 85%, at least 90%, at least 95%, at least 97%, at least 99%, or 100% identical to SEQ ID NOs: 1, 3 to 15, 18 to 21, 25 to 28, 32 to 35, 38 to 41, and 43 to 46.
   21. The nucleic acid construct, plant cell or plant of any preceding clause, wherein the polypeptide comprising the VHPO comprises an amino acid sequence at least 80%, at least 85%, at least 90%, at least 95%, at least 97%, at least 99%, or 100% identical to any one of Mbb1, Mbb3 and Mbb4 of A. taxiformis (SEQ ID NOs: 1, 3 and 4).
   22. The nucleic acid construct, plant cell or plant of any preceding clause, wherein the polypeptide comprising the VHPO comprises an amino acid sequence at least 80%, at least 85%, at least 90%, at least 95%, at least 97%, at least 99%, or 100% identical to any one of SEQ ID NOs: 15, 18 to 21, 25 to 28, 32 to 35, and 38 to 40 (VHPO of A. armata).
   23. The nucleic acid construct, plant cell or plant of any preceding clause, wherein the polypeptide comprising the VHPO comprises an amino acid sequence at least 80%, at least 85%, at least 90%, at least 95%, at least 97%, at least 99%, or 100% identical to any one of Chondrus crispus vBPO (SEQ ID NOs: 5 to 7).
   24. The nucleic acid construct, plant cell or plant of any preceding clause, wherein the polypeptide comprising the VHPO comprises an amino acid sequence at least 80%, at least 85%, at least 90%, at least 95%, at least 97%, at least 99%, or 100% identical to any one of Acaryochloris marina vBPO (SEQ ID NO: 8); Alteromonas naphthalenivorans vBPO (SEQ ID NO: 9), Ascophyllum nodosum vBPO (SEQ ID NO: 10), Corallina officinalis vBPO (SEQ ID NO: 11), Corallina pilulifera (SEQ ID NO: 12), Gracilaria changii vBPO (SEQ ID NO: 13), and Laminaria digitata vBPO (SEQ ID NO: 14).
   25. The nucleic acid construct, plant cell or plant of any preceding clause, wherein the plant or plant cell is a terrestrial plant.
   26a. The nucleic acid construct, plant cell or plant of any preceding clause, wherein the plant or plant cell is a monocotyledon.
   26b. The nucleic acid construct, plant cell or plant of clause 26a, wherein the monocotyledon is selected from corn, millet, sorghum or pasture grasses.
   27. The nucleic acid construct, plant cell or plant of any preceding clause, wherein the plant or plant cell is a dicotyledon.
   28. The nucleic acid construct, plant cell or plant of clause 27, wherein the dicotyledon is alfalfa or clover.
   29. The nucleic acid construct, plant cell or plant of any preceding clause, wherein the coding sequence is operably linked to a promoter for driving expression of the polypeptide in a plant cell.
   30. The nucleic acid construct, plant cell or plant of clause 29, wherein the promoter is a constitutive or inducible promoter.
   31. The nucleic acid construct, plant cell or plant of clause 29 or 30, wherein the promoter is selected from AlcR/AlcA (ethanol inducible); GR fusions, GVG, and pOp/LhGR (dexamethasone inducible); XVE/OlexA (beta-estradiol inducible); and heat shock induction.
   32. The nucleic acid construct, plant cell or plant of any preceding clause, wherein the VHPO is monomeric
   33. The nucleic acid construct, plant cell or plant of any preceding clause, wherein the coding sequence is operably linked to a nucleic acid sequence encoding a targeting signal.
   34. The nucleic acid construct, plant cell or plant of any preceding clause, wherein the polypeptide includes a targeting signal.
   35. The nucleic acid construct, plant cell or plant of clause 33 or 34, wherein the targeting signal is a peroxisomal targeting signal, optionally wherein the peroxisomal targeting signal is SEQ ID NO: 90.
   36. The nucleic acid construct, plant cell or plant of clause 33 or 34, wherein the targeting signal is a mitochondrial targeting signal, optionally wherein the mitochondrial targeting signal is SEQ ID NO: 89.
   37. The nucleic acid construct, plant cell or plant of clause 33 or 34, wherein the targeting signal is a chloroplast targeting signal, optionally wherein the chloroplast targeting signal is SEQ ID NO: 88.
   38. An animal feed or animal supplement comprising the modified plant cell or plants of any of clauses 2 to 37.
   39. A method for reducing methane production in an animal, comprising growing or culturing the modified plants or plant cells of any of clauses 2 to 37 under conditions suitable for the production of halogenated hydrocarbons, and feeding an effective amount of the modified plants or plant cells to the animal.
   39b. The method of clause 39, wherein the plant is subjected to stress such as heat shock or drought.
   39c. The method of clause 39, wherein the growing or culturing of the modified plant or plant cells does not include providing substrates or cofactors relevant to the production of a halogenated hydrocarbon.
   39d. The method of clause 39, wherein the growing or culturing of the modified plant or plant cells does include providing substrates or cofactors relevant to the production of a halogenated hydrocarbon.
   39e. The method of clause 39c or clause 39d, wherein the halogenated hydrocarbon is a brominated hydrocarbon.
   39f. The method of clause 39e, wherein the brominated hydrocarbon is bromoform.
   39g. The method of any one of clauses 39c, 39d, 39e, or 39f, wherein the substrates or cofactors relevant to the production of a halogenated hydrocarbon include at least one compound selected from the group consisting of KBr, sodium orthovanadate, pentane-2,4-dione, and H₂O₂.
   40. A method for reducing methane production in an animal comprising administering to the animal an effective amount of modified plant cells, plants, or animal feed of any of clauses 2 to 37.
   41. The method of clause 39 or 40, wherein the animal is a ruminant.
   42. The method of any of clauses 39 to 41, wherein the animal is bovine.
   43. The method of any of clauses 39 to 42, wherein the animal is a cow, sheep or goat.
   44. The method of any of clauses 39 to 43, wherein methane production is reduced.
   45. The method of any of clauses 39 to 44, wherein feed efficiency is increased.
   46. A method of preparing a modified plant cell, comprising:
transforming a plant cell with the nucleic acid construct of any of clauses 1 to 37.
   46b. A method of preparing a modified plant cell comprising:
transfecting a plant cell with a plasmid comprising the nucleic acid construct of any one of clauses 1 to 37.
   47. The method of clause 46 or 46b, wherein the plant cell is transformed by Agrobacterium-mediated infiltration.
   48. The method of clause 47, wherein the plant cell is transformed by particle bombardment.
   48b. The method of clause 46, wherein the plant cell is transformed by using a CRISPR/Cas system, a zinc finger nuclease, a TALEN, a lentivirus, an adenovirus, an AAV, and/or a meganuclease.
   49. A method of producing an animal feed for reduction of methane production in an animal comprising:
growing the modified plant cell or plant of any of clauses 2 to 37; and
processing the modified plant cell or plant into an animal feed.

1. A nucleic acid construct for inducing expression of a polypeptide in a plant cell, the nucleic acid construct comprising a coding sequence encoding a polypeptide comprising a vanadate-dependent haloperoxidase (VHPO), or a functional fragment or homologue thereof;
wherein the coding sequence is operably linked to one or more regulatory elements, suitable to drive expression of the polypeptide in a plant cell.

2. A modified plant cell comprising a nucleic acid coding sequence encoding a polypeptide comprising a vanadate-dependent haloperoxidase (VHPO), or a functional fragment or homologue thereof, wherein the modified plant cell comprises the construct of claim 1.

3. A plant comprising the modified plant cell of claim 2.

4. The modified plant cell of claim 2, wherein the plant cell produces a brominated hydrocarbon, suitably bromoform.

5. The nucleic acid construct of claim 1, wherein the VHPO polypeptide is derived from a species selected from Asparagopsis spp. (e.g. A. taxiformis, A. armata), Acaryochloris marina, Alaria esculenta, Ascophyllum nodosum, Alteromonas naphthalenivorans; Caulerpa spp. (e.g. Caulerpa taxifolia); Chaetomorpha spp. (e.g. Chaetomorpha linum), Chondrus crispus, Colpomenia sinuosa, Corallina officinalis, Corallina pilulifera, Cystoseira trinodis, Furcellaria spp. Gracilaria spp. (e.g. Gracilaria changii, Gracilaria vermiculophylla); Hormophysa triquetra, Hypnea pannosa, Laminaria spp. (e.g. L. digitata, L. saccharina), Laurencia filiformis, Macrocystis pyrifera, Sargassum flavicans, Zonaria farlowii, Cladophora patentiramea, Dictyota bartayresii, Gigartina spp. (e.g. Gigartina stellata), Oedogonium spp., Padina spp. (e.g. Padina australis), Pterocladia capillacea and Ulva spp. (e.g. U. intestinalis, U. linza, U. lactuca).

6. The nucleic acid construct of claim 1, wherein the VHPO polypeptide is derived from a microalgal or macroalgal species.

7. The nucleic acid construct of claim 6, wherein the macroalgal species is a member of the genus Asparagopsis or Chondrus.

8. The nucleic acid construct of claim 1, wherein the VHPO is a vanadate-dependent bromoperoxidase (VBPO).

9. The nucleic acid construct of claim 1, wherein the coding sequence comprises a sequence at least 80%, at least 85%, at least 90%, at least 95%, at least 97%, at least 99%, or 100% identical to any one of SEQ ID NOs: 47, 49 to 54, 57 to 60, 64 to 67, 71 to 74, 77 to 80, and 82 to 87.

10. The nucleic acid construct of claim 1, wherein the coding sequence comprises a sequence at least 80%, at least 85%, at least 90%, at least 95%, at least 97%, at least 99%, or 100% identical to any one of SEQ ID NOs: 47, 49 and 50 (Mbb1, Mbb3 or Mbb4 of A. taxiformis).

11. The nucleic acid construct of claim 1, wherein the coding sequence comprises a sequence at least 80%, at least 85%, at least 90%, at least 95%, at least 97%, at least 99%, or 100% identical to any one of SEQ ID NOs: 54, 57 to 60, 64 to 67, 71 to 74, and 77 to 79 (VHPO of A. armata).

12. The nucleic acid construct of claim 1, wherein the polypeptide comprising the VHPO comprises an amino acid sequence at least 80%, at least 85%, at least 90%, at least 95%, at least 97%, at least 99%, or 100% identical to SEQ ID NOs: 1, 3 to 15, 18 to 21, 25 to 28, 32 to 35, 38 to 41, and 43 to 46.

13. The nucleic acid construct of claim 1, wherein the polypeptide comprising the VHPO comprises an amino acid sequence at least 80%, at least 85%, at least 90%, at least 95%, at least 97%, at least 99%, or 100% identical to any one of SEQ ID NOs: 1, 3 and 4 (Mbb1, Mbb3 and Mbb4 of A. taxiformis).

14. The nucleic acid construct of claim 1, wherein the polypeptide comprising the VHPO comprises an amino acid sequence at least 80%, at least 85%, at least 90%, at least 95%, at least 97%, at least 99%, or 100% identical to any one of SEQ ID NOs: 15, 18 to 21, 25 to 28, 32 to 35, and 38 to 40 (VHPO of A. armata).

15. The nucleic acid construct of claim 1, wherein the polypeptide comprising the VHPO comprises an amino acid sequence at least 80%, at least 85%, at least 90%, at least 95%, at least 97%, at least 99%, or 100% identical to any one of SEQ ID NOs: 5 to 7 (Chondrus crispus vBPO).

16. The nucleic acid construct of claim 1, wherein the polypeptide comprising the VHPO comprises an amino acid sequence at least 80%, at least 85%, at least 90%, at least 95%, at least 97%, at least 99%, or 100% identical to any one of Acaryochloris marina vBPO (SEQ ID NO: 8); Alteromonas naphthalenivorans vBPO (SEQ ID NO: 9), Ascophyllum nodosum vBPO (SEQ ID NO: 10), Corallina officinalis vBPO (SEQ ID NO: 11), Corallina pilulifera (SEQ ID NO: 12), Gracilaria changii vBPO (SEQ ID NO: 13), and Laminaria digitata vBPO (SEQ ID NO: 14).

17. The plant of claim 3, wherein the plant is a terrestrial plant.

18. The plant of claim 3, wherein the plant is a monocotyledon, and wherein the monocotyledon is selected from corn, millet, sorghum or pasture grasses.

19. The plant of claim 3, wherein the plant is a dicotyledon, and wherein the dicotyledon is alfalfa or clover.

20. The nucleic acid construct of claim 1, wherein the coding sequence is operably linked to a promoter for driving expression of the polypeptide in a plant cell.

21. The nucleic acid construct of claim 1, wherein the coding sequence is operably linked to a nucleic acid sequence encoding a targeting signal.

22. The nucleic acid construct of claim 21, wherein:
i) the targeting signal is a peroxisomal targeting signal, and wherein the peroxisomal targeting signal is SEQ ID NO: 90;
ii) the targeting signal is a mitochondrial targeting signal, and wherein the mitochondrial targeting signal is SEQ ID NO: 89; or
iii) the targeting signal is a chloroplast targeting signal, and wherein the chloroplast targeting signal is SEQ ID NO: 88.

23. An animal feed or animal supplement comprising the modified plant cell of claim 2.

24. (canceled)

25. (canceled)


SEQUENCE LISTING (97 sequences)

SEQ ID NO: 1 - protein, 590 aa, Asparagopsis taxiformis
MTDTQNPNRAEVAFKVRVSAAELARARGSPTHLSNNSESRFRNPDGTRSLLANFTKGLPHTKETALVVSAIDYDSFVRAIDSGDPRDFADLPLGPQGVEPRFTSGIASDPEVGTRAWESGGAGLVFDLEGPDAQAVTMPPAPELDSDELVTEVTECYWMSLLRDVPFPTFDSDPHIQSAAESINNTQWIQFKNNPPAHLTAAERSRLRGPITTANVFRGITPGDEVGPYLSQFLLVGTTGIANGNEVGDGFIQYGGMRMDQRVRVAKSHIDYMTTFGAYLDVQNAANVSGRELYNEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKKLLQKVHDHNKKQNLLSDRGNPRANDFNPDGDVSKGNLLMPMAFPEGSPMHPAYGAGHATVAGACVTVLKAFFDGGYRLPFCYITDENGTGLKAVEIDEPLTVDGELNKICSNISIGRNWAGVHYFTDYIESIRIGEEIAIGILQEQKLTFSENFSMTLNKFDGSTIRI

SEQ ID NO: 2 - protein, 813 aa, Asparagopsis taxiformis
MAKQKSLFTLLSATLESHLSTHAFQLLALIIYALANALMFVWGAHDEFHHHTDANNLRWYICIARGAGYTLNLNTALVILLAARLFATYLRETPLQHILPLDKSFPAFHIVVAYTIAAAVVIHASFHLAWLVAYDMWETGMWGFTMSAATGVVLLVVFIVMFISAMPKYRKKHFRIFYLIHSVGALLFFGLLVFHGMYNRVPETYKWIAAPLIIYTIDRVLRRYKISTAELELTGEHSSLKGSDILELRVPKPFDYQAGQYAEVCVKSINSEWHPFTIASSPHEDSMCFYIKALGDWTTNLRDAFEARVENDLYEPLKVQIRGPFGAPAQHVSGYCRVVLISGGVGSTPFAAICKHLHHLNKSENHSKALESQVSSKRQSQVQWRIRDAISILFDVSLDDSRNDEAANQQRRQQLADMLNMSPKGDVHNVSFKRTQSKRSDDFDAVEDSKQMNFSLCRQDSKSSFNLDDCSDAIRYYNRPSFQFFSSSPRHVINWYEYRTRLLAFLHTTRFTFALLLTLVARIVILCIVSIFNLGHIGLYNSHIDATWVIATNSILGLILGVALLTTILLEISFMRVRFFYRVWRCVDFFIFLPITFLCNISNFASWNGHAQPKFLIFLDLIIVLPIMLLLLCHRMYRSVGSRNLLDDTAECRQGCKCNKTIPDVDFVWTTPRDSDDEWLRNELYPLATGTELRLHRYVTRENMADLEDPERFITTANAGRPQWDEIFAQIAEQTPSHSKIGVFFCGPHPMGAAVQKSMRKVEVMSNLRGSYLRKTESAVLVDDLILRDEGEVKLLREYGCNIRFVFREENFS

SEQ ID NO: 3 - protein, 581 aa, Asparagopsis taxiformis
MTPPTRRQTALVKRIAAADLAANRHHPLHVRNADETTYKLPNHVCSYISAFTKGLPHHLDGRLVSPHHFQLFVHAVQTGHVTDIAAIPLGPPHSSSFRSGIAKGAAVRAWESMAAGNTFDLQGPDAQAVTMPPCPRLNSHELVTEMTELYYMALLRDVPFTHFNKHHLVQSAVHDMNHTHWTRSNRCCLPRPERRRHRSKFTTDNIFRGVTPGDITGPYISQFLLVGTKGLGDAHEETEGYVQYGATRIDQRVRTVEPKIDYMTTWAAYLDVQNGADLRRHSTYAKPPSYRFISTPRDLATYVHYDALYQAYLNACIILLDINAPYDGGLPFTADDAVDKQQGFATFGAPHVLTLVTEVATRALKAVRFQKYAVHRRLRPEAVGGLLEQYRRYGGGHSDLAYIIRPIRHLADSLSSDLMFMVAKENGRQNSSITDNGHPRSHDEGSDDTHLLSMAYAEGSPMHPSYGAGHATVAGACVTILKAFFDHTFKLPFAYVSSSDGRKLKTVKLSRKLTVEDELNKLAANISIGRSWAGVHYYSDYVESIRLGEEVAIGMLKEQKLTYSEKFTMTIPKFDGSVIEI

SEQ ID NO: 4 - protein, 586 aa, Asparagopsis taxiformis
MAEERRQNALDIRIQAARLAKKRDHPTHEANGDEDRYPETLIGSFTKGLPHEKETGLLSNPADFADFVRAINTGAIKDIRRLKLGIDEDPRFISGIASKGDKHPFADTRAWESMAAGLTYDLEGPDAQAVTMPPAPKLDSDELVTEITESYWMALIRDVPFTEFERDGNTAAAAASISRTRWVQFNEQPSQRPSTLTDEEIARLRGPYTKKNVFRGVTIGENVGPYLSQFLLVGTKGIGDVQQISDGYIQYGSMRMDQRVRVAAPKRDYMTTWASWLDVQNAGDLRGREIYDDDTPFRFITTPRDLATWVHFDALYQAYLNACIILLDIKAPFDPHIPFQADDDVDKQQGFATFGGPHILSLCTEVATRALKAVRFQKYNVHRRLRPEAIGGLVERFKKTNGDAKFAPVKKLVNDLDGDMLRRIEQHNCEQNKLSDDGHARRDDYSPEGESNQTYLLPMAFPEGSPMHPSYGAGHATVAGACVTVLKAFFDHEYELDFCYVPTTDGKRLEKVNINEKLTVEGELNKLCANICIGRNWAGVHYYSDYFESIKVGEEIAIGILQEQKLTYGEDFFMTLSKFDGEKIRI

SEQ ID NO: 5 - protein, 607 aa, Chondrus crispus
MSAARRQEAFQTRVRAARLARDRDHQVFEPNGEEDRYFRPNSQIFSYLGSYTKGLPHNRRTALITSPNHFVRFVRAIDSGEPADFIKVPLGPRPALQFESEEECRAANLFKSGIAGRNNTFPSSCLRAWESMGAGLVFDLEGPDAQAVGMPPAPTLDSDELVAEISEVYWMALLRDVRFTDFTNPGPIGRAINTLNKTAWIRAARDPPDSLTPQERARLRGPFNPSNIFRGTLPGDDVGPYLSQFLLVGTKGIGGAQDMADGFISYGAHRIDQRVRHARKELDYMTSWEAFLDVQNGADVTGRDTFESGSDAFRFITTPRDLATYVHFDALYQAYLNACLIMLDSGVRFDKGIPFGEPDFKDHQRGFAHFGGPHILSLVTEVATRALKAVRFQKFNTHRRLRPEAVGGLIERFNSNPDDPQFQDVKPLFEALDEDMMRRVARHNREQNQGSDFGMPRADDFNPAGDTLETMLLPMAFPEGSPMHPSYGAGHATVAGACVTVLKAFFQHDTELDFCFVPSDDGSRLVDASHNMNKKLTVEGELNKVCSNISIGRNWAGVHYFTDYIESILLGEQIALGILEEQMLTFPETFTMTVPLFSGELKVLRST

SEQ ID NO: 6 - protein, 666 aa, Chondrus crispus
MATPCFAQAPAPTLRRSPRRLPLTPTSFLPRPFLPKPFPRSPRHPHPRLNAASARTSAAQPPDRAATPCATRQQQALELRRAAAQLAHDRAHPAHVSNGSEELHLHPKTGAPLLLANFTKGLPHERDTGLAADAAHYDLFVAAVHSGLPDDIRAIPLGPQAAPGVRLPRSLRFRSGIARSSHLFPHTDVRAWESMAAGNAFDLQGPDAQAVTMPPAPALASPELALEMTENYWMALLRDVRFSQFAADPTVAAAMESMNATEWVALACGDASAVAEEERKRLRGPFTAQNVFRGNTQGDCKGPYISQFLLSGSAGLGQVNSGADGYIQYGAMRMDQRVRVALEGLDYMTTWESWLDVQNGADLRGRELYEDGERFRFICTPRDLATYVHYDALYQAYLNACIIMLNKKIPFDKGLPFQKDDDIDKQQGFALFGPPHILTLCTEVATRALKGIRFQKYNVHRRLRPEAVGGRVERYHHNCEDPLFADVKPLYDALDKDMLERVAAHNAEQNAKDDFGNSRFEDYSPTGSSGRTYLLPMAFPEGSPMHPAYGAGHAAVAGACTTILKAFFDHEHELDFAYVPTADGSKLEDVVDSLGEKLTVEGELNKVCSNISVGRNWAGVHYFTDYRESIILGEKIALGLLEEQKLTYAEEFSMTIPLFDGSTVTI

SEQ ID NO: 7 - protein, 631 aa, Chondrus crispus
MSDMSTEISPRQQEAYDIRVAAATLALARDRKRKVFEPNGEEDRYFRPNSQILSYLGSYTKGLPHNRSTALVTSPNHFVRFVQAIDSGEPADFIKVPLGPRPALEFTTEEECIAANLFKSGIATRNDSFDPACLRAWESMGAGLVFDLEGPDAQAVGMPPAPTLDSDELVAEISELYWMALLRDVRFTDFTNPGPIRRAINTLNRTTWIRAARDPPDSLTPQERARLRGPFNPSSIFRGTLPGDDVGPYLSQFLLVGTEGIGGAQDRADGFISYGAHRIDQRVRHATKELDYMTSWEAFLDVQNGADVAGRDTFESGSDAFRFITTPRDLATYVHFDALYQAYLNACLILLDNKVKFDQGIPMGEPDFNGNQGIPFQDPDFKDHQRGFAHFGGPHILSLVTEVATRALKAVRFQKFNTHRRLRPEAVGGLIERFNSNPDDPQFQDVKPLFEALDEDMLRRVASHNREQNERSDFGMPRADDFNPAGDTLETMLLPMAFPEGSPMHPSYGAGHATVAGACVTVLKAFFQHDATLDFCYVPSDDGSRLDDASHTLNKKLTVEGELNKVCSNISIGRNWAGVHYFTDYIESILLGEQIALGILEEQMLTFPETFTMTVPLFSGGFRTLISTSDP

SEQ ID NO: 8 - protein, 626 aa, Acaryochloris marina
MTDRRQDSYAVRVEAAELARSRNHPDHQANGDEERYAGAKYFMSFTKGLPHNEETGLLGDPKDFVEFRRAIDDGFIDPFSSDLIRHGALYKVEGGEVKRVDREVDGDPPNNFRQWEAPTAGVVFDLEGPDAQAVTMPPAPPLLDGNGNPNPELVLEMAEVYELAILRDQPFNDFENGRSNAAITQSIDRLNALSYISNQTGRPRKVNTSGQLDAQTVFRGSSPGVEAGPYLSQFLLIGNTDLNRDADGIGGGKVEDGLITYGALQINQRVPIAEPGKDYMRSMSEYVEVQRGIRPPRETYGSTNGGSVAQPNRPPRRFISTPRDLSTYVHYDALYEAYLNACIILLEMGTPFDPGFDHLSGVGAAASSFESRRNAGGFALYGGPHILTLVTEVATRALKAVRFQKFNNHIRLRPEALAARIELVNSFDGLSQADKDTVPEVLKKYIGLFSNALKPTLNALGGNYLLPMAFPEGSPMHPAYGAGHATVAGACVTILKAFFDTSAVLAKSGQSIAFKRLENGDKPIAFRAPDLPGSGPGEGVPKDDLKPFKPNHFLTLEGELNKLAANISIGRDMAGVHYFTDYYDSLRMGEEIAIGILEEQALTYSTDPFVLSVPTFDGDVRRIGRR

SEQ ID NO: 9 - protein, 588 aa, Alteromonas naphthalenivorans
MNETQNQRSKDSLDYRIRIASLNRHQSIHSQVMHINNGDEKSSINYAMSFTKGLEHNPETGLVQNAETCEALIDAINGGEISPFHAIDVPTAEGKRRLWEAPTAGFVFDVQGPDAQEVTMAPPPGLGSSELTFEMAEVYELAVLRDTALNKFSELSDEIAGPCERLNKLSYAMRGFCGRPRLTAKNACGDKVLTPGTVFRGSSPGVGIGPYLSCFMLMGGGNTKSSVESGLINYGAQTINQKVPKASDKNFMLEFDEWVAVQNGYSRNNTPKTGTGNYEENSGELKLTFIKTPRDLATYVHFDALYQAYLNACLILLSQKESQDFAPFDSNFVNLSGEFPNSRAGGFALFGGPHILSLVTEVATRALKAVRYQKFQVHRRLRPEVLAARIAKSDNDAFKAAVGGAASTNFENMKADLSDTLAKINELFHTEDLLPMAFAEGSPMHPSYGAGHSTVAGACVTVLKAFFDTNAQLARKGESVCFVKPEIAERDNWQPVKIVATDNDDTLTIEDSDEVITLEAELNKLAANISIGRNMGGVHFFSDYYDSLRMGEEIALGILKDQAESYPYDAFTLSVKTFDGDEKLITRS

SEQ ID NO: 10 - protein, 641 aa, Ascophyllum nodosum
MMKASGGIITALLAAFPPCLSQTVNELADMQTRPLLSGSVCRVRDTVDFLSPTKRAKITFKRRIGIAAGELAVGPTCHLNNGDEANVPLFDGQFHKSLPHDDVGRVNPEAYQLLLDCIESNDINVCDQVPSGVESDGRKLVNPLGGGGHQVDGADSDNIFIKQPDNLLSERLAAQQAEVYWMALLRDIPFSQFGTNNTVQMAAANLQGFDAFNGLSISRDADGNIDPMQDLFRTDWPGVSSGPMVSQFMLANFDIDGIVVEPKAKTLVPEMEYMTGVDTWLNIQNGGPPEDTLFVDEPLFIRNGRDLAALSFNDVLYTEAFRTILILFNESILAEAGPYGSSTRQEGFTTLGTSHYIHAMAAGSSSTRHAWYAKWQVHRVLRPEAYGGLLHFVINNLIDDVPLPASIVSNTELLNAVESLNQAQNGGTNQVFLLPMAVGEGSPVHPAYPSGHAINLGAYLTVLKAFLGFELGQRCFPSPMISNDAGTDRIPFVPSDGDRVGTCINEDGEEEVGLTYEGELNKVTSNVAIGRSHLGVHWRMDGVFGAEMGEAGAIRRLQQELGGLPEARDTEGPIPPASYKFRLYSGTMIELFPDNRYMLGDQMCKGFFTGDDFCVPADEEETAELEDLVLGSVVDQTCAEL

SEQ ID NO: 11 - protein, 598 aa, Corallina officinalis
MGIPADNLQSRAKASFDTRVSAAELALARGVVPSLANGEELLYRNPDPENGDPSFIVSFTKGLPHDDNGAIIDPDDFLAFVRAINSGDEKEIADLTLGPARDPDTGLPIWRSDLANSLELEVRGWENSSAGLTFDLEGPDAQSIAMPPAPVLTSPELIAEIAELYLMALGREIEFSEFDSPKNAEYIQFAIDQLNGLEWFNTPAMLGDPPAEIRRRRGEVTVGNLFRGILPGSEVGPYLSQYIIVGSKQIGSATGGNKTLVSPNAADEFDGEIAYGSITISQRVRIATPGRDFMTDLKVFLDVQDAADFRGFESYEPGARLIRTIRDLATWVHFDALYEAYLNACLILLANRVPFDPNIPFQQEDKLDNQDVFVNFGDAHVLSLVTEVATRALKAVRYQKFNIHRRLRPEATGGLISVNKIAAEKGESVFPEVDLAVEELEDILEKAEISNRKQNIADGDPDPDPSFLLPQAFAEGSPFHPSYGSGHAVVAGACVTILKAFFDSNFQIDQVFEVDKDEDKLVKSSFKGTLTVAGELNKLADNIAIGRNMAGVHYFSDQFESILLGEQVAIGILEEQSLTYGENFFFNLPKFDGTTIQI

SEQ ID NO: 12 - protein, 597 aa, Corallina pilulifera
MGIPADNLQSRAKASFDTRVAAAELALARGVVPSFANGEELLYRNCETGDPSFIASFTKGLPHDDNGAIIDPDDFLAFVRAINSGDEKEIADLTLGPARDPETGLPIWRSDLANSLELEVRGWENSSAGLTFDLEGPDAQSVAMPPAPVLMSPELIAEMAELYLMALGRDIEFSEFESPKNAAFIRSAIERLNGLEWFNTPAKLGDPPAEIRRRRGEVTVGNLFRGILPGSEVGPYLSQYIIVGSKQIGSATVGNKTFVSPNAADEFDGEIAYGSITISQRVRIATPGRDFMTDLKVFLDVQDGADFRGFESYEPGARLIRTIRDLATWVHFDALYEAYLNACLILLANGVPFDPNLPFQQEDKLDNHDVFVNFGSAHVLSLVTEVATRALKAVRYQKFNIHRRLRPEATGGLISVNKKSFLAGSDIIFPEVSELVEELSSILDDVAESNEKQNRADGIVSPDKSFLLPMAFAEGSPFHPSYGSGHAVVAGACVTILKAFFDANFQIDKVFEVDTDEDKLVKSSFKGTLTVAGELNKLADNVAIGRNMAGVHYFSDQFESLLLGEQIAIGILEEQSLTYGENFFFNLPKFDGTTIQI

SEQ ID NO: 13 - protein, 605 aa, Gracilaria changii
MASHIPRFTKAYAVREQAALMAKSRPHPLHVSNNEELRYRIPTSGEPSHIGSYTKGLPHDRNTGVLSNPDDFQSFIRAMDAGDQFSIRDVPLGPQNEQQAFQSGIAKGVSARAWESMAAGLTYDLEGPDAQSVTMPPAPALDSDELLTEVTELYWMALLRDVPFSEFNGDKNNDIQEAVDSLNSTPWVQIAKNGVVPTTLTDAERVRLRGPFTVDNVFRGTTRGDRDGPYLSQFMLIGNTGIGNGNRIEDGFIRYGAHRVDQRVRVAEMGRDFMTTWSAFVDVQNGADVRGREVYNNDVRYRFISTPRDLATYVHFDALYQAYLNACLILLGMRVPFDKGLPFQREDDIDHQQTFATFGAPHILSLVTEVATRALKAVRFQKFNVHRRLRPEAVGGLLHRYMQETTNDLYRGAKQLAENLDANLLDKVKRHNAKQNMKSDGDAPRANDLGECEDADEITLLPMAFPEGSPMHPSYGAGHATVAGACVTILKAFFDHGYELRMLDEDGETMIPFAFVPTADGSGLENRIGEYRNLTALTVEGELNKVCSNICIGRNWGGVHYFSDYRESIRVGEQVAIGLLEEQKLTYGENFSMTVPLFDGSVHRI

SEQ ID NO: 14 - protein, 646 aa, Laminaria digitata
MKISSLGLTALLAAFAPCLGYEEPPEPTQPLLSGNVCRVRDSLDFLDPVPRAKVTLLKRLAIAKDEISVGPTCHINNGDEENVPLFAGQYHKTLPHDKFGQVDEDAYKKLLECVFTSDINECEKVPSGAGRRGGAKLTNPLGGTAHQVTGADSDNVFITTPDSLLSERLAAQQAEVYWMALLRDIPFGEFAKNDYVRLAAENLQGLPAFKGLNIPRSEGGKIDPVTDLFRTTWPGVTTGPVVSQFMLSDFLIDSIKVTPKADPLTPGVDYMTAFQPWLDVQNGASKLETTFDEENPRFIRNGRDLATIALRDQLYTEAFRAALILFTEGALGGEVGPYAEAERQQGFATFGEPHILTAMASASSVTRHAWYAKWQVHRMLRPEAYGALVHNTLMRDVITPLPDSILRNTELLNRVEVHNQRMNPDGEKTFLLPMAAAQGSPTHPAYPSGHAINNGAYITALKAFLGYEAGQKCFPNPVVSNDEGTKRIKYKPSGREIVGECVNEKGKLVEGLTYEGELNKISANVLLGRSHIGVHWRMDGVYGALMGETSCVRRLQQELPGLPEAREVEGKKRRGDIPPATYKFRLYSGKILELYGRNLYKLDGKLCEGAFTGDDFCDPIDEDDYSSFDDIVEEHAQFSLHGHTEL

SEQ ID NO: 15 - protein, 592 aa, Asparagopsis armata
MATPDTQNPNRAEASFKIRLSAAELARSRGSPTHISNNSEARFRNPDGTRSLLANFTKGLPHINETGLVQSALDYDQFVRAIDSGDARDFAALPLGPQNVTPRFTSGIATNPEQSGVRAWESMGAGRTFDLEGPDAQAVTMPPAPELDSEELVTEVTECYWMSLLRDVPFPTFHCDEDVAAAAKSINDTQWIKFKENPPTHLTSAERSRLRGPIDASNVFRGITPGDDVGPYLSQFLLVGTTGIANGNEVSDGFIQYGGMRMDQRVRVASPKVDYMTTFGSWLDVQNAADVTGRERYESDPGFRFIHTPRDLATYVHFDALYQAYLNACIILLDIGAPFDSGVPFQLDNDIDKQQAFATFGGPHILSLVTEVATRALKAVRFQKFNVHRRLRPETVGGRVDRYYKTKAPEFSGAAKLVDSLDSDLLDRVYYHNKKQNKLSDNGAPRSADFNPDGGLSEGNLLMPMAFPEGSPMHPAYGAGHATVAGACVTVLKAFFDGGFPLPFAYVTNENGCGLEPVEVSEPLTVDGELNKICSNISIGRNWAGVHYFTDYIESIRIGEEIAIGILQEQKLTFSENFSMTLNKFDGSTIRI

SEQ ID NO: 16 - protein, 830 aa, Asparagopsis armata
MNTAFPFRRKWSPSLWSARLESYLSTHAFELLTVITYITALSLMFVWGAHDEFHNQSPDTNLRWYISIARGAGYTLNLNTALVILLASRLFFTFIRDTPLHKILPVDKSFPAFHILVGYTIAASLFIHVPFHFTWIIAYDKWSAGLWDFTMSAVTGVVLLLIFIIMFMFALPFYRKKYFRLFYISHMVGALLFFPLLIFHGMYRRVPETYKWVALPLVIYILDRIARRFKISTVQLKLTSEHSALKGKDILELRIPKPFTYRAGQYAEIRVPSLSREWHPFTIASAPHEKCMCFFIKSLGNWTKELRGAFQNRMEGEDPNPLLVQVRGPFGAPAQHVSSYERVVLISGGVGSTPFAAISKSLHFLNKNKTCPSTDFSKTNLSEMKHIEQRISETISAVYDIDLANSSDEPESEEQRKQYITDMMNLTAQNSEELDVSTSRTSEQCVSSRSCPNIGDGLRILDVENQLEESSVSSMSTCDDEEDYSSDFSEEMRLEKELAARKRKMARQKIARLYERRSILFSFLHTTRVTLALQLSLIARIVLVCIVSIFNIGEFNFGDSRIGAKWVIGADAVLSVLHAVTLTLTILLEISFMGPRFFRSSWQFMDFFVFVPLSILCSVSGVLSWTGTEMSGTLVFLHFIVLLPIMLIFMSARLYRSIGSLNLFHDRKCNPNCACGKKIPDVDFIWTTRHTVDDQWLREELFPLEDGTKLRLHRYITGKESEDFESAETFITTSNIGRPPWDEIFAKIAQKSPSHAEIGVFFCGPHSMGAAVRKSMRKTEIRSNLRGAYLETTNVVDLVADLLLRNTDHAAELRRHGCNIRFVFREENFG

SEQ ID NO: 17 - protein, 813 aa, Asparagopsis armata
MPSPSSSTCPKSTQDLLTARLESHLSTHAFELLALVVYATALLLMFLWGAHDEFHHHTSDDNMRWYISIARAAGYTLNLNTALVILLAARLFSTYIRETPLHLIIPLDKSFPAFHIIVAYAIAISLVLHVAFHFAWIIAYDKWEPGLWGYTMSASTGSVLLIVFLVMFITALPTFRKKRFRFFYLTHMFGVILFFPLLIFHGMYNRVPETYKWITAPLIIYIIDRLLRRFKVSTVPLQLTSDHSSLKSADILELRVPKPFHYRAGQYAEVCVPSISSEWHPFTIASAPHEETMTFFIKTLGNWTQALRHQFQTRMDSDDCKPLTVQIRGPYGAPAQHVGRYQRVVLVSGGVGATPFAAISKHLHHLNVAEHHKKTLDVDIPSKHTIQLGNRIRETISNMYGLGLGESGCAEDSNQQRRQHLADMLNMSPDGAVQKLGNEISHPSPTGSSDVSDTCSSENVDLSFSSSIGSQMATLGTYEKECSSRKGGREDKMGRSAYSKHNLMFLNGKRSRLLSLLHTTRITLLLLFTLIARLVLTCVVSIFDLGHFSTTSGHIEAKWVIVTNVVLGLIVGLTLFTIIVLEVSFMGTRFFHRNWRCVDFLVFVPLSFLCNLYGIVTWAGRDQSSFLTFVDYIIITPIIFLLLCHRLYRSIGTRNLFDDNGSCRGSEHSVPEVDFIWTVAHEGDDEWLREDLYTLATGTELRLHRYVTREKEVEVEGGEEFITSARAGRPKWDEIFLKIAEESRSCSEIGVFFCGPSGMGKQVRESLRRAEVFSSLRGSYLRKTGGGEVGDEVRLLKRFGSNVRFVFREENFS

SEQ ID NO: 18 - protein, 582 aa, Asparagopsis armata
MPPPTRRQSALVTRIAAADEAANRSHPPHLRNGDETLYKTTSNALSYLATFTKGLPHHPDGRLVSAQHFQLFARAVQTGTPQHIADIPLGPPSTFSTFRSGIAKDAPLRAWESMAAGNTFDLQGPDPQSVTIPPCPRLESNELVTEMTELYYMALLRDVPFSHFKSHHLVAAAVDDINHTHWIQSHKCCIPELERHRHREPFSSSNVFRGTTPGERNGPYISQFLIVGTKGLGEIHDEADGYIQYGAIRIDQRVRSVETEIDYMTTWAAFLDVQNAADLRRHSTYAKSSSYRFLYTPRDLATYVHHDSLYQAYLNACIILLDMKVPFDSGLPFTTEDHIDKQQGFVNFGGPHILTLVTEVATRALKAVRFQKYAVHRRLRPEAVGGLLEQYRRYGGGYSELANTIRPIRHLARSLSSNLMSLVAAANGHQNATMSDNGFPRSHDTGSGETHLLPMAYPEGSPMHPSYGAGHATVAGACVTILKGFFDHTYKLPFAYVSSCNGKMLKKAKVAKKLSVEGELNKLASNISIGRSWAGVHYYSDYVESIRLGEEVAIGILKEQKLTYSEKFSMTIPKFDGTTIKI

SEQ ID NO: 19 - protein, 591 aa, Asparagopsis armata
MSEERHASALRVRSEAADLAASRPLPTHLANGDEHRYHDTHLASYTKGLPHEKDTGLSSSPDHFAAFVRAIETGAQEDITSLPLGIDEDPRFISAIASEGSDHPIADTRAWESMAAGNTYDLEGPDAQAVTMPPAPKIDSEELLTEITECYWMALLRDVPFTEFERDGTTAAAAASISRTRWVQFQEQPGQRPCDLSDAEIARLRGPFRKTNIFRGVTPGEQIGPYLSQFLLVGTKGIANIQEISDGFIQYGGMRMDQRVRVATPNRDYMTTWASWLDVQNAGDVRGRETYDNDTPFRFITTPRDLATYVHFDALYQAYLNACLILLDTKAPFDPEIPFVEDDDIDKQQGFATFGGPHILSLVTEVATRALKAVRFQKFGTHRRLRPEAVGGLIERYRKTEGAEPFEPIQKIATEIDNDLMTRISQHNAAQNELSDNGGARSGDYNPEGCESAETYLLPMAFPEGSPMHPSYGAGHAAVAGACVTILKAFFDHTHELEFCYVPSTDGQNLEEVEINEPLTVEHELNKLCANISIGRNWAGVHYYSDYIESARLGEEVAIGILEEQSLTYGEEFSMTIPKFDGTTIKISNSK

SEQ ID NO: 20 - protein, 589 aa, Asparagopsis armata
MSTTRQAAALQVRTDAAKLAASRKLPTHTPNGDETRFSDSYIASYTKGLPHDKKTGLLSSTDDFTAFVRAINTGAQEDIRSLRLGIDETPRFISGIASPALDLPIADTRAWESMAAGNTFDLEGPDAQAVTMPPAPKIDSEELLTEITECYWMALLRDVPFSEFHRNGTVAAAAASISRTRWVQFQEQPGQRPSDLTNAEIQRLRGPFNKSNVFRGDMPGEQVGPYLSQFLLVGTQGIGKMHQPEEGFIQYGGMRMDQRVRFATPNRDYMTTWASWLDVQNAGDVRSRETYDKDTDYRFITTPRDLATYVHFDALYQAYLNACLILLDIKAPFDHGLPFVQDDDIDKQQGFATFGGPHILSLVTEVATRALKAVRFQKFGTHRRLRPEAVGGLIERYRKTNGAAPFAAAKKLATEIDNELMTRISRHNAKQNEFSDNGKARRNDFNPEGGASAETYLLPMAFPEGSPMHPSYGAGHAAVAGACVTILKAFFDHKYKLKFCFEPSTDGRKLEKVNISEPLTVEHELNKLCANISIGRNWAGVHYYSDYEESAKLGEEVAIGILQEQSLTYGEKFSMTLPKFDGTCITISN

SEQ ID NO: 21 - protein, 592 aa, Asparagopsis armata; identical to SEQ ID NO: 15

SEQ ID NO: 22 - protein, 830 aa, Asparagopsis armata; identical to SEQ ID NO: 16

SEQ ID NO: 23 - protein, 824 aa, Asparagopsis armata
MAPRSGKQRCSPTSLTLLSARLESYLSTNAFSLLALFIYSLANILMFVWGAHDEFHHHTSQKNLRWYICIARGAGYTLNLNTALVILLAARLFATYIRETPLQHILPIDKSFPAFHIIVAYTIAAALFIHVSFHLVWIIAYDKWELGMWGFTMSATTGFALLTVFVVMFVSALPRFRKKQFRTFYLIHMVGALLFFTLLIFHGMYNRVPETYKWIAGPLAIYTIDRVLRRFKISTAKLELTGEHSSLKGSDILELRVPKTFEYRAGQYAEVCVPSINNEWHPFTIASAPHEKSMCFYIKAIGDWTRHLRDAFEARIENDLYEPLKVQIRGPYGAPAQHVRGYRRVVLISGGVGSTPFAAICKNLHHLNKSENHSKALESHGSSKRQSQIHWHIRDAISNLFDVCLDDSRSDAASNQQRIQQVADMLNMSPKGNVHNVSFKRNLSKTSDDCSSVEEITKQIRADEEISLSQKEKQSSYNLDACSESIRYYNRPSANVFNVAQRRIFNFYEYRSRLLAFLHTTRFTFALLLTLVARIVILCVVSIFNLGHIGISDSHIDAEWVIATNSLLGLVLGIALLTTVLLEISFMRLRFFYRGWRCVDFFIFLPITFLCNISNFMSWSGRQQAKFLIFLDLIIVVPIMFLLLCHRLYRSIGARNLLDDTQCRQSCTCNKTIPDVDFVWTTPRDSEDEWLRSELYPLATGTDLRLHRYVTREKDLDLEEPEHFITTANTGRPQWDEIFAQIAEQTPSHNEVGVFFCGPHPMGAAVQKSMRKVEVMSNLRGSYLRKTDSELLVEDLSLRDEKEVKLLREHGCNIRFVFREENFS

SEQ ID NO: 24 - protein, 813 aa, Asparagopsis armata
MPSPSSSTCPKSTQDLLTARLESHLSTHAFELLALVVYATALLLMFLWGAHDEFHHHTSDDNMRWYISIARAAGYTLNLNTALVILLAARLFSTYIRETPLHLIIPLDKSFPAFHIIVAYAIAISLVLHVAFHFAWIIAYDKWEPGLWGYTMSASTGSVLLIVFLVMFITALPTFRKKRFRFFYLTHMFGVILFFPLLIFHGMYNRVPETYKWITAPLIIYIIDRLLRRFKVSTVPLQLTSDHSSLKSADILELRVPKPFHYRAGQYAEVCVPSISSEWHPFTIASAPHEETMTFFIKTLGNWTQALRHQFQTRMDSDDCKPLTVQIRGPYGAPAQHVGRYQRVVLVSGGVGATPFAAISKHLHHLNVAEHHKKTLDVDIPSKHTIQLGNRIRETISNMYGLGLGESGCAEDSNQQRRQHLADMLNMSPDGAVQKLGNEISHPSPTGSSDVSDTCSSENVDLSFSSSIGSQMATLGTYEKECSSRKGGREDMLGRSAYSKNNLMFLKGKRSRLLSLLHTTRITLLLLFTLIARLVLTCVVSIFDLGHFSTTSGHIEAKWVIVTNVVLGLIVGLTLFTIIVLEVSFMGTRFFHRNWRCVDFLVFVPLSFLCNLYGIVTWAGRDQSSFLTFVDYIIITPIIFLLLCHRLYRSIGTRNLFDDNGSCRGSEHSVPEVDFIWTVAHEGDDEWLREDLYTLATGTELRLHRYVTREKEVEVEGGEEFITSARAGRPKWDEIFLKIAEESRSCSEIGVFFCGPSGMGKQVRESLRRAEVFSSLRGSYLRKTGGGEVGDEVRLLKRFGSNVRFVFREENFS

SEQ ID NO: 25 - protein, 582 aa, Asparagopsis armata; identical to SEQ ID NO: 18

SEQ ID NO: 26 - protein, 591 aa, Asparagopsis armata; identical to SEQ ID NO: 19

SEQ ID NO: 27 - protein, 589 aa, Asparagopsis armata; identical to SEQ ID NO: 20

SEQ ID NO: 28 - protein, 592 aa, Asparagopsis armata
MATPDTQNPNRAEASFKIRLSAAELARSRGSPTHISNNSEARFRNPDGTRSLLANFTKGLPQINETGLVQSALDYDQFVRAIDSGDARDFAALPLGPQNVTPRFTSGIATNPEQSGVRAWESMGAGRTFDLEGPDAQAVTMPPAPELDSEELVTEVTECYWMSLLRDVPFPTFHCDEDVAAAAKSINDTQWIKFKENPPTHLTSAERSRLRGPIDASNVVRGITPGDDVGPYLSQFLLVGTTGIANGNEVSDGFIQYGGMRMDQRVRVASPKVDYMTTFGSWLDVQNAADVTGRERYESDPGFRFIHTPRDLATYVHFDALYQAYLNACIILLDIGAPFDSGVPFQLDNDIDKQQAFATFGGPHILSLVTEVATRALKAVRFQKFNVHRRLRPETVGGRVDRYYKTKAPEFSGAAKLVDSLDSDLLDRVYYHNKKQNKLSDNGAPRSADFNPDGGLSEGNLLMPMAFPEGSPMHPAYGAGHATVAGACVTVLKAFFDGGFPLPFAYVTNENGCGLEPVEVSEPLTVDGELNKICSNISIGRNWAGVHYFTDYIESIRIGEEIAIGILQEQKLTFSENFSMTLNKFDGSTIRI

SEQ ID NO: 29 - protein, 830 aa, Asparagopsis armata; identical to SEQ ID NO: 16

SEQ ID NO: 30 - protein, 824 aa, Asparagopsis armata; identical to SEQ ID NO: 23

SEQ ID NO: 31 - protein, 813 aa, Asparagopsis armata; identical to SEQ ID NO: 17

SEQ ID NO: 32 - protein, 582 aa, Asparagopsis armata; identical to SEQ ID NO: 18

SEQ ID NO: 33 - protein, 591 aa, Asparagopsis armata; identical to SEQ ID NO: 19

SEQ ID NO: 34 - protein, 589 aa, Asparagopsis armata
MSTTRQAAALQVRTDAAKLAASRKLPTHTPNGDETRFSDSYIASYTKGLPHDKKTGLLSSTDDFTAFVRAINTGAQEDIRSLRLGIDETPRFISGIASPALDLPIADTRAWESMAAGNTFDLEGPDAQAVTMPPAPKIDSEELLTEITECYWMALLRDVPFSEFHRNGTVAAAAASISRTRWVQFQEQPGQRPSDLTNAEIQRLRGPFNKSNVFRGDMPGEQVGPYLSQFLLVGTQGIGKMHQPEEGFIQYGGMRMDQRVRFATPNRDYMTTWASWLDVQNAGDVRSRETYDKDTEYRFITTPRDLATYVHFDALYQAYLNACLILLDIKAPFDHGLPFVQDDDIDKQQGFATFGGPHILSLVTEVATRALKAVRFQKFGTHRRLRPEAVGGLIERYRKTNGAAPFAAAKKLATEIDNELMTRISRHNAKQNEFSDNGKARRNDFNPEGGASAETYLLPMAFPEGSPMHPSYAAGHAAVAGACVTILKAFFDHKYKLKFCFEPSTDGRKLEKVNISEPLTVEHELNKLCANISIGRNWAGVHYYSDYEESAKLGEEVAIGILQEQSLTYGEKFSMTLPKFDGTCITISN

SEQ ID NO: 35 - protein, 592 aa, Asparagopsis armata; identical to SEQ ID NO: 15

SEQ ID NO: 36 - protein, 830 aa, Asparagopsis armata; identical to SEQ ID NO: 16

SEQ ID NO: 37 - protein, 813 aa, Asparagopsis armata; identical to SEQ ID NO: 17

SEQ ID NO: 38 - protein, 582 aa, Asparagopsis armata; identical to SEQ ID NO: 18

SEQ ID NO: 39 - protein, 591 aa, Asparagopsis armata; identical to SEQ ID NO: 19

SEQ ID NO: 40 - protein, 589 aa, Asparagopsis armata; identical to SEQ ID NO: 20

SEQ ID NO: 41 - protein, 590 aa, Asparagopsis taxiformis; identical to SEQ ID NO: 1

SEQ ID NO: 42 - protein, 852 aa, Asparagopsis taxiformis
MDTVSPGDSRQKAQGKMRPMRSFAILSANLEAFLSTHAFSLLFLIFYLTCTALMFVWGFTTQWKKTDNEAMIYFISIARGAGYSLNLNTSLVILIAARLTFTLIRETPLQYILPLDKIFPLFHMFVGYMIAFLVLIHGTFHMTWIIRFQGWNSGLWGFTMSFVTGFIILPVFVVMLITSLPHFRKNKFRLFYLFHLYGAALFFSILIFHGMYFERPETYKWITPSLIIYLLDRFIRRMKISSHDLDIALEKSILKDGDILELRVPKPFDYRPGHYAEVMVPSINKEWHPFSIASAPHEETMCFYIKVLGDWTTEVRNAFKARLDGTITEPLKVRVRGPYGAPCQDVDGFKRVVLVSGGVGATPFSAIVKELHYLKTQAKNKNDGPGRIARDIDVRIRDSIANLYDVEVQDMEKATHIAQQKSIYVTEMLELTARRTSSLHRVRSSVMDRMHGSSSTKTVLSTDAAIPSLEDGPVGGGQPMSRMQRLSSSLSKAGNIRRVMSTLSENPVTWDQVMSAQNDPLTNTTDLRRQMARLYDKRGMLLAFMHTARFTFFLLLTLIVRIIIISVAATLDSPYFQLNDQFDGYTRLWPVVVDALFSVPVAAALVVSITLEISFMRMRYFDTAARTFDALVLMPAAVIGCVLAILTWARQDPESVLVKVIEYVLFIPLLFLGLSWRLYRITRSRSLLSDPACRCDNSDHIPDVDFIWSFPYEQSDQWLQDELGPLAKGTELRLHRYVTREKELDVEAAKSFITSANTGRPKWDDIFQTIAEETPSNGAIGVFFCGPHVMGNQVRDAMLKVEVLSNLRAAYLRNTPDEVLMTDLNLPNAEAVERLRSLGCSVHFVYREENFG

SEQ ID NO: 43 - protein, 581 aa, Asparagopsis taxiformis
MTPPTRRQTALVKRIAAADLAANRHHPHHVRNADESTYKLPNHVCSYISAFTKGLPHHLDGRLVSPHHFQLFVHAVQTGHVTDIAAIPLGPPHSSSFRSGIAKGAAVRAWESMAAGNTFDLQGPDAQAVTMPPCPRLNSHELVTEMTELYYMALLRDVPFTHFNKHHLVQSAVHDMNHTHWTRSNRCCLPRPERRRHRSKFTTDNIFRGVTPGDITGPYISQFLLVGTKGLGDAHEETEGYVQYGATRIDQRVRTVEPKIDYMTTWAAYLDVQNGADLRRHSTYAKPPSYRFISTPRDLATYVHYDALYQAYLNACIILLDINAPYDGGLPFTADDAVDKQQGFATFGAPHVLTLVTEVATRALKAVRFQKYAVHRRLRPEAVGGLLEQYRRYGGGHSDLAYIIRPIRHLADSLSSDLMFMVAKENGRQNSSITDNGHPRSHDEGSDDTHLLSMAYAEGSPMHPSYGAGHATVAGACVTILKAFFDHTFKLPFAYVSSSDGRKLKTVKLSRKLTVEDELNKLAANISIGRSWAGVHYYSDYVESIRLGEEVAIGMLKEQKLTYSEKFTMTIPKFDGSVIEI

SEQ ID NO: 44 - protein, 586 aa, Asparagopsis taxiformis; identical to SEQ ID NO: 4

SEQ ID NO: 45 - protein, 685 aa, Asparagopsis taxiformis
MFHNAFVASAPVGVARIRGASITTKHSPFNAFRRQSSAASVKMAQDNSDQEQRRKDAKQVRINAANLAFNRGVRPHKSNGDESDFKETLIGTFTKGLRHNTETGQIFSRDLFDPFRASLATGDPADFKKIRLGPKLPKDTPGRWQSTLANGAPGLPFESQVKARAWESAGAGLAFDLEGPDAQAVTMPPAPKLASAELEFEIAELYFMALARDIPFSLWDSPDTKAFFDNAIATLSALPWVSEEFGKGLTDEERFRREGKLNLTTGNIFRGYGAGVRDGPYISQYLIRGSQGLGTDGTATGQVMYGSLGLDLRVRSIEPRKDWMTTWEAFIDVQDGADVRQGEFPDYKCAPDGSPIFRFIADGRDLSTYVHYDALYEAYLNACFALLADGVGVDTGLPFRDDDEHDKQTAFATFGGPHILSLVCEVATRALKAVRYQKFQIHLRARPEAVGGLIHAQKSERCDFGEEVRALVARLDGCGLLDLVADKNMQQNQTFADRVKDRSASSPSYLLPMAFPEGSPMHPSYGAGHGTVAGACTIILKAFFNTVQFDGKCAVKDAKPSNVEFTPLPNEIFEADAHGCLQPVCLPDGMTLTVDGELNKIANNVAFGRSLAGVHWSSDNIESLILGEEVARGILEEQVLTFAEQHSFTYVGFDGHLNVIRSQPKHELNVDKRTVEVLRSDDPVA

SEQ ID NO: 46 - protein, 596 aa, Corallina officinalis
MGIPADNLQSRAKASFDTRVAAAELALARGAVPSFANGEELLYRNSETGDPSFIGSFTKGLPHDDNGAIIDPDDFLAFVRAINSGDEKEIAALTLGPARDPETGLPIWRSDLANSLDLEVRGWENSSAGLTFDLEGPDAQSVAMPPAPVLTSPELIAEMAELYLMALGRDIEFSEFDSPKNAAFIRSAIERLNGLEWFNTPAKLGDPPAEIRRRRGEVTVGNLFRGILPGSEVGPYLSQFIIVGSKQIGSATVGNKTLVSPNAADEFDGEIAYGSITISQRVRIATPGRDFMTDLKVFLDVQDAADFRGFESYEPGARLIRTIRDLATWVHFDSLYEAYLNACLILLANGVPFDPNLPFQQEDKLDNQDVFVNFGSAHVLSLVTEVATRALKAVRYQKFNIHRRLRPEATGGLISVNKNAFLKSESVFPEVDVLVEELSSILDDSASSNEKQNIADGDVSPGKSFLLPMAFAEGSPFHPSYGSGHAVVAGACVTILKAFFDANFQIDQVFEVDTDEDKLVKSSFPGPLTVAGELNKLADNVAIGRNMAGVHYFSDQFESLLLGEQIAIGILEEQSLTYGENFFFNLPKFDGTTIQI

SEQ ID NO: 47 - RNA, 1773 nt, Asparagopsis taxiformis
  source 1..1773: mol_type = mRNA
atgaccgacacacagaatcccaatcgtgcagaagtggccttcaaagtccgcgtatcagcagctgagctggcacgcgctcgtggctccccaacccatctatccaacaactcggagagtcgcttcagaaaccctgatggtacccgttcgctgctcgccaatttcaccaagggtctcccgcataccaaggaaaccgctctagtggtgtccgcaatcgactacgatagctttgttcgcgcaattgacagcggtgatcctcgtgatttcgcagacctcccgctcggtccgcagggtgttgagcccagattcacctccggtatcgctagcgacccagaggtcggaacccgtgcctgggaaagtggtggtgctggtcttgtcttcgatctcgaaggtcccgatgctcaggctgtcactatgccgcccgcgcccgagctggacagcgatgaattggttactgaagtcacagaatgctactggatgtctctgctgagagacgttcccttcccgacatttgacagtgatccccatattcagtcagctgcagagtccatcaataacacccaatggattcaattcaagaacaacccaccagcacatctcacagctgctgagcgttcccgtctcagaggccccatcaccactgccaacgtcttcagaggtatcacaccgggagacgaagtcggaccctatctgtcacagttcctccttgtcggtaccaccggtattgccaatggcaatgaagttggcgacggattcatccagtacggtggcatgcgtatggaccagcgcgtgcgtgtcgccaagtcacacattgattacatgaccacattcggcgcctatctcgacgtgcaaaatgccgccaacgtctccggtcgcgaattgtacaatgaagaggaaccccgtttccgcttcattcacactcctcgtgatctggccacttacgtgcatttcgacgccttgtatcaggcctacctcaacgcttgcatcatcttgctcgacatcggtgcgccattcgactctggaattcccttccagctcgataacgacattgataagcagcaaggatttgccactttcggcggtccgcacatcctctctctagttaccgaagtggccacccgagccctcaaggctgttcgcttccagaaattcaacgtccatcgtcgtctgcgtccggaggccattggtgcccgagtcgatcgatactgcgcaacaaaggcacctgaatttgctggcgctgccaagctctccgaagcgctggacaagaagttgttgcagaaggtgcacgatcataacaagaaacaaaacttgttgagcgatcgaggaaacccgcgtgcaaacgacttcaaccccgacggagatgtgagcaaaggtaatctgctgatgcccatggcgttcccagagggaagtcctatgcatcccgcgtacggcgctggacatgcgactgttgcaggcgcttgtgtcacagtgttgaaggcgttcttcgatggtggttacaggttgccgttctgctacatcaccgacgagaatggaacaggtcttaaggcagtggaaattgacgagccgttgaccgtcgacggcgagttgaataagatttgctccaatatttcgattggtcgcaattgggctggcgtgcattacttcactgattacatcgagagtattcgcattggtgaggagattgccattggtattctgcaggagcaaaagctgacgttcagtgaaaacttctcaatgacgctgaacaagtttgacggttcgaccatccgtatctag

SEQ ID NO: 48 - RNA, 2442 nt, Asparagopsis taxiformis
  source 1..2442: mol_type = mRNA
atggctaagcagaagtctctcttcacgctgctcagcgccacactagaatctcatctctccacccacgctttccaactgctcgcgctgatcatctatgcactcgccaacgcgctcatgtttgtctggggcgcccatgatgaattccaccatcacaccgacgccaataatctgcgatggtacatctgtatcgcccgcggcgcaggttacacactcaatctcaacacggcgctcgttattctgctcgccgcccgactctttgcaacttatcttcgagaaacgcccctacagcatattctgcccctcgacaagagctttccagcttttcacatcgttgtagcctacactattgccgccgccgttgttattcacgcctcgtttcatctagcctggctcgtcgcctatgacatgtgggaaactggcatgtggggatttacgatgagcgccgcgaccggagtcgtcctgctcgttgtctttattgtcatgtttatttcagcgatgcccaaataccggaaaaaacattttcgcatcttttatcttattcattccgttggtgctctgctctttttcggtctgctcgtatttcatggaatgtacaacagagtaccggagacgtacaaatggattgctgcaccgcttatcatctataccattgatagagtacttcgacggtacaagatatccaccgctgagctcgaattaactggagaacattcctcgctgaaagggagtgatattctggaattgcgagtaccaaagccgtttgattatcaagctggacagtatgcagaagtttgcgtcaaatcaattaacagtgagtggcatccgtttacaattgccagttcgcctcatgaggactcaatgtgcttctatattaaagcgcttggtgattggacaacaaatttacgagatgcttttgaagcccgagttgagaatgatctgtacgaaccacttaaggtacagattcgaggcccattcggagctccagctcaacatgtgagtgggtattgtcgagtcgtgcttatttccggtggtgtcggctccaccccattcgcagctatttgcaagcatctgcatcatttgaacaagtcggaaaatcattccaaagctctcgaatcgcaagtttcctccaaaaggcagtctcaagtgcaatggcgtattcgagatgccatttccatacttttcgacgtcagtctggatgatagtcgaaatgatgaagccgcaaatcagcaaagacgacagcaactggctgatatgctcaacatgtcgccaaagggtgatgtgcataacgtttcattcaaacgcactcaatccaagaggtcagatgactttgatgccgtggaggactcgaaacaaatgaatttttcattgtgtcgccaagatagcaaatcgagtttcaatcttgatgactgttctgatgccatccgatactacaatcgtccttcctttcagttcttcagttcatccccaaggcatgtaatcaattggtatgaataccgtacaaggctactcgcatttctgcacactactcggtttacgtttgcacttttgctcacgctcgtcgcaagaattgttatcttgtgcatagtgtccatattcaatctcggacacattggactttacaatagtcatatcgatgctacgtgggtgattgccactaactccatattgggactcatacttggagtcgctctgttaacaactattctattggaaatcagttttatgcgcgtgcgcttcttctaccgagtctggcgctgtgtagactttttcatctttctacctataactttcctgtgcaacatcagcaatttcgccagctggaacggtcatgcacagccgaaattcctaatattcctggatttgatcattgtactgcccatcatgctgcttttgttatgccatcgtatgtaccggagcgtcgggtcacgaaaccttctggacgacacggccgaatgtcgccagggctgtaaatgcaacaaaacgatcccggatgttgattttgtgtggacgacgccacgcgacagcgacgacgaatggctgcgaaatgaactgtatccactggccacggggacggagcttcggttgcatcggtatgtgactcgcgagaatatggccgacttggaggacccagagcgcttcattacaacggcaaacgctggacgaccacagtgggacgagattttcgcacagattgcagagcaaacgccaagtcacagcaagatcggggtgttcttttgcgggccgcatccgatgggtgctgccgtgcaaaagagtatgcgcaaagtggaggtcatgagtaatctgcgtgggtcgtacctgcgcaagacagagagtgcggttctggtcgatgatttgatactgagggatgagggggaagtgaagttattgagggaatatgggtgtaatattcgatttgttttccgcgaggaaaactttagctag

SEQ ID NO: 49 - RNA, 1746 nt, Asparagopsis taxiformis
  source 1..1746: mol_type = mRNA
atgacgccgccaacacgccgccaaacagcgctcgtcaagcgcattgccgccgcagacctggccgccaatcgtcaccatccgcttcatgtgcgcaatgccgacgaaaccacctacaaattgccaaaccatgtctgttcctacatatccgctttcaccaagggtttgccccatcatctcgacggccgcctcgtttccccacaccactttcaactcttcgtccacgccgtgcaaactggccatgtcacagacattgccgccattccactcggacccccacactcctcctccttccgttccggcatcgccaagggcgccgccgtgcgcgcatgggagtccatggccgcgggtaacacctttgatttgcaagggcctgatgcacaggccgttaccatgcccccttgtccgcgtctcaattcacacgagctcgtcacagagatgacagagctttattatatggcactgttgcgcgacgtgccgtttacgcatttcaacaaacatcatctcgttcagtccgctgtgcacgacatgaatcatacccattggactcggtccaacagatgctgtttacctcgccccgagcgccgtcgacatcgctccaaattcaccactgataacatcttccgcggtgtaacgcccggtgacataaccggtccctacatttcccagtttctccttgtcggcacaaagggtctcggcgatgcacacgaagaaacagaaggctacgttcaatacggcgccacaagaatcgaccagcgagtgcgcactgtcgaacccaaaattgactacatgacgacatgggccgcttatctcgacgtgcagaatggtgccgacttacgccgtcactccacctatgccaaaccaccgtcttaccgcttcattagcaccccgcgcgacctcgcaacctatgtgcattacgacgcgttgtatcaagcgtatctcaacgcgtgcatcatcctgctcgacatcaacgcgccatacgatggcggactgcccttcaccgccgacgatgccgtcgacaagcagcagggctttgcgacatttggcgcgccacatgtgctgacacttgtgacagaagtggcgacacgcgcgctcaaagctgtgcgctttcaaaagtatgccgtgcatcggcgtctgcggccggaagctgtcggcggtctattggagcagtatagacgctacggtggtggacattcggatctcgcatacatcattcgtccgattcgtcatctcgccgactcgctcagttccgatctgatgttcatggtcgccaaggagaacggacgccagaatagctcgattacggataacggccatccgcgctcgcatgacgaaggttcggatgatacgcatctgctttccatggcgtatgcggagggaagcccgatgcatcctagttatggcgcgggacatgcaacggttgcgggcgcttgtgtaacaatcctgaaagcgttcttcgatcatacgttcaaactgccgtttgcgtacgtgtcgtcgagcgatgggaggaaattgaagacggtcaagttgagcaggaaattgaccgtggaggatgagttgaacaagttggcagcgaatataagcattggacgcagttgggctggcgtccattactattcggattatgttgagagcattcggttgggcgaggaggtggccatcgggatgctcaaggagcagaagttaacttattcggagaaattcacgatgacaatacccaaatttgacggctccgtcattgaaatttag

SEQ ID NO: 50 - RNA, 1761 nt, Asparagopsis taxiformis
  source 1..1761: mol_type = mRNA
atggcagaggaacgtcgccaaaacgctctagacatccgcatccaagccgccaggcttgcgaagaaacgtgaccatccaacccatgaggcaaacggcgatgaggatcgttacccagaaacactcatcggctccttcactaaaggtcttccacatgagaaagaaaccggtctcctctccaaccccgccgatttcgccgactttgttcgcgctatcaacacaggcgcgatcaaagacataagacgcctcaaacttggaatcgatgaggatcctcgcttcatatctggtattgcctccaagggcgacaagcatcctttcgccgatacgcgcgcctgggaatccatggccgccggtctcacatacgatctagaaggccctgatgcccaagctgtcaccatgccgccggcgccgaaactcgacagtgatgaactcgtcacagagattacggagagctattggatggctcttattcgtgatgtgccgttcacagagtttgaacgtgatggaaatacagccgcagccgcagcttccatcagtcgcactcgttgggtacaattcaatgagcagccttcacagcgtccgtcaacattgaccgatgaagagattgctagattgaggggtccgtataccaagaaaaatgtattccgtggtgtgacgatcggtgagaacgtcggcccgtatctgtctcagttcttactggtaggcacgaaaggtatcggcgatgtacagcaaatttcggatggttacattcagtacggaagtatgcgaatggatcagcgtgtccgcgtcgcagcgccgaaacgcgattatatgacaacttgggcatcttggctagatgtgcagaacgctggtgatttacgtggacgtgaaatttacgatgatgatacaccgttccgcttcattacaaccccacgcgaccttgcgacatgggttcatttcgacgcgttataccaggcatatttgaatgcttgcatcatccttctcgacatcaaggcgccgttcgatccacacatcccgttccaggctgatgacgatgtagataagcagcaaggatttgcaacgtttggcggcccgcacatcctatcattgtgcacggaagtggctacgagagcactcaaggcagttcgcttccaaaagtacaacgtgcatcgtcgtcttcgtccggaggctatcggcgggcttgtggaacgcttcaagaagacaaacggagatgccaagtttgccccagtgaagaagctggtgaatgacctggacggcgatatgctgcgacgcattgagcagcacaactgtgagcagaataagctttcagatgatggacatgcacgccgagacgactattcaccggagggtgagagtaaccagacgtaccttctgcccatggcgttcccggaggggagcccgatgcacccgtcatatggtgcaggtcacgccacagttgccggtgcgtgtgtgacggtgctcaaggcattcttcgaccatgaatacgagctggacttctgctacgtaccgaccacggatggcaagcgtctggagaaggtaaatatcaatgaaaaattgacagtggaaggcgaactgaacaagctgtgcgctaatatctgtattggtcgaaactgggctggcgtgcattactactcggactactttgagagtatcaaagttggtgaggagattgcaattggtattttgcaagaacagaagcttacgtatggtgaagactttttcatgacgctgtccaagtttgacggtgaaaagatccgtatctaa

SEQ ID NO: 51 - RNA, 1824 nt, Chondrus crispus
  source 1..1824: mol_type = mRNA
atgtcggccgcccgtagacaggaagccttccagacccgtgtccgtgccgcccgcctggctcgcgaccgtgaccaccaagtcttcgaacccaacggtgaggaggaccgctacttccgccccaactcccagatcttcagctacctcggctcctacaccaagggcttgcctcacaaccgccgcacggccctcatcacctcgccgaaccacttcgtccgcttcgtccgagccatcgactcgggcgagccagccgacttcatcaaggtcccgctcggcccgcgcccggccctccagttcgagtccgaggaagagtgccgcgccgccaacctcttcaaatccggcatcgccggccgcaacaacaccttcccgtcctcctgcctgcgcgcatgggagagcatgggcgccggcctcgtcttcgacctggagggcccggacgcccaggccgtcggcatgccgcccgcccccacgctggacagcgacgagctcgtcgccgagatctccgaggtctactggatggccctgctgcgcgacgtccgcttcactgatttcaccaaccccggcccgatcggcagggccatcaacaccctcaacaagaccgcctggatccgggcggcacgggacccccccgactcgctcacgccgcaggagcgcgcccgcctccgcggcccgttcaacccgagcaacatcttccgcggaaccctccccggagatgacgtcggcccgtacctctcccagttcttgctcgtgggaactaagggcattggcggcgcacaggacatggctgacgggtttattagctacggcgcccaccgcatcgaccagcgcgtgcgtcacgcgaggaaggagctcgactacatgacctcttgggaggccttcttggacgtccagaacggcgccgatgtcaccggtcgcgatacctttgagtcgggctctgacgccttccgcttcatcacaaccccccgtgatctcgccacgtacgtccactttgacgcgctctaccaggcctacctcaatgcctgcctgatcatgcttgacagcggcgtgaggtttgataagggtattccgtttggtgagccagacttcaaggatcatcagagaggcttcgctcatttcggaggaccgcatattctttccctcgtcacagaagttgccacaagggctttgaaggctgttcgcttccaaaagttcaacacgcatcgccgtctgcgtcccgaggccgttggtggcctgatcgagcgcttcaacagcaaccctgacgaccctcagttccaggatgtgaagccgttgttcgaagcgctggatgaggatatgatgcgccgcgttgctcgtcacaacagggaacagaaccaaggctctgactttggcatgccgcgtgccgacgacttcaaccccgccggtgacactctcgagacgatgctgttgcccatggctttccccgagggaagtccgatgcatccgtcgtacggtgccggccatgccactgtggccggggcttgtgttaccgtgttgaaggcattcttccagcacgacaccgagctcgatttctgcttcgttcccagtgatgacggaagccgtcttgtcgacgccagccacaacatgaataagaaattgactgtagagggagagttgaacaaagtctgctccaacatcagcattggccgcaactgggctggcgttcattactttaccgattacattgagtcgattctcctcggggaacagatcgcgctaggcatcttggaggagcagatgttgactttccccgaaaccttcacaatgaccgtccctctcttcagcggcgaattgaaggtcctccgctcgacctga

SEQ ID NO: 52 - RNA, 2001 nt, Chondrus crispus
  source 1..2001: mol_type = mRNA
atggcaaccccgtgcttcgcccaggcgccggcccccaccctacgacgatcgccgcgccgtctccccctcaccccgacctccttcctgccgaggccgttcctgccaaagcccttcccccgctccccccgccacccgcatccgcgcctcaacgccgcctccgcgcgcacctccgccgcccaaccccccgaccgcgcagccacgccgtgcgctacccgccagcagcaagcgctggagctgcgccgcgcggcggcgcagctcgcgcacgaccgtgcgcaccccgcgcacgtgtcgaacgggtcggaggagcttcacctgcacccgaagacgggcgcgccgctgctgctggccaacttcacgaaggggctgccgcacgagcgcgacacggggctggcagccgacgccgcgcactacgacctcttcgtggcggcggtccattccggcttgccggacgacatccgcgccatcccgctcggcccgcaggccgcgcccggcgtccgcctcccgcgctcgctccgcttccgctccggcatcgcacgcagctcgcacctcttcccgcacactgacgtccgcgcctgggagagcatggccgcggggaacgcgttcgacctgcaggggccggacgcgcaggccgtcaccatgccgcccgcgcccgccctcgcctcccccgagctcgcgctggagatgacggagaactactggatggccctgctgcgcgacgtccgcttctcgcagtttgccgcggaccccaccgtcgccgccgccatggagagcatgaacgctaccgagtgggtcgccctcgcgtgcggcgacgcgtcggccgtcgcggaggaggagcgcaagcgcctgcgcgggccgtttaccgcgcagaacgtgttccgtgggaatacgcaaggcgattgcaaggggccctatatctcccagtttctgctgtctggaagcgcggggctgggccaagtgaacagcggcgcggatggatacatccagtacggagcaatgcgtatggatcagcgcgttcgtgtcgccctcgaggggcttgactatatgaccacgtgggagagctggctggatgtgcagaacggggccgacttgcggggaagggagttgtatgaggacggcgaacgctttagatttatttgcaccccgagggacttggccacttatgtccattatgatgccctgtatcaggcttacttgaacgcctgcattatcatgctcaacaagaagatcccctttgacaagggcctgccgttccagaaggatgacgacatcgacaagcagcagggcttcgccttattcggcccgccgcacattctcaccctgtgcacggaggtcgccaccagggccctcaagggcatccgcttccaaaagtacaacgtccaccgccgtctgcgtccggaggctgtcggcggccgcgtggagcgctaccaccacaactgcgaggacccgctcttcgccgacgtcaagccgctgtacgacgccctggataaggacatgctcgagcgtgttgccgcgcacaacgccgagcagaacgccaaggacgactttgggaacagccgcttcgaagactattccccgacgggtagctccggcaggacctatctgctgcccatggcgtttccggagggcagccccatgcacccggcgtacggcgcagggcatgcggcagtggccggcgcgtgcaccactatcctcaaggcgttcttcgaccacgagcacgagcttgactttgcgtacgtgcccaccgcggatggcagcaaattggaggatgttgttgactctttgggggagaagctgaccgtcgagggagagctcaacaaggtctgctcgaatatcagcgtcgggcgcaactgggctggtgtgcactactttacagactatcgcgagtccatcatccttggggaaaagattgccctcggcctgctggaggagcagaagcttacgtatgctgaggagttctccatgacgattcctctctttgatggttccactgtcaccatttag

SEQ ID NO: 53 - RNA, 1896 nt, Chondrus crispus
  source 1..1896: mol_type = mRNA
atgtcggacatgtcgactgagatcagccctagacaacaggaagcctacgatatccgtgtcgccgccgccaccctggctctggctcgcgaccgtaaacgcaaagtcttcgaacccaacggtgaggaggaccgctacttccgccccaactcccagatcctcagctacctcggctcctacaccaagggcctgcctcacaaccgcagcacggccctcgtcacctcgccgaaccacttcgtccgcttcgtccaagccatcgactcgggcgagccagccgacttcatcaaggtcccgctcggcccgcgcccggccctcgagttcacgaccgaggaagagtgcatcgccgccaacctcttcaagtccggcatcgccacccgcaacgacagcttcgatcccgcctgcctgcgcgcatgggagagcatgggcgccggtctcgtcttcgacctggagggcccggacgcccaggccgtcggcatgccgcccgcccccacgctggacagcgacgagctcgtcgccgagatctccgagctgtactggatggccctgctgcgcgacgtccgcttcactgacttcactaaccccggcccgatccgcagggccatcaacaccctcaacaggaccacctggatccgcgcggcacgggacccccctgactcgctcacgccgcaggagcgcgcccgcctccgcggcccgttcaacccgagcagcatcttccgcggaacactccccggagatgacgtcggcccgtacctctcccagttcctgctcgtgggaactgagggtattggcggcgcgcaggacagggctgacgggtttattagctacggtgcccaccgcatcgaccagcgcgtgcgtcacgcgacaaaggagctcgactacatgacctcttgggaggcctttttggacgtccagaacggcgctgacgttgccggtcgcgatacctttgagtcgggctctgacgccttccgcttcattacgaccccccgtgatctcgccacgtatgtccactttgacgcgctctaccaggcctacctcaatgcctgcctgatcctgctcgacaacaaagtgaaatttgatcagggtattccgatgggtgagccagacttcaacggtaatcagggtattccgtttcaagatccagacttcaaggatcatcagcgaggctttgctcatttcggaggaccgcatattctctccctcgtcacagaagttgccacaagggctttgaaggccgttcgcttccaaaagttcaacacgcatcgccgtctgcgtcccgaagccgttggtggcctgatcgagcgcttcaacagcaaccctgacgaccctcagttccaggatgtgaagccgttgttcgaagcgctggatgaggatatgctgcgccgcgttgctagtcacaaccgtgaacagaacgaacgctctgactttggcatgccgcgggccgacgacttcaaccccgccggtgacactctcgagacgatgctgttgcccatggctttccccgagggaagtccgatgcatccgtcgtacggtgccggccatgccactgtggccggggcttgtgttaccgtgttgaaggcattcttccaacatgacgccacgctcgacttctgctacgttcccagtgatgacggaagccgtcttgacgacgccagccacaccttgaataagaaattgactgtagagggagagttgaacaaggtctgctccaacatcagcattggccgcaactgggctggcgttcattactttaccgactacattgagtcgattctgctcggcgaacagatcgcgctaggcatcttggaggagcagatgttgactttccccgaaaccttcacaatgaccgtccctctcttcagtggcggcttcaggaccctcatctcgacctctgatccttga

SEQ ID NO: 54 - RNA, 1779 nt, Asparagopsis armata
  source 1..1779: mol_type = mRNA
ttaaatgcgaattgtgcttccatcaaacttgttgagggtcatcgagaaattctcactgaaggtcaacttctgttcctgaagaatgccaatagcaatttcctcaccaatgcggatactttcaatgtagtcagtgaagtaatgcacaccagcccagttacgtccaatggaaatattagagcaaatcttgttcagctctccgtcaactgtgagcggctcagacacttcaaccggctcgaggccgcagccattttcgttagtgacgtatgcaaacggcagggggaatccaccatcaaagaatgccttgagaacagttacacaggctcctgcaacagtggcatgtccagcaccataagccggatgcatcgggcttccctctgggaatgccattggcatgagaaggttgccttcgctcaggccgccatcaggattgaaatctgcactacgcggagcaccgttgtcgctcaacttattttgcttcttgttatggtaatacactcgatccaacaggtcagaatccagggaatctaccaatttagcagctccggaaaattcgggtgctttggttttgtagtatcggtcgacacgaccgccgacagtctccgggcgtagacggcgatggacgttgaacttctggaagcgcactgccttgagagcgcgagtggccacctcagtaacgagagagagaatatgcggtccgccaaacgtggcaaatgcctgctgcttatctatatcattgtcgagctggaacgggactccggaatcgaacggtgcaccaatgtcgagcagaatgatgcaggcgttaagataggcctggtacagagcgtcgaaatggacgtatgtcgccagatcacgcggtgtgtgaatgaagcggaaaccagggtctgactcgtatcgctcacgcccggtgacatccgcagcgttctgcacgtcaagccaggatccaaaggtggtcatgtagtcaaccttgggggatgccacgcgtacccgctggtccatgcgcataccaccgtactggatgaaaccatcgctgacctcgtttccgtttgcgattcctgtggtaccgaccaagaggaactgggacaaatatgggcctacatcatctccaggagtgatgcccctgaagacattggaggcatcgatggggcctctgagacgggagcgttcggcagatgtgagatgggtgggagggttttccttgaatttaatccattgagtgtcgttgatactcttggcagcggctgcgacatcttcgtcgcaatggaatgttgggaagggtacatccctgagcagggacatccaataacattcggtgacttcagtgaccaactcctcgctgtctagctctggtgcgggcggcatagtgacggcctgtgcatcaggcccttcgagatcgaatgtgcgtccagcacccatactttcccaggcacgaactcccgactgctctgggtttgtggcgatgccggatgtgaatctgggggtgacattctgggggccgagtgggagagctgcgaaatcgcgggcatctccgctgtcaatagcgcgtacaaattggtcataatcgagggcagattgaaccagacctgtttcgttgatatgggggagacctttcgtgaagttagccaagagagatcgtgtaccatctgggtttctgaagcgagcctctgaattgttggagatgtgagttggagaaccacgggagcgagcaagctcggcggcagagaggcgaatcttgaaggaggcctctgcgcgattgggattctgtgtgtcgggggttgccat

SEQ ID NO: 55 - RNA, 2493 nt, Asparagopsis armata
  source 1..2493: mol_type = mRNA
atgaacacagcatttcccttcagaagaaaatggtccccaagtctttggagtgctcgcctcgagtcctatctctccactcacgccttcgagcttctcacagttatcacatacataactgcactctctctcatgttcgtatggggagctcacgatgagttccacaatcaatctcccgacacaaatctccgctggtacatttctattgctcgcggagcaggttacacactcaacctcaacacagcacttgtcattctacttgcatctcgcctcttcttcacattcatccgtgatactcccctgcacaagattcttcctgttgacaaaagcttccccgccttccatatccttgttggatatacgatagccgcatctctcttcattcatgtcccgttccatttcacatggatcattgcttacgataaatggagtgcaggactttgggatttcaccatgagcgctgtcacaggcgtcgtattgttacttatcttcataatcatgttcatgttcgctctaccattctacagaaaaaaatatttccggctcttctacatttcccacatggttggagcactgctgttctttcctcttctcatcttccatggtatgtaccgtcgtgttccggagacttacaagtgggttgctctacctctcgtgatctacattctggacagaatcgcccgtcgcttcaaaatctccaccgttcagctgaagctcacttccgagcattcagctctcaaaggaaaagacattttggaacttcgtattccgaaacccttcacatatcgcgctggtcagtatgcagagatccgtgtaccatccttgagtagggagtggcatcccttcacaattgctagtgcaccccatgaaaaatgcatgtgctttttcatcaaatcactcgggaattggaccaaggagcttcgaggggcatttcaaaaccgtatggaaggagaagatccgaacccattgctagttcaagtccgtggtccatttggcgctcccgctcaacatgtctcttcgtatgaaagagtggtcctcatatctggcggagttggttcaacgcccttcgcagccatctccaaaagtttgcatttcttgaacaagaacaaaacctgcccttctactgatttttcgaaaaccaatttgtctgaaatgaagcatattgagcagcgtatctcggaaactatttctgctgtatatgatatcgatttggcaaacagctccgatgaaccggaatctgaagaacaaaggaagcagtatattactgacatgatgaacttgacggcccaaaattccgaagaactcgatgtttccacttcaagaacaagtgaacaatgtgtatcatcgaggagctgcccaaatattggagatggtctccggatattggatgtcgaaaatcaattagaagaatcttcagtgagcagcatgtctacatgcgacgatgaagaagattattcatcagatttcagtgaagaaatgagacttgaaaaggaacttgccgccaggaaacgtaagatggctcgccaaaaaattgcccgtctctatgagagacgttcaatattattctcttttcttcataccactcgggtaactctggcacttcaactctctctaattgcacgcattgttcttgtgtgcattgtatcaatcttcaacattggtgaattcaactttggggacagccgcataggtgccaaatgggttataggtgccgatgcagtgttgagtgttttgcatgcagtcactctaactttgaccatactcttggaaattagcttcatgggaccccgcttcttccgttcttcttggcagtttatggactttttcgttttcgtcccgctcagtattctgtgcagcgtcagtggtgtactatcttggactgggacggaaatgtcaggaacattagtttttcttcacttcattgtattgctgccaattatgcttattttcatgagtgctcgtctgtatcggagtattgggtccctgaatctatttcatgatcggaagtgtaacccaaactgcgcatgcggaaagaaaatcccagacgttgactttatctggacaacacgtcacaccgtagatgaccaatggttacgagaagaactctttcctctagaggacggcacaaaactccgtcttcaccgatatattacaggcaaggaaagtgaagactttgaaagtgctgagacctttattactacaagcaacattggaagaccgccctgggacgaaatattcgccaaaattgcccaaaagtcaccaagtcacgcagaaatcggagtgtttttctgcggaccacactcaatgggagcagctgtgaggaaaagcatgcgaaaaactgaaatacggagtaacttgcgtggggcatatctggagacaactaatgttgtcgatcttgttgccgatcttcttctgagaaatactgaccatgcagcagagcttcggaggcatgggtgcaatattcgatttgtatttcgtgaagagaacttcggttga

SEQ ID NO: 56 - RNA, 2442 nt, Asparagopsis armata
  source 1..2442: mol_type = mRNA
tcagctaaagttctcctcccgaaatacgaatcgaacattgcttccaaaccgcttcagtaatcttacctcgtccccaacctcccctccacccgttttcctcaagtatgatcccctcaaactactgaaaacttccgcccgccgcaaactctcccgaacctgcttccccatcccactgggtccacagaagaacaccccgatctcactgcagctccgcgactcctccgcgatcttcaaaaatatttcatcccacttcggacgccccgccctagcagatgtgataaactcctcccctccctccacctccacttccttctcccgtgtcacgtaccgatgcagtcgcagctccgtccccgtcgcaagcgtgtacagatcttcgcgaagccactcgtcgtccccctcgtgcgcgaccgtccagataaagtccacttccggaacgctgtgttctgacccccggcaactcccgttatcgtcgaaaaggtttcgcgtcccaatgcttcggtataatcgatgacagagaagtaggaagataatcggtgtgataataatatagtcgacaaatgtcaggaaggacgattggtctcgtccagcccacgttacaattccgtatagattgcacagaaatgacagaggaacgaacaccaaaaagtcaacgcatcgccaattcctgtggaagaatcgcgtccccataaaactcacttccagcacaattattgtgaatagagtcaatccaacaattagtcccagaacgacgtttgtcacgataacccacttagcctctatgtgtccgctggttgtgctgaaatgaccaagatcgaagatagatacaacacacgtcagcactaatcgcgcaattagtgtgaacagcagcagcaatgttattcgagtcgtgtgaagtaaggaaagaagtctagagcgctttccattcagaaacatcaaattatgcttcgagtacgcagatcgccccatcttatcttctctcccgccttttctagagctgcactctttctcgtatgttcccagcgtggccatttgactcccaatacttgatgaaaacgagagatcgacattttccgaactgcacgtatcactcacatcagaagagcctgtcggagacggatgagagatttcatttcctaatttctgcaccgctccatccggggacatgttcagcatatccgccagatgctgtcttctttgttggttggaatcctctgcgcagccgctttcacccaaccccaagccgtacatattggagatcgtttctctgatccggttcccgagttgtattgtatgcttagatggaatgtcaacatcgagcgtctttttgtgatgttcagcgacattcaggtggtgcagatgtttcgagatggcggcaaaaggggtggcgccaacacctcctgacaccaacactacccgttgatatctgccgacgtgctgagcgggagcgccgtacggtccgcggatctgtaccgtaagtggtttgcagtcgtctgaatccatgcgtgtttggaattggtggcgcagtgcttgagtccaattgccaagagttttgatgaagaaggtcatggtttcttcgtggggtgcgcttgcgattgtgaatggatgccactctgaactgatcgaagggacacagacctctgcatattgccccgctctgtaatggaagggctttgggactcggagttcgagaatgtcagcactcttgagagaagaatgatcgctggtgagctgcaagggaactgtggacactttgaaacgacgaagaagtctatcaataatgtagatgatgagtggggcggtgatccatttgtaagtttctggaacgcggttgtacattccatgaaagattagtagagggaagaagagtatcactccgaacatatgtgtgagatagaagaatcggaagcgtttcttccgaaatgttgggagggcagttatgaacatgactagaaacacaatgaggaggacagatcctgtcgatgcgctcatagtgtatccccaaaggcctggttcccatttatcgtaggcaataatccacgcgaaatggaaagcgacatggaggacgagggagatggcaatagcgtaagcgacaataatatggaaggctgggaagcttttgtcaagaggaatgattagatgcaatggggtctcgcgaatataagttgaaaagagtctggcagcgagaagaatgaccaatgcagtattgagatttagggtgtaacctgctgcgcgggcgatggagatgtaccatcgcatgttatcatcggaagtgtggtgatggaattcgtcatgggctccccataggaacatgaggaggagtgcggtggcatacacgacgagagcgagaagttcgaaggcatgtgtggagaggtgggactcgagtcgagcggtgaggagatcttgagtggactttggacatgtggatgaggatggagagggcat

SEQ ID NO: 57 - RNA, 1749 nt, Asparagopsis armata
  source 1..1749: mol_type = mRNA
atgcctcccccaactcgtcgtcaatccgctctcgtcactcgcattgcagctgcagatgaagccgcaaaccgttcacatcctcctcatctaagaaatggcgatgagacgctgtacaagactacctcaaatgctctctcctacctcgcgaccttcacaaagggtctccctcatcatcccgacggtcgtctagtctccgcacagcattttcaactcttcgcccgtgctgtccaaacaggaactccacagcacattgcagatattcccctcggacctccatccaccttttccacattccgctccggaattgcaaaggatgccccactccgcgcttgggaatccatggccgctggaaatacattcgaccttcaagggcctgatccccaatcagtcacaatacccccatgtccacgtctcgaatccaatgagctcgtcacagagatgacagagctttactacatggcactcctcagggatgtcccattctctcatttcaaaagtcatcacctcgtcgccgctgccgtcgacgatatcaatcacacccattggattcaatcccataaatgttgcatcccagaactggaacgtcacagacacagagaacccttctcatcctctaatgtcttccgtggcaccacaccaggcgagcgcaatggtccttacatctcacaattcctcattgttggaaccaaagggctcggagaaatccacgacgaggcagatggctacatccaatacggtgccatccgaatcgatcagagagttcgatcagtcgagacagaaatcgactacatgaccacctgggccgccttccttgacgtccagaatgccgcagatttgcgcagacattctacatatgccaaatcaagctcctatcgcttcctgtacacaccacgcgacctcgctacctacgttcatcatgactcgctctaccaagcctatctcaacgcatgcatcatcctcctcgacatgaaagtcccattcgactccggtctccccttcaccacggaagaccacatcgacaaacaacagggctttgtaaactttggaggcccccatatcctcacactcgtcacagaggttgctacccgagccctcaaggcagtccgtttccaaaaatacgccgtccacagacgtcttcgcccagaggctgtcggcggcctcttagaacaatacagacgatacggtggcggatattcagagctagcaaatactattcgtcccatacgccatctcgcccgttctctaagttcaaacctcatgtcgctcgtcgcagcagcgaacggccatcagaacgcaacaatgagcgacaacggatttccacgctcccatgatacaggctccggtgagactcatttgctgccaatggcctatccggagggaagtccgatgcaccctagctatggtgccgggcatgccacagtggccggggcgtgcgtaacgatattgaaaggctttttcgatcacacctacaagctcccgttcgcctatgtctcatcctgcaatgggaagatgctgaagaaggcaaaggtcgcgaagaaactttcggtggaaggagagctgaataaactggcgtctaatatcagtattggacgtagctgggctggcgtacactactactccgactatgtggagagtattcgattgggagaggaagtggcgattggaatcctgaaagagcaaaagttaacttattcggaaaaattctcaatgacaattcccaaatttgatggcacaaccatcaaaatatag

SEQ ID NO: 58 - RNA, 1776 nt, Asparagopsis armata
  source 1..1776: mol_type = mRNA
atgtccgaagaacgccatgcatccgccttgagagtccgtagtgaagccgcagatctcgctgcttcacgccccctccccacccaccttgcaaacggcgatgagcatcgctaccacgacacccacctcgcctcctacacgaaaggtcttccacacgaaaaggataccggcctctcctcctctccagatcacttcgcagcctttgtacgcgccatcgaaacaggcgctcaagaagatattacaagtctccccctaggtatcgatgaagacccccgtttcatatccgccatcgcttcagagggctccgaccatcccatagccgacactagagcgtgggaatccatggcagctggtaatacatacgatctggagggaccggacgctcaggcagtcaccatgccgcctgccccaaaaatcgatagtgaagaactcttgacggagatcaccgagtgctactggatggcccttcttcgcgacgtcccgttcacagagttcgaaagggatggcactaccgctgccgcagccgcttcgatcagccgaacgcgctgggtgcagttccaagagcagcccgggcaacgcccctgcgatctgtctgatgcagaaattgcacgtttgcgaggtccattcaggaaaacaaacatcttccgcggcgtgacccctggtgaacaaatcggtccctatctctctcagttccttctcgttggcacaaagggtatcgcgaacattcaggaaatttcagatggattcattcaatacggaggtatgcgtatggaccaacgtgttcgtgtggcaaccccaaatagggactacatgaccacctgggcatcctggctcgacgtgcagaacgcaggtgatgtacgaggacgtgaaacgtacgacaacgacactccattccgtttcattactacaccgcgcgatcttgccacctacgtccatttcgatgcactttaccaggcatacctgaacgcatgtctcattcttctcgacaccaaggcccctttcgatccagaaatcccttttgtggaagatgatgacatcgataaacaacagggattcgctacctttggaggtccccatatcctctcgcttgtcacagaggtcgcaactagggcattgaaggctgtgcgcttccagaagtttggtacccaccgccgtctacgccccgaggctgtcggcggtcttatcgagcgataccgaaagacggagggagcagagccctttgagcccatccagaaaatcgccactgagatcgacaatgaccttatgacacgaatctcgcagcacaacgccgcgcagaatgaactttccgacaacggtggtgcccgaagcggcgattataatcctgagggctgcgaaagcgcagaaacttatcttctcccgatggcattcccggagggtagcccaatgcatcccagctatggtgcagggcatgctgctgttgcgggtgcatgtgtgacgatcctaaaggcgttcttcgaccacactcacgagcttgaattctgttatgtgcccagcacagacggccagaatcttgaggaggtggaaatcaatgaaccgctcacagtggagcatgagctcaacaagctttgtgccaacatcagtattggtaggaattgggctggtgtgcactattactcggattacatagagagcgcaaggttgggtgaagaggttgcaattggtattctagaagaacagagtctgacgtacggtgaggagttctccatgactattcccaagtttgatggtacgactatcaagatttcaaactcaaagtaa

SEQ ID NO: 59 - RNA, 1770 nt, Asparagopsis armata
  source 1..1770: mol_type = mRNA
tcagttggagatcgtaatgcacgttccatcaaacttggggagggtcatggagaacttctcaccatacgtcaagctctgttcttgcaaaataccgatggcaacctcttcacccaactttgcactctcttcgtaatccgagtagtagtgcacaccggcccagtttcttccaatactgatgttggcacacaacttgttcagctcgtgttcaactgtgagcggttcactgatgttaaccttctctagcttcctgccgtctgtgctgggttcaaagcagaactttaacttgtatttgtgatcaaagaaggccttgaggatcgtgacacaggcgccagcgacagcggcgtgtccagcaccgtagctgggatgcattgggcttccctccgggaacgccattggaagaaggtaggtttctgcgcttgcgccaccctcggggttgaaatcatttcttcgtgctttaccgttgtcggaaaactcattctgcttggcgttgtgacgcgagatgcgcgtcataagctcattgtcgatctcggtagcgagcttcttagcggctgcgaatggcgctgcgccgttcgtcttgcggtagcgctcgatgagtccgccgacagcttcggggcgtaggcgacgatgcgtaccgaacttctggaagcgcacagccttaagtgctctggttgcaacctctgtgacaagagaaaggatgtgaggcccgccaaaggtggcaaatccctgttgtttatcgatgtcgtcgtcctgcacaaaaggaaggccatgatcgaagggagcctttatgtcgagaagaatgagacaggcattgaggtatgcttgatagagggcgtcgaaatggacgtaggttgcgagatcgcgcggggtggtaatgaaacggtaatcagtatctttgtcgtatgtttcacgtgagcggacgtcccctgcattctgtacgtcgagccaggaggcccaagtagtcatatagtcgcggttgggagtcgcaaagcgaacgcgctggtccattcgcatacctccgtattgaatgaaaccttcttctggctggtgcattttcccgataccctgtgtaccgacaagcaagaactgggagaggtaggggcctacctgttcaccaggcatgtcgccgcggaagacgtttgatttgttgaaggggccgcgcagacgttggatttcagcatttgtgagatcggatgggcgctggccgggctgctcctgaaactgtacccagcgtgtacgactaatcgatgcggctgcggcagcaactgttccgtttcgatgaaattctgagaagggcacgtcgcgaagaagggccatccaatagcattctgtgatctctgtcaagagctcttcgctgtcgattttaggtgcaggaggcatggtgactgcctgtgcatcaggcccttcgagatcgaaggtattgcctgcggccatggattcccaggcgcgagtgtcagcgatggggaggtcgagtgctggggatgcgatgccggagatgaaacgaggtgtttcatcaatacctaggcggaggctcctgatgtcttcctgtgcgcctgtgttgatagcacgcacaaaggcagtgaagtcatctgtagatgagagaaggcctgtctttttgtcgtgtggtaaaccctttgtgtaggatgcgatgtatgaatctgagaaacgggtctcatcgccattgggggtgtgggtagggagtttgcgggaggctgctagttttgcggcatctgtgcgtacttgtagtgcggctgcttgacgtgtggtagacat

SEQ ID NO: 60 - RNA, 1779 nt, Asparagopsis armata; identical to SEQ ID NO: 54
  source 1..1779: mol_type = mRNA

SEQ ID NO: 61 - RNA, 2493 nt, Asparagopsis armata; identical to SEQ ID NO: 55
  source 1..2493: mol_type = mRNA

SEQ ID NO: 62 - RNA, 2475 nt, Asparagopsis armata
  source 1..2475: mol_type = mRNA
ctaactaaagttttcctctcgaaagacaaaccgaatattgcatccgtgttctctcaataacttaacctccttttcatccctcaatgacaagtcttccactaacaattcactgtcagtctttcgcaaatacgatcctcggaggttgctcatcacttcaactttcctcatactcttctgaactgctgcacccataggatgtggcccgcagaagaacactccaacttcattgtggctaggagtttgttcggcaatttgtgcgaaaatctcatcccactgaggccgtccagtattcgcagtagtgatgaaatgttcaggttcttccaggtccaagtccttttcacgtgttacatatcggtgcaatcgtagatcagttcctgtggccagcgggtacagctcactccgcagccactcatcctcactatcacgaggagtagtccatacaaagtcaacgtctggaatagttttgttacaggtgcaactctgacgacattgtgtgtcatccaaaaggttgcgtgcgccaatacttcggtacaggcgatggcaaagcaagaggaacatgattggaacgacaataatcaagtccagaaaaataagaaatttcgcttgttgacgtccactccaactcatgaaattgctgatattacagagaaaggtaatagggagaaagataaagaagtcgacgcaacgccaacctctgtagaagaagcgcagtcgcatgaaactgatttccagtagaacggtagtcaacagcgcaatcccgagtacgagtcccaataaggaattagtggcaatgacccattcggcatcaatatgactgtcactgattccaatatgcccaagattgaaaatagatacaacgcacaaaataacaattcttgcaaccagagtgagaagaagagcaaatgtgaatcttgttgtatgcagaaaagcgagcagtcttgagcggtactcatagaaattgaaaatcctccgctgcgcaacattgaagacgttggctgaaggtcgattataatatcgaatagattcagaacatgcatcaagattataactgctttgcttctctttctgagacagcgatatttcctcatccgctctgatctgcttggttatttcttcaaccgaactgcaatcatcagacgtctttgatagattgcgtttaaaagaaacgttatggacgtttccctttggagacatattgagcatatcggcgacttgttgtattcgctgttggttcgatgcagcatcactgcggctatcatccagacatacatcgaagagattggaaatggcatcacggatgtgccaatgaatttgagactgtcttttcgaagatccatgtgattccaaagctttcgaatggttttcagacttgttgagatggtgtaggtttttgcagattgctgcgaaaggtgttgaaccgacccctccggagataagaacgacgcgacgatatcctctcacgtgctgggctggagcaccatatggtccgcggatctggactttgagcggttcatataggtcattctcaattcgagcttcgaacgcatctcgaagatgtctagtccaatcaccaatcgccttgatgtagaagcacatagatttctcatgaggtgcacttgcaattgtaaaaggatgccactcattatttattgaaggcacgcatacctctgcatattgaccagcacggtattcgaacgtttttgggactcgcagctccagaatatcacttcccttcagggaagagtgctcaccagtgagttcgagcttcgctgttgaaatcttgaatcgtctcagaaccctatcaattgtgtagatagcgagaggtccggcaatccatttgtacgtttcggggactctgttgtacatgccgtgaaagataagcagagtgaagaatagtaatgcaccaaccatatggatgaggtagaaggtacggaattgcttctttcgaaatctaggaagagcagagacgaacatgacgacaaatactgtgagaagagcaaaacctgtagtggcactcatagtaaacccccacatgcctaattcccatttatcataggcaatgatccacacgagatggaaggaaacatgtatgaagagtgcggctgcaatggtgtaggccacgatgatgtggaaagctgggaaacttttgtcgattgggaggatgtgttgcagaggagtttcacggatgtaggtcgcgaaaagtcgggcggcgagaaggatgacgagagcggtattgagattgagtgtgtatccagcaccacgggcaatgcagatgtaccatcgtagatttttttgagaagtatggtgatgaaattcgtcatgggcaccccagacgaacatgagaatgtttgcgagtgagtagatgaaaagtgcgagaagcgagaatgcatttgttgatagataggattctaggcgtgcgctgaggagagtgagagaggtgggagagcatcgttgcttgccggagcggggagccat

SEQ ID NO: 63 - RNA, 2442 nt, Asparagopsis armata
  source 1..2442: mol_type = mRNA
atgccctctccatcctcatccacatgtccaaagtccactcaagatctcctcaccgctcgactcgagtcccacctctccacacatgccttcgaacttctcgctctcgtcgtgtatgccaccgcactcctcctcatgttcctatggggagcccatgacgaattccatcaccacacttccgatgataacatgcgatggtacatctccatcgcccgcgcagcaggttacaccctaaatctcaatactgcattggtcattcttctcgctgccagactcttttcaacttatattcgcgagaccccattgcatctaatcattcctcttgacaaaagcttcccagccttccatattattgtcgcttacgctattgccatctccctcgtcctccatgtcgctttccatttcgcgtggattattgcctacgataaatgggaaccaggcctttggggatacactatgagcgcatcgacaggatctgtcctcctcattgtgtttctagtcatgttcataactgccctcccaacatttcggaagaaacgcttccgattcttctatctcacacatatgttcggagtgatactcttcttccctctactaatctttcatggaatgtacaaccgcgttccagaaacttacaaatggatcaccgccccactcatcatctacattattgatagacttcttcgtcgtttcaaagtgtccacagttcccttgcagctcaccagcgatcattcttctctcaagagtgctgacattctcgaactccgagtcccaaagcccttccattacagagcggggcaatatgcagaggtctgtgtcccttcgatcagttcagagtggcatccattcacaatcgcaagcgcaccccacgaagaaaccatgaccttcttcatcaaaactcttggcaattggactcaagcactgcgccaccaattccaaacacgcatggattcagacgactgcaaaccacttacggtacagatccgcggaccgtacggcgctcccgctcagcacgtcggcagatatcaacgggtagtgttggtgtcaggaggtgttggcgccaccccttttgccgccatctcgaaacatctgcaccacctgaatgtcgctgaacatcacaaaaagacgctcgatgttgacattccatctaagcatacaatacaactcgggaaccggatcagagaaacgatctccaatatgtacggcttggggttgggtgaaagcggctgcgcagaggattccaaccaacaaagaagacagcatctggcggatatgctgaacatgtccccggatggagcggtgcagaaattaggaaatgaaatctctcatccgtctccgacaggctcttctgatgtgagtgatacgtgcagttcggaaaatgtcgatctctcgttttcatcaagtattgggagtcaaatggccacgctgggaacatacgagaaagagtgcagctctagaaaaggcgggagagaagatatgctggggcgatctgcgtactcgaagaataatttgatgtttcttaagggaaagcgctctagacttctttccttacttcacacgactcgaataacattgctgctgctgttcacactaattgcgcgattagtgctgacgtgtgttgtatctatcttcgatcttggtcatttcagcacaaccagcggacacatagaggctaagtgggttatcgtgacaaacgtcgttctgggactaattgttggattgactctattcacaataattgtgctggaagtgagttttatggggacgcgattcttccacaggaattggcgatgcgttgactttttggtgttcgttcctctgtcatttctgtgcaatctatacggaattgtaacgtgggctggacgagaccaatcgtccttcctgacatttgtcgactatattattatcacaccgattatcttcctacttctctgtcatcgattataccgaagcattgggacgcgaaaccttttcgacgataacgggagttgccgggggtcagaacacagcgttccggaagtggactttatctggacggtcgcgcacgagggggacgacgagtggcttcgcgaagatctgtacacgcttgcgacggggacggagctgcgactgcatcggtacgtgacacgggagaaggaagtggaggtggagggaggggaggagtttatcacatctgctagggcggggcgtccgaagtgggatgaaatatttttgaagatcgcggaggagtcgcggagctgcagtgagatcggggtgttcttctgtggacccagtgggatggggaagcaggttcgggagagtttgcggcgggcggaagttttcagtagtttgaggggatcatacttgaggaaaacgggtggaggggaggttggggacgaggtaagattactgaagcggtttggaagcaatgttcgattcgtatttcgggaggagaactttagctga

SEQ ID NO: 64 - RNA, 1749 nt, Asparagopsis armata; identical to SEQ ID NO: 57
  source 1..1749: mol_type = mRNA

SEQ ID NO: 65 - RNA, 1776 nt, Asparagopsis armata
  source 1..1776: mol_type = mRNA
ttactttgagtttgaaatcttgatagtcgtaccatcaaacttgggaatagtcatggagaactcctcaccgtacgtcagactctgttcttctagaataccaattgcaacctcttcacccaaccttgcgctctctatgtaatccgagtaatagtgcacaccagcccaattcctaccaatactgatgttggcacaaagcttgttgagctcatgctccactgtgagcggttcattgatttccacctcctcaagattctggccgtctgtgctgggcacataacagaattcaagctcgtgagtgtggtcgaagaacgcctttaggatcgtcacacatgcacccgcaacagcagcatgccctgcaccatagctgggatgcattgggctaccctccgggaatgccatcgggagaagataagtttctgcgctttcgcagccctcaggattataatcgccgcttcgggcaccaccgttgtcggaaagttcattctgcgcggcgttgtgctgcgagattcgtgtcataaggtcattgtcgatctcagtggcgattttctggatgggctcaaagggctctgctccctccgtctttcggtatcgctcgataagaccgccgacagcctcggggcgtagacggcggtgggtaccaaacttctggaagcgcacagccttcaatgccctagttgcgacctctgtgacaagcgagaggatatggggacctccaaaggtagcgaatccctgttgtttatcgatgtcatcatcttccacaaaagggatttctggatcgaaaggggccttggtgtcgagaagaatgagacatgcgttcaggtatgcctggtaaagtgcatcgaaatggacgtaggtggcaagatcgcgcggtgtagtaatgaaacggaatggagtgtcgttgtcgtacgtttcacgtcctcgtacatcacctgcgttctgcacgtcgagccaggatgcccaggtggtcatgtagtccctatttggggttgccacacgaacacgttggtccatacgcatacctccgtattgaatgaatccatctgaaatttcctgaatgttcgcgataccctttgtgccaacgagaaggaactgagagagatagggaccgatttgttcaccaggggtcacgccgcggaagatgtttgttttcctgaatggacctcgcaaacgtgcaatttctgcatcagacagatcgcaggggcgttgcccgggctgctcttggaactgcacccagcgcgttcggctgatcgaagcggctgcggcagcggtagtgccatccctttcgaactctgtgaacgggacgtcgcgaagaagggccatccagtagcactcggtgatctccgtcaagagttcttcactatcgatttttggggcaggcggcatggtgactgcctgagcgtccggtccctccagatcgtatgtattaccagctgccatggattcccacgctctagtgtcggctatgggatggtcggagccctctgaagcgatggcggatatgaaacgggggtcttcatcgatacctagggggagacttgtaatatcttcttgagcgcctgtttcgatggcgcgtacaaaggctgcgaagtgatctggagaggaggagaggccggtatccttttcgtgtggaagacctttcgtgtaggaggcgaggtgggtgtcgtggtagcgatgctcatcgccgtttgcaaggtgggtggggagggggcgtgaagcagcgagatctgcggcttcactacggactctcaaggcggatgcatggcgttcttcggacat

SEQ ID NO: 66 - RNA, 1770 nt, Asparagopsis armata; identical to SEQ ID NO: 59
  source 1..1770: mol_type = mRNA

SEQ ID NO: 67 - RNA, 1779 nt, Asparagopsis armata
  source 1..1779: mol_type = mRNA
atggcaacccccgacacacagaatcccaatcgcgcagaggcctccttcaagattcgcctctctgccgccgagcttgctcgctcccgtggttctccaactcacatctccaacaattcagaggctcgcttcagaaacccagatggtacacgatctctcttggctaacttcacgaaaggtctcccccagatcaacgaaacaggtctggttcaatctgccctcgattatgaccaatttgtacgcgctattgacagcggagatgcccgcgatttcgcagctctcccactcggcccccagaatgtcacccccagattcacatccggcatcgccacaaacccagagcagtcgggagttcgtgcctgggaaagtatgggtgctggacgcacattcgatctcgaagggcctgatgcacaggccgtcactatgccgcccgcaccagagctagacagcgaggagttggtcactgaagtcaccgaatgttattggatgtccctgctcagggatgtacccttcccaacattccattgcgacgaagatgtcgcagccgctgccaagagtatcaacgacactcaatggattaaattcaaggaaaaccctcccacccatctcacatctgccgaacgctcccgtctcagaggccccatcgatgcctccaatgtcgtcaggggcatcactcctggagatgatgtaggcccatatttgtcccagttcctcttggtcggtaccacaggaatcgcaaacggaaacgaggtcagcgatggtttcatccagtacggtggtatgcgcatggaccagcgggtacgcgtggcatcccccaaggttgactacatgaccacctttggatcctggcttgacgtgcagaacgctgcggatgtcaccgggcgtgagcgatacgagtcagaccctggtttccgcttcattcacacaccgcgtgatctggcgacatacgtccatttcgacgctctgtaccaggcctatcttaacgcctgcatcattctgctcgacattggtgcaccgttcgattccggagtcccgttccagctcgacaatgatatagataagcagcaggcatttgccacgtttggcggaccgcatattctctctctcgttactgaggtggccactcgcgctctcaaggcagtgcgcttccagaagttcaacgtccatcgccgtctacgcccggagactgtcggcggtcgtgtcgaccgatactacaaaaccaaagcacccgaattttccggagctgctaaattggtagattccctggattctgacctgttggatcgagtgtattaccataacaagaagcaaaataagttgagcgacaacggtgctccgcgtagtgcagatttcaatcctgatggcggcctgagcgaaggcaaccttctcatgccaatggcattcccagagggaagcccgatgcatccggcttatggtgctggacatgccactgttgcaggagcctgtgtaactgttctcaaggcattctttgatggtggattccccctgccgtttgcatacgtcactaacgaaaatggctgcggcctcgagccggttgaagtgtctgagccgctcacagttgacggagagctgaacaagatttgctctaatatttccattggacgtaactgggctggtgtgcattacttcactgactacattgaaagtatccgcattggtgaggaaattgctattggcattcttcaggaacagaagttgaccttcagtgagaatttctcgatgaccctcaacaagtttgatggaagcacaattcgcatttaa

SEQ ID NO: 68 - RNA, 2493 nt, Asparagopsis armata; identical to SEQ ID NO: 55
  source 1..2493: mol_type = mRNA

SEQ ID NO: 69 - RNA, 2475 nt, Asparagopsis armata
  source 1..2475: mol_type = mRNA
atggctccccgctccggcaagcaacgatgctctcccacctctctcactctcctcagcgcacgcctagaatcctatctatcaacaaatgcattctcgcttctcgcacttttcatctactcactcgcaaacattctcatgttcgtctggggtgcccatgacgaatttcatcaccatacttctcaaaaaaatctacgatggtacatctgcattgcccgtggtgctggatacacactcaatctcaataccgctctcgtcatccttctcgccgcccgacttttcgcgacctacatccgtgaaactcctctgcaacacatcctcccaatcgacaaaagtttcccagctttccacatcatcgtggcctacaccattgcagccgcactcttcatacatgtttccttccatctcgtgtggatcattgcctatgataaatgggaattaggcatgtgggggtttactatgagtgccactacaggttttgctcttctcacagtatttgtcgtcatgttcgtctctgctcttcctagatttcgaaagaagcaattccgtaccttctacctcatccatatggttggtgcattactattcttcactctgcttatctttcacggcatgtacaacagagtccccgaaacgtacaaatggattgccggacctctcgctatctacacaattgatagggttctgagacgattcaagatttcaacagcgaagctcgaactcactggtgagcactcttccctgaagggaagtgatattctggagctgcgagtcccaaaaacgttcgaataccgtgctggtcaatatgcagaggtatgcgtgccttcaataaataatgagtggcatccttttacaattgcaagtgcacctcatgagaaatctatgtgcttctacatcaaggcgattggtgattggactagacatcttcgagatgcgttcgaagctcgaattgagaatgacctatatgaaccgctcaaagtccagatccgcggaccatatggtgctccagcccagcacgtgagaggatatcgtcgcgtcgttcttatctccggaggggtcggttcaacacctttcgcagcaatctgcaaaaacctacaccatctcaacaagtctgaaaaccattcgaaagctttggaatcacatggatcttcgaaaagacagtctcaaattcattggcacatccgtgatgccatttccaatctcttcgatgtatgtctggatgatagccgcagtgatgctgcatcgaaccaacagcgaatacaacaagtcgccgatatgctcaatatgtctccaaagggaaacgtccataacgtttcttttaaacgcaatctatcaaagacgtctgatgattgcagttcggttgaagaaataaccaagcagatcagagcggatgaggaaatatcgctgtctcagaaagagaagcaaagcagttataatcttgatgcatgttctgaatctattcgatattataatcgaccttcagccaacgtcttcaatgttgcgcagcggaggattttcaatttctatgagtaccgctcaagactgctcgcttttctgcatacaacaagattcacatttgctcttcttctcactctggttgcaagaattgttattttgtgcgttgtatctattttcaatcttgggcatattggaatcagtgacagtcatattgatgccgaatgggtcattgccactaattccttattgggactcgtactcgggattgcgctgttgactaccgttctactggaaatcagtttcatgcgactgcgcttcttctacagaggttggcgttgcgtcgacttctttatctttctccctattacctttctctgtaatatcagcaatttcatgagttggagtggacgtcaacaagcgaaatttcttatttttctggacttgattattgtcgttccaatcatgttcctcttgctttgccatcgcctgtaccgaagtattggcgcacgcaaccttttggatgacacacaatgtcgtcagagttgcacctgtaacaaaactattccagacgttgactttgtatggactactcctcgtgatagtgaggatgagtggctgcggagtgagctgtacccgctggccacaggaactgatctacgattgcaccgatatgtaacacgtgaaaaggacttggacctggaagaacctgaacatttcatcactactgcgaatactggacggcctcagtgggatgagattttcgcacaaattgccgaacaaactcctagccacaatgaagttggagtgttcttctgcgggccacatcctatgggtgcagcagttcagaagagtatgaggaaagttgaagtgatgagcaacctccgaggatcgtatttgcgaaagactgacagtgaattgttagtggaagacttgtcattgagggatgaaaaggaggttaagttattgagagaacacggatgcaatattcggtttgtctttcgagaggaaaactttagttag

SEQ ID NO: 70 - RNA, 2442 nt, Asparagopsis armata
  source 1..2442: mol_type = mRNA
atgccctctccatcctcatccacatgtccaaagtccactcaagatctcctcaccgctcgactcgagtcccacctctccacacatgccttcgaacttctcgctctcgtcgtgtatgccaccgcactcctcctcatgttcctatggggagcccatgacgaattccatcaccacacttccgatgataacatgcgatggtacatctccatcgcccgcgcagcaggttacaccctaaatctcaatactgcattggtcattcttctcgctgccagactcttttcaacttatattcgcgagaccccattgcatctaatcattcctcttgacaaaagcttcccagccttccatattattgtcgcttacgctattgccatctccctcgtcctccatgtcgctttccatttcgcgtggattattgcctacgataaatgggaaccaggcctttggggatacactatgagcgcatcgacaggatctgtcctcctcattgtgtttctagtcatgttcataactgccctcccaacatttcggaagaaacgcttccgattcttctatctcacacatatgttcggagtgatactcttcttccctctactaatctttcatggaatgtacaaccgcgttccagaaacttacaaatggatcaccgccccactcatcatctacattattgatagacttcttcgtcgtttcaaagtgtccacagttcccttgcagctcaccagcgatcattcttctctcaagagtgctgacattctcgaactccgagtcccaaagcccttccattacagagcggggcaatatgcagaggtctgtgtcccttcgatcagttcagagtggcatccattcacaatcgcaagcgcaccccacgaagaaaccatgaccttcttcatcaaaactcttggcaattggactcaagcactgcgccaccaattccaaacacgcatggattcagacgactgcaaaccacttacggtacagatccgcggaccgtacggcgctcccgctcagcacgtcggcagatatcaacgggtagtgttggtgtcaggaggtgttggcgccaccccttttgccgccatctcgaaacatctgcaccacctgaatgtcgctgaacatcacaaaaagacgctcgatgttgacattccatctaagcatacaatacaactcgggaaccggatcagagaaacgatctccaatatgtacggcttggggttgggtgaaagcggctgcgcagaggattccaaccaacaaagaagacagcatctggcggatatgctgaacatgtccccggatggagcggtgcagaaattaggaaatgaaatctctcatccgtctccgacaggctcttctgatgtgagtgatacgtgcagttcggaaaatgtcgatctctcgttttcatcaagtattgggagtcaaatggccacgctgggaacatacgagaaagagtgcagctctagaaaaggcgggagagaagataagatggggcgatctgcgtactcgaagcataatttgatgtttctgaatggaaagcgctctagacttctttccttacttcacacgactcgaataacattgctgctgctgttcacactaattgcgcgattagtgctgacgtgtgttgtatctatcttcgatcttggtcatttcagcacaaccagcggacacatagaggctaagtgggttatcgtgacaaacgtcgttctgggactaattgttggattgactctattcacaataattgtgctggaagtgagttttatggggacgcgattcttccacaggaattggcgatgcgttgactttttggtgttcgttcctctgtcatttctgtgcaatctatacggaattgtaacgtgggctggacgagaccaatcgtccttcctgacatttgtcgactatattattatcacaccgattatcttcctacttctctgtcatcgattataccgaagcattgggacgcgaaaccttttcgacgataacgggagttgccgggggtcagaacacagcgttccggaagtggactttatctggacggtcgcgcacgagggggacgacgagtggcttcgcgaagatctgtacacgcttgcgacggggacggagctgcgactgcatcggtacgtgacacgggagaaggaagtggaggtggagggaggggaggagtttatcacatctgctagggcggggcgtccgaagtgggatgaaatatttttgaagatcgcggaggagtcgcggagctgcagtgagatcggggtgttcttctgtggacccagtgggatggggaagcaggttcgggagagtttgcggcgggcggaagttttcagtagtttgaggggatcatacttgaggaaaacgggtggaggggaggttggggacgaggtaagattactgaagcggtttggaagcaatgttcgattcgtatttcgggaggagaactttagctga

SEQ ID NO: 71 - RNA, 1749 nt, Asparagopsis armata
  source 1..1749: mol_type = mRNA
ctatattttgatggttgtgccatcaaatttgggaattgtcattgagaatttttccgaataagttaacttttgctctttcaggattccaatcgccacttcctctcccaatcgaatactctccacatagtcggagtagtagtgtacgccagcccagctacgtccaatactgatattagacgccagtttattcagctctccttccaccgaaagtttcttcgcgacctttgccttcttcagcatcttcccattgcaggatgagacataggcgaacgggagcttgtaggtgtgatcgaaaaagcctttcaatatcgttacgcacgccccggccactgtggcatgcccggcaccatagctagggtgcatcggacttccctccggataggccattggcagcaaatgagtctcaccggagcctgtatcatgggagcgtggaaatccgttgtcgctcattgttgcgttctgatggccgttcgctgctgcgacgagcgacatgaggtttgaacttagagaacgggcgagatggcgtatgggacgaatagtatttgctagctctgaatatccgccaccgtatcgtctgtattgttctaagaggccgccgacagcctctgggcgaagacgtctgtggacggcgtatttttggaaacggactgccttgagggctcgggtagcaacctctgtgacgagtgtgaggatatgggggcctccaaagtttacaaagccctgttgtttgtcgatgtggtcttccgtggtgaaggggagaccggagtcgaatgggactttcatgtcgaggaggatgatgcatgcgttgagataggcttggtagagcgagtcatgatgaacgtaggtagcgaggtcgcgtggtgtgtacaggaagcgataggagcttgatttggcatatgtagaatgtctgcgcaaatctgcggcattctggacgtcaaggaaggcggcccaggtggtcatgtagtcgatttctgtctcgactgatcgaactctctgatcgattcggatggcaccgtattggatgtagccatctgcctcgtcgtggatttctccgagccctttggttccaacaatgaggaattgtgagatgtaaggaccattgcgctcgcctggtgtggtgccacggaagacattagaggatgagaagggttctctgtgtctgtgacgttccagttctgggatgcaacatttatgggattgaatccaatgggtgtgattgatatcgtcgacggcagcggcgacgaggtgatgacttttgaaatgagagaatgggacatccctgaggagtgccatgtagtaaagctctgtcatctctgtgacgagctcattggattcgagacgtggacatgggggtattgtgactgattggggatcaggcccttgaaggtcgaatgtatttccagcggccatggattcccaagcgcggagtggggcatcctttgcaattccggagcggaatgtggaaaaggtggatggaggtccgaggggaatatctgcaatgtgctgtggagttcctgtttggacagcacgggcgaagagttgaaaatgctgtgcggagactagacgaccgtcgggatgatgagggagaccctttgtgaaggtcgcgaggtaggagagagcatttgaggtagtcttgtacagcgtctcatcgccatttcttagatgaggaggatgtgaacggtttgcggcttcatctgcagctgcaatgcgagtgacgagagcggattgacgacgagttgggggaggcat

SEQ ID NO: 72 - RNA, 1776 nt, Asparagopsis armata; identical to SEQ ID NO: 65
  source 1..1776: mol_type = mRNA

SEQ ID NO: 73 - RNA, 1770 nt, Asparagopsis armata
  source 1..1770: mol_type = mRNA
atgtctaccacacgtcaagcagccgcactacaagtacgcacagatgccgcaaaactagcagcctcccgcaaactccctacccacacccccaatggcgatgagacccgtttctcagattcatacatcgcatcctacacaaagggtttaccacacgacaaaaagacaggccttctctcatctacagatgacttcactgcctttgtgcgtgctatcaacacaggcgcacaggaagacatcaggagcctccgcctaggtattgatgaaacacctcgtttcatctccggcatcgcatccccagcactcgacctccccatcgctgacactcgcgcctgggaatccatggccgcaggcaataccttcgatctcgaagggcctgatgcacaggcagtcaccatgcctcctgcacctaaaatcgacagcgaagagctcttgacagagatcacagaatgctattggatggcccttcttcgcgacgtgcccttctcagaatttcatcgaaacggaacagttgctgccgcagccgcatcgattagtcgtacacgctgggtacagtttcaggagcagcccggccagcgcccatccgatctcacaaatgctgaaatccaacgtctgcgcggccccttcaacaaatcaaacgtcttccgcggcgacatgcctggtgaacaggtaggcccctacctctcccagttcttgcttgtcggtacacagggtatcgggaaaatgcaccagccagaagaaggtttcattcaatacggaggtatgcgaatggaccagcgcgttcgctttgcgactcccaaccgcgactatatgactacttgggcctcctggctcgacgtacagaatgcaggggacgtccgctcacgtgaaacatacgacaaagatactgagtaccgtttcattaccaccccgcgcgatctcgcaacctacgtccatttcgacgccctctatcaagcatacctcaatgcctgtctcattcttctcgacataaaggctcccttcgatcatggccttccttttgtgcaggacgacgacatcgataaacaacagggatttgccacctttggcgggcctcacatcctttctcttgtcacagaggttgcaaccagagcacttaaggctgtgcgcttccagaagttcggtacgcatcgtcgcctacgccccgaagctgtcggcggactcatcgagcgctaccgcaagacgaacggcgcagcgccattcgcagccgctaagaagctcgctaccgagatcgacaatgagcttatgacgcgcatctcgcgtcacaacgccaagcagaatgagttttccgacaacggtaaagcacgaagaaatgatttcaaccccgagggtggcgcaagcgcagaaacctaccttcttccaatggcgttcccggagggaagcccaatgcatcccagctacgctgctggacacgccgctgtcgctggcgcctgtgtcacgatcctcaaggccttctttgatcacaaatacaagttaaagttctgctttgaacccagcacagacggcaggaagctagagaaggttaacatcagtgaaccgctcacagttgaacacgagctgaacaagttgtgtgccaacatcagtattggaagaaactgggccggtgtgcactactactcggattacgaagagagtgcaaagttgggtgaagaggttgccatcggtattttgcaagaacagagcttgacgtatggtgagaagttctccatgaccctccccaagtttgatggaacgtgcattacgatctccaactga

SEQ ID NO: 74 - RNA, 1779 nt, Asparagopsis armata; identical to SEQ ID NO: 54
  source 1..1779: mol_type = mRNA

SEQ ID NO: 75 - RNA, 2493 nt, Asparagopsis armata
  source 1..2493: mol_type = mRNA
tcaaccgaagttctcttcacgaaatacaaatcgaatattgcacccatgcctccgaagctctgctgcatggtcagtatttctcagaagaagatcggcaacaagatcgacaacattagttgtctccagatatgccccacgcaagttactccgtatttcagtttttcgcatgcttttcctcacagctgctcccattgagtgtggtccgcagaaaaacactccgatttctgcgtgacttggtgacttttgggcaattttggcgaatatttcgtcccagggcggtcttccaatgttgcttgtagtaataaaggtctcagcactttcaaagtcttcactttccttgcctgtaatatatcggtgaagacggagttttgtgccgtcctctagaggaaagagttcttctcgtaaccattggtcatctacggtgtgacgtgttgtccagataaagtcaacgtctgggattttctttccgcatgcgcagtttgggttacacttccgatcatgaaatagattcagggacccaatactccgatacagacgagcactcatgaaaataagcataattggcagcaatacaatgaagtgaagaaaaactaatgttcctgacatttccgtcccagtccaagatagtacaccactgacgctgcacagaatactgagcgggacgaaaacgaaaaagtccataaactgccaagaagaacggaagaagcggggtcccatgaagctaatttccaagagtatggtcaaagttagagtgactgcatgcaaaacactcaacactgcatcggcacctataacccatttggcacctatgcggctgtccccaaagttgaattcaccaatgttgaagattgatacaatgcacacaagaacaatgcgtgcaattagagagagttgaagtgccagagttacccgagtggtatgaagaaaagagaataatattgaacgtctctcatagagacgggcaattttttggcgagccatcttacgtttcctggcggcaagttccttttcaagtctcatttcttcactgaaatctgatgaataatcttcttcatcgtcgcatgtagacatgctgctcactgaagattcttctaattgattttcgacatccaatatccggagaccatctccaatatttgggcagctcctcgatgatacacattgttcacttgttcttgaagtggaaacatcgagttcttcggaattttgggccgtcaagttcatcatgtcagtaatatactgcttcctttgttcttcagattccggttcatcggagctgtttgccaaatcgatatcatatacagcagaaatagtttccgagatacgctgctcaatatgcttcatttcagacaaattggttttcgaaaaatcagtagaagggcaggttttgttcttgttcaagaaatgcaaacttttggagatggctgcgaagggcgttgaaccaactccgccagatatgaggaccactctttcatacgaagagacatgttgagcgggagcgccaaatggaccacggacttgaactagcaatgggttcggatcttctccttccatacggttttgaaatgcccctcgaagctccttggtccaattcccgagtgatttgatgaaaaagcacatgcatttttcatggggtgcactagcaattgtgaagggatgccactccctactcaaggatggtacacggatctctgcatactgaccagcgcgatatgtgaagggtttcggaatacgaagttccaaaatgtcttttcctttgagagctgaatgctcggaagtgagcttcagctgaacggtggagattttgaagcgacgggcgattctgtccagaatgtagatcacgagaggtagagcaacccacttgtaagtctccggaacacgacggtacataccatggaagatgagaagaggaaagaacagcagtgctccaaccatgtgggaaatgtagaagagccggaaatatttttttctgtagaatggtagagcgaacatgaacatgattatgaagataagtaacaatacgacgcctgtgacagcgctcatggtgaaatcccaaagtcctgcactccatttatcgtaagcaatgatccatgtgaaatggaacgggacatgaatgaagagagatgcggctatcgtatatccaacaaggatatggaaggcggggaagcttttgtcaacaggaagaatcttgtgcaggggagtatcacggatgaatgtgaagaagaggcgagatgcaagtagaatgacaagtgctgtgttgaggttgagtgtgtaacctgctccgcgagcaatagaaatgtaccagcggagatttgtgtcgggagattgattgtggaactcatcgtgagctccccatacgaacatgagagagagtgcagttatgtatgtgataactgtgagaagctcgaaggcgtgagtggagagataggactcgaggcgagcactccaaagacttggggaccattttcttctgaagggaaatgctgtgttcat

SEQ ID NO: 76 - RNA, 2442 nt, Asparagopsis armata; identical to SEQ ID NO: 70
  source 1..2442: mol_type = mRNA

SEQ ID NO: 77 - RNA, 1749 nt, Asparagopsis armata; identical to SEQ ID NO: 57
  source 1..1749: mol_type = mRNA

SEQ ID NO: 78 - RNA, 1776 nt, Asparagopsis armata; identical to SEQ ID NO: 65
  source 1..1776: mol_type = mRNA

SEQ ID NO: 79 - RNA, 1770 nt, Asparagopsis armata; identical to SEQ ID NO: 59
  source 1..1770: mol_type = mRNA

SEQ ID NO: 80 - RNA, 1773 nt, Asparagopsis taxiformis
  source 1..1773: mol_type = mRNA
ctagatacggatggtcgaaccgtcaaacttgttcagcgtcattgagaagttttcactgaacgtcagcttttgctcctgcagaataccaatggcaatctcctcaccaatgcgaatactctcgatgtaatcagtgaagtaatgcacgccagcccaattgcgaccaatcgaaatattggagcaaatcttattcaactcgccgtcgacggtcaacggctcgtcaatttccactgccttaagacctgttccattctcgtcggtgatgtagcagaacggcaacctgtaaccaccatcgaagaacgccttcaacactgtgacacaagcgcctgcaacagtcgcatgtccagcgccgtacgcgggatgcataggacttccctctgggaacgccatgggcatcagcagattacctttgctcacatctccgtcggggttgaagtcgtttgcacgcgggtttcctcgatcgctcaacaagttttgtttcttgttatgatcgtgcaccttctgcaacaacttcttgtccagcgcttcggagagcttggcagcgccagcaaattcaggtgcctttgttgcgcagtatcgatcgactcgggcaccaatggcctccggacgcagacgacgatggacgttgaatttctggaagcgaacagccttgagggctcgagtggccacttcggtaactagagagaggatgtgcggaccgccgaaagtggcaaatccttgctgcttatcaatgtcgttatcgagctggaagggaattccagagtcgaatggcgcaccgatgtcgagcaagatgatgcaagcgttgaggtaggcctgatacaaggcgtcgaaatgcacgtaagtggccagatcacgaggagtgtgaatgaagcggaaacggggttcctcttcattgtacaattcgcgaccggagacgttggcggcattttgcacgtcgagataggcgccgaatgtggtcatgtaatcaatgtgtgacttggcgacacgcacgcgctggtccatacgcatgccaccgtactggatgaatccgtcgccaacttcattgccattggcaataccggtggtaccgacaaggaggaactgtgacagatagggtccgacttcgtctcccggtgtgatacctctgaagacgttggcagtggtgatggggcctctgagacgggaacgctcagcagctgtgagatgtgctggtgggttgttcttgaattgaatccattgggtgttattgatggactctgcagctgactgaatatggggatcactgtcaaatgtcgggaagggaacgtctctcagcagagacatccagtagcattctgtgacttcagtaaccaattcatcgctgtccagctcgggcgcgggcggcatagtgacagcctgagcatcgggaccttcgagatcgaagacaagaccagcaccgccactttcccaggcacgggttccgacctctgggtcgctagcgataccggaggtgaatctgggctcaacgccctgcggaccgagcgggaggtctgcgaaatcacgaggatcaccgctgtcaattgcgcgaacaaagctatcgtagtcgattgcggacaccactagagcggtttccttggtatgcgggagacccttggtgaaattggcgagcagcgaacgggtaccatcagggtttctgaagcgactctccgagttgttggacagatgggttggggagccacgagcgcgtgccagctcagctgctgatacgcggactttgaaggccacttctgcacgattgggattctgtgtgtcggtcat

SEQ ID NO: 81 - RNA, 2559 nt, Asparagopsis taxiformis
  source 1..2559: mol_type = mRNA
atggataccgtttcccccggagactctcgccagaaagcacagggaaagatgcgccccatgcgctccttcgccatcctctcggctaatctcgaagctttcctctctacacatgccttctccctcctcttcctcatcttctacctcacctgtacagccctcatgttcgtttggggcttcaccacacagtggaagaagaccgacaacgaagccatgatctatttcatttccattgccagaggtgctggttacagtctcaatctcaacacaagcctcgtcatcctcattgccgctagactcactttcactcttatccgcgaaactcccctccaatacattctcccactcgataagattttcccgcttttccacatgtttgtcggctacatgattgcattcctcgttctcatccacggcactttccatatgacatggatcatccgcttccaaggatggaactcgggtctctggggtttcacaatgagtttcgtcaccggtttcatcatccttccagtctttgtcgtcatgctcatcacttcccttcctcacttccgcaagaacaagttccgcctcttctacctcttccatctttacggcgccgctctcttcttctccatcctcatcttccacggtatgtattttgagaggccagagacctacaaatggattacaccctccctcatcatctatctcctcgatcgtttcatcagacgtatgaagatcagcagtcatgatctggacattgccctggaaaaatccatcctcaaggacggtgatattctcgaactccgtgttcccaaacccttcgactatcgtcccggccattatgcagaggttatggttccgtccattaacaaggaatggcatccattctccatcgctagtgcaccacacgaagaaaccatgtgcttctacatcaaggtgctcggtgattggaccaccgaagttcgtaatgctttcaaggctcgtctcgatggtacaatcactgaaccactcaaagtacgtgttcgtggcccgtacggagctccatgccaggatgtcgacggtttcaagcgtgtcgtcctcgtctccggaggtgttggtgccacaccattttccgccattgttaaggagctgcactacctcaagacgcaggctaagaacaagaacgatggcccgggacgcattgcccgcgacattgatgttcgcattcgtgacagtatcgctaatctctacgatgtcgaagtgcaggacatggagaaggctactcacattgcgcagcagaagagcatctacgttactgaaatgcttgagctcaccgccaggaggacctcttccctgcaccgagtgaggtccagtgtcatggatcgcatgcatggatcgtcatctacaaagacagtgctcagtaccgatgctgctattcccagcttggaggatggccctgttggtggtggccaaccgatgagcaggatgcagcgtttgtcatcttcattgagcaaggctggaaacattcgacgagtgatgagcaccttgtcggagaacccggttacttgggatcaagtgatgtctgcgcagaacgatccattgaccaacacgaccgatcttcgtcgtcagatggcgcgcctttacgataagagaggaatgctcttggcgttcatgcacacagctcgcttcacctttttcctgctcctcactttgattgtacgaattattattatttccgttgccgccactctggattcgccctatttccagctcaacgatcaatttgatggctacacgcgcttgtggcctgtcgttgtcgacgccctcttctccgttccagttgctgctgcactggtggtctccattacacttgagattagtttcatgcgcatgcgatactttgacactgcggcgagaacctttgacgccttagtcctgatgccagcggccgtcatcggctgtgtgcttgccatccttacttgggcacgacaggacccggaatctgttcttgtcaaggttattgaatatgtactgttcatcccgctattgttcctaggtctgagctggcgcttgtaccgcatcacacggtcgaggagtcttctctcggatccggcatgccgatgcgataactcggaccacattcccgatgttgactttatctggtcgttcccctacgagcagagtgatcaatggttgcaggatgagctcggtccgctagcaaagggtactgaactacgacttcataggtatgtgactagggagaaggaacttgatgtggaggcggcgaaatcatttattaccagtgccaatactggacgtccaaagtgggatgatattttccagacaatcgccgaggaaacgccaagtaatggagcaattggtgtgttcttctgtggaccccacgtgatgggcaatcaagttcgcgatgccatgctcaaggtcgaggtgctgagcaatttgcgagctgcttacttgcgaaatacgcccgacgaggtgttgatgaccgacttgaatttgccaaatgctgaagcagtggagaggctgcgatcgttgggatgcagcgttcatttcgtctatagggaagaaaactttggttga

SEQ ID NO: 82 - RNA, 1746 nt, Asparagopsis taxiformis
  source 1..1746: mol_type = mRNA
atgacgccgccaacacgccgccaaacagcgctcgtcaagcgcattgccgccgcagacctggccgccaatcgtcaccatccgcatcatgtgcgcaatgccgacgaatccacctacaaattgccaaaccatgtctgttcctacatatccgctttcaccaagggtttgccccatcatctcgacggccgcctcgtttccccacaccactttcaactcttcgtccacgccgtgcaaactggccatgtcacagacattgccgccattccactcggacccccacactcctcctccttccgttccggcatcgccaagggcgccgccgtgcgcgcatgggagtccatggccgcgggtaacacctttgatttgcaagggcctgatgcacaggccgttaccatgcccccttgtccgcgtctcaattcacacgagctcgtcacagagatgacagagctttattatatggcactgttgcgcgacgtgccgtttacgcatttcaacaaacatcatctcgttcagtccgctgtgcacgacatgaatcatacccattggactcggtccaacagatgctgtttacctcgccccgagcgccgtcgacatcgctccaaattcaccactgataacatcttccgcggtgtaacgcccggtgacataaccggtccctacatttcccagtttctccttgtcggcacaaagggtctcggcgatgcacacgaagaaacagaaggctacgttcaatacggcgccacaagaatcgaccagcgagtgcgcactgtcgaacccaaaattgactacatgacgacatgggccgcttatctcgacgtgcagaatggtgccgacttacgccgtcactccacctatgccaaaccaccgtcctaccgcttcattagcaccccgcgcgacctcgcaacctatgtgcattacgacgcgttgtatcaagcgtatctcaacgcgtgcatcatcctgctcgacatcaacgcgccatacgatggcggactgcccttcaccgccgacgatgccgtcgacaagcagcagggctttgcgacatttggcgcgccacatgtgctgacacttgtgacagaagtggcgacacgcgcgctcaaagctgtgcgctttcaaaagtatgccgtgcatcggcgtctgcggccggaagctgtcggcggtctattggagcagtatagacgctacggtggtggacattcggatctggcatacatcattcgtccgattcgtcatctcgccgactcgctcagttccgatctgatgttcatggtcgccaaggagaacggacgccagaatagctcgattacggataacggccatccgcgctcgcatgacgaaggttcggatgatacgcatctgctttccatggcgtatgcggagggaagcccgatgcatcctagttatggcgcgggacatgcaacggttgcgggcgcttgtgtaacaatcctgaaagcgttcttcgatcatacgttcaaactgccgtttgcgtacgtgtcgtcgagcgatgggaggaaattgaagacggtcaagttgagcaggaaattgaccgtggaggatgagttgaacaagttggcagcgaatataagcattggacgcagttgggctggcgtccattactattcggattatgttgagagcattcggttgggcgaggaggtggccatcgggatgctcaaggagcagaagttaacttattcggagaaattcacgatgacaatacccaaatttgacggctccgtcattgaaatttag

SEQ ID NO: 83 - RNA, 1761 nt, Asparagopsis taxiformis
  source 1..1761: mol_type = mRNA
atggcagaggaacgtcgccaaaacgctctagacatccgcatccaagccgccaggcttgcgaagaaacgtgaccatccaacccatgaggcaaacggcgatgaggatcgttacccagaaacactcatcggctccttcactaaaggtcttccacatgagaaagaaaccggtctcctctccaaccccgccgatttcgccgactttgttcgcgctatcaacacaggcgcgatcaaagacataagacgcctcaaacttggaatcgatgaggatcctcgcttcatatctggtattgcctccaagggcgacaagcatcctttcgccgatacgcgcgcctgggaatccatggccgccggtctcacgtacgatctagaaggccctgatgcccaagctgtcaccatgccgccggcgccgaaactcgacagtgatgaactcgtcacagagattacggagagctattggatggctcttattcgtgatgtgccgttcacagagtttgaacgtgatggaaatacagccgcagccgcagcttccatcagtcgcactcgttgggtacagttcaatgagcagccttcacagcgtccgtcaacattgaccgatgaagagattgctagattgaggggtccgtataccaagaaaaatgtattccgtggtgtgacgatcggtgagaacgtcggcccgtatctgtctcagttcttactggtaggcacgaaaggtatcggcgatgtacagcaaatttcggatggttacattcagtacggaagtatgcgaatggatcagcgtgtccgcgtcgcagcgccgaaacgcgattatatgacaacttgggcatcttggctagatgtgcagaacgctggtgatttacgtggacgtgaaatttacgatgatgatacaccgttccgcttcattacaaccccacgcgaccttgcgacatgggttcatttcgacgcgttataccaggcatatttgaatgcttgcatcatccttctcgacatcaaggcgccgttcgatccacacatcccgttccaggctgatgacgatgtagataagcagcaaggatttgcaacgtttggcggcccgcacatcctatcattgtgcacggaagtggctacgagagcactcaaggcagttcgcttccaaaagtacaacgtgcatcgtcgtcttcgtccggaggctatcggcgggcttgtggaacgcttcaagaagacaaacggagatgccaagtttgccccagtgaagaagctggtgaatgacctggacggcgatatgctgcgacgcattgagcagcacaactgtgagcagaataagctttcagatgatggacatgcacgccgagacgactattcaccggagggtgagagtaaccagacgtaccttctgcccatggcgttcccggaggggagcccgatgcacccgtcatatggtgcaggtcacgccacagttgccggtgcgtgtgtgacggtgctcaaggcattcttcgaccatgaatacgagctggacttctgctacgtaccgaccacggatggcaagcgtctggagaaggtaaatatcaatgaaaaattgacagtggaaggcgaactgaacaagctgtgcgctaatatctgtattggtcgaaactgggctggcgtgcattactactcggactactttgagagtatcaaagttggtgaggagattgcaattggtattttgcaagaacagaagcttacgtatggtgaagactttttcatgacgctgtccaagtttgacggtgaaaagatccgtatctaa

SEQ ID NO: 84 - RNA, 2058 nt, Asparagopsis taxiformis
  source 1..2058: mol_type = mRNA
tcaagcaacaggatcatcacttcgcaacacctccacagtgcgcttgtcaacattcagctcgtgctttggctggctacgaatgacattaaggtggccatcgaagccgacgtaagtgaagctatgttgctccgcgaaggtcagcacctgctcctcaagaattcccctcgcgacttcctctccgaggatcagagactcaatgttgtcggacgaccagtgtactcctgcaagtgatcgaccaaatgcgacattgtttgcaatcttgttcagctcaccatcaacagtcagcgtcataccgtccggaagacacacgggctgcagacatccatgcgcgtcggcctcaaagatctcattcggcaagggggtaaactccacattagacggcttcgcgtccttcacagcgcactttccgtcgaactgcaccgtgttaaagaatgccttcagaataatcgtgcaagcgccagccactgtcccgtgtcctgccccatacgagggatgcataggagaaccttcggggaacgccattggcagtaagtacgacggagaagacgcgctgcggtctttcacacggtcagcaaacgtctggttctgctgcatgttcttgtcagcaacaaggtcgaggagaccgcatccgtcgagccgagcgacaagagcgcgaacttcctcgccgaagtcgcatcgctcactcttctgcgcgtggatcagaccgccgactgcttcaggccgggcccggagatgaatctggaacttctggtaacggactgccttgagagccctggtcgcaacctcacagacaagggacaggatgtgcggaccgccgaaagtcgcgaacgctgtttgtttgtcgtgctcgtcgtcatcgcggaacggtaagcccgtatcgaccccgacaccgtccgccagcagagcaaagcatgcattcaagtaggcttcgtacaacgcatcgtaatggacatatgtcgaaaggtcgcgtccatctgcaataaaccggaatatcggactcccatcaggagcacacttgtagtcggggaattcaccctgcctgacatccgccccgtcctgaacatcgatgaaagcctcccacgttgtcatccaatcttttcttggctcaattgatctcactcgcaagtcaagcccaagcgaaccgtacataacctgtcctgttgcagtaccatccgttccaaggccctggctaccgcggatcagatattgtgatatgtacggaccatctcggaccccagctccgtagcctctgaaaatatttccagtagtcaagttaagcttcccctcccgtctgaacctctcctcatcagtgagccccttcccgaactcttcagagacccacggcaacgcgctcaaggtagcaatcgcattatcaaagaaggccttcgtatcgggagagtcccacaaactgaaaggaatatcccgcgcaagcgccatgaagtaaagctctgcaatttcaaactcaagctccgcggacgcgagcttcggcgcgggcggcatagtgacagcctgcgcgtcgggtccttccaggtcaaacgcgaggcctgcgcctgcgctttcccacgcgcgcgccttgacctgcgactcgaacggcaggccgggcgcgccgtttgcaagcgtggattgccagcggccgggagtgtcttttggcagcttcgggcccagccggattttcttgaagtccgcgggatcgcccgtggccagcgacgcgcggaaggggtcgaaaagatcacggctgaaaatctgaccggtctcggtgttgtgccgcagacccttcgtgaaagtgccgatgagggtttccttgaaatctgactcatctccgttgctcttgtgtgggcgaactccgcggttgaatgccaggtttgctgcgttgatcctgacctgcttggcgtctttgcgacgctgctcctggtcggagttgtcctgggccattttcacggaggcggcggaagactgtcgacggaaagcattgaaaggggaatgcttcgtggtgattgatgcgcctctgatacgtgcaaccccaactggcgcagaggctacgaaagcgttatggaacat

SEQ ID NO: 85 - DNA, 1773 bp, Asparagopsis taxiformis
  source 1..1773: mol_type = other DNA
atgaccgacacacagaatcccaatcgtgcagaagtggccttcaaagtccgcgtatcagcagctgagctggcacgcgctcgtggctccccaacccatctgtccaacaactcggagagtcgcttcagaaaccctgatggtacccgttcgctgctcgccaatttcaccaagggtctcccgcataccaaggaaaccgctctagtggtgtccgcaatcgactacgatagctttgttcgcgcaattgacagcggtgatcctcgtgatttcgcagacctcccgctcggtccgcagggcgttgagcccagattcacctccggtatcgctagcgacccagaggtcggaacccgtgcctgggaaagtggcggtgctggtcttgtcttcgatctcgaaggtcccgatgctcaggctgtcactatgccgcccgcgcccgagctggacagcgatgaattggttactgaagtcacagaatgctactggatgtctctgctgagagacgttcccttcccgacatttgacagtgatccccatattcagtcagctgcagagtccatcaataacacccaatggattcaattcaagaacaacccaccagcacatctcacagctgctgagcgttcccgtctcagaggccccatcaccactgccaacgtcttcagaggtatcacaccgggagacgaagtcggaccctatctgtcacagttcctccttgtcggtaccaccggtattgccaatggcaatgaagttggcgacggattcatccagtacggtggcatgcgtatggaccagcgcgtgcgtgtcgccaagtcacacattgattacatgaccacattcggcgcctatctcgacgtgcaaaatgccgccaacgtctccggtcgcgaattgtacaatgaagaggaaccccgtttccgcttcattcacactcctcgtgatctggccacttacgtgcatttcgacgccttgtatcaggcctacctcaacgcttgcatcatcttgctcgacatcggtgcgccattcgactctggaattcccttccagctcgataacgacattgataagcagcaaggatttgccactttcggcggtccgcacatcctctctctagttaccgaagtggccactcgagccctcaaggctgttcgcttccagaaattcaacgtccatcgtcgtctgcgtccggaggccattggtgcccgagtcgatcgatactgcgcaacaaaggcacctgaatttgctggcgctgccaagctctccgaagcgctggacaagaagttgttgcagaaggtgcacgatcataacaagaaacaaaacttgttgagcgatcgaggaaacccgcgtgcaaacgacttcaaccccgacggagatgtgagcaaaggtaatctgctgatgcccatggcgttcccagagggaagtcctatgcatcccgcgtacggcgctggacatgcgactgttgcaggcgcttgtgtcacagtgttgaaggcgttcttcgatggtggttacaggttgccgttctgctacatcaccgacgagaatggaacaggtcttaaggcagtggaaattgacgagccgttgaccgtcgacggcgagttgaataagatttgctccaatatttcgattggtcgcaattgggctggcgtgcattacttcactgattacatcgagagtattcgcattggtgaggagattgccattggtattctgcaggagcaaaagctgacgttcagtgaaaacttctcaatgacgctgaacaagtttgacggttcgaccatccgtatctag

SEQ ID NO: 86 - DNA, 1776 bp, Asparagopsis armata
  source 1..1776: mol_type = other DNA
atgtccgaagaacgccatgcatccgccttgagagtccgtagtgaagccgcagatctcgctgcttcacgccccctccccacccaccttgcaaacggcgatgagcatcgctaccacgacacccacctcgcctcctacacgaaaggtcttccacacgaaaaggataccggcctctcctcctctccagatcacttcgcagcctttgtacgcgccatcgaaacaggcgctcaagaagatattacaagtctccccctaggtatcgatgaagacccccgtttcatatccgccatcgcttcagagggctccgaccatcccatagccgacactagagcgtgggaatccatggcagctggtaatacatacgatctggagggaccggacgctcaggcagtcaccatgccgcctgccccaaaaatcgatagtgaagaactcttgacggagatcaccgagtgctactggatggcccttcttcgcgacgtcccgttcacagagttcgaaagggatggcactaccgctgccgcagccgcttcgatcagccgaacgcgctgggtgcagttccaagagcagcccgggcaacgcccctgcgatctgtctgatgcagaaattgcacgtttgcgaggtccattcaggaaaacaaacatcttccgcggcgtgacccctggtgaacaaatcggtccctatctctctcagttccttctcgttggcacaaagggtatcgcgaacattcaggaaatttcagatggattcattcaatacggaggtatgcgtatggaccaacgtgttcgtgtggcaaccccaaatagggactacatgaccacctgggcatcctggctcgacgtgcagaacgcaggtgatgtacgaggacgtgaaacgtacgacaacgacactccattccgtttcattactacaccgcgcgatcttgccacctacgtccatttcgatgcactttaccaggcatacctgaacgcatgtctcattcttctcgacaccaaggcccctttcgatccagaaatcccttttgtggaagatgatgacatcgataaacaacagggattcgctacctttggaggtccccatatcctctcgcttgtcacagaggtcgcaactagggcattgaaggctgtgcgcttccagaagtttggtacccaccgccgtctacgccccgaggctgtcggcggtcttatcgagcgataccgaaagacggagggagcagagccctttgagcccatccagaaaatcgccactgagatcgacaatgaccttatgacacgaatctcgcagcacaacgccgcgcagaatgaactttccgacaacggtggtgcccgaagcggcgattataatcctgagggctgcgaaagcgcagaaacttatcttctcccgatggcattcccggagggtagcccaatgcatcccagctatggtgcagggcatgctgctgttgcgggtgcatgtgtgacgatcctaaaggcgttcttcgaccacactcacgagcttgaattctgttatgtgcccagcacagacggccagaatcttgaggaggtggaaatcaatgaaccgctcacagtggagcatgagctcaacaagctttgtgccaacatcagtattggtaggaattgggctggtgtgcactattactcggattacatagagagcgcaaggttgggtgaagaggttgcaattggtattctagaagaacagagtctgacgtacggtgaggagttctccatgactattcccaagtttgatggtacgactatcaagatttcaaactcaaagtaa

SEQ ID NO: 87 - DNA, 2001 bp, Chondrus crispus
  source 1..2001: mol_type = other DNA
atggcaaccccgtgcttcgcccaggcgccggcccccaccctacgacgatcgccgcgccgtctccccctcaccccgacctccttcctgccgaggccgttcctgccaaagcccttcccccgctccccccgccacccgcatccgcgcctcaacgccgcctccgcgcgcacctccgccgcccaaccccccgaccgcgcagccacgccgtgcgctacccgccagcagcaagcgctggagctgcgccgcgcggcggcgcagctcgcgcacgaccgtgcgcaccccgcgcacgtgtcgaacgggtcggaggagcttcacctgcacccgaagacgggcgcgccgctgctgctggccaacttcacgaaggggctgccgcacgagcgcgacacggggctggcagccgacgccgcgcactacgacctcttcgtggcggcggtccattccggcttgccggacgacatccgcgccatcccgctcggcccgcaggccgcgcccggcgtccgcctcccgcgctcgctccgcttccgctccggcatcgcacgcagctcgcacctcttcccgcacactgacgtccgcgcctgggagagcatggccgcggggaacgcgttcgacctgcaggggccggacgcgcaggccgtcaccatgccgcccgcgcccgccctcgcctcccccgagctcgcgctggagatgacggagaactactggatggccctgctgcgcgacgtccgcttctcgcagtttgccgcggaccccaccgtcgccgccgccatggagagcatgaacgctaccgagtgggtcgccctcgcgtgcggcgacgcgtcggccgtcgcggaggaggagcgcaagcgcctgcgcgggccgtttaccgcgcagaacgtgttccgtgggaatacgcaaggcgattgcaaggggccctatatctcccagtttctgctgtctggaagcgcggggctgggccaagtgaacagcggcgcggatggatacatccagtacggagcaatgcgtatggatcagcgcgttcgtgtcgccctcgaggggcttgactatatgaccacgtgggagagctggctggatgtgcagaacggggccgacttgcggggaagggagttgtatgaggacggcgaacgctttagatttatttgcaccccgagggacttggccacttatgtccattatgatgccctgtatcaggcttacttgaacgcctgcattatcatgctcaacaagaagatcccctttgacaagggcctgccgttccagaaggatgacgacatcgacaagcagcagggcttcgccttattcggcccgccgcacattctcaccctgtgcacggaggtcgccaccagggccctcaagggcatccgcttccaaaagtacaacgtccaccgccgtctgcgtccggaggctgtcggcggccgcgtggagcgctaccaccacaactgcgaggacccgctcttcgccgacgtcaagccgctgtacgacgccctggataaggacatgctcgagcgtgttgccgcgcacaacgccgagcagaacgccaaggacgactttgggaacagccgcttcgaagactattccccgacgggtagctccggcaggacctatctgctgcccatggcgtttccggagggcagccccatgcacccggcgtacggcgcagggcatgcggcagtggccggcgcgtgcaccactatcctcaaggcgttcttcgaccacgagcacgagcttgactttgcgtacgtgcccaccgcggatggcagcaaattggaggatgttgttgactctttgggggagaagctgaccgtcgagggagagctcaacaaggtctgctcgaatatcagcgtcgggcgcaactgggctggtgtgcactactttacagactatcgcgagtccatcatccttggggaaaagattgccctcggcctgctggaggagcagaagcttacgtatgctgaggagttctccatgacgattcctctctttgatggttccactgtcaccatttag

SEQ ID NO: 88 - DNA, 144 bp, synthetic construct
  source 1..144: mol_type = other DNA
atggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatg

SEQ ID NO: 89 - DNA, 96 bp, synthetic construct
  source 1..96: mol_type = other DNA
atggctctccgcaccctggcatcgaagaaggtcctatccttcccgttcggcggcgcgggccggccgttggcggcggcggcgtctgcgaggggggtg

SEQ ID NO: 90 - DNA, 30 bp, synthetic construct
  source 1..30: mol_type = other DNA
ccgctggtggtgcaactatacagtgtcttg

SEQ ID NO: 91 - DNA, 1993 bp, synthetic construct
  source 1..1993: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 92 - DNA, 677 bp, synthetic construct
  source 1..677: mol_type = other DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 93 - DNA, 24 bp, synthetic construct
  source 1..24: mol_type = other DNA
attgctgcagcggccgaattcaag

SEQ ID NO: 94 - DNA, 60 bp, synthetic construct
  source 1..60: mol_type = other DNA
ggcggaggtggttctggcggaggtggttctggcggaggtggttctggcggaggtggttct

SEQ ID NO: 95 - DNA, 87 bp, synthetic construct
  source 1..87: mol_type = other DNA
tatccgtacgacgtgccagactatgcgggctatccctatgacgtcccggactatgcaggatacccttacgatgttcctgattacgct

SEQ ID NO: 96 - DNA, 276 bp, synthetic construct
  source 1..276: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatga

SEQ ID NO: 97 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa